(19) United States
(12) Patent Application Publication
(10) Pub. No.: US 2026/0285850 A1
(43) Pub. Date: Sep. 24, 2026

(54) SUBSTITUTED OXAZOLIDINONES AND IMIDAZOLINONES AS HERBICIDES

(71) Applicant: FMC CORPORATION, Philadelphia, PA (US)

(72) Inventors: Saptarshi DE, Hyderabad (IN); Thomas Martin STEVENSON, Newark, DE (US); Stephen Frederick MCCANN, Newark, DE (US); Kashinath KOMIRISHETTY, Hyderabad (IN)

(21) Appl. No.: 19/167,427

(22) PCT Filed: Mar. 21, 2024
(86) PCT No.: PCT/US2024/020840

§ 371 Date: Sep. 22, 2025

Related U.S. Application Data
(60) Provisional application No. 63/454,096, filed on Mar. 23, 2023.

(51) Int. Cl. C07D 407/12; A01N 43/50; A01N 43/76; A01P 13/00; C07D 233/32; C07D 263/20; C07D 413/12

(52) U.S. Cl. CPC C07D 407/12; A01N 43/50; A01N 43/76; A01P 13/00; C07D 233/32; C07D 263/20; C07D 413/12

(57) ABSTRACT

Disclosed are compounds of Formula 1, including all stereoisomers, N-oxides, and salts thereof, agricultural compositions containing them and their use as herbicides

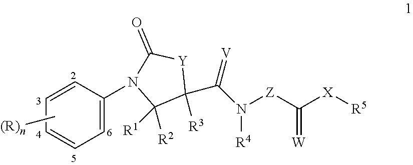

Z is selected from a group of a fully saturated, or a fully or partly unsaturated, five membered rings as shown below, optionally substituted by group R⁴

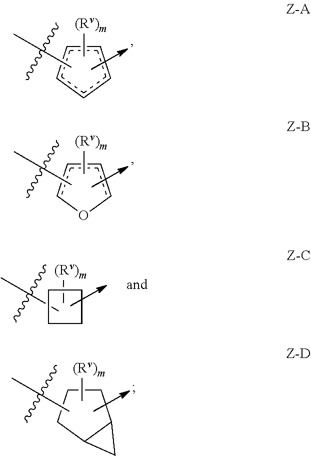

and R, R¹, R², R³, R⁴, R⁵, V, W, Y, Z and n are as defined in the disclosure.

### FIELD OF THE INVENTION

This invention relates to certain oxazolidinones herbicides, their N-oxides, salts and compositions, and methods of their use for controlling undesirable vegetation.

### BACKGROUND OF THE INVENTION

The control of undesired vegetation is extremely important in achieving high crop efficiency. Achievement of selective control of the growth of weeds especially in such useful crops as rice, soybean, sugar beet, maize, potato, wheat, barley, tomato and plantation crops, among others, is very desirable. Unchecked weed growth in such useful crops can cause significant reduction in productivity and thereby result in increased costs to the consumer. The control of undesired vegetation in noncrop areas is also important. Many products are commercially available for these purposes, but the need continues for new compounds that are more effective, less costly, less toxic, environmentally safer or have different sites of action.

### SUMMARY OF THE INVENTION

This invention is directed to compounds of Formula 1, all stereoisomers, N-oxides, and salts thereof, agricultural compositions containing them and their use as herbicides:

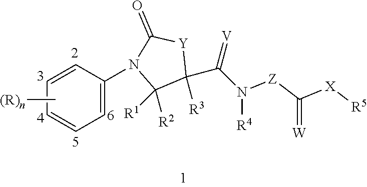

wherein
   Y is O or NH;
   Z is selected from a group of a fully saturated, or a fully or partly unsaturated, five membered rings as shown below, optionally substituted by group R^{v}

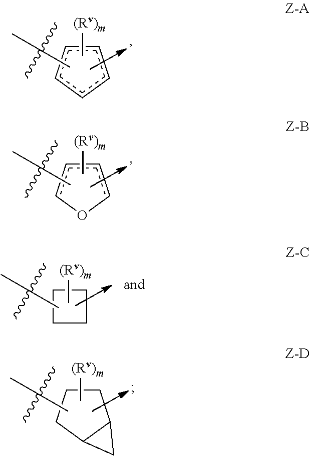

m is 0, 1 or 2;
R^{v} is halogen, cyano, CO₂R⁸, or (C₁-C₂)-alkyl or (C₁-C₂)-alkoxy, each of which is substituted by n radicals independently selected from the group consisting of halogens;
each R is independently H, halogen, cyano, nitro, hydroxy, C₁-C₆ alkyl, C₁-C₆ haloalkyl, C₂-C₆ alkenyl, C₂-C₆ haloalkenyl, C₂-C₆ alkynyl, C₂-C₆ haloalkynyl, C₃-C₇ cycloalkyl, C₃-C₇ halocycloalkyl, C₁-C₆ alkoxy, C₁-C₆ haloalkoxy, S(O)ₚR⁷ or CO₂R⁸;
n is 0, 1, 2, 3, 4 or 5;
p is 0, 1 or 2;
V and W are each independently O or S;
X is a direct bond, O, S or NR⁶;
R¹ and R² are each independently hydrogen, halogen, cyano, C₁-C₆ alkyl, C₁-C₆ haloalkyl, C₁-C₆ cyanoalkyl, C₁-C₆ alkoxy, C₁-C₆ haloalkoxy or C₁-C₆ cyanoalkoxy;
R³ is H, halogen, cyano, nitro, hydroxy, C₁-C₆ alkyl, C₂-C₅ alkenyl, C₂-C₅ alkynyl, C₂-C₅ alkenyloxy, C₂-C₅ alkynyloxy, C₃-C₇ cycloalkoxy, C₃-C₇ cycloalkoxyalkyl, C₃-C₆ cycloalkyl, C₄-C₇ cycloalkylalkyl, C₁-C₆ haloalkyl, C₂-C₅ haloalkenyl, C₂-C₅ haloalkynyl, C₂-C₅ alkoxyalkyl, C₂-C₅ haloalkoxyalkyl, C₁-C₅ alkoxy, C₁-C₆ haloalkoxy, C₁-C₅ alkylthio, C₁-C₄ alkylsulfinyl, C₁-C₄ alkylsulfonyl, C₁-C₄ alkylsulfonate, C₁-C₄ haloalkylthio, C₁-C₄ haloalkylsulfinyl, C₁-C₄ haloalkylsulfonyl or C₂-C₅ alkoxycarbonyl, optionally each of which is further substituted by at least one radical from the group consisting of halogen, cyano, C₁-C₄ alkoxy, C₁-C₅ alkylthio, C₁-C₄ alkylsulfinyl, C₁-C₄ alkylsulfonyl, and hydroxy;
R⁴ is independently H, C₁-C₆ alkyl, C₃-C₇ cycloalkyl, C₁-C₆ haloalkyl, C₁-C₃ alkoxy, C₁-C₃ haloalkoxy; or W¹G¹;
W¹ is a direct bond, C₁-C₄ alkanediyl or C₁-C₄ alkenediyl;
G¹ is S(O)ₚR⁷, SO₂NR¹⁰R¹¹, CO₂R⁸, CONR¹⁰R¹¹ or COR¹²;
R⁵ is H, C₁-C₁₂ alkyl, NH₂, N═CR⁸R¹², C₃-C₇ cycloalkyl, C₃-C₁₂ cycloalkylalkyl, C₂-C₈ alkenyl, C₅-C₆ cycloalkenyl, C₂-C₈ alkynyl, (CH₂)_{q}S(O)ₚR⁷, (CH₂)_{q}OR⁹, (CH₂)_{q}COR¹², each of which is optionally substituted by one or more radicals from the group consisting of halogen, cyano, C₁-C₆ alkyl, C₁-C₆ alkoxy, hydroxy and aryl;
q is 0, 1, 2, 3, 4 or 5;
R⁶ is hydrogen, cyano, OR⁹, S(O)ₚR⁷, SO₂NR¹⁰R¹¹, CO₂R⁸, CONR¹⁰R¹¹, COR¹², NR¹⁰R¹¹, NR¹⁰COR¹², NR¹⁰CONR¹⁰R¹¹, NR¹⁰CO₂R⁸, NR¹⁰SO₂R⁷, NR¹⁰SO₂NR¹⁰R¹¹, C(R⁷)═NOR⁹, optionally substituted aryl, optionally substituted heteroaryl or optionally substituted heterocyclyl; or
R⁶ is C₁-C₁₂ alkyl, C₃-C₈ cycloalkyl, C₄-C₁₂ cycloalkylalkyl, C₂-C₁₂ alkenyl, C₅-C₇ cycloalkenyl or C₂-C₁₂ alkynyl, each of which is optionally substituted by one or more radicals from the group consisting of halogen, cyano, nitro, OR⁹, S(O)ₚR⁷, SO₂NR¹⁰R¹¹, CO₂R⁸, CONR¹⁰R¹¹, COR¹², NR¹⁰R¹¹, NR¹⁰COR¹², NR¹⁰CONR¹⁰R¹¹, NR¹⁰CO₂R⁸, NR¹⁰SO₂R⁷, NR¹⁰SO₂NR¹⁰R¹¹, C(R⁷)═NOR⁹, optionally substituted aryl, optionally substituted heteroaryl or optionally substituted heterocyclyl; or
R⁵ and R⁶ can be taken together with the nitrogen atom to which they are attached to form a 3- to 7-membered ring, containing carbon atoms and optionally 1 to 3 oxygen, sulfur or nitrogen atoms as ring members, wherein up to 2 carbon atom ring members are independently selected from C(═O) and C(═S), and the sulfur atom ring member is selected from S, S(O) or S(O)₂, said ring being optionally substituted with at least one substituent independently selected from the group consisting of halogen, cyano, nitro, C₁-C₆ alkyl, C₁-C₆ haloalkyl, OR⁹, S(O)ₚR⁷, SO₂NR¹⁰R¹¹, CO₂R⁸, CONR¹⁰R¹¹, COR¹², NR¹⁰R¹¹, NR¹⁰COR¹², NR¹⁰CONR¹⁰R¹¹, NR¹⁰CO₂R⁸, NR¹⁰SO₂R⁷, NR¹⁰SO₂NR¹⁰R¹¹, and C(R⁷)—NOR⁹;
R⁷, R⁸, R⁹, R¹⁰, R¹¹ and R¹² are each independently H, C₁-C₆ alkyl, C₁-C₆ alkoxy, C₁-C₆ haloalkyl, C₃-C₇ cycloalkyl or aryl, each of which is optionally substituted by one or more radicals from the group consisting of F, Cl, C₁-C₂ alkoxy or aryl.

More particularly, this invention pertains to a compound of Formula 1 (including all stereoisomers), an N-oxide or a salt thereof. This invention also relates to an herbicidal composition comprising a compound of the invention (i.e. in a herbicidally effective amount) and at least one component selected from the group consisting of surfactants, solid diluents and liquid diluents. This invention further relates to a method for controlling the growth of undesired vegetation comprising contacting the vegetation or its environment with a herbicidally effective amount of a compound of the invention (e.g., as a composition described herein).

This invention also includes a herbicidal mixture comprising (a) a compound selected from Formula 1, N-oxides, and salts thereof, and (b) at least one additional active ingredient selected from (b1) through (b16), and salts of compounds of (b1) through (b16), as described below.

### DETAILS OF THE INVENTION

As used herein, the terms “comprises,” “comprising,” “includes,” “including,” “has,” “having,” “contains”, “containing,” “characterized by” or any other variation thereof, are intended to cover a non-exclusive inclusion, subject to any limitation explicitly indicated. For example, a composition, mixture, process or method that comprises a list of elements is not necessarily limited to only those elements but may include other elements not expressly listed or inherent to such composition, mixture, process or method.

The transitional phrase “consisting of” excludes any element, step, or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Where applicants have defined an invention or a portion thereof with an open-ended term such as “comprising,” it should be readily understood that (unless otherwise stated) the description should be interpreted to also describe such an invention using the terms “consisting essentially of” or “consisting of.”

Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

As referred to herein, the term “seedling”, used either alone or in a combination of words means a young plant developing from the embryo of a seed.

As referred to herein, the term “broadleaf” used either alone or in words such as “broadleaf weed” means dicot or dicotyledon, a term used to describe a group of angiosperms characterized by embryos having two cotyledons.

As used herein, the term “alkylating” refers reaction in which nucleophile displaces a leaving group such as halide or sulfonate from a carbon-containing radical. Unless otherwise indicated, the term “alkylating” does not limit the carbon-containing radical to alkyl.

In the above recitations, the term “alkyl”, used either alone or in compound words such as “alkylthio” or “haloalkyl” includes straight-chain or branched alkyl, such as, methyl, ethyl, n-propyl, i-propyl, or the different butyl, pentyl or hexyl isomers. “Alkenyl” includes straight-chain or branched alkenes such as ethenyl, 1-propenyl, 2-propenyl, and the different butenyl, pentenyl and hexenyl isomers. “Alkenyl” also includes polyenes such as 1,2-propadienyl and 2,4-hexadienyl. “Alkynyl” includes straight-chain or branched alkynes such as ethynyl, 1-propynyl, 2-propynyl and the different butynyl, pentynyl and hexynyl isomers. “Alkynyl” can also include moieties comprised of multiple triple bonds such as 2,5-hexadiynyl.

“Alkoxy” includes, for example, methoxy, ethoxy, n-propyloxy, isopropyloxy and the different butoxy, pentoxy and hexyloxy isomers. “Alkoxyalkyl” denotes alkoxy substitution on alkyl. Examples of “alkoxyalkyl” include CH₃OCH₂, CH₃OCH₂CH₂, CH₃CH₂OCH₂, CH₃CH₂CH₂CH₂OCH₂ and CH₃CH₂OCH₂CH₂. “Hydroxyalkyl” denotes a hydroxy substitution on alkyl. “Hydroxycycloalkyl” denotes a hydroxy substitution on cycloalkyl. “Hydroxyhaloalkyl” denotes a hydroxy substitution on haloalkyl. “Alkoxycycloalkyl” denotes an alkoxy substitution on cycloalkyl. “Alkoxyhaloalkyl” denotes an alkoxy substitution on haloalkyl.

“Alkoxyalkoxy” denotes alkoxy substitution on alkoxy. “Alkylthio” includes branched or straight-chain alkylthio moieties such as methylthio, ethylthio, and the different propylthio, butylthio, pentylthio and hexylthio isomers. “Alkylthioalkyl” denotes alkylthio substitution on alkyl. Examples of “alkylthioalkyl” include CH₃SCH₂, CH₃SCH₂CH₂, CH₃CH₂SCH₂, CH₃CH₂CH₂CH₂SCH₂ and CH₃CH₂SCH₂CH₂. “Alkylsulfinyl” includes both enantiomers of an alkylsulfinyl group. Examples of “alkylsulfinyl” include CH₃S(O)—, CH₃CH₂S(O)—, CH₃CH₂CH₂S(O)—, (CH₃)₂CHS(O)— and the different butylsulfinyl, pentylsulfinyl and hexylsulfinyl isomers. Examples of “alkylsulfonyl” include CH₃S(O)₂—, CH₃CH₂S(O)₂—, CH₃CH₂CH₂S(O)₂—, (CH₃)₂CHS(O)₂—, and the different butylsulfonyl, pentylsulfonyl and hexylsulfonyl isomers. Examples of “alkylsulfonate” include CH₃S(O)₂O—, CH₃CH₂S(O)₂O—, CH₃CH₂CH₂S(O)₂O—, (CH₃)₂CHS(O)₂O—, and the different butylsulfonate, pentylsulfonate and hexylsulfonate isomers. “Cyanoalkyl” denotes an alkyl group substituted with one cyano group. Examples of “cyanoalkyl” include NCCH₂ and NCCH₂CH₂ (alternatively identified as CH₂CH₂CN). “Nitroalkyl” denotes an alkyl group substituted with one nitro group. Examples of “nitroalkyl” include NO₂NCH₂ and NO₂NCH₂CH₂ (alternatively identified as CH₂CH₂NO₂). “Cyano” means NC—, and “formyl” means HC(═O)—. “Alkylamino” includes an NH radical substituted with straight-chain or branched alkyl. Examples of “alkylamino” include CH₃CH₂NH, CH₃CH₂CH₂NH, and (CH₃)₂CHCH₂NH. Examples of “dialkylamino” include (CH₃)₂N, (CH₃CH₂CH₂)₂N and CH₃CH₂ (CH₃)N. “Alkylsily” includes a silyl radical substituted with straight-chain or branched alkyl. “trialkylsily” includes a silyl radical substituted with three straight-chain or branched alkyl. Examples of “trialkylsily” include (CH₃)₃Si—, and (CH₃CH₂)₃Si—. “trialkylsilyalkynyl” denotes trialkylsily substitution on alkynyl. Examples of “trialkylsilyalkynyl” include (CH₃)₃SiC≡C—, and (CH₃CH₂)₃SiC≡C—.

“Cycloalkyl” includes, for example, cyclopropyl, cyclobutyl, cyclopentyl and cyclohexyl. The term “cycloalkylalkyl” denotes cycloalkyl substitution on an alkyl moiety. Examples of “cycloalkylalkyl” include cyclopropylmethyl, cyclopentylethyl, and other cycloalkyl moieties bonded to straight-chain or branched alkyl groups. The term “alkylcycloalkyl” denotes an alkyl group bonded to a cycloalkyl moiety. The term “cycloalkoxy” denotes cycloalkyl group bonded through oxygen. Examples of “cycloalkoxy” include cyclopropoxy, cyclobutoxy, and cyclopentoxy. The term “cycloalkoxyalkyl” denotes cycloalkoxy substitution on an alkyl moiety. Examples of “cycloalkoxyalkyl” include cyclopropoxymethyl, cyclobutoxyethyl, and cyclopentoxymethyl, and other cycloalkoxy moieties bonded to straight-chain or branched alkyl groups. The term “oxacycloalkyl” denotes a cycloalkyl with one carbon ring member replaced with an oxygen atom. Examples of “oxacycloalkyl” include oxacyclopropy, oxacyclobutyl and oxacyclopentyl.

The term “halogen”, either alone or in compound words such as “haloalkyl”, or when used in descriptions such as “alkyl substituted with halogen” includes fluorine, chlorine, bromine or iodine. Further, when used in compound words such as “haloalkyl”, or when used in descriptions such as “alkyl substituted with halogen” said alkyl may be partially or fully substituted with halogen atoms which may be the same or different. Examples of “haloalkyl” or “alkyl substituted with halogen” include F₃C, ClCH₂, CF₃CH₂ and CF₃CCl₂. The terms “haloalkoxy”, “haloalkoxyalkyl”, “haloalkylthio”, “haloalkenyl”, “haloalkynyl”, “halocycloalkyl”, “haloalkylcycloalkyl”, “haloalkylsulfinyl”, “haloalkylsulfonyl” and the like, are as defined analogously to the term “haloalkyl”. Examples of “haloalkoxy” include CF₃O—, CCl₃CH₂O—, HCF₂CH₂CH₂O— and CF₃CH₂O—. Examples of “haloalkoxyalkyl” include CF₃OCH₂—, CCl₃CH₂OCH₂—, HCF₂CH₂CH₂OCH₂— and CF₃CH₂OCH₂—. Examples of “haloalkylthio” include CCl₃S—, CF₃S—, CCl₃CH₂S— and ClCH₂CH₂CH₂S—. Examples of “haloalkenyl” include (Cl)₂C═CH—(Cl)₂C═CHCH₂— and CF₃CH₂CH═CHCH₂—. Examples of “haloalkynyl” include HC≡CCHCl—, CF₃C≡C—, CCl₃C≡C— and FCH₂C≡CCH₂—. Examples of “halocycloalkyl” include 1-chlorocyclopropyl, 2-chlorocyclopropyl, 2-fluorocyclopropyl, 1-chlorocyclobutyl, 1-Fluorocyclobutyl and 2-Fluorocyclobutyl. Examples of “haloalkylcycloalkyl” include 1-(chloromethyl)cyclopropyl, 2-(chloromethyl)cyclopropyl, 2-(fluoromethyl)cyclopropyl, 1-(chloromethyl)cyclobutyl, 2-(fluoroethyl)cyclobutyl and 2-(fluoromethyl)cyclobutyl.

“Alkylcarbonyl” denotes a straight-chain or branched alkyl moiety bonded to a C(═O) moiety. Examples of “alkylcarbonyl” include CH₃C(═O)—, CH₃CH₂C(═O)—, CH₃CH₂CH₂C(═O)—, (CH₃)₂CHC(═O)— and the different butoxy- or pentoxycarbonyl isomers. “Alkoxycarbonyl” denotes a straight-chain or branched alkoxy moieties bonded to a C(═O) moiety. Examples of “alkoxycarbonyl” include CH₃OC(═O)—, CH₃CH₂OC(═O)—, CH₃CH₂CH₂OC(═O)—, (CH₃)₂CHOC(═O)— and the different butoxy- or pentoxycarbonyl isomers. C(═O) or C(O) designates carbonyl. The term “alkoxycarbonylalkyl” denotes a straight-chain or branched alkoxycarbonyl moiety bonded through an alkyl moiety. The term “alkylcarbonylalkyl” denotes a straight or branched alkylcarbonyl moiety bonded through an alkyl moiety. The term “alkylcarbonyloxy” denotes an alkylcarbony moiety bonded through oxygen. Examples of alkylcarbonyloxy include CH₃C(═O)O—, CH₃CH₂C(═O)O—, CH₃CH₂CH₂C(═O)O— and (CH₃)₂CHC(═O)—. The term “alkenyloxy” denotes an alkenyl moiety bonded through oxygen. Examples of “alkenyloxy” include CH₂CHCH₂O—, 1-propenyloxy or CH₃CHCHO—, 2-butenyloxy or CH₃CHCHCH₂O—, and the different butenyloxy, pentenyloxy and hexenyloxy isomers. The term “alkynyloxy” denotes an alkynyl moiety bonded through oxygen. Examples of “alkenyloxy” may also contain more than one double bond. Examples of “alkynyloxy” include CHCCH₂O—, 1-propynyloxy or CH₃CCO—, 2-butynyloxy or CH₃CCCH₂O—, and the different butynyloxy, pentynyloxy and hexynyloxy isomers. Examples of “alkynyloxy” may also contain more than one triple bond. The term alkanediyl or alkenediyl refers to a linear or branched alkane or alkene linking chain respectively. Examples of alkanediyl include —CH₂—, —CH₂CH(CH₃)— or —CH₂CH₂CH₂—. Examples of alkenediyl include —CH═CH—, —CH₂C═CH— or —CH═C(CH₃)—. The term “adjacent” in the context of locating a substituent means “next to” or “immediately next to”.

The total number of carbon atoms in a substituent group is indicated by the “Cᵢ-Cⱼ” prefix where i and j are numbers from 1 to 8. For example, C₁-C₄ alkylsulfonyl designates methylsulfonyl through butylsulfonyl; C₃-C₈ alkylcarbonylalkyl can be, for example, CH₃COCH₂—, CH₃COCH₂CH₂— or CH₃CH₂CH₂COCH₂CH₂CH₂CH₂—; C₄-C₇ alkylcycloalkyl can be, for example, methylcyclopropyl, methylcyclobutyl, ethylcyclopropyl, or propylcyclobutyl; C₂ alkoxyalkyl designates CH₃OCH₂—; C₃ alkoxyalkyl designates, for example, CH₃CH(OCH₃)—, CH₃OCH₂CH₂— or CH₃CH₂OCH₂—; and C₄ alkoxyalkyl designates the various isomers of an alkyl group substituted with an alkoxy group containing total of four carbon atoms, a examples including CH₃CH₂CH₂OCH₂— and CH₃CH₂OCH₂CH₂—.

For Z groups, the arrow with a wavy line represents a bond to the group C═W in Formula 1 and the bond connecting Z to NR⁴ is represented by a solid line with a wavy line. Some non-limiting examples can be found in Embodiment 2. The attachment of substituent(s) R^{v}, the bond connecting Z to NR⁴ and the arrow are illustrated as floating. Each R^{v}, NR⁴ and C═W group can be attached to the Z ring through any of the available ring member carbons by replacement of a hydrogen atom.

When a group contains a substituent which can be hydrogen, then when this substituent is taken as hydrogen, it is recognized that this is equivalent to said group being unsubstituted. When one or more positions on a group are said to be “not substituted” or “unsubstituted”, then hydrogen atoms are attached to take up any free valency. Unless otherwise indicated as being optionally substituted, the term “phenyl” means unsubstituted phenyl. Unless otherwise indicated as being optionally substituted, the term “benzyl” means unsubstituted benzyl.

When a compound is substituted with a substituent bearing a subscript that indicates the number of said substituents can exceed 1, said substituents (when they exceed 1) are independently selected from the group of defined substituents, e.g., (R)ₙ, wherein n is 0, 1, 2, 3 or 4. When n is 0, then hydrogen may be at the position even if not recited in the substituent definition. When a functional group or a compound is shown to be optionally substituted with a substituent, the said functional group or compound may be unsubstituted or substituted. When one or more positions on a group are said to be “not substituted” or “unsubstituted”, then hydrogen atoms are attached to take up any free valency. When a variable is H and the H is substituted by a group, it means that the said H is replaced with this group. For example, when “R³ is H and optionally further substituted by halogen”, it means that R³ is H or halogen.

The term “ring system” denotes two or more fused rings. The term “bicyclic ring system” denotes a ring system consisting of two fused rings.

Compounds of this invention can exist as one or more stereoisomers. The various stereoisomers include enantiomers, diastereomers, atropisomers and geometric isomers. Stereoisomers are isomers of identical constitution but differing in the arrangement of their atoms in space and include enantiomers, diastereomers, cis-trans isomers (also known as geometric isomers) and atropisomers. Atropisomers result from restricted rotation about single bonds where the rotational barrier is high enough to permit isolation of the isomeric species. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers. The compounds of the invention may be present as a mixture of stereoisomers, individual stereoisomers or as an optically active form.

Compounds of Formula 1 typically exist in more than one form, and Formula 1 thus include all crystalline and non-crystalline forms of the compounds they represent. Non-crystalline forms include embodiments which are solids such as waxes and gums as well as embodiments which are liquids such as solutions and melts. Crystalline forms include embodiments which represent essentially a single crystal type and embodiments which represent a mixture of polymorphs (i.e. different crystalline types). The term “polymorph” refers to a particular crystalline form of a chemical compound that can crystallize in different crystalline forms, these forms having different arrangements and/or conformations of the molecules in the crystal lattice. Although polymorphs can have the same chemical composition, they can also differ in composition due to the presence or absence of co-crystallized water or other molecules, which can be weakly or strongly bound in the lattice. Polymorphs can differ in such chemical, physical and biological properties as crystal shape, density, hardness, color, chemical stability, melting point, hygroscopicity, suspensibility, dissolution rate and biological availability. One skilled in the art will appreciate that a polymorph of a compound of Formula 1 can exhibit beneficial effects (e.g., suitability for preparation of useful formulations, improved biological performance) relative to another polymorph or a mixture of polymorphs of the same compound of Formula 1. Preparation and isolation of a particular polymorph of a compound of Formula 1 can be achieved by methods known to those skilled in the art including, for example, crystallization using selected solvents and temperatures. For a comprehensive discussion of polymorphism see R. Hilfiker, Ed., Polymorphism in the Pharmaceutical Industry, Wiley-VCH, Weinheim, 2006.

One skilled in the art will appreciate that not all nitrogen-containing heterocycles can form N-oxides since the nitrogen requires an available lone pair for oxidation to the oxide; one skilled in the art will recognize those nitrogen-containing heterocycles which can form N-oxides. One skilled in the art will also recognize that tertiary amines can form N-oxides. Synthetic methods for the preparation of N-oxides of heterocycles and tertiary amines are very well known by one skilled in the art including the oxidation of heterocycles and tertiary amines with peroxy acids such as peracetic and m-chloroperbenzoic acid (MCPBA), hydrogen peroxide, alkyl hydroperoxides such as t-butyl hydroperoxide, sodium perborate, and dioxiranes such as dimethyldioxirane. These methods for the preparation of N-oxides have been extensively described and reviewed in the literature, see for example: T. L. Gilchrist in Comprehensive Organic Synthesis, vol. 7, pp 748-750, S. V. Ley, Ed., Pergamon Press; M. Tisler and B. Stanovnik in Comprehensive Heterocyclic Chemistry, vol. 3, pp 18-20, A. J. Boulton and A. McKillop, Eds., Pergamon Press; M. R. Grimmett and B. R. T. Keene in Advances in Heterocyclic Chemistry, vol. 43, pp 149-161, A. R. Katritzky, Ed., Academic Press; M. Tisler and B. Stanovnik in Advances in Heterocyclic Chemistry, vol. 9, pp 285-291, A. R. Katritzky and A. J. Boulton, Eds., Academic Press; and G. W. H. Cheeseman and E. S. G. Werstiuk in Advances in Heterocyclic Chemistry, vol. 22, pp 390-392, A. R. Katritzky and A. J. Boulton, Eds., Academic Press.

One skilled in the art recognizes that because in the environment and under physiological conditions salts of chemical compounds are in equilibrium with their corresponding nonsalt forms, salts share the biological utility of the nonsalt forms. Thus, a wide variety of salts of a compound of Formula 1 are useful for control of undesired vegetation (i.e. are agriculturally suitable). The salts of a compound of Formula 1 include acid-addition salts with inorganic or organic acids such as hydrobromic, hydrochloric, nitric, phosphoric, sulfuric, acetic, butyric, fumaric, lactic, maleic, malonic, oxalic, propionic, salicylic, tartaric, 4-toluenesulfonic or valeric acids. When a compound of Formula 1 contains an acidic moiety, salts also include those formed with organic or inorganic bases such as pyridine, triethylamine or ammonia, or amides, hydrides, hydroxides or carbonates of sodium, potassium, lithium, calcium, magnesium or barium. Accordingly, the present invention comprises compounds selected from Formula 1, N-oxides and agriculturally suitable salts thereof.

A wide variety of synthetic methods are known in the art to enable preparation of aromatic and nonaromatic heterocyclic rings and ring systems; for extensive reviews see the eight volume set of Comprehensive Heterocyclic Chemistry, A. R. Katritzky and C. W. Rees editors-in-chief, Pergamon Press, Oxford, 1984 and the twelve volume set of Comprehensive Heterocyclic Chemistry II, A. R. Katritzky, C. W. Rees and E. F. V. Scriven editors-in-chief, Pergamon Press, Oxford, 1996.

Embodiments of the present invention as described in the Summary of the Invention include those described below. In the following Embodiments, Formula 1 includes stereoisomers, N-oxides and salts thereof, and reference to “a compound of Formula 1” includes the definitions of substituents specified in the Summary of the Invention unless further defined in the Embodiments.
Embodiment 1. A compound of Formula 1, stereoisomers, N-oxides, and salts thereof, agricultural compositions containing them and their use as herbicides as described in the Summary of the Disclosure.
Embodiment 2. A compound of Formula 1 or Embodiment 1 wherein Z-A is selected from Z-1 through Z-29, and Z-B is selected from Z-30 through Z-62, Z-C is selected from Z-63 through 64, and Z-D is selected from Z-65 through Z-74 as shown below;

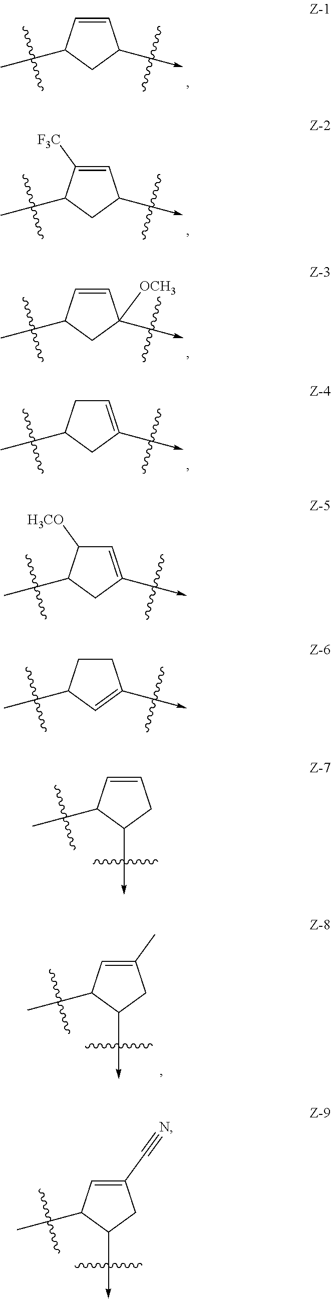

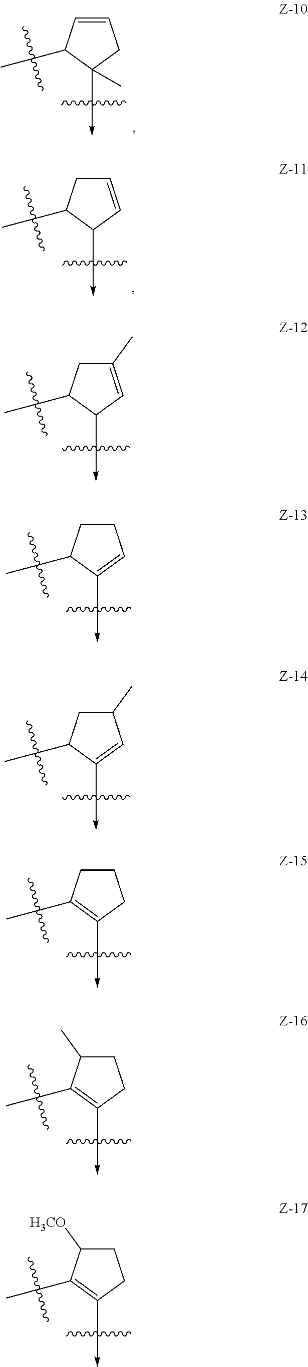

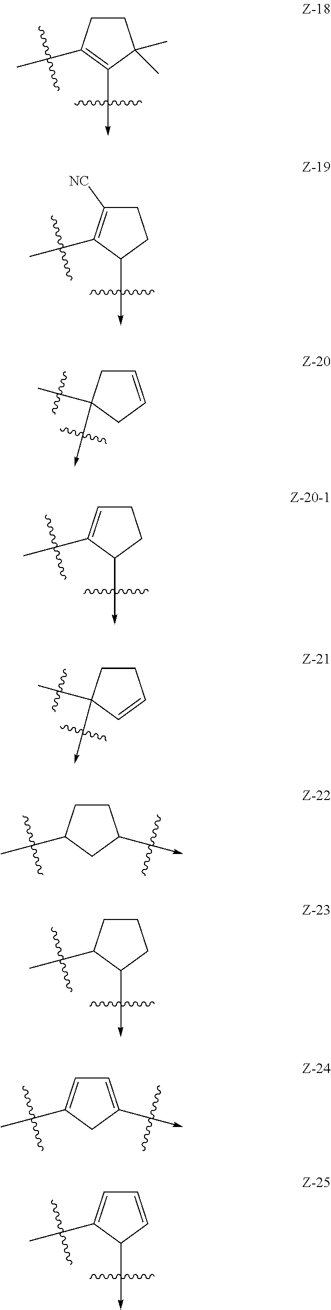

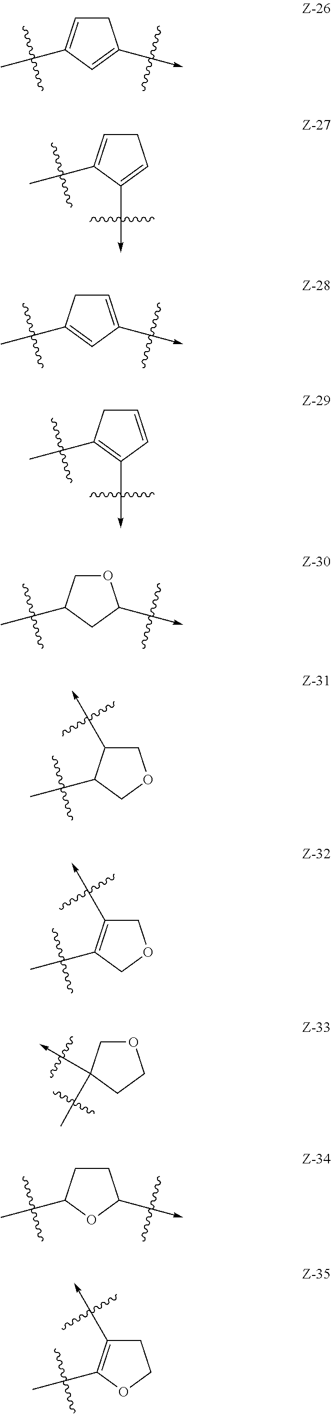

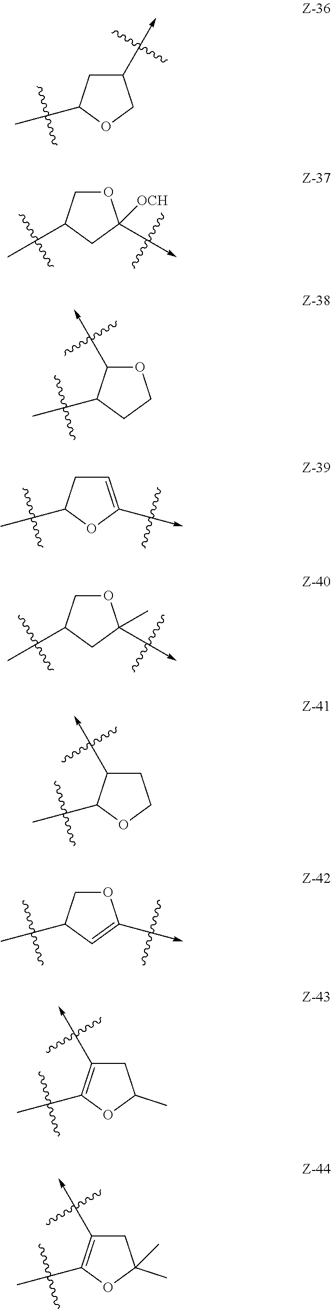

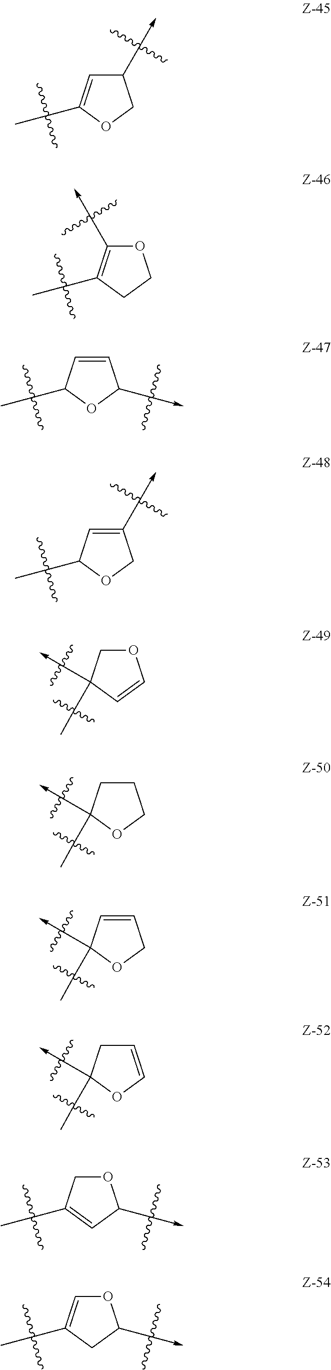

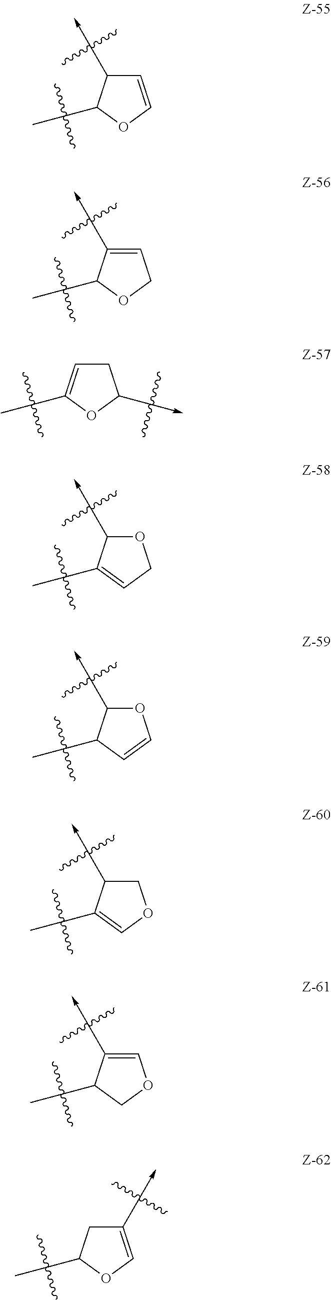

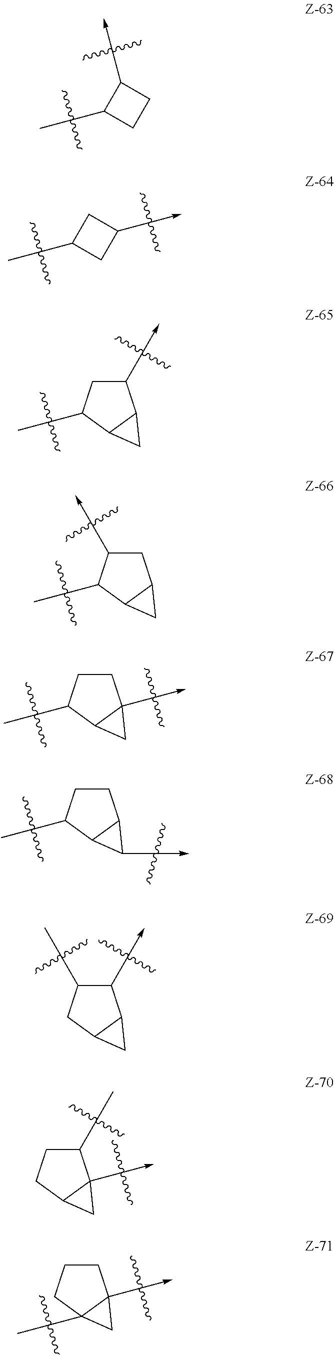

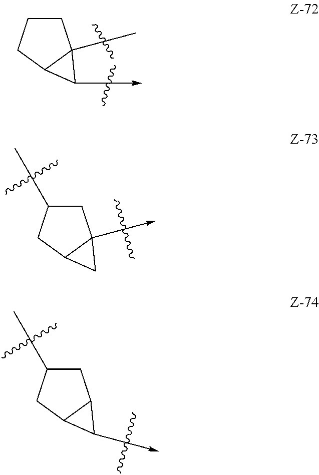 [SMILES: *C1C2CCCC12* ; *C1CC2CC2(*)C1 ; *C1CC2C(*)C2C1]

Embodiment 2a. A compound of Embodiment 2 wherein Z is selected from the group Z-A.
Embodiment 2aa. A compound of Embodiment 2a wherein Z is selected from the group Z-1 to Z-12.
Embodiment 2b. A compound of Embodiment 2a wherein Z is Z-1, Z-4, Z-6, Z-12, Z-22 or Z-24.
Embodiment 2c. A compound of Embodiment 2 wherein Z is selected from the group Z-B.
Embodiment 2d. A compound of Embodiment 2b wherein Z is Z-30, Z-34, Z-36, Z-42, Z-47, Z-48, Z-53, Z-54, Z-55, Z-57 or Z-62.
Embodiment 2e. A compound of Embodiment 2 wherein Z is Z-1, Z-4, Z-22 or Z-30.
Embodiment 2f. A compound of Embodiment 2 wherein Z is Z-63 or Z-64.
Embodiment 2g. A compound of Embodiment 2 wherein Z is Z-65 through Z-74.
   m
Embodiment 3. A compound of Embodiment 1 wherein m is 0 or 1.
Embodiment 3a. A compound of Embodiment 3 wherein m is 0.
Embodiment 3b. A compound of Embodiment 3 wherein m is 1.

### R

Embodiment 4. A compound of Embodiment 2 wherein R is independently H, halogen, cyano, C₁-C₆ alkyl, C₁-C₆ haloalkyl, C₁-C₆ alkoxy or C₁-C₆ haloalkoxy.
Embodiment 4a. A compound of Embodiment 4 wherein R is independently H, F, Cl, Br, cyano, C₁-C₂ alkyl, C₁-C₂ haloalkyl, C₁-C₂ alkoxy or C₁-C₂ haloalkoxy.
Embodiment 4b. A compound of Embodiment 4a wherein R is independently H, F, Cl, Me, CF₃, OMe or OCF₃.
Embodiment 4c. A compound of Embodiment 4 wherein R is independently halogen.
Embodiment 4d. A compound of Embodiment 4c wherein R is independently F or Cl.
Embodiment 4e. A compound of Embodiment 4 wherein R is at 3- and 5-position.
   n
Embodiment 5a. A compound of Embodiment 2 wherein n is 1, 2, 3 or 4.
Embodiment 5b. A compound of Embodiment 5a wherein n is 1, 2 or 3.
Embodiment 5c. A compound of Embodiment 5b wherein n is 1 or 2.
Embodiment 5d. A compound of Embodiment 5c wherein n is 2.
   p
Embodiment 6a. A compound of Embodiment 2 wherein n is 0.
Embodiment 6b. A compound of Embodiment 2 wherein n is 1.
Embodiment 6c. A compound of Embodiment 2 wherein n is 2.

### V and W

Embodiment 7. A compound of Embodiment 2 wherein V and W are each independently O or S.
Embodiment 7a. A compound of Embodiment 7 wherein V and W are both O.

### X

Embodiment 8a. A compound of Embodiment 2 wherein X is direct bond, O or S;
Embodiment 8aa. A compound of Embodiment 8a wherein X is O or S;
Embodiment 8b. A compound of Embodiment 8a wherein X is direct bond.
Embodiment 8c. A compound of Embodiment 8a wherein X is O.
Embodiment 8d. A compound of Embodiment 8a wherein X is S.
Embodiment 8e. A compound of Embodiment 2 wherein X is NR⁶.
   R¹ and R²
Embodiment 9. A compound of Embodiment 2 wherein R¹ and R² are each independently hydrogen, halogen, cyano, C₁-C₆ alkyl, C₁-C₆ haloalkyl, C₁-C₆ cyanoalkyl, C₁-C₆ alkoxy, C₁-C₆ haloalkoxy or C₁-C₆ cyanoalkoxy.
Embodiment 9a. A compound of Embodiment 9 wherein R¹ and R² are each independently hydrogen, halogen, cyano, C₁-C₃ alkyl, C₁-C₃ haloalkyl, C₁-C₃ cyanoalkyl, C₁-C₃ alkoxy, C₁-C₃ haloalkoxy or C₁-C₃ cyanoalkoxy.
Embodiment 9b. A compound of Embodiment 9a wherein R¹ and R² are each independently hydrogen, halogen, cyano, C₁-C₂ alkyl, C₁-C₂ haloalkyl, C₁-C₂ cyanoalkyl, C₁-C₂ alkoxy, C₁-C₂ haloalkoxy or C₁-C₂ cyanoalkoxy.
Embodiment 9c. A compound of Embodiment 9b wherein R¹ and R² are each independently hydrogen, halogen, cyano, Me, CF₃, CH₂CN, OMe, OCF₃ or OCH₂CN.
Embodiment 9d. A compound of Embodiment 9 wherein R¹ and R² are each independently hydrogen, halogen or cyano.
Embodiment 9e. A compound of Embodiment 9d wherein R¹ and R² are each independently hydrogen, F, Cl, Br or cyano.
Embodiment 9f. A compound of Embodiment 9c wherein R¹ and R² are each independently hydrogen or Me.
Embodiment 9g. A compound of Embodiment 9f wherein R¹ and R² are both hydrogens.

### R³

Embodiment 10. A compound of Embodiment 2 wherein R³ is H, halogen, cyano, nitro, hydroxy, C₁-C₆ alkyl, C₂-C₅ alkenyl, C₂-C₅ alkynyl, C₂-C₅ alkenyloxy, C₂-C₅ alkynyloxy, C₃-C₇ cycloalkoxy, C₃-C₇ cycloalkoxyalkyl, C₃-C₆ cycloalkyl, C₄-C₇ cycloalkylalkyl, C₁-C₆ haloalkyl, C₂-C₅ haloalkenyl, C₂-C₅ haloalkynyl, C₂-C₅ alkoxyalkyl, C₂-C₅ haloalkoxyalkyl, C₁-C₅ alkoxy, C₁-C₆ haloalkoxy, C₁-C₅ alkylthio, C₁-C₄ alkylsulfinyl, C₁-C₄ alkylsulfonyl, C₁-C₄ alkylsulfonate, C₁-C₄ haloalkylthio, C₁-C₄ haloalkylsulfinyl, C₁-C₄ haloalkylsulfonyl or C₂-C₅ alkoxycarbonyl, optionally each of which is further substituted by at least one radical from the group consisting of halogen, cyano, C₁-C₄ alkoxy, C₁-C₅ alkylthio, C₁-C₄ alkylsulfinyl, C₁-C₄ alkylsulfonyl, and hydroxy.
Embodiment 10a. A compound of Embodiment 10 wherein R³ is H, halogen, cyano, nitro, hydroxy, C₁-C₆ alkyl, C₂-C₅ alkenyl, C₂-C₅ alkynyl, C₂-C₅ alkenyloxy, C₂-C₅ alkynyloxy, C₃-C₇ cycloalkoxy, C₃-C₇ cycloalkoxyalkyl, C₃-C₆ cycloalkyl, C₄-C₇ cycloalkylalkyl, C₁-C₆ haloalkyl, C₂-C₅ haloalkenyl, C₂-C₅ haloalkynyl, C₂-C₅ alkoxyalkyl, C₂-C₅ haloalkoxyalkyl, C₁-C₅ alkoxy, C₁-C₆ haloalkoxy, C₁-C₅ alkylthio, C₁-C₄ alkylsulfinyl or C₁-C₄ alkylsulfonyl.
Embodiment 10b. A compound of Embodiment 10a wherein R³ is H, halogen, cyano, hydroxy, C₁-C₃ alkyl, C₂-C₃ alkenyl, C₂-C₃ alkynyl, C₃-C₅ cycloalkoxy, C₃-C₅ cycloalkoxyalkyl, C₃-C₅ cycloalkyl, C₄-C₇ cycloalkylalkyl, C₁-C₃ haloalkyl, C₂-C₃ alkoxyalkyl, C₂-C₃ haloalkoxyalkyl, C₁-C₃ alkoxy, C₁-C₃ haloalkoxy, C₁-C₃ alkylthio, C₁-C₃ alkylsulfinyl or C₁-C₃ alkylsulfonyl.
Embodiment 10c. A compound of Embodiment 10b wherein R³ is H, C₁-C₃ alkyl, C₂-C₃ alkenyl, C₂-C₃ alkynyl, C₃-C₅ cycloalkyl, C₄-C₇ cycloalkylalkyl, C₁-C₃ haloalkyl, C₂-C₃ alkoxyalkyl, C₂-C₃ haloalkoxyalkyl, C₁-C₃ alkoxy, C₁-C₃ haloalkoxy.
Embodiment 10d. A compound of Embodiment 10c wherein R³ is H, Me, Et, CH═CH₂, C≡CH, cyclopropyl, cyclopropylmethyl, CF₃, OMe or OCF₃.
Embodiment 10e. A compound of Embodiment 10d wherein R³ is H, Me, Et, CH═CH₂, C≡CH, cyclopropyl, CF₃, OMe or OCF₃.
Embodiment 10f. A compound of Embodiment 10e wherein R³ is H, Me, Et, CH═CH₂, C≡CH, cyclopropyl, CF₃, OMe or OCF 3.
Embodiment 10g. A compound of Embodiment 10f wherein R³ is Me.

### R⁴

Embodiment 11. A compound of Embodiment 2 wherein R⁴ is H, C₁-C₆ alkyl, C₃-C₇ cycloalkyl, C₁-C₆ haloalkyl, C₁-C₃ alkoxy, C₁-C₃ haloalkoxy.
Embodiment 11a. A compound of Embodiment 11 wherein R⁴ is H.

### R⁵

Embodiment 12. A compound of Embodiment 2 wherein R⁵ is H, C₁-C₁₂ alkyl, NH₂, N═CR⁸R¹², C₃-C₇ cycloalkyl, C₃-C₁₂ cycloalkylalkyl, C₂-C₈ alkenyl, C₅-C₆ cycloalkenyl, C₂-C₈ alkynyl, (CH₂)_{q}S(O)ₚR⁷, (CH₂)_{q}OR⁹, (CH₂)_{q}COR¹², each of which is optionally substituted by one or more radicals from the group consisting of halogen, cyano, C₁-C₆ alkyl, C₁-C₆ alkoxy, hydroxy and aryl.
Embodiment 12a. A compound of Embodiment 12 wherein R⁵ is H, C₁-C₇ alkyl, C₃-C₇ cycloalkyl, C₃-C₇ cycloalkylalkyl, C₂-C₅ alkenyl, C₅-C₆ cycloalkenyl, C₂-C₅ alkynyl, (CH₂)_{q}S(O)ₚR⁷, (CH₂)_{q}OR⁹, (CH₂)_{q}COR¹², each of which is optionally substituted by one or more radicals from the group consisting of halogen, cyano, C₁-C₃ alkyl, C₁-C₃ alkoxy, hydroxy and aryl.
Embodiment 12b. A compound of Embodiment 12 wherein R⁵ is H, C₁-C₅ alkyl, C₃-C₅ cycloalkyl, C₃-C₇ cycloalkylalkyl, (CH₂)_{q}S(O)ₚR⁷, (CH₂)_{q}OR⁹, (CH₂)_{q}COR¹², each of which is optionally substituted by one or more radicals from the group consisting of F, Cl, cyano, C₁-C₃ alkyl, C₁-C₃ alkoxy, hydroxy and aryl.
Embodiment 12c. A compound of Embodiment 12b wherein R⁵ is H, Me, Et, c-Pr, c-Bu, CH₂-c-Pr, (CH₂)_{q}S(O)ₚMe, (CH₂)_{q}OMe, (CH₂)_{q}COMe, each of which is optionally substituted by one or more radicals from the group consisting of F, Cl, cyano, C₁-C₃ alkyl, C₁-C₃ alkoxy, hydroxy and Ph.
Embodiment 12d. A compound of Embodiment 12c wherein R⁵ is H, Me, Et, i-pr, i-Bu, t-Bu, c-Pent, OMe, CH₂CF₃, CH₂CN, (CH₂)₂OMe, CH₂CO₂Me, CH₂CO₂Et, CH₂CO₂(4-F-Ph), CH₂SMe, CH₂Ph, NCHPh, (CH₂)₂SO₂Me, SO₂Me, SO₂Et, SO₂ (n-Pr), SO₂ (c-Pr), SO₂ (t-Bu), SO₂CF₃ or SO₂Ph.

### R⁶

Embodiment 13. A compound of Embodiment 2 wherein R⁶ is H, cyano, OR⁹, S(O)ₚR⁷, SO₂NR¹⁰R¹¹, CO₂R⁸, CONR¹⁰R¹¹, COR¹², NR¹⁰R¹¹, NR¹⁰COR¹², NR¹⁰CONR¹⁰R¹¹, NR¹⁰CO₂R⁸, NR¹⁰SO₂R⁷, NR¹⁰SO₂NR¹⁰R¹¹, C(R⁷)═NOR⁹, optionally substituted aryl, optionally substituted heteroaryl or optionally substituted heterocyclyl; or
   R⁶ is C₁-C₁₂ alkyl, C₃-C₈ cycloalkyl, C₄-C₁₂ cycloalkylalkyl, C₂-C₁₂ alkenyl, C₅-C₇ cycloalkenyl or C₂-C₁₂ alkynyl, each of which is optionally substituted by one or more radicals from the group consisting of halogen, cyano, nitro, OR⁹, S(O)ₚR⁷, SO₂NR¹⁰R¹¹, CO₂R⁸, CONR¹⁰R¹¹, COR¹², NR¹⁰R¹¹, NR¹⁰COR¹², NR¹⁰CONR¹⁰R¹¹, NR¹⁰CO₂R⁸, NR¹⁰SO₂R⁷, NR¹⁰SO₂NR¹⁰R¹¹, C(R⁷)═NOR⁹, optionally substituted aryl, optionally substituted heteroaryl or optionally substituted heterocyclyl; or
   R⁵ and R⁶ can be taken together with the nitrogen atom to which they are attached to form a 3- to 7-membered ring, containing carbon atoms and optionally 1 to 3 oxygen, sulfur or nitrogen atoms as ring members, wherein up to 2 carbon atom ring members are independently selected from C(═O) and C(═S), and the sulfur atom ring member is selected from S, S(O) or S(O)₂, said ring being optionally substituted with at least one substituent independently selected from the group consisting of halogen, cyano, nitro, C₁-C₆ alkyl, C₁-C₆ haloalkyl, OR⁹, S(O)ₚR⁷, SO₂NR¹⁰R¹¹, CO₂R⁸, CONR¹⁰R¹¹, COR¹², NR¹⁰R¹¹, NR¹⁰COR¹², NR¹⁰CONR¹⁰R¹¹, NR¹⁰CO₂R⁸, NR¹⁰SO₂R⁷, NR¹⁰SO₂NR¹⁰R¹¹, C(R⁷)—NOR⁹.
Embodiment 13a. A compound of Embodiment 13 wherein R⁶ is H, cyano, OR⁹, S(O)ₚR⁷, SO₂NR¹⁰R¹¹, CO₂R⁸, CONR¹⁰R¹¹, COR¹², NR¹⁰R¹¹, NR¹⁰COR¹², NR¹⁰CONR¹⁰R¹¹, NR¹⁰CO₂R⁸, NR¹⁰SO₂R⁷, NR¹⁰SO₂NR¹⁰R¹¹, C(R⁷)═NOR⁹; or C₁-C₁₂ alkyl, C₃-C₈ cycloalkyl, C₄-C₁₂ cycloalkylalkyl, C₂-C₁₂ alkenyl, C₅-C₇ cycloalkenyl or C₂-C₁₂ alkynyl, each of which is optionally substituted by one or more radicals from the group consisting of halogen, cyano, nitro, OR⁹, S(O)ₚR⁷, SO₂NR¹⁰R¹¹, CO₂R⁸, CONR¹⁰R¹¹, COR¹², NR¹⁰R¹¹, NR¹⁰COR¹², NR¹⁰CONR¹⁰R¹¹, NR¹⁰CO₂R⁸, NR¹⁰SO₂R⁷, NR¹⁰SO₂NR¹⁰R¹¹, C(R⁷)═NOR⁹.
Embodiment 13aa. A compound of Embodiment 13a wherein R⁶ is H, cyano, OR⁹, S(O)pR⁷, SO2NR¹⁰R¹¹, CO2R⁸, CONR¹⁰R¹¹, COR¹², NR¹⁰R¹¹, NR¹⁰COR¹², NR¹⁰CONR¹⁰R¹¹, NR¹⁰CO₂R⁸, NR¹⁰SO₂R⁷, NR¹⁰SO₂NR¹⁰R¹¹, C(R⁷)═NOR⁹; or Me, Et, c-Pr, CH₂-c-Pr, CH₂CH═CH₂, CH₂C≡CH.
Embodiment 13b. A compound of Embodiment 13a wherein R⁶ is H, cyano, OR⁹, S(O)pR⁷, SO₂NR¹⁰R¹¹, CO₂R⁸, CONR¹⁰R¹¹ or COR¹².
Embodiment 13c. A compound of Embodiment 13a wherein R⁶ is C₁-C₄ alkyl, C₃-C₈ cycloalkyl, C₄-C₈ cycloalkylalkyl, C₂-C₄ alkenyl, C₅-C₇ cycloalkenyl or C₂-C₆ alkynyl, each of which is optionally substituted by one or more radicals from the group consisting of halogen, cyano, OR⁹, S(O)ₚR⁷, SO₂NR¹⁰R¹¹, CO₂R⁸, CONR¹⁰R¹¹, COR¹², NR¹⁰R¹¹ or NR¹⁰COR¹².
Embodiment 13d. A compound of Embodiment 13b wherein R⁶ is H, OR⁹, S(O)pR⁷, SO₂NR¹⁰R¹¹, CO₂R⁸ or COR¹².
Embodiment 13e. A compound of Embodiment 13c wherein R⁶ is C₁-C₄ alkyl, C₃-C₈ cycloalkyl, C₄-C₈ cycloalkylalkyl, C₂-C₄ alkenyl, C₅-C₇ cycloalkenyl or C₂-C₆ alkynyl.
Embodiment 13f. A compound of Embodiment 13d wherein R⁶ is H, OR⁹ or S(O)ₚR⁷.
Embodiment 13g. A compound of Embodiment 13e wherein R⁶ is C₁-C₄ alkyl or C₃-C₈ cycloalkyl.
Embodiment 13 h. A compound of Embodiment 13d wherein R⁶ is H, OMe or S(O)₂R⁷.
Embodiment 13i. A compound of Embodiment 13e wherein R⁶ is Me or c-pr.
Embodiment 13j. A compound of Embodiment 13 wherein R⁵ and R⁶ are taken together with the nitrogen atom to which they are attached to form a 3- to 7-membered ring, containing carbon atoms and optionally 1 to 3 oxygen, sulfur or nitrogen atoms as ring members, wherein up to 2 carbon atom ring members are independently selected from C(═O) and C(═S), and the sulfur atom ring member is selected from S, S(O) or S(O)₂, said ring being optionally substituted with at least one substituent independently selected from the group consisting of halogen, cyano, C₁-C₃ alkyl, C₁-C₃ haloalkyl, oxo, OR⁹, S(O)pR⁷, CO₂R⁸ or NR¹⁰R¹¹.
Embodiment 13k. A compound of Embodiment 13j wherein the 3- to 7-membered ring contains carbon atoms and optionally 1 to 2 oxygen, sulfur or nitrogen atoms as ring members, wherein up to 2 carbon atom ring members are independently selected from C(═O) and C(═S), and the sulfur atom ring member is selected from S, S(O) or S(O)₂, said ring being optionally substituted with at least one substituent independently selected from the group consisting of halogen, cyano, C₁-C₃ alkyl, C₁-C₃ haloalkyl, oxo, OR⁹, S(O)ₚR⁷, CO₂R⁸ or NR¹⁰R¹¹.
Embodiment 131. A compound of Embodiment 13k wherein the 3- to 7-membered ring contains carbon atoms and optionally 1 to 2 oxygen, sulfur or nitrogen atoms as ring members, wherein up to 2 carbon atom ring members are independently selected from C(═O) and C(═S), and the sulfur atom ring member is selected from S, S(O) or S(O)₂, said ring being optionally substituted with at least one substituent independently selected from the group consisting of halogen, cyano, C₁-C₃ alkyl, C₁-C₃ haloalkyl.
Embodiment 13m. A compound of Embodiment 131 wherein the 3- to 7-membered ring is selected from the group of morpholinyl, thiomorpholinyl, oxazinyl, thiazinyl, piperidinyl, piperazinyl and isomers thereof, said ring being optionally substituted with at least one substituent independently selected from the group consisting of halogen, cyano, C₁-C₃ alkyl, and C₁-C₃ haloalkyl.
   R⁷, R⁸, R⁹, R¹⁰, R¹¹ and R¹²
Embodiment 14. A compound of Embodiment 2 wherein R⁷, R⁸, R⁹, R¹⁰, R¹¹ and R¹² are each independently H, C₁-C₆ alkyl, C₁-C₆ haloalkyl, C₃-C₇ cycloalkyl or aryl, each of which is optionally substituted by one or more radicals from the group consisting of F, Cl, C₁-C₂ alkoxy and aryl.
Embodiment 14a. A compound of Embodiment 14 wherein R⁷, R⁸, R⁹, R¹⁰, R¹¹ and R¹² are each independently H, C₁-C₃ alkyl, C₁-C₃ haloalkyl, C₃-C₅ cycloalkyl or aryl, each of which is optionally substituted by one or more radicals from the group consisting of F, Cl, C₁-C₂ alkoxy and aryl.
Embodiment 14b. A compound of Embodiment 14 wherein R⁷, R⁸, R⁹, R¹⁰, R¹¹ and R¹² are each independently H, C₁-C₃ alkyl, C₁-C₃ haloalkyl, C₃-C₅ cycloalkyl or phenyl, each of which is optionally substituted by one or more radicals from the group consisting of F, Cl, C₁-C₂ alkoxy and aryl.
Embodiment 14c. A compound of Embodiment 14 wherein R⁷, R⁸, R⁹, R¹⁰, R¹¹ and R¹² are each independently H, Me, Et, Pr, i-pr, c-pr, t-Bu, CF₃, OMe, OEt, CF₃, CH₂CF₃, c-Pr, Ph each of which is optionally substituted by one or more radicals from the group consisting of F, Cl, C₁-C₂ alkoxy and aryl.
Embodiment 15a. A compound of Formula 1 or Embodiment 1 wherein the stereochemistry of the carbon atom with * is (1′) depicted as Formula 1′ below.

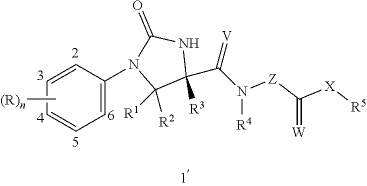

Embodiment 15b. A compound of Formula 1 or Embodiment 1 wherein the stereochemistry of the carbon atom with * is (1″), depicted as Formula 1″ below.

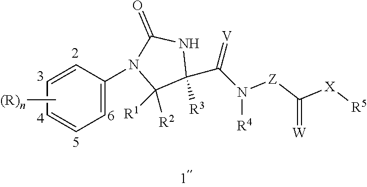

Embodiment 15c. A compound of Formula 1 or Embodiment 1 wherein the stereochemistry of the carbon atom with * is racemic, depicted as Formula 1 below.

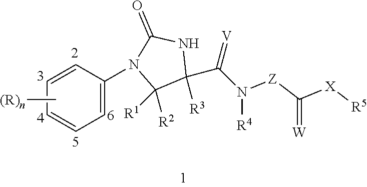

Embodiments of this invention, including Embodiments 1-14c above as well as any other embodiments described herein, can be combined in any manner, and the descriptions of variables in the embodiments pertain not only to the compounds of Formula 1 but also to the starting compounds and intermediate compounds useful for preparing the compounds of Formula 1. In addition, embodiments of this invention, including Embodiments 1-14c above as well as any other embodiments described herein, and any combination thereof, pertain to the compositions and methods of the present invention.

Combinations of Embodiments 1-14c are illustrated by:
Embodiment A. A compound of Formula 1 or Embodiment 1 wherein
   Z-A is selected from Z-1 through Z-29, Z-B is selected from Z-30 through Z-62, Z-C is selected from Z-63 through 64, and Z-D is selected from Z-65 through Z-74;

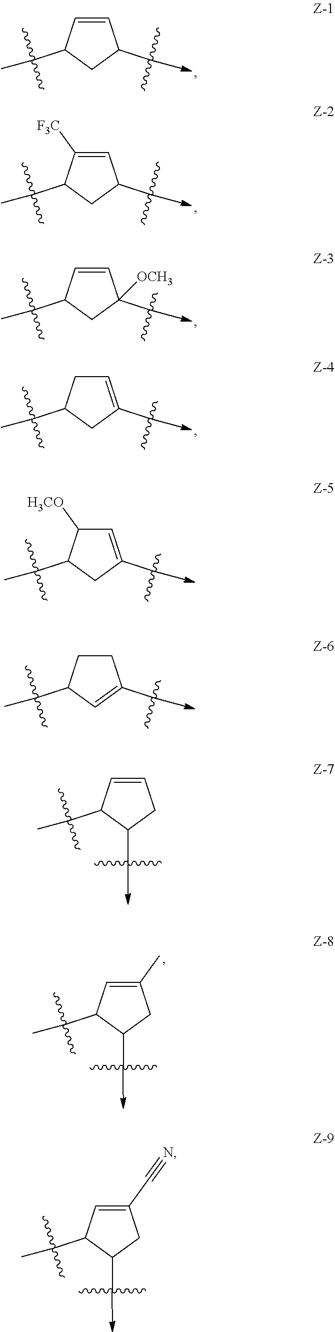

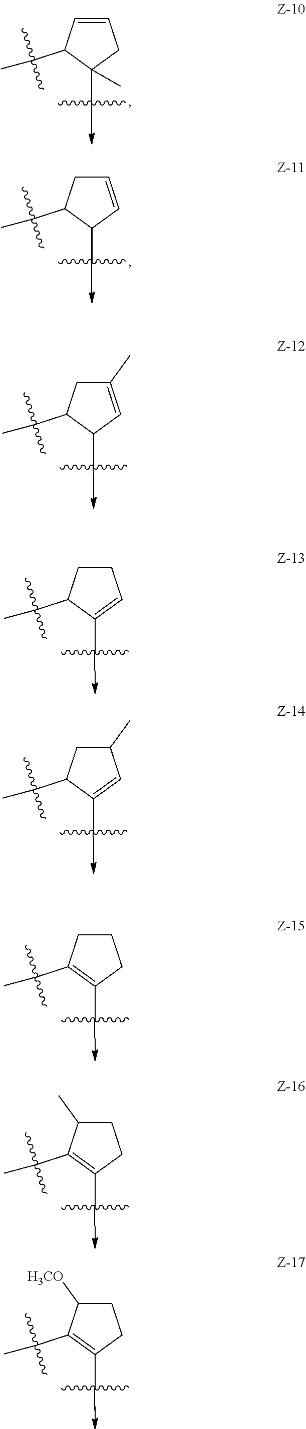

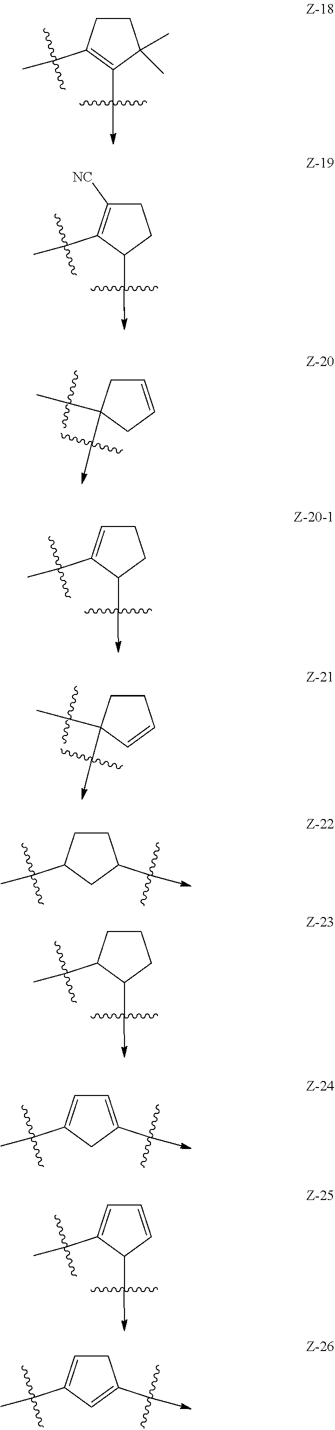

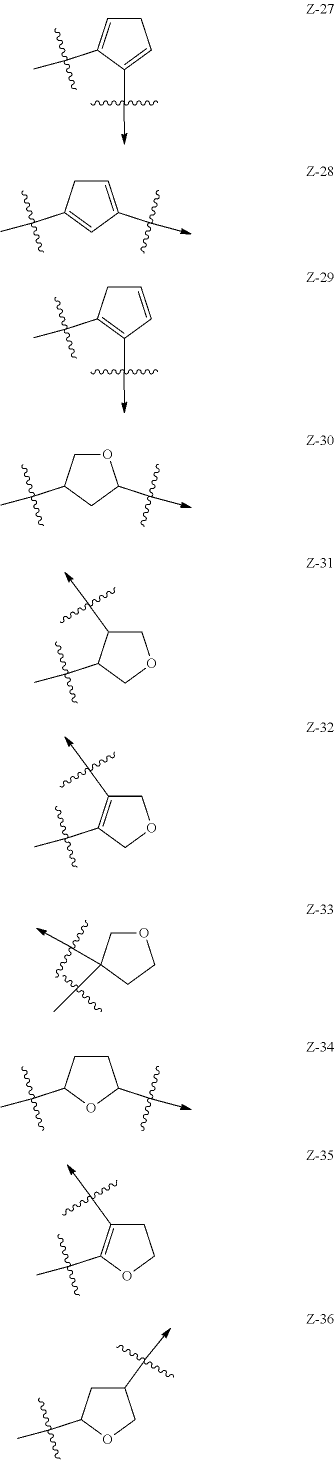

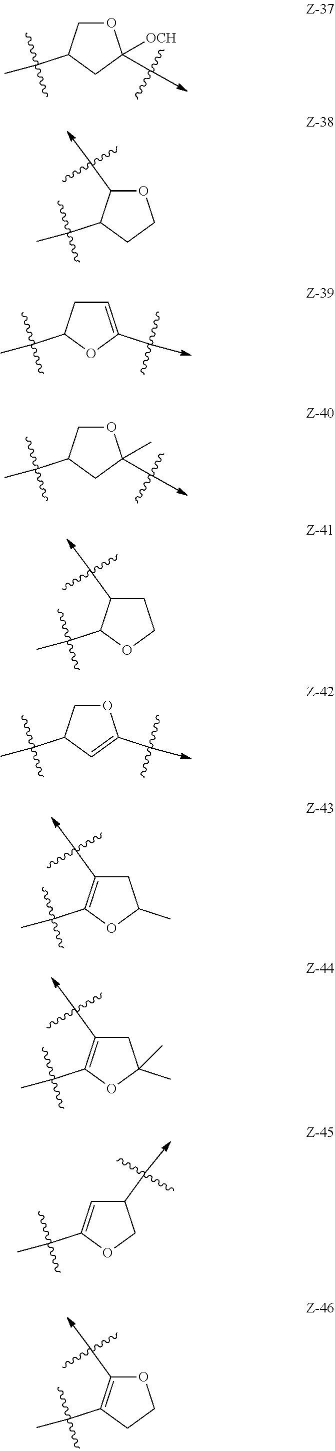

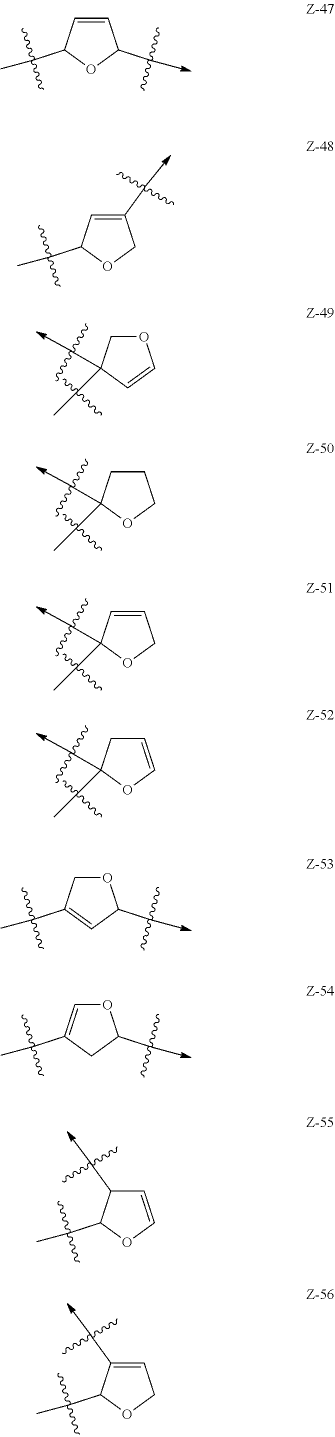

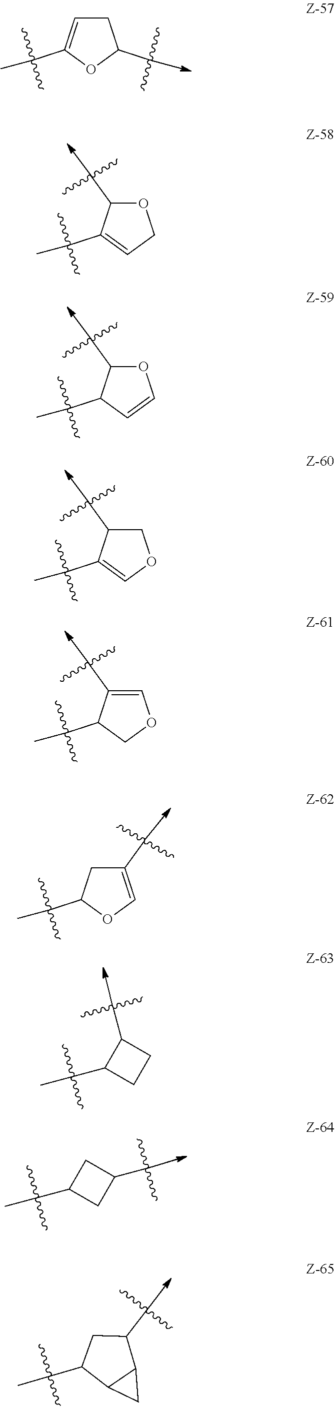

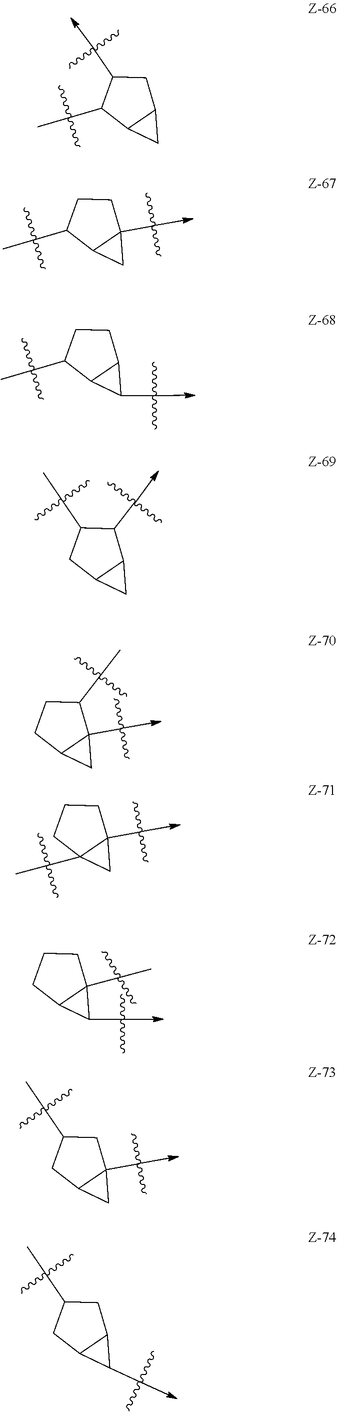

m is 0 or 1;
R is independently H, halogen, cyano, C₁-C₆ alkyl, C₁-C₆ haloalkyl, C₁-C₆ alkoxy or C₁-C₆ haloalkoxy;
n is 1, 2 or 3;
R¹ and R² are each independently hydrogen, halogen, cyano, C₁-C₃ alkyl, C₁-C₃ haloalkyl, C₁-C₃ cyanoalkyl, C₁-C₃ alkoxy, C₁-C₃ haloalkoxy or C₁-C₃ cyanoalkoxy; and
R³ is H, halogen, cyano, hydroxy, C₁-C₃ alkyl, C₂-C₃ alkenyl, C₂-C₃ alkynyl, C₃-C₅ cycloalkoxy, C₃-C₅ cycloalkoxyalkyl, C₃-C₅ cycloalkyl, C₄-C₇ cycloalkylalkyl, C₁-C₃ haloalkyl, C₂-C₃ alkoxyalkyl, C₂-C₃ haloalkoxyalkyl, C₁-C₃ alkoxy, C₁-C₃ haloalkoxy, C₁-C₃ alkylthio, C₁-C₃ alkylsulfinyl or C₁-C₃ alkylsulfonyl.
Embodiment A1. A compound of Embodiment A wherein
   Z is selected from the group Z-A;
   R is independently H, F, Cl, Br, cyano, C₁-C₂ alkyl, C₁-C₂ haloalkyl, C₁-C₂ alkoxy or C₁-C₂ haloalkoxy;
   V and W are both O;
   X is O or S;
   R³ is H, C₁-C₃ alkyl, C₂-C₃ alkenyl, C₂-C₃ alkynyl, C₃-C₅ cycloalkyl, C₄-C₇ cycloalkylalkyl, C₁-C₃ haloalkyl, C₂-C₃ alkoxyalkyl, C₂-C₃ haloalkoxyalkyl, C₁-C₃ alkoxy, C₁-C₃ haloalkoxy;
   R⁴ is H, C₁-C₆ alkyl, C₃-C₇ cycloalkyl, C₁-C₆ haloalkyl, C₁-C₃ alkoxy, C₁-C₃ haloalkoxy; and
   R⁵ is H, C₁-C₁₂ alkyl, NH₂, N═CR⁸R¹², C₃-C₇ cycloalkyl, C₃-C₁₂ cycloalkylalkyl, C₂-C₈ alkenyl, C₅-C₆ cycloalkenyl, C₂-C₈ alkynyl, (CH₂)_{q}S(O)ₚR⁷, (CH₂)_{q}OR⁹, (CH₂)_{q}COR¹², each of which is optionally substituted by one or more radicals from the group consisting of halogen, cyano, C₁-C₆ alkyl, C₁-C₆ alkoxy, hydroxy and aryl.
Embodiment A2. A compound of Embodiment A1 wherein
   Z is Z-1, Z-4, Z-6, Z-12, Z-22 or Z-24;
   R is independently H, F, Cl, Me, CF₃, OMe or OCF₃;
   n is 2;
   R is at 3- and 5-position;
   R¹ and R² are each independently hydrogen, halogen, cyano, Me, CF₃, CH₂CN, OMe, OCF₃ or OCH₂CN;
   R³ is H, Me, Et, CH═CH₂, C≡CH, cyclopropyl, cyclopropylmethyl, CF₃, OMe or OCF₃;
   R⁵ is H, Me, Et, c-Pr, c-Bu, CH₂-c-Pr, (CH₂)_{q}S(O)ₚMe, (CH₂)_{q}OMe, (CH₂)_{q}COMe, each of which is optionally substituted by one or more radicals from the group consisting of F, Cl, cyano, C₁-C₃ alkyl, C₁-C₃ alkoxy, hydroxy and Ph; and
   R⁷, R⁸, R⁹, R¹⁰, R¹¹ and R¹² are each independently H, C₁-C₃ alkyl, C₁-C₃ haloalkyl, C₃-C₅ cycloalkyl or phenyl, each of which is optionally substituted by one or more radicals from the group consisting of F, Cl, C₁-C₂ alkoxy or aryl.
Embodiment A3. A compound of Embodiment A wherein
   Z is selected from the group Z-A;
   R is independently H, F, Cl, Br, cyano, C₁-C₂ alkyl, C₁-C₂ haloalkyl, C₁-C₂ alkoxy or C₁-C₂ haloalkoxy;
   V and W are both O;
   X is NR⁶;
   R³ is H, C₁-C₃ alkyl, C₂-C₃ alkenyl, C₂-C₃ alkynyl, C₃-C₅ cycloalkyl, C₄-C₇ cycloalkylalkyl, C₁-C₃ haloalkyl, C₂-C₃ alkoxyalkyl, C₂-C₃ haloalkoxyalkyl, C₁-C₃ alkoxy, C₁-C₃ haloalkoxy;
   R⁴ is H, C₁-C₆ alkyl, C₃-C₇ cycloalkyl, C₁-C₆ haloalkyl, C₁-C₃ alkoxy, C₁-C₃ haloalkoxy.
Embodiment A4. A compound of Embodiment A3 wherein
   Z is Z-1, Z-4, Z-6, Z-12, Z-22 or Z-24;
   R is independently H, F, Cl, Me, CF₃, OMe or OCF₃;
   n is 2;
   R is at 3- and 5-position;
   R¹ and R² are each independently hydrogen, halogen, cyano, Me, CF₃, CH₂CN, OMe, OCF₃ or OCH₂CN;
   R³ is H, Me, Et, CH═CH₂, C≡CH, cyclopropyl, cyclopropylmethyl, CF₃, OMe or OCF₃; and
   R⁷, R⁸, R⁹, R¹⁰, R¹¹ and R¹² are each independently H, C₁-C₃ alkyl, C₁-C₃ haloalkyl, C₃-C₅ cycloalkyl or phenyl, each of which is optionally substituted by one or more radicals from the group consisting of F, Cl, C₁-C₂ alkoxy or aryl.
Embodiment A5. A compound of Embodiment A3 wherein
   R⁵ and R⁶ can be taken together with the nitrogen atom to which they are attached to form a 3- to 7-membered ring, wherein the 3- to 7-membered ring contains carbon atoms and optionally 1 to 2 oxygen, sulfur or nitrogen atoms as ring members, wherein up to 2 carbon atom ring members are independently selected from C(═O) and C(═S), and the sulfur atom ring member is selected from S, S(O) or S(O)₂, said ring being optionally substituted with at least one substituent independently selected from the group consisting of halogen, cyano, C₁-C₃ alkyl, C₁-C₃ haloalkyl.
Embodiment A6. A compound of Embodiment A5 wherein
   Z is Z-1, Z-4, Z-6, Z-12, Z-22 or Z-24;
   R is independently H, F, Cl, Me, CF₃, OMe or OCF₃;
   n is 2;
   R is at 3- and 5-position;
   R¹ and R² are each independently hydrogen, halogen, cyano, Me, CF₃, CH₂CN, OMe, OCF₃ or OCH₂CN;
   R³ is H, Me, Et, CH═CH₂, C≡CH, cyclopropyl, cyclopropylmethyl, CF₃, OMe or OCF₃; and
   R⁷, R⁸, R⁹, R¹⁰, R¹¹ and R¹² are each independently H, C₁-C₃ alkyl, C₁-C₃ haloalkyl, C₃-C₅ cycloalkyl or phenyl, each of which is optionally substituted by one or more radicals from the group consisting of F, Cl, C₁-C₂ alkoxy or aryl.
Embodiment B1. A compound of Embodiment A wherein
   Z is selected from the group Z-B;
   R is independently H, F, Cl, Br, cyano, C₁-C₂ alkyl, C₁-C₂ haloalkyl, C₁-C₂ alkoxy or C₁-C₂ haloalkoxy.
   V and W are both O;
   X is O or S;
   R³ is H, C₁-C₃ alkyl, C₂-C₃ alkenyl, C₂-C₃ alkynyl, C₃-C₅ cycloalkyl, C₄-C₇ cycloalkylalkyl, C₁-C₃ haloalkyl, C₂-C₃ alkoxyalkyl, C₂-C₃ haloalkoxyalkyl, C₁-C₃ alkoxy, C₁-C₃ haloalkoxy;
   R⁴ is H, C₁-C₆ alkyl, C₃-C₇ cycloalkyl, C₁-C₆ haloalkyl, C₁-C₃ alkoxy, C₁-C₃ haloalkoxy; and
   R⁵ is H, C₁-C₁₂ alkyl, NH₂, N═CR⁸R¹², C₃-C₇ cycloalkyl, C₃-C₁₂ cycloalkylalkyl, C₂-C₈ alkenyl, C₅-C₆ cycloalkenyl, C₂-C₈ alkynyl, (CH₂)_{q}S(O)ₚR⁷, (CH₂)_{q}OR⁹, (CH₂)_{q}COR¹², each of which is optionally substituted by one or more radicals from the group consisting of halogen, cyano, C₁-C₆ alkyl, C₁-C₆ alkoxy, hydroxy and aryl.
Embodiment B2. A compound of Embodiment B1 wherein
   Z is Z-30, Z-34, Z-36, Z-42, Z-47, Z-48, Z-53, Z-54, Z-55, Z-57 or Z-62;
   R is independently H, F, Cl, Me, CF₃, OMe or OCF₃;
   n is 2;
   R is at 3- and 5 position;
   R¹ and R² are each independently hydrogen, halogen, cyano, Me, CF₃, CH₂CN, OMe, OCF₃ or OCH₂CN;
   R³ is H, Me, Et, CH═CH₂, C≡CH, cyclopropyl, cyclopropylmethyl, CF₃, OMe or OCF₃.
   R⁵ is H, Me, Et, c-Pr, c-Bu, CH₂-c-Pr, (CH₂)_{q}S(O)ₚMe, (CH₂) OMe, (CH₂)_{q}COMe, each of which is optionally substituted by one or more radicals from the group consisting of F, Cl, cyano, C₁-C₃ alkyl, C₁-C₃ alkoxy, hydroxy and Ph; and
   R⁷, R⁸, R⁹, R¹⁰, R¹¹ and R¹² are each independently H, C₁-C₃ alkyl, C₁-C₃ haloalkyl, C₃-C₅ cycloalkyl or phenyl, each of which is optionally substituted by one or more radicals from the group consisting of F, Cl, C₁-C₂ alkoxy or aryl.

Specific embodiments include compounds of Formula 1 selected from the group consisting of:
2,2,2-Trifluoroethyl (4S)-4-[[[(5S)-3-(3,5-difluorophenyl)-5-methyl-2-oxo-5-oxazolidinyl]carbonyl]amino]-1-cyclopentene-1-carboxylate;
2,2,2-Trifluoroethyl (4S)-4-[[[(5R)-3-(3,5-difluorophenyl)-5-methyl-2-oxo-5-oxazolidinyl]carbonyl]amino]-1-cyclopentene-1-carboxylate;
2-(Methylthio)ethyl (4S)-4-[[[(5S)-3-(3,5-difluorophenyl)-5-methyl-2-oxo-5-oxazolidinyl]carbonyl]amino]-1-cyclopentene-1-carboxylate;
2-(Methylthio)ethyl (4S)-4-[[[(5R)-3-(3,5-difluorophenyl)-5-methyl-2-oxo-5-oxazolidinyl]carbonyl]amino]-1-cyclopentene-1-carboxylate;
Methyl (4S)-4-[[[(5S)-3-(3,5-difluorophenyl)-5-methyl-2-oxo-5-oxazolidinyl]carbonyl]amino]-1-cyclopentene-1-carboxylate;
Methyl (4S)-4-[[[(5R)-3-(3,5-difluorophenyl)-5-methyl-2-oxo-5-oxazolidinyl]carbonyl]amino]-1-cyclopentene-1-carboxylate;
Methyl (4S)-4-[[[3-(3,5-difluorophenyl)-5-methyl-2-oxo-5-oxazolidinyl]carbonyl]amino]-1-cyclopentene-1-carboxylate;
1-Methylethyl (1S,4R)-4-[[[3-(3-chloro-5-fluorophenyl)-5-methyl-2-oxo-5-oxazolidinyl]carbonyl]amino]-2-cyclopentene-1-carboxylate;
Methyl (1S,4R)-4-[[[3-(3-chloro-5-fluorophenyl)-5-methyl-2-oxo-5-oxazolidinyl]carbonyl]amino]-2-cyclopentene-1-carboxylate;
2-Methylpropyl (4S)-4-[[[3-(3,5-difluorophenyl)-5-methyl-2-oxo-5-oxazolidinyl]carbonyl]amino]-1-cyclopentene-1-carboxylate;
(1S,4R)-4-[[[3-(3,5-Difluorophenyl)-5-methyl-2-oxo-5-oxazolidinyl]carbonyl]amino]-2-cyclopentene-1-carboxylic acid;
(4S)-4-[[[3-(3,5-Difluorophenyl)-5-methyl-2-oxo-5-oxazolidinyl]carbonyl]amino]-1-cyclopentene-1-carboxylic acid;
(1S,4R)-4-[[[3-(3-Chloro-5-fluorophenyl)-5-methyl-2-oxo-5-oxazolidinyl]carbonyl]amino]-2-cyclopentene-1-carboxylic acid; and
Ethyl (1S,4R)-4-[[[3-(3,5-difluorophenyl)-5-methyl-2-oxo-5-oxazolidinyl]carbonyl]amino]-2-cyclopentene-1-carboxylate;

Specific embodiments also include a compound of Formula 1 selected from the group consisting of: Compound No. 89, 96, 98, 128, 130, 140, 145, 161, 164 and 169.

Combinations of Embodiments 1-14c are illustrated by the following Embodiments P1 through P15:
Embodiment P1. A compound of Formula 1A, all stereoisomers, N-oxides, and salts thereof

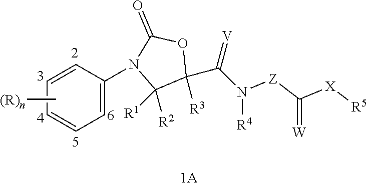

wherein
   Z is selected from a group of a fully saturated, or a fully or partly unsaturated, five membered rings as shown below, optionally substituted by group R⁴

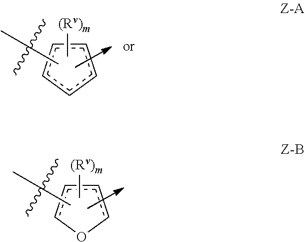

m is 0, 1 or 2;
each R is independently H, halogen, cyano, nitro, hydroxy, C₁-C₆ alkyl, C₁-C₆ haloalkyl, C₂-C₆ alkenyl, C₂-C₆ haloalkenyl, C₂-C₆ alkynyl, C₂-C₆ haloalkynyl, C₃-C₇ cycloalkyl, C₃-C₇ halocycloalkyl, C₁-C₆ alkoxy, C₁-C₆ haloalkoxy, S(O)ₚR⁷ or CO₂R⁸;
n is 0, 1, 2, 3, 4 or 5;
p is 0, 1 or 2;
V and W are each independently O or S;
X is a direct bond, O, S or NR⁶;
R¹ and R² are each independently hydrogen, halogen, cyano, C₁-C₆ alkyl, C₁-C₆ haloalkyl, C₁-C₆ cyanoalkyl, C₁-C₆ alkoxy, C₁-C₆ haloalkoxy or C₁-C₆ cyanoalkoxy;
R³ is H, halogen, cyano, nitro, hydroxy, C₁-C₆ alkyl, C₂-C₅ alkenyl, C₂-C₅ alkynyl, C₂-C₅ alkenyloxy, C₂-C₅ alkynyloxy, C₃-C₇ cycloalkoxy, C₃-C₇ cycloalkoxyalkyl, C₃-C₆ cycloalkyl, C₄-C₇ cycloalkylalkyl, C₁-C₆ haloalkyl, C₂-C₅ haloalkenyl, C₂-C₅ haloalkynyl, C₂-C₅ alkoxyalkyl, C₂-C₅ haloalkoxyalkyl, C₁-C₅ alkoxy, C₁-C₆ haloalkoxy, C₁-C₅ alkylthio, C₁-C₄ alkylsulfinyl, C₁-C₄ alkylsulfonyl, C₁-C₄ alkylsulfonate, C₁-C₄ haloalkylthio, C₁-C₄ haloalkylsulfinyl, C₁-C₄ haloalkylsulfonyl or C₂-C₅ alkoxycarbonyl, optionally each of which is further substituted by at least one radical from the group consisting of halogen, cyano, C₁-C₄ alkoxy, C₁-C₅ alkylthio, C₁-C₄ alkylsulfinyl, C₁-C₄ alkylsulfonyl, and hydroxy;
R⁴ is independently H, C₁-C₆ alkyl, C₃-C₇ cycloalkyl, C₁-C₆ haloalkyl, C₁-C₃ alkoxy, C₁-C₃ haloalkoxy; or W¹G¹;
W¹ is a direct bond, C₁-C₄ alkanediyl or C₁-C₄ alkenediyl;
G¹ is S(O)ₚR⁷, SO₂NR¹⁰R¹¹, CO₂R⁸, CONR¹⁰R¹¹ or COR¹²;
R⁵ is H, C₁-C₁₂ alkyl, NH₂, N═CR⁸R¹², C₃-C₇ cycloalkyl, C₃-C₁₂ cycloalkylalkyl, C₂-C₈ alkenyl, C₅-C₆ cycloalkenyl, C₂-C₈ alkynyl, (CH₂) S(O)ₚR⁷, (CH₂)_{q}OR⁹, (CH₂)_{q}COR¹², each of which is optionally substituted by one or more radicals from the group consisting of halogen, cyano, C₁-C₆ alkyl, C₁-C₆ alkoxy, hydroxy and aryl;
q is 0, 1, 2, 3, 4 or 5;
R⁶ is hydrogen, cyano, OR⁹, S(O)ₚR⁷, SO₂NR¹⁰R¹¹, CO₂R⁸, CONR¹⁰R¹¹, COR¹², NR¹⁰R¹¹, NR¹⁰COR¹², NR¹⁰CONR¹⁰R¹¹, NR¹⁰CO₂R⁸, NR¹⁰SO₂R⁷, NR¹⁰SO₂NR¹⁰R¹¹, C(R⁷)═NOR⁹, optionally substituted aryl, optionally substituted heteroaryl or optionally substituted heterocyclyl; or
R⁶ is C₁-C₁₂ alkyl, C₃-C₈ cycloalkyl, C₄-C₁₂ cycloalkylalkyl, C₂-C₁₂ alkenyl, C₅-C₇ cycloalkenyl or C₂-C₁₂ alkynyl, each of which is optionally substituted by one or more radicals from the group consisting of halogen, cyano, nitro, OR⁹, S(O)ₚR⁷, SO₂NR¹⁰R¹¹, CO₂R⁸, CONR¹⁰R¹¹, COR¹², NR¹⁰R¹¹, NR¹⁰COR¹², NR¹⁰CONR¹⁰R¹¹, NR¹⁰CO₂R⁸, NR¹⁰SO₂R⁷, NR¹⁰SO₂NR¹⁰R¹¹, C(R⁷)═NOR⁹, optionally substituted aryl, optionally substituted heteroaryl or optionally substituted heterocyclyl; or
R⁵ and R⁶ can be taken together with the nitrogen atom to which they are attached to form a 3- to 7-membered ring, containing carbon atoms and optionally 1 to 3 oxygen, sulfur or nitrogen atoms as ring members, wherein up to 2 carbon atom ring members are independently selected from C(═O) and C(═S), and the sulfur atom ring member is selected from S, S(O) or S(O)₂, said ring being optionally substituted with at least one substituent independently selected from the group consisting of halogen, cyano, nitro, C₁-C₆ alkyl, C₁-C₆ haloalkyl, OR⁹, S(O)ₚR⁷, SO₂NR¹⁰R¹¹, CO₂R⁸, CONR¹⁰R¹¹, COR¹², NR¹⁰R¹¹, NR¹⁰COR¹², NR¹⁰CONR¹⁰R¹¹, NR¹⁰CO₂R⁸, NR¹⁰SO₂R⁷, NR¹⁰SO₂NR¹⁰R¹¹, C(R⁷)═NOR⁹;
R⁷, R⁸, R⁹, R¹⁰, R¹¹ and R¹² are each independently H, C₁-C₆ alkyl, C₁-C₆ haloalkyl, C₃-C₇ cycloalkyl or aryl, each of which is optionally substituted by one or more radicals from the group consisting of F, Cl, C₁-C₂ alkoxy or aryl.
Embodiment P2. The compound of Embodiment P1 wherein
   Z-A is selected from Z-1 though Z-29, and Z-B is selected from Z-30 though Z-62;

[chemical structure]

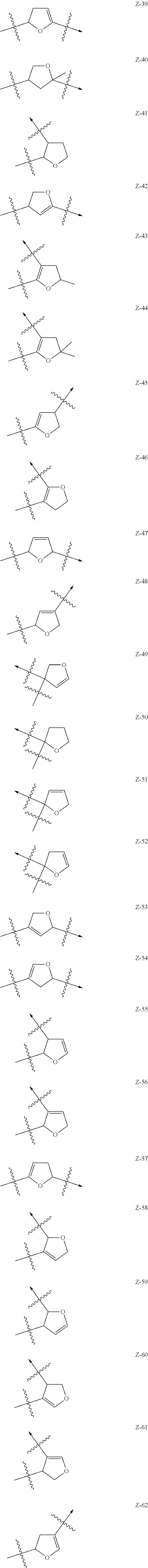

m is 0 or 1;
R is independently H, halogen, cyano, C₁-C₆ alkyl, C₁-C₆ haloalkyl, C₁-C₆ alkoxy or C₁-C₆ haloalkoxy;
n is 1, 2 or 3;
R¹ and R² are each independently hydrogen, halogen, cyano, C₁-C₃ alkyl, C₁-C₃ haloalkyl, C₁-C₃ cyanoalkyl, C₁-C₃ alkoxy, C₁-C₃ haloalkoxy or C₁-C₃ cyanoalkoxy; and
R³ is H, halogen, cyano, hydroxy, C₁-C₃ alkyl, C₂-C₃ alkenyl, C₂-C₃ alkynyl, C₃-C₅ cycloalkoxy, C₃-C₅ cycloalkoxyalkyl, C₃-C₅ cycloalkyl, C₄-C₇ cycloalkylalkyl, C₁-C₃ haloalkyl, C₂-C₃ alkoxyalkyl, C₂-C₃ haloalkoxyalkyl, C₁-C₃ alkoxy, C₁-C₃ haloalkoxy, C₁-C₃ alkylthio, C₁-C₃ alkylsulfinyl or C₁-C₃ alkylsulfonyl.
Embodiment P3. The compound of Embodiment P2 wherein
   Z is selected from the group Z-A;
   R is independently H, F, Cl, Br, cyano, C₁-C₂ alkyl, C₁-C₂ haloalkyl, C₁-C₂ alkoxy or C₁-C₂ haloalkoxy;
   V and W are both O;
   X is O or S;
   R³ is H, C₁-C₃ alkyl, C₂-C₃ alkenyl, C₂-C₃ alkynyl, C₃-C₅ cycloalkyl, C₄-C₇ cycloalkylalkyl, C₁-C₃ haloalkyl, C₂-C₃ alkoxyalkyl, C₂-C₃ haloalkoxyalkyl, C₁-C₃ alkoxy, C₁-C₃ haloalkoxy;
   R⁴ is H, C₁-C₆ alkyl, C₃-C₇ cycloalkyl, C₁-C₆ haloalkyl, C₁-C₃ alkoxy, C₁-C₃ haloalkoxy; and
   R⁵ is H, C₁-C₁₂ alkyl, NH₂, N═CR⁸R¹², C₃-C₇ cycloalkyl, C₃-C₁₂ cycloalkylalkyl, C₂-C₈ alkenyl, C₅-C₆ cycloalkenyl, C₂-C₈ alkynyl, (CH₂)_{q}S(O)ₚR⁷, (CH₂)_{q}OR⁹, (CH₂)_{q}COR¹², each of which is optionally substituted by one or more radicals from the group consisting of halogen, cyano, C₁-C₆ alkyl, C₁-C₆ alkoxy, hydroxy and aryl.
Embodiment P4. The compound of Embodiment P3 wherein
   Z is Z-1, Z-4, Z-6, Z-12, Z-22 or Z-24;
   R is independently H, F, Cl, Me, CF₃, OMe or OCF₃;
   n is 2;
   R is at 3- and 5-position;
   R¹ and R² are each independently hydrogen, halogen, cyano, Me, CF₃, CH₂CN, OMe, OCF₃ or OCH₂CN;
   R³ is H, Me, Et, CH═CH₂, C≡CH, cyclopropyl, cyclopropylmethy, CF₃, OMe or OCF₃.
   R⁵ is H, Me, Et, c-Pr, c-Bu, CH₂-c-Pr, (CH₂)_{q}S(O)ₚMe, (CH₂)_{q}OMe, (CH₂)_{q}COMe, each of which is optionally substituted by one or more radicals from the group consisting of F, Cl, cyano, C₁-C₃ alkyl, C₁-C₃ alkoxy, hydroxy and Ph; and R⁷, R⁸, R⁹, R¹⁰, R¹¹ and R¹² are each independently H, C₁-C₃ alkyl, C₁-C₃ haloalkyl, C₃-C₅ cycloalkyl or phenyl, each of which is optionally substituted by one or more radicals from the group consisting of F, Cl, C₁-C₂ alkoxy or aryl.
Embodiment P5. The compound of Embodiment P2 wherein
   Z is selected from the group Z-A;
   R is independently H, F, Cl, Br, cyano, C₁-C₂ alkyl, C₁-C₂ haloalkyl, C₁-C₂ alkoxy or C₁-C₂ haloalkoxy;
   V and W are both O;
   X is NR⁶;
   R³ is H, C₁-C₃ alkyl, C₂-C₃ alkenyl, C₂-C₃ alkynyl, C₃-C₅ cycloalkyl, C₄-C₇ cycloalkylalkyl, C₁-C₃ haloalkyl, C₂-C₃ alkoxyalkyl, C₂-C₃ haloalkoxyalkyl, C₁-C₃ alkoxy, C₁-C₃ haloalkoxy;
   R⁴ is H, C₁-C₆ alkyl, C₃-C₇ cycloalkyl, C₁-C₆ haloalkyl, C₁-C₃ alkoxy, C₁-C₃ haloalkoxy.
Embodiment P6. The compound of Embodiment P5 wherein
   Z is Z-1, Z-4, Z-6, Z-12, Z-22 or Z-24;
   R is independently H, F, Cl, Me, CF₃, OMe or OCF₃;
   n is 2;
   R is at 3- and 5 position;
   R¹ and R² are each independently hydrogen, halogen, cyano, Me, CF₃, CH₂CN, OMe, OCF₃ or OCH₂CN;
   R³ is H, Me, Et, CH═CH₂, C≡CH, cyclopropyl, cyclopropylmethy, CF₃, OMe or OCF₃; and
   R⁷, R⁸, R⁹, R¹⁰, R¹¹ and R¹² are each independently H, C₁-C₃ alkyl, C₁-C₃ haloalkyl, C₃-C₅ cycloalkyl or phenyl, each of which is optionally substituted by one or more radicals from the group consisting of F, Cl, C₁-C₂ alkoxy or aryl
Embodiment P7. The compound of Embodiment P5 wherein
   R⁵ and R⁶ can be taken together with the nitrogen atom to which they are attached to form a 3- to 7-membered ring, wherein the 3- to 7-membered ring contains carbon atoms and optionally 1 to 2 oxygen, sulfur or nitrogen atoms as ring members, wherein up to 2 carbon atom ring members are independently selected from C(═O) and C(═S), and the sulfur atom ring member is selected from S, S(O) or S(O)₂, said ring being optionally substituted with at least one substituent independently selected from the group consisting of halogen, cyano, C₁-C₃ alkyl, C₁-C₃ haloalkyl.
Embodiment P8. The compound of Embodiment P7 wherein
   Z is Z-1, Z-4, Z-6, Z-12, Z-22 or Z-24;
   R is independently H, F, Cl, Me, CF₃, OMe or OCF₃;
   n is 2;
   R is at 3- and 5-position;
   R¹ and R² are each independently hydrogen, halogen, cyano, Me, CF₃, CH₂CN, OMe, OCF₃ or OCH₂CN;
   R³ is H, Me, Et, CH═CH₂, C≡CH, cyclopropyl, cyclopropylmethy, CF₃, OMe or OCF₃; and
   R⁷, R⁸, R⁹, R¹⁰, R¹¹ and R¹² are each independently H, C₁-C₃ alkyl, C₁-C₃ haloalkyl, C₃-C₅ cycloalkyl or phenyl, each of which is optionally substituted by one or more radicals from the group consisting of F, Cl, C₁-C₂ alkoxy or aryl.
Embodiment P9. The compound of Embodiment P2 wherein
   Z is selected from the group Z-B;
   R is independently H, F, Cl, Br, cyano, C₁-C₂ alkyl, C₁-C₂ haloalkyl, C₁-C₂ alkoxy or C₁-C₂ haloalkoxy;
   V and W are both O;
   X is O or S;
   R³ is H, C₁-C₃ alkyl, C₂-C₃ alkenyl, C₂-C₃ alkynyl, C₃-C₅ cycloalkyl, C₄-C₇ cycloalkylalkyl, C₁-C₃ haloalkyl, C₂-C₃ alkoxyalkyl, C₂-C₃ haloalkoxyalkyl, C₁-C₃ alkoxy, C₁-C₃ haloalkoxy;
   R⁴ is H, C₁-C₆ alkyl, C₃-C₇ cycloalkyl, C₁-C₆ haloalkyl, C₁-C₃ alkoxy, C₁-C₃ haloalkoxy; and
   R⁵ is H, C₁-C₁₂ alkyl, NH₂, N═CR⁸R¹², C₃-C₇ cycloalkyl, C₃-C₁₂ cycloalkylalkyl, C₂-C₈ alkenyl, C₅-C₆ cycloalkenyl, C₂-C₈ alkynyl, (CH₂)_{q}S(O)ₚR⁷, (CH₂)_{q}OR⁹, (CH₂)_{q}COR¹², each of which is optionally substituted by one or more radicals from the group consisting of halogen, cyano, C₁-C₆ alkyl, C₁-C₆ alkoxy, hydroxy and aryl.
Embodiment P10. The compound of Embodiment P9 wherein
   Z is Z-30, Z-34, Z-36, Z-42, Z-47, Z-48, Z-53, Z-54, Z-55, Z-57 or Z-62;
   R is independently H, F, Cl, Me, CF₃, OMe or OCF₃;
   n is 2;
   R is at 3- and 5-position;
   R¹ and R² are each independently hydrogen, halogen, cyano, Me, CF₃, CH₂CN, OMe, OCF₃ or OCH₂CN;
   R³ is H, Me, Et, CH═CH₂, C≡CH, cyclopropyl, cyclopropylmethy, CF₃, OMe or OCF₃;
   R⁵ is H, Me, Et, c-Pr, c-Bu, CH₂-c-Pr, (CH₂)_{q}S(O)ₚMe, (CH₂)_{q}OMe, (CH₂) COMe, each of which is optionally substituted by one or more radicals from the group consisting of F, Cl, cyano, C₁-C₃ alkyl, C₁-C₃ alkoxy, hydroxy and Ph; and
   R⁷, R⁸, R⁹, R¹⁰, R¹¹ and R¹² are each independently H, C₁-C₃ alkyl, C₁-C₃ haloalkyl, C₃-C₅ cycloalkyl or phenyl, each of which is optionally substituted by one or more radicals from the group consisting of F, Cl, C₁-C₂ alkoxy or aryl.
Embodiment P11. The compound of Embodiment P1 1 selected from the group consisting of
2,2,2-Trifluoroethyl (4S)-4-[[[(5S)-3-(3,5-difluorophenyl)-5-methyl-2-oxo-5-oxazolidinyl]carbonyl]amino]-1-cyclopentene-1-carboxylate;
2,2,2-Trifluoroethyl (4S)-4-[[[(5R)-3-(3,5-difluorophenyl)-5-methyl-2-oxo-5-oxazolidinyl]carbonyl]amino]-1-cyclopentene-1-carboxylate;
2-(Methylthio)ethyl (4S)-4-[[[(5S)-3-(3,5-difluorophenyl)-5-methyl-2-oxo-5-oxazolidinyl]carbonyl]amino]-1-cyclopentene-1-carboxylate;
2-(Methylthio)ethyl (4S)-4-[[[(5R)-3-(3,5-difluorophenyl)-5-methyl-2-oxo-5-oxazolidinyl]carbonyl]amino]-1-cyclopentene-1-carboxylate;
Methyl (4S)-4-[[[(5S)-3-(3,5-difluorophenyl)-5-methyl-2-oxo-5-oxazolidinyl]carbonyl]amino]-1-cyclopentene-1-carboxylate;
Methyl (4S)-4-[[[(5R)-3-(3,5-difluorophenyl)-5-methyl-2-oxo-5-oxazolidinyl]carbonyl]amino]-1-cyclopentene-1-carboxylate;
Methyl (4S)-4-[[[3-(3,5-difluorophenyl)-5-methyl-2-oxo-5-oxazolidinyl]carbonyl]amino]-1-cyclopentene-1-carboxylate;
1-Methylethyl (1S,4R)-4-[[[3-(3-chloro-5-fluorophenyl)-5-methyl-2-oxo-5-oxazolidinyl]carbonyl]amino]-2-cyclopentene-1-carboxylate;
Methyl (1S,4R)-4-[[[3-(3-chloro-5-fluorophenyl)-5-methyl-2-oxo-5-oxazolidinyl]carbonyl]amino]-2-cyclopentene-1-carboxylate;
2-Methylpropyl (4S)-4-[[[3-(3,5-difluorophenyl)-5-methyl-2-oxo-5-oxazolidinyl]carbonyl]amino]-1-cyclopentene-1-carboxylate;
(1S,4R)-4-[[[3-(3,5-Difluorophenyl)-5-methyl-2-oxo-5-oxazolidinyl]carbonyl]amino]-2-cyclopentene-1-carboxylic acid;
(4S)-4-[[[3-(3,5-Difluorophenyl)-5-methyl-2-oxo-5-oxazolidinyl]carbonyl]amino]-1-cyclopentene-1-carboxylic acid;
(1S,4R)-4-[[[3-(3-Chloro-5-fluorophenyl)-5-methyl-2-oxo-5-oxazolidinyl]carbonyl]amino]-2-cyclopentene-1-carboxylic acid; and
Ethyl (1S,4R)-4-[[[3-(3,5-difluorophenyl)-5-methyl-2-oxo-5-oxazolidinyl]carbonyl]amino]-2-cyclopentene-1-carboxylate.
   Embodiment P12. A herbicidal composition comprising a compound of Embodiment P1 and at least one component selected from the group consisting of surfactants, solid diluents and liquid diluents.
   Embodiment P13. A herbicidal composition comprising a compound of Embodiment P1, at least one additional active ingredient selected from the group consisting of other herbicides and herbicide safeners, and at least one component selected from the group consisting of surfactants, solid diluents and liquid diluents.
   Embodiment P14. A herbicidal mixture comprising (a) a compound of Embodiment P1, and (b) at least one additional active ingredient selected from (b1) photosystem II inhibitors, (b2) acetohydroxy acid synthase (AHAS) inhibitors, (b3) acetyl-CoA carboxylase (ACCase) inhibitors, (b4) auxin mimics, (b5) 5-enol-pyruvylshikimate-3-phosphate (EPSP) synthase inhibitors, (b6) photosystem I electron diverters, (b7) protoporphyrinogen oxidase (PPO) inhibitors, (b8)glutamine synthetase (GS) inhibitors, (b9) very long chain fatty acid (VLCFA) elongase inhibitors, (b10) auxin transport inhibitors, (b11) phytoene desaturase (PDS) inhibitors, (b12) 4-hydroxyphenyl-pyruvate dioxygenase (HPPD) inhibitors, (b13) homogentisate solanesyltransferase (HST) inhibitors, (b14) cellulose biosynthesis inhibitors, (b15) dehydrooritate dehydrogenase (DHODH) inhibitors, (b16) other herbicides including mitotic disruptors, organic arsenicals, asulam, bromobutide, cinmethylin, cumyluron, dazomet, difenzoquat, dymron, etobenzanid, flurenol, fosamine, fosamine-ammonium, hydantocidin, metam, methyldymron, oleic acid, oxaziclomefone, pelargonic acid and pyributicarb, (b17) herbicide safeners, and salts of compounds of (b1) through (b17).
   Embodiment P15. A method for controlling the growth of undesired vegetation comprising contacting the vegetation or its environment with a herbicidally effective amount of a compound of Embodiment P1.

This invention also relates to a method for controlling undesired vegetation comprising applying to the locus of the vegetation herbicidally effective amounts of the compounds of the invention (e.g., as a composition described herein). Of note as embodiments relating to methods of use are those involving the compounds of embodiments described above. Compounds of the invention are particularly useful for selective control of weeds in crops such as wheat, barley, maize, soybean, sunflower, cotton, oilseed rape and rice, and specialty crops such as sugarcane, citrus, fruit and nut crops.

Also noteworthy as embodiments are herbicidal compositions of the present invention comprising the compounds of embodiments described above.

This invention also includes a herbicidal mixture comprising (a) a compound selected from Formula 1, N-oxides, and salts thereof, and (b) at least one additional active ingredient selected from (b1) photosystem II inhibitors, (b2) acetohydroxy acid synthase (AHAS) inhibitors, (b3) acetyl-CoA carboxylase (ACCase) inhibitors, (b4) auxin mimics, (b5) 5-enol-pyruvylshikimate-3-phosphate (EPSP) synthase inhibitors, (b6) photosystem I electron diverters, (b7) protoporphyrinogen oxidase (PPO) inhibitors, (b8) glutamine synthetase (GS) inhibitors, (b9) very long chain fatty acid (VLCFA) elongase inhibitors, (b10) auxin transport inhibitors, (b11) phytoene desaturase (PDS) inhibitors, (b12) 4-hydroxyphenyl-pyruvate dioxygenase (HPPD) inhibitors, (b13) homogentisate solanesyltransferase (HST) inhibitors, (b14) cellulose biosynthesis inhibitors, (b15) “DHODH (dihydroorotate dehydrogenase) inhibitors”, (b16) other herbicides including mitotic disruptors, organic arsenicals, asulam, bromobutide, cinmethylin, cumyluron, dazomet, difenzoquat, dymron, etobenzanid, flurenol, fosamine, fosamine-ammonium, hydantocidin, metam, methyldymron, oleic acid, oxaziclomefone, pelargonic acid and pyributicarb, (b17) herbicide safeners, and salts of compounds of (b1) through (b17).

“Photosystem II inhibitors” (b1) are chemical compounds that bind to the D-1 protein at the Q_{B}-binding niche and thus block electron transport from Q_{A} to Q_{B} in the chloroplast thylakoid membranes. The electrons blocked from passing through photosystem II are transferred through a series of reactions to form toxic compounds that disrupt cell membranes and cause chloroplast swelling, membrane leakage, and ultimately cellular destruction. The Q_{B}-binding niche has three different binding sites: binding site A binds the triazines such as atrazine, triazinones such as hexazinone, and uracils such as bromacil, binding site B binds the phenylureas such as diuron, and binding site C binds benzothiadiazoles such as bentazon, nitriles such as bromoxynil and phenyl-pyridazines such as pyridate. Examples of photosystem II inhibitors include ametryn, amicarbazone, atrazine, bentazon, bromacil, bromofenoxim, bromoxynil, chlorbromuron, chloridazon, chlorotoluron, chloroxuron, cumyluron, cyanazine, daimuron, desmedipham, desmetryn, dimefuron, dimethametryn, diuron, ethidimuron, fenuron, fluometuron, hexazinone, ioxynil, isoproturon, isouron, lenacil, linuron, metamitron, methabenzthiazuron, metobromuron, metoxuron, metribuzin, monolinuron, neburon, pentanochlor, phenmedipham, prometon, prometryn, propanil, propazine, pyridafol, pyridate, siduron, simazine, simetryn, tebuthiuron, terbacil, terbumeton, terbuthylazine, terbutryn and trietazine.

“AHAS inhibitors” (b2) are chemical compounds that inhibit acetohydroxy acid synthase (AHAS), also known as acetolactate synthase (ALS), and thus kill plants by inhibiting the production of the branched-chain aliphatic amino acids such as valine, leucine and isoleucine, which are required for protein synthesis and cell growth. Examples of AHAS inhibitors include amidosulfuron, azimsulfuron, bensulfuron-methyl, bispyribac-sodium, cloransulam-methyl, chlorimuron-ethyl, chlorsulfuron, cinosulfuron, cyclosulfamuron, diclosulam, ethametsulfuron-methyl, ethoxysulfuron, flazasulfuron, florasulam, flucarbazone-sodium, flumetsulam, flupyrsulfuron-methyl, flupyrsulfuron-sodium, foramsulfuron, halosulfuron-methyl, imazamethabenz-methyl, imazamox, imazapic, imazapyr, imazaquin, imazethapyr, imazosulfuron, iodosulfuron-methyl (including sodium salt), iofensulfuron (2-iodo-N-[[(4-methoxy-6-methyl-1,3,5-triazin-2-yl)amino]carbonyl]benzenesulfonamide), mesosulfuron-methyl, metazosulfuron (3-chloro-4-(5,6-dihydro-5-methyl-1,4,2-dioxazin-3-yl)-N-[[(4,6-dimethoxy-2-pyrimidinyl)amino]carbonyl]-1-methyl-1H-pyrazole-5-sulfonamide), metosulam, metsulfuron-methyl, nicosulfuron, oxasulfuron, penoxsulam, primisulfuron-methyl, propoxycarbazone-sodium, propyrisulfuron (2-chloro-N-[[(4,6-dimethoxy-2-pyrimidinyl)amino]carbonyl]-6-propylimidazo[1,2-b]pyridazine-3-sulfonamide), prosulfuron, pyrazosulfuron-ethyl, pyribenzoxim, pyriftalid, pyriminobac-methyl, pyrithiobac-sodium, rimsulfuron, sulfometuron-methyl, sulfosulfuron, thiencarbazone, thifensulfuron-methyl, triafamone (N-[2-[(4,6-dimethoxy-1,3,5-triazin-2-yl)carbonyl]-6-fluorophenyl]-1,1-difluoro-N-methylmethanesulfonamide), triasulfuron, tribenuron-methyl, trifloxysulfuron (including sodium salt), triflusulfuron-methyl and tritosulfuron.

“ACCase inhibitors” (b3) are chemical compounds that inhibit the acetyl-CoA carboxylase enzyme, which is responsible for catalyzing an early step in lipid and fatty acid synthesis in plants. Lipids are essential components of cell membranes, and without them, new cells cannot be produced. The inhibition of acetyl COA carboxylase and the subsequent lack of lipid production leads to losses in cell membrane integrity, especially in regions of active growth such as meristems. Eventually shoot and rhizome growth ceases, and shoot meristems and rhizome buds begin to die back. Examples of ACCase inhibitors include alloxydim, butroxydim, clethodim, clodinafop, cycloxydim, cyhalofop, diclofop, fenoxaprop, fluazifop, haloxyfop, pinoxaden, profoxydim, propaquizafop, quizalofop, sethoxydim, tepraloxydim and tralkoxydim, including resolved forms such as fenoxaprop-P, fluazifop-P, haloxyfop-P and quizalofop-P and ester forms such as clodinafop-propargyl, cyhalofop-butyl, diclofop-methyl and fenoxaprop-P-ethyl.

Auxin is a plant hormone that regulates growth in many plant tissues. “Auxin mimics” (b 4) are chemical compounds mimicking the plant growth hormone auxin, thus causing uncontrolled and disorganized growth leading to plant death in susceptible species. Examples of auxin mimics include aminocyclopyrachlor (6-amino-5-chloro-2-cyclopropyl-4-pyrimidinecarboxylic acid) and its methyl and ethyl esters and its sodium and potassium salts, 4-amino-3-chloro-5-fluoro-6-(7-fluoro-1H-indol-6-yl)-2-Pyridinecarboxylic 2-propyn-1-yl ester (CAS No. 2251111-17-6), 4-amino-3-chloro-5-fluoro-6-(7-fluoro-1H-indol-6-yl)-2-Pyridinecarboxylic cyanomethyl ester (CAS No. 2251111-18-7), aminopyralid, benazolin-ethyl, chloramben, clacyfos, clomeprop, clopyralid, dicamba, 2,4-D, 2,4-DB, dichlorprop, fluroxypyr, halauxifen (4-amino-3-chloro-6-(4-chloro-2-fluoro-3-methoxyphenyl)-2-pyridinecarboxylic acid), halauxifen-methyl (methyl 4-amino-3-chloro-6-(4-chloro-2-fluoro-3-methoxyphenyl)-2-pyridinecarboxylate), MCPA, MCPB, mecoprop, picloram, quinclorac, quinmerac, 2,3,6-TBA, triclopyr, and methyl 4-amino-3-chloro-6-(4-chloro-2-fluoro-3-methoxyphenyl)-5-fluoro-2-pyridinecarboxylate.

“EPSP synthase inhibitors” (b5) are chemical compounds that inhibit the enzyme, 5-enol-pyruvylshikimate-3-phosphate synthase, which is involved in the synthesis of aromatic amino acids such as tyrosine, tryptophan and phenylalanine. EPSP inhibitor herbicides are readily absorbed through plant foliage and translocated in the phloem to the growing points. Glyphosate is a relatively nonselective postemergence herbicide that belongs to this group. Glyphosate includes esters and salts such as ammonium, isopropylammonium, potassium, sodium (including sesquisodium) and trimesium (alternatively named sulfosate).

“Photosystem I electron diverters” (b6) are chemical compounds that accept electrons from Photosystem I, and after several cycles, generate hydroxyl radicals. These radicals are extremely reactive and readily destroy unsaturated lipids, including membrane fatty acids and chlorophyll. This destroys cell membrane integrity, so that cells and organelles “leak”, leading to rapid leaf wilting and desiccation, and eventually to plant death. Examples of this second type of photosynthesis inhibitor include diquat, paraquat and 1-(2-carboxyethyl)-4-(2-pyrimidinyl)pyridazinium (CAS No. 2285384-11-2).

“PPO inhibitors” (b7) are chemical compounds that inhibit the enzyme protoporphyrinogen oxidase, quickly resulting in formation of highly reactive compounds in plants that rupture cell membranes, causing cell fluids to leak out. Examples of PPO inhibitors include acifluorfen-sodium, azafenidin, benzfendizone, bifenox, butafenacil, carfentrazone, carfentrazone-ethyl, chlomethoxyfen, 3-[2-chloro-5-[3,6-dihydro-3-methyl-2,6-dioxo-4-(trifluoromethyl)-1(2H)-pyrimidinyl]-4-fluorophenyl]-4,5-dihydro-5-methyl-5-Isoxazolecarboxylic ethyl ester (CAS No. 1949837-17-5), cinidon-ethyl, fluazolate, flufenpyr-ethyl, flumiclorac-pentyl, flumioxazin, fluoroglycofen-ethyl, fluthiacet-methyl, fomesafen, halosafen, lactofen, oxadiargyl, oxadiazon, oxyfluorfen, pentoxazone, profluazol, pyraclonil, pyraflufen-ethyl, saflufenacil, sulfentrazone, thidiazimin, trifludimoxazin (dihydro-1,5-dimehyl-6-thioxo-3-[2,2,7-trifluoro-3,4-dihydro-3-oxo-4-(2-propyn-1-yl)-2H-1,4-benzoxazin-6-yl]-1,3,5-triazine-2,4(1H,3H)-dione) and tiafenacil (methyl N-[2-[[2-chloro-5-[3,6-dihydro-3-methyl-2,6-dioxo-4-(trifluoromethyl)-1(2H)-pyrimidinyl]-4-fluorophenyl]thio]-1-oxopropyl]-β-alaninate).

“GS inhibitors” (b8) are chemical compounds that inhibit the activity of the glutamine synthetase enzyme, which plants use to convert ammonia into glutamine. Consequently, ammonia accumulates and glutamine levels decrease. Plant damage probably occurs due to the combined effects of ammonia toxicity and deficiency of amino acids required for other metabolic processes. The GS inhibitors include glufosinate and its esters and salts such as glufosinate-ammonium and other phosphinothricin derivatives, glufosinate-P ((2S)-2-amino-4-(hydroxymethylphosphinyl) butanoic acid) and bilanaphos.

“VLCFA elongase inhibitors” (b9) are herbicides having a wide variety of chemical structures, which inhibit the elongase. Elongase is one of the enzymes located in or near chloroplasts which are involved in biosynthesis of VLCFAs. In plants, very-long-chain fatty acids are the main constituents of hydrophobic polymers that prevent desiccation at the leaf surface and provide stability to pollen grains. Such herbicides include acetochlor, alachlor, anilofos, butachlor, cafenstrole, dimethachlor, dimethenamid, diphenamid, fenoxasulfone (3-[[(2,5-dichloro-4-ethoxyphenyl)methyl]sulfonyl]-4,5-dihydro-5,5-dimethylisoxazole), fentrazamide, flufenacet, indanofan, mefenacet, metazachlor, metolachlor, naproanilide, napropamide, napropamide-M ((2R)—N,N-diethyl-2-(1-naphthalenyloxy) propanamide), pethoxamid, piperophos, pretilachlor, propachlor, propisochlor, pyroxasulfone, and thenylchlor, including resolved forms such as S-metolachlor and chloroacetamides and oxyacetamides.

“Auxin transport inhibitors” (b10) are chemical substances that inhibit auxin transport in plants, such as by binding with an auxin-carrier protein. Examples of auxin transport inhibitors include diflufenzopyr, naptalam (also known as N-(1-naphthyl) phthalamic acid and 2-[(1-naphthalenylamino)carbonyl]benzoic acid).

“PDS inhibitors” (b11) are chemical compounds that inhibit carotenoid biosynthesis pathway at the phytoene desaturase step. Examples of PDS inhibitors include beflubutamid, diflufenican, fluridone, flurochloridone, flurtamone norflurzon and picolinafen.

“HPPD inhibitors” (b12) are chemical substances that inhibit the biosynthesis of synthesis of 4-hydroxyphenyl-pyruvate dioxygenase. Examples of HPPD inhibitors include benzobicyclon, benzofenap, bicyclopyrone (4-hydroxy-3-[[2-[(2-methoxyethoxy)methyl]-6-(trifluoromethyl)-3-pyridinyl]carbonyl]bicyclo[3.2.1]oct-3-en-2-one), fenquinotrione (2-[[8-chloro-3,4-dihydro-4-(4-methoxyphenyl)-3-oxo-2-quinoxalinyl]carbonyl]-1,3-cyclohexanedione), flusulfinam, iptriazopyrid, isoxachlortole, isoxaflutole, mesotrione, pyrasulfotole, pyrazolynate, pyrazoxyfen, sulcotrione, tefuryltrione, tembotrione, tolpyralate (1-[[1-ethyl-4-[3-(2-methoxyethoxy)-2-methyl-4-(methylsulfonyl)benzoyl]-1H-pyrazol-5-yl]oxy]ethyl methyl carbonate), topramezone, 5-chloro-3-[(2-hydroxy-6-oxo-1-cyclohexen-1-yl)carbonyl]-1-(4-methoxyphenyl)-2(1H)-quinoxalinone, 4-(2,6-diethyl-4-methylphenyl)-5-hydroxy-2,6-dimethyl-3(2H)-pyridazinone, 4-(4-fluorophenyl)-6-[(2-hydroxy-6-oxo-1-cyclohexen-1-yl)carbonyl]-2-methyl-1,2,4-triazine-3,5(2H,4H)-dione, 5-[(2-hydroxy-6-oxo-1-cyclohexen-1-yl)carbonyl]-2-(3-methoxyphenyl)-3-(3-methoxypropyl)-4(3H)-pyrimidinone, 2-methyl-N-(4-methyl-1,2,5-oxadiazol-3-yl)-3-(methylsulfinyl)-4-(trifluoromethyl)benzamide and 2-methyl-3-(methylsulfonyl)-N-(1-methyl-1H-tetrazol-5-yl)-4-(trifluoromethyl)benzamide.

“HST (homogentisate solanesyltransferase) inhibitors” (b13) disrupt a plant's ability to convert homogentisate to 2-methyl-6-solanyl-1,4-benzoquinone, thereby disrupting carotenoid biosynthesis. Examples of HST inhibitors include cyclopyrimorate (6-chloro-3-(2-cyclopropyl-6-methylphenoxy)-4-pyridazinyl 4-morpholinecarboxylate), haloxydine, pyriclor, 3-(2-chloro-3,6-difluorophenyl)-4-hydroxy-1-methyl-1,5-naphthyridin-2(1H)-one, 7-(3,5-dichloro-4-pyridinyl)-5-(2,2-difluoroethyl)-8-hydroxypyrido[2,3-b]pyrazin-6(5H)-one and 4-(2,6-diethyl-4-methylphenyl)-5-hydroxy-2,6-dimethyl-3(2H)-pyridazinone.

HST inhibitors also include compounds of Formulae A and B.

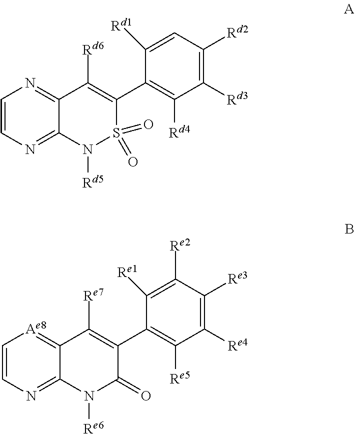

wherein R^{d1} is H, Cl or CF₃; R^{d2} is H, Cl or Br; R^{d3} is H or Cl; R^{d4} is H, Cl or CF₃; R^{d5} is CH₃, CH₂CH₃ or CH₂CHF₂; and R^{d6} is OH, or —OC(═O)-i-Pr; and R^{e1} is H, F, Cl, CH₃ or CH₂CH₃; R^{e2} is H or CF₃; R^{e3} is H, CH₃ or CH₂CH₃; R^{e4} is H, F or Br; R^{e5} is Cl, CH₃, CF₃, OCF₃ or CH₂CH₃; R^{e6} is H, CH₃, CH₂CHF₂ or C≡CH; R^{e7} is OH, —OC(═O)Et, —OC(═O)-i-Pr or —OC(═O)-t-Bu; and A^{e8} is N or CH.

“Cellulose biosynthesis inhibitors” (b14) inhibit the biosynthesis of cellulose in certain plants. They are most effective when applied preemergence or early postemergence on young or rapidly growing plants. Examples of cellulose biosynthesis inhibitors include chlorthiamid, dichlobenil, flupoxam, indaziflam (N²-[(1R,2S)-2,3-dihydro-2,6-dimethyl-1H-inden-1-yl]-6-(1-fluoroethyl)-1,3,5-triazine-2,4-diamine), isoxaben and triaziflam.

“DHODH (dihydroorotate dehydrogenase) inhibitors” (b15) act through inhibiting catalysis of the fourth step of pyrimidine biosynthesis in plant systems. Inhibition of pyrimidine biosynthesis leads to the cessation of plant growth. Examples of DOHDH inhibitors include tetflupyrolimet ((3S,4S)—N-(2-fluorophenyl)-1-methyl-2-oxo-4-[3-(trifluoromethyl)phenyl]-3-pyrrolidinecarboxamide) and (3S,4R)—N-(2,3-difluorophenyl)-1-methyl-4-[1-methyl-5-(trifluoromethyl)-1H-pyrazol-3-yl]-2-oxo-3-pyrrolidinecaboxamide.

“Other herbicides” (b16) include herbicides that act through a variety of different modes of action such as mitotic disruptors (e.g., flamprop-M-methyl and flamprop-M-isopropyl), organic arsenicals (e.g., DSMA, and MSMA), 7,8-dihydropteroate synthase inhibitors, chloroplast isoprenoid synthesis inhibitors and cell-wall biosynthesis inhibitors. Other herbicides include those herbicides having unknown modes of action or do not fall into a specific category listed in (b1) through (b14) or act through a combination of modes of action listed above. Examples of other herbicides include aclonifen, asulam, amitrole, bixlozone, broclozone, bromobutide, cinmethylin, clomazone, cumyluron, daimuron, difenzoquat, dimesulfazet, epyrifenacil, etobenzanid, fluometuron, flurenol, fosamine, fosamine-ammonium, dazomet, dymron, ipfencarbazone (1-(2,4-dichlorophenyl)-N-(2,4-difluorophenyl)-1,5-dihydro-N-(1-methylethyl)-5-oxo-4H-1,2,4-triazole-4-carboxamide), metam, methyldymron, oleic acid, oxaziclomefone, pelargonic acid, pyributicarb, 2,5-anhydro-3,4-dideoxy-4-[[[(5S)-3-(3,5-difluorophenyl)-5-ethenyl-4,5-dihydro-5-isoxazolyl]carbonyl]amino]-threo-Pentonic methyl ester (CAS No. 27499989-21-6) and 5-[[(2,6-difluorophenyl) methoxy]methyl]-4,5-dihydro-5-methyl-3-(3-methyl-2-thienyl) isoxazole. “Other herbicides” (b16) also include a compound of Formula (b16A)

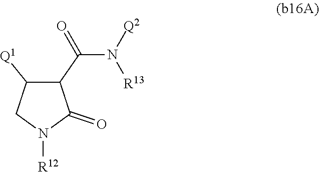

wherein
R¹² is H, C₁-C₆ alkyl, C₁-C₆ haloalkyl or C₄-C₈ cycloalkyl;
R¹³ is H, C₁-C₆ alkyl or C₁-C₆ alkoxy;
Q¹ is an optionally substituted ring system selected from the group consisting of phenyl, thienyl, pyridinyl, benzodioxolyl, naphthyl, naphthalenyl, benzofuranyl, furanyl, benzothiophenyl and pyrazolyl, wherein when substituted said ring system is substituted by 1 to 3 R¹⁴;
Q² is an optionally substituted ring system selected from the group consisting of phenyl, pyridinyl, benzodioxolyl, pyridinonyl, thiadiazolyl, thiazolyl, and oxazolyl, wherein when substituted said ring system is substituted by 1 to 3 R¹⁵;
each R¹⁴ is independently halogen, C₁-C₆ alkyl, C₁-C₆ haloalkyl, C₁-C₆ alkoxy, C₁-C₆ haloalkoxy, C₃-C₈ cyaloalkyl, cyano, C₁-C₆ alkylthio, C₁-C₆ alkylsulfinyl, C₁-C₆ alkylsulfonyl, SF₅, NHR¹⁷; or phenyl optionally substituted by 1 to 3 R¹⁶; or pyrazolyl optionally substituted by 1 to 3 R¹⁶;
each R¹⁵ is independently halogen, C₁-C₆ alkyl, C₁-C₆ haloalkyl, C₁-C₆ alkoxy, C₁-C₆ haloalkoxy, cyano, nitro, C₁-C₆ alkylthio, C₁-C₆ alkylsulfinyl, C₁-C₆ alkylsulfonyl;
each R¹⁶ is independently halogen, C₁-C₆ alkyl or C₁-C₆ haloalkyl; R¹⁷ is C₁-C₄ alkoxycarbonyl.

In one Embodiment wherein “other herbicides” (b16) also include a compound of Formula (b16A), it is preferred that R¹² is H or C₁-C₆ alkyl; more preferably R¹² is H or methyl. Preferably R¹³ is H. Preferably Q¹ is either a phenyl ring or a pyridinyl ring, each ring substituted by 1 to 3 R¹⁴; more preferably Q¹ is a phenyl ring substituted by 1 to 2 R¹⁴. Preferably Q² is a phenyl ring substituted by 1 to 3 R¹⁵; more preferably Q² is a phenyl ring substituted by 1 to 2 R¹⁵. Preferably each R¹⁴ is independently halogen, C₁-C₄ alkyl, C₁-C₃ haloalkyl, C₁-C₃ alkoxy or C₁-C₃ haloalkoxy; more preferably each R¹⁴ is independently chloro, fluoro, bromo, C₁-C₂ haloalkyl, C₁-C₂ haloalkoxy or C₁-C₂ alkoxy. Preferrably each R¹⁵ is independently halogen, C₁-C₄ alkyl, C₁-C₃ haloalkoxy; more preferably each R¹⁵ is independently chloro, fluoro, bromo, C₁-C₂ haloalkyl, C₁-C₂ haloalkoxy or C₁-C₂ alkoxy. Specifically preferred as “other herbicides” (b16) include any one of the following (b16A-1) through (b16A-15):

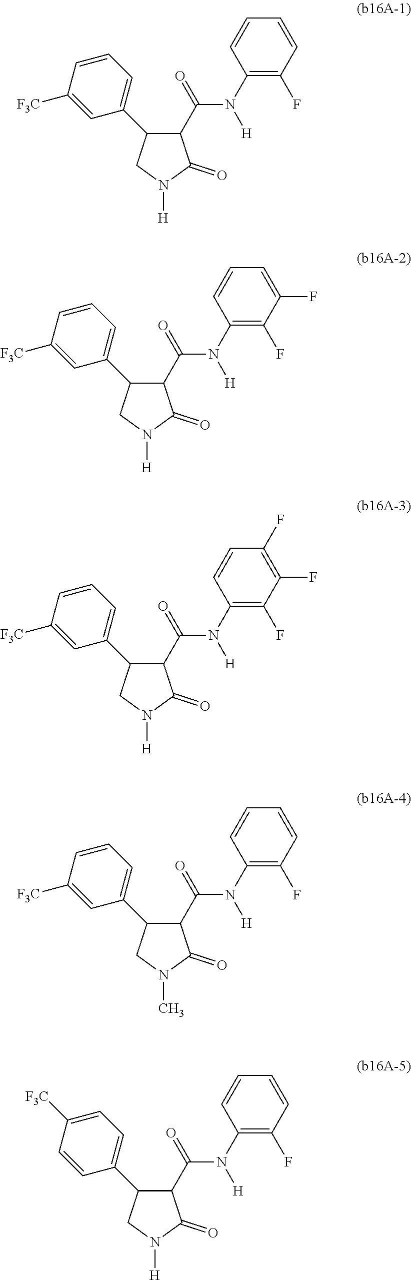 [SMILES: O=C1NCC(c2cccc(C(F)(F)F)c2)C1C(=O)Nc1ccccc1F ; O=C1NCC(c2cccc(C(F)(F)F)c2)C1C(=O)Nc1cccc(F)c1F ; O=C1NCC(c2cccc(C(F)(F)F)c2)C1C(=O)Nc1ccc(F)c(F)c1F ; CN1CC(c2cccc(C(F)(F)F)c2)C(C(=O)Nc2ccccc2F)C1=O ; O=C1NCC(c2ccc(C(F)(F)F)cc2)C1C(=O)Nc1ccccc1F]

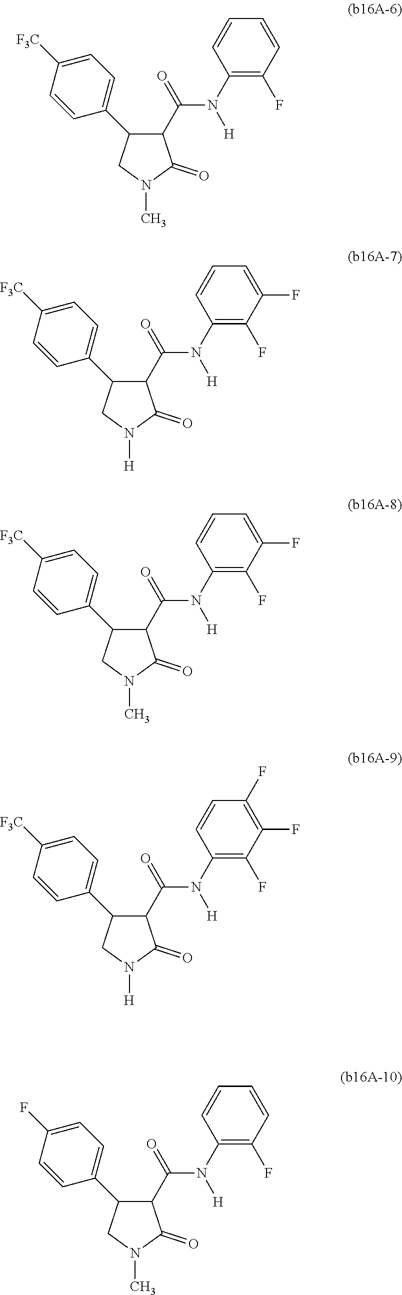 [SMILES: CN1CC(c2ccc(C(F)(F)F)cc2)C(C(=O)Nc2ccccc2F)C1=O ; O=C1NCC(c2ccc(C(F)(F)F)cc2)C1C(=O)Nc1cccc(F)c1F ; CN1CC(c2ccc(C(F)(F)F)cc2)C(C(=O)Nc2cccc(F)c2F)C1=O ; O=C1NCC(c2ccc(C(F)(F)F)cc2)C1C(=O)Nc1ccc(F)c(F)c1F ; CN1CC(c2ccc(F)cc2)C(C(=O)Nc2ccccc2F)C1=O]

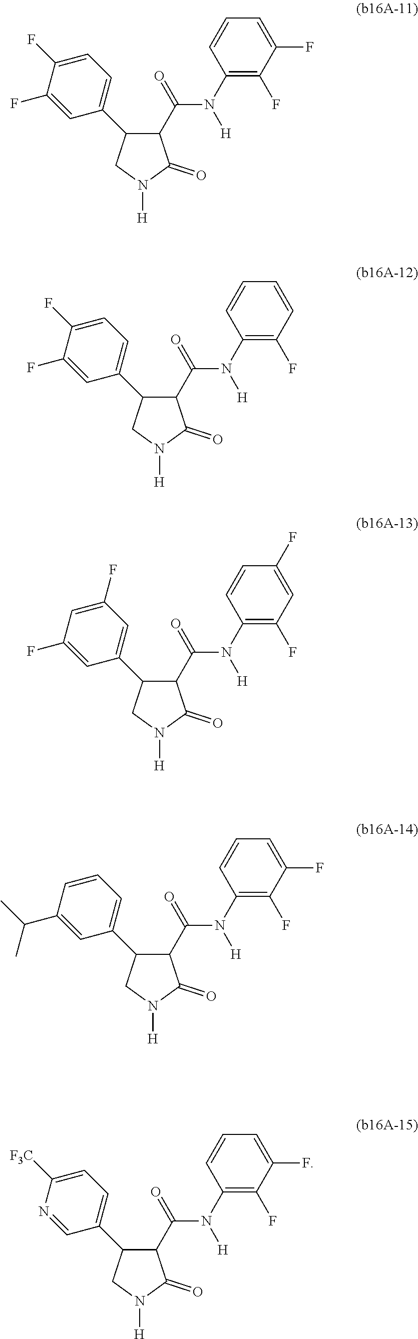 [SMILES: O=C1NCC(c2ccc(F)c(F)c2)C1C(=O)Nc1cccc(F)c1F ; O=C1NCC(c2ccc(F)c(F)c2)C1C(=O)Nc1ccccc1F ; O=C1NCC(c2cc(F)cc(F)c2)C1C(=O)Nc1ccc(F)cc1F ; CC(C)c1cccc(C2CNC(=O)C2C(=O)Nc2cccc(F)c2F)c1 ; O=C1NCC(c2ccc(C(F)(F)F)nc2)C1C(=O)Nc1cccc(F)c1F]

Another Embodiment wherein “other herbicides” (b16) also include a compound of Formula (b16C),

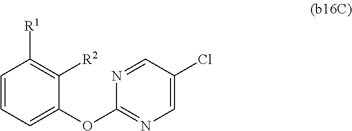

wherein R¹ is Cl, Br or CN; and R² is C(═O) CH₂CH₂CF₃, CH₂CH₂CH₂CH₂CF₃ or 3-CHF₂-isoxazol-5-yl.

“Herbicide safeners” (b17) are substances added to a herbicide formulation to eliminate or reduce phytotoxic effects of the herbicide to certain crops. These compounds protect crops from injury by herbicides but typically do not prevent the herbicide from controlling undesired vegetation. Examples of herbicide safeners include but are not limited to benoxacor, cloquintocet-mexyl, cumyluron, cyometrinil, cyprosulfamide, daimuron, dichlormid, dicyclonon, dietholate, dimepiperate, fenchlorazole-ethyl, fenclorim, flurazole, fluxofenim, furilazole, isoxadifen-ethyl, mefenpyr-diethyl, mephenate, methoxyphenone, naphthalic anhydride, oxabetrinil, N-(aminocarbonyl)-2-methylbenzenesulfonamide and N-(aminocarbonyl)-2-fluorobenzenesulfonamide, 1-bromo-4-[(chloromethyl) sulfonyl]benzene, 2-(dichloromethyl)-2-methyl-1,3-dioxolane (MG 191), 4-(dichloroacetyl)-1-oxa-4-azospiro[4.5]decane (MON 4660), 2,2-dichloro-1-(2,2,5-trimethyl-3-oxazolidinyl)-ethanone and 2-methoxy-N-[[4-[[(methylamino)carbonyl]amino]phenyl]sulfonyl]-benzamide.

Preferred for better control of undesired vegetation (e.g., lower use rate such as from greater-than-additive effects, broader spectrum of weeds controlled, or enhanced crop safety) or for preventing the development of resistant weeds are mixtures of a compound of this invention with a herbicide selected from the group consisting of 4-amino-3-chloro-5-fluoro-6-(7-fluoro-1H-indol-6-yl)-2-Pyridinecarboxylic 2-propyn-1-yl ester (CAS No. 2251111-17-6), 4-amino-3-chloro-5-fluoro-6-(7-fluoro-1H-indol-6-yl)-2-Pyridinecarboxylic cyanomethyl ester (CAS No. 2251111-18-7), 2,5-anhydro-3,4-dideoxy-4-[[[(5S)-3-(3,5-difluorophenyl)-5-ethenyl-4,5-dihydro-5-isoxazolyl]carbonyl]amino]-threo-Pentonic methyl ester (CAS No. 27499989-21-6), atrazine, azimsulfuron, beflubutamid, beflubutamid-M, bixlozone, broclozone, benzisothiazolinone, 1-(2-carboxyethyl)-4-(2-pyrimidinyl)pyridazinium (CAS No. 2285384-11-2) and salts thereof, carfentrazone-ethyl, chlorimuron-ethyl, 3-[2-chloro-5-[3,6-dihydro-3-methyl-2,6-dioxo-4-(trifluoromethyl)-1(2H)-pyrimidinyl]-4-fluorophenyl]-4,5-dihydro-5-methyl-5-isoxazolecarboxylic ethyl ester (CAS No. 1949837-17-5), chlorsulfuron-methyl, clomazone, clopyralid potassium, cloransulam-methyl, 2-[(2,4-dichlorophenyl)methyl]-4,4-dimethyl-isoxazolidinone, 2-[(2,5-dichlorophenyl)methyl]-4,4-dimethyl-isoxazolidinone, ethametsulfuron-methyl, flumetsulam, 4-(4-fluorophenyl)-6-[(2-hydroxy-6-oxo-1-cyclohexen-1-yl)carbonyl]-2-methyl-1,2,4-triazine-3,5-(2H,4H)-dione, flupyrsulfuron-methyl, fluthiacet-methyl, fomesafen, imazethapyr, lenacil, mesotrione, metribuzin, metsulfuron-methyl, pethoxamid, picloram, pyroxasulfone, quinclorac, rimsulfuron, S-metolachlor, sulfentrazone, thifensulfuron-methyl, triflusulfuron-methyl and tribenuron-methyl.

One or more of the following methods and variations as described in Schemes 1-5 can be used to prepare the compounds of Formula 1. The definitions of R, R¹, R², R³, R⁴, R⁵, X, V, W, Z, and n in the compounds of Formulae 1-8 below are as defined above in the Summary of the Invention unless otherwise noted. Compound of Formulae 1a is a subset of the compound of Formula 1 and the substituents for Formula 1a are as defined above for Formula 1 unless otherwise noted in the disclosure including the schemes.

As shown in Scheme 1, a compound of Formula 1a (i.e. a compound of Formula 1 wherein V is O) can be prepared by reaction of acids of Formula 2 with an amine of Formula 3 in the presence of a dehydrative coupling reagent such as propylphosphonic anhydride, dicyclohexylcarbodiimide, N-(3-dimethylaminopropyl)-N′-ethylcarbodiimide, 1-[Bis(dimethylamino)methylene]-1H-1,2,3-triazolo[4,5-b]pyridinium 3-oxide hexafluorophosphate (HATU), benzotriazol-1-yloxytripyrrolidinophosphonium hexafluorophosphate (PyBOP), N,N′-carbonyldiimidazole, 2-chloro-1,3-dimethylimidazolium chloride or 2-chloro-1-methylpyridinium iodide etc. These reactions are typically run at temperatures ranging from 0-60° C. in a solvent such as dichloromethane, acetonitrile, N,N-dimethylformamide or ethyl acetate in the presence of a base such as triethylamine, N,N-diisopropylamine, or 1,8-diazabicyclo[5.4.0]undec-7-ene. See Org. Process Res. Dev. 2016, 20, 2, 140-177 for amide coupling conditions.

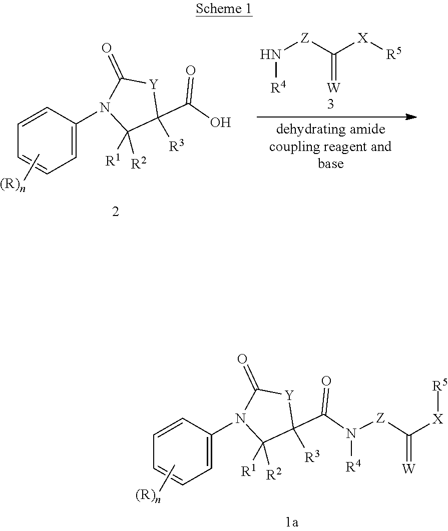

As shown in Scheme 2, compounds of Formula 2 (where R′ is lower alkyl) can be prepared by hydrolysis of esters of Formula 4 under mild conditions. Hydrolysis is carried out with selective mild base, typically in the presence of a solvent. Suitable bases for the reaction include, but are not limited to, potassium trimethylsilanoate or trimethyltinoxide. A wide variety of co-solvents are suitable for the reaction including, but not limited to, methanol, ethanol and tetrahydrofuran. The reaction is conducted at temperatures ranging from −20° C. to the boiling point of the solvent, and typically from 0 to 100° C. For detail procedures, see Tetrahedron letter, 1984, 25, 51, 5831-5834 or Angewandte Chemie, 2005, 44, 9, 1378-1382.

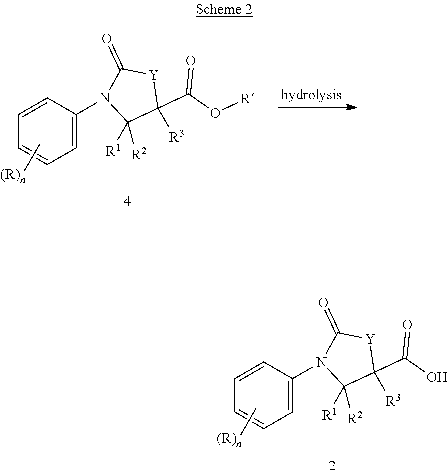

As shown in Scheme 3, compounds of Formula 4 (where R′ is lower alkyl) can be prepared from β-amino alcohol of Formula 5 by treatment with carbonylating reagent such as N,N′-carbonyldiimidazole (CDI), triphosgene, or dialkylcarbonate, typically in the presence of a base and a suitable co-solvent. Suitable organic bases for this reaction include, but are not limited to, piperidine, morpholine, triethylamine, 4-methylmorpholine or N,N-diisopropylethylamine. This transformation can be accomplished neat or in solvents such as tetrahydrofuran, toluene or dichloromethane. Typically, the reaction is carried out in the range of from 0° C. to 80° C. For detail procedures, see Tetrahedron, 2020, 76, 47, 131553 and references therein.

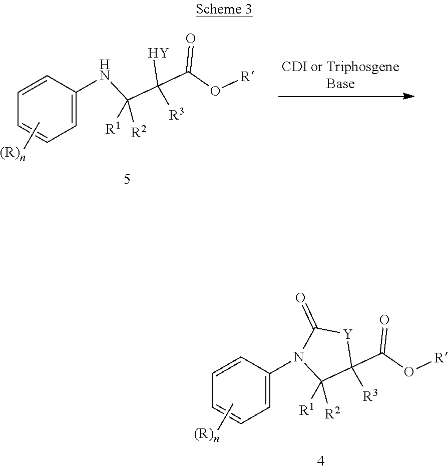

As shown in Scheme 4, β-amino alcohol a compound of Formula 5a (i.e. a compound of Formula 5 wherein Y is O) can be synthesized from ring opening of epoxide of Formula 6 by a suitable aniline. Regio selective ring opening can be achieved by either simply refluxing the epoxide of Formula 6 with aniline in suitable solvent such as ethanol or by treating in presence of indium halide (InCl₃ or InBr₃) in anhydrous dichloromethane at room temperature. Epoxide of formula 6 are either commercially available or can be prepared as method described in the literature procedures in US20040044249 or WO2020102816. Anilines can be purchased commercially. Conditions for indium halide promoted epoxide opening reactions can be found in New Journal of Chemistry, 2001, 25(2), 221-222; Tetrahedron Letters, 2004, 45, 7495-7498.

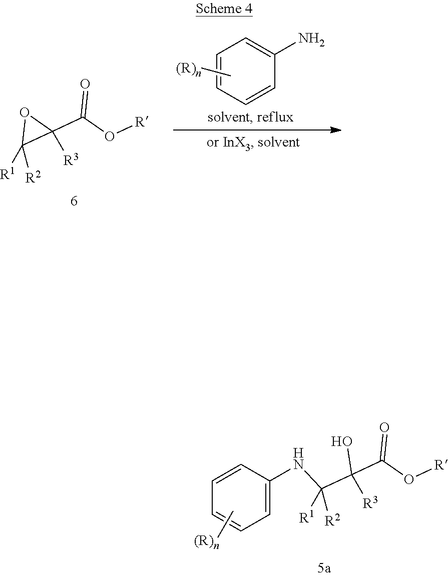

As shown in Scheme 4A, a compound of Formula 5b (i.e. a compound of Formula 5 wherein Y is NH) can be synthesized by reduction of the nitro compound of Formula 7. Suitable reducing agents include, but are not limited to, zinc in acetic acid or Raney-Ni in ethanol. Conditions for the reactions can be found in Chemitsry—An Asian Journal, 2013, 8(5), 877-882; Organic & Biomolecular Chemistry, 2005, 3(8), 1362-1364.

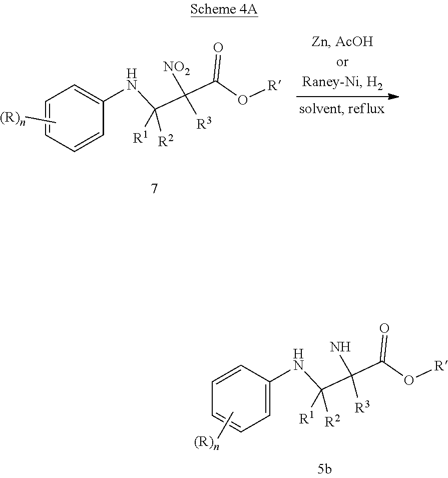

As shown in Scheme 4B, the nitro compound of Formula 7 can be prepared by an aminomethylation reaction from the corresponding nitroacetate of Formula 9 and a carbonyl compound of Formula 8. Conditions for the reactions of this type can be Chemitsry—An Asian Journal, 2013, 8(5), 877-882.

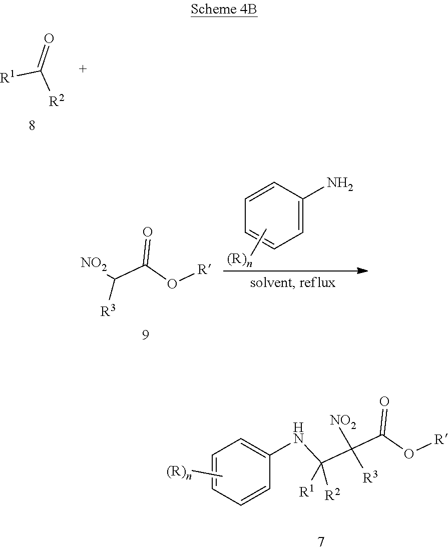

As shown in the Scheme 5, amines of Formula 3 can be prepared by reaction of acids of Formula 7 with an amine, alcohol or thiol of Formula 8 in the presence of a dehydrative coupling reagent such as oxalyl chloride, thionyl chloride, propylphosphonic anhydride, dicyclohexylcarbodiimide, N-(3-dimethylaminopropyl)-N′-ethylcarbodiimide, 1-[Bis(dimethylamino)methylene]-1H-1,2,3-triazolo[4,5-b]pyridinium 3-oxide hexafluorophosphate (HATU), benzotriazol-1-yloxytripyrrolidinophosphonium hexafluorophosphate (PyBOP), N,N′-carbonyldiimidazole, 2-chloro-1,3-dimethylimidazolium chloride or 2-chloro-1-methylpyridinium iodide. These reactions are typically run at temperatures ranging from 0-60° C. in a solvent such as dichloromethane, acetonitrile, N,N-dimethylformamide or ethyl acetate in the presence of a base such as triethylamine, N,N-diisopropylamine, or 1,8-diazabicyclo[5.4.0]undec-7-ene. Specific examples of synthesis can be found in Journal of Peptide Science, 2008, 14(2), 241-249. Amines of the Formula 3 wherein X is O are also either commercially available or can be prepared from commercially available Vince lactam, by alkylation as described in Org. Process Res. Dev. 2018, 22, 337-343, WO2017133667, Org. Lett. 2017, 19, 7, 1602-1605 or from commercially available alcohols using thionyl chloride as described in Tetrahedron Lett. 2001, 42, 1347-1350. Amines containing a tetrahydrofuran ring can be prepared by the method described in WO2021170464. Amine containing cyclobutanes can be purchased from commercially available vendors. Amine containing bicyclic substitutions can be made by the procedures described in WO2017133669 or Tetrahedron, 2010, 66, 3599-3607. N-Boc protection can then be subsequently removed by acid treatment to give the desired chiral amine of Formula 3 as the corresponding salt form. Acids used in this reaction include trifluoracetic acid or any other inorganic acids.

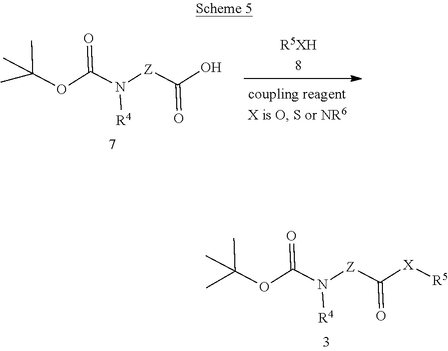

It is recognized by one skilled in the art that various functional groups can be converted into others to provide different compounds of Formula 1. For a valuable resource that illustrates the interconversion of functional groups in a simple and straightforward fashion, see Larock, R. C., Comprehensive Organic Transformations: A Guide to Functional Group Preparations, 2^{nd} Ed., Wiley-VCH, New York, 1999.

For example, intermediates for the preparation of a compound of Formula 1 may contain aromatic nitro groups, which can be reduced to amino groups, and then be converted via reactions well known in the art such as the Sandmeyer reaction, to various halides, providing a compound of Formula 1. The above reactions can also in many cases be performed in alternate order.

For example, derivatives of the Formula 1, where Rⁿ is halogen, in particular iodine or bromine, can be reacted with alkene, acetylenes, phenyl, 5- or 6-membered heteroaryl, with transition metal catalysis, e.g. with specifically of palladium (0) or a palladium (II) catalyst, in an appropriate solvent in presence of suitable base at temperatures between 20° C. and 150° C. to give compounds of the Formula 1 wherein Rⁿ is substituted or unsubstituted alkene, alkyne, phenyl, 5- or 6-membered heteroaryl etc. Compounds of Formula 1, wherein Rⁿ is CN, can be hydrolyzed under acidic or basic conditions to give carboxylic acids prepared that can be subsequently transformed into acid chlorides and, in turn, these can be converted into amides, by simple organic transformations. Derivatives of Formula 1 wherein Rⁿ is halogen can also be converted in to corresponding alkoxyalkyl or aminoalkyl or diaminoalkyl substituted compounds through treatment with a suitable alcohol or amine in an appropriate solvent in presence of suitable base at temperatures between 0° C. to 150° C.

It is recognized that some reagents and reaction conditions described above for preparing a compound of Formula 1 may not be compatible with certain functionalities present in the intermediates. In these instances, the incorporation of protection/deprotection sequences or functional group interconversions into the synthesis will aid in obtaining the desired products. The use and choice of the protecting groups will be apparent to one skilled in chemical synthesis (see, for example, Greene, T. W.; Wuts, P. G. M. Protective Groups in Organic Synthesis, 4th ed.; Wiley: Hoboken, New Jersey, 1991. One skilled in the art will recognize that, in some cases, after the introduction of a given reagent as it is depicted in any individual scheme, it may be necessary to perform additional routine synthetic steps not described in detail to complete the synthesis of a compound of Formula 1. One skilled in the art will also recognize that it may be necessary to perform a combination of the steps illustrated in the above schemes in an order other than that implied by the particular presented to prepare a compound of Formula 1.

One skilled in the art will also recognize that a compound of Formula 1 and the intermediates described herein can be subjected to various electrophilic, nucleophilic, radical, organometallic, oxidation, and reduction reactions to add substituents or modify existing substituents.

Without further elaboration, it is believed that one skilled in the art using the preceding description can utilize the present invention to its fullest extent. The following Examples are, therefore, to be construed as merely illustrative, and not limiting of the disclosure in any way whatsoever. Steps in the following Examples illustrate a procedure for each step in an overall synthetic transformation, and the starting material for each step may not have necessarily been prepared by a particular preparative run whose procedure is described in other examples or steps. Percentages are by weight except for chromatographic solvent mixtures or where otherwise indicated. Parts and percentages for chromatographic solvent mixtures are by volume unless otherwise indicated. All NMR spectra are reported in ppm downfield from tetramethylsilane in CDCl₃ at 500 MHz unless otherwise indicated where s means singlet, brs means broad singlet, d means doublet, t means triplet, q means quarte, p means pentet and m means multiplet.

### Synthesis Example 1

### Preparation of Methyl (1S,4R)-4-[[[3-(3,5-difluorophenyl)-5-methyl-2-oxo-5-oxazolidinyl]carbonyl]amino]-2-cyclopentene-1-carboxylate (Compound 27)

### Step A: Preparation of methyl 3-(3,5-difluoroanilino)-2-hydroxy-2-methyl-propanoate

To a solution of commercially available methyl 2-methyloxirane-2-carboxylate (1 g, 8.612 mmol, 1 equiv.) in dichloromethane (20 mL) at room temperature, 3,5-difluoroaniline (1.112 g, 8.612 mmol, 1 equiv.) was added and the reaction mixture was stirred for 10 min. Indium chloride (InCl₃) (0.952 g, 4.306 mmol, 0.5 equiv.) was added to the reaction mixture portion wise. After completion of addition, the reaction mixture was stirred at room temperature for 14 h. After completion of the reaction, the reaction mixture was quenched with ice cold water and extracted with dichloromethane (2×100 mL) and organic layer was washed with water and brine solution. The combined organic layers were dried over anhydrous sodium sulphate and concentrated. The crude was purified by column chromatography on silica gel by eluting with ethyl acetate/petrleum ether (0-15%) to afford desired methyl 3-(3,5-difluoroanilino)-2-hydroxy-2-methyl-propanoate (1.2 g, 50% yield) was isolated as an off-white solid.

¹H NMR δ 1.46 (s, 3H), 3.19-3.22 (dd, 1H), 3.45 (s, 1H), 3.49-3.53 (dd, 1H) 3.77 (s, 3H) 4.24 (bs, 1H), 6.09-6.17 (m, 3H).

### Step B: Synthesis of methyl 3-(3,5-difluorophenyl)-5-methyl-2-oxo-oxazolidine-5-carboxylate

To a solution of methyl 3-(3,5-difluoroanilino)-2-hydroxy-2-methyl-propanoate (i.e. the product of Step A) (1.5 g, 6.117 mmol, 1 equiv.) in dichloromethane (20 mL) at 0° C., triethylamine (0.853 mL, 6.117 mmol, 1 equiv.) and a solution of triphosgene (1.815 g, 6.117 mmol, 1 equiv.) in dichloromethane (5 mL) were added respectively in dropwise fashion. After completion of addition, the reaction mixture was slowly warmed to room temperature and stirred for another 14 h. After completion of the reaction, the reaction mixture was quenched with ice cold water and extracted with dichloromethane (2×100 mL). The combined organic layers were washed with water and brine solution, dried over anhydrous sodium sulphate and concentrated. The crude material was purified by column chromatography on silica gel by eluting with ethyl acetate/petroleum ether (0-30%) to afford the desired methyl 3-(3,5-difluorophenyl)-5-methyl-2-oxo-oxazolidine-5-carboxylate (1.01 g, 91% yield) was isolated as a white solid.

¹H NMR δ ppm 1.79 (s, 3H), 3.80-3.82 (d, 1H), 3.86 (s, 3H), 4.29-4.31 (d, 1H), 6.59-6.63 (m, 1H), 7.11-7.16 (m, 2H).

### Step C: Preparation of 3-(3,5-difluorophenyl)-5-methyl-2-oxo-oxazolidine-5-carboxylic acid

To a solution of methyl 3-(3,5-difluorophenyl)-5-methyl-2-oxo-1,3-oxazolidine-5-carboxylate (i.e. the product of Step B) (1 g, 3.687 mmol, 1 equiv.) in tetrahydrofuran (10 mL) at 0° C., potassium trimethylsilanolate (473 mg, 3.687 mmol, 1 equiv.) was added and reaction was stirred for 1 h. The solution was quenched with 2 N HCl and extracted with ethyl acetate (2×100 mL). The combined organic layer was washed with water and brine solution and dried over anhydrous sodium sulphate and concentrated. The crude 3-(3,5-difluorophenyl)-5-methyl-2-oxo-1,3-oxazolidine-5-carboxylic acid (650 mg, 68.5% yield) was pure enough to use in the next step.

¹H NMR (500 MHz, DMSO-d6) δ 1.64 (m, 3H), 3.99-4.02 (d, 2H), 4.28-4.30 (d, 1H), 6.96-6.99 (m, 1H), 7.30-7.33 (m, 2H), 13.78 (bs, 1H).

### Step D: Preparation of Methyl (1S,4R)-4-[[[3-(3,5-difluorophenyl)-5-methyl-2-oxo-5-oxazolidinyl]carbonyl]amino]-2-cyclopentene-1-carboxylate

To a stirred solution of 3-(3,5-difluorophenyl)-5-methyl-2-oxo-1,3-oxazolidine-5-carboxylic acid (i.e. the product of Step C) (5 g, 19.441 mmol, 1 equiv.), methyl (1S,4R)-4-aminocyclopent-2-ene-1-carboxylate hydrochloride (4.144 g, 23.329 mmol, 1.2 equiv.), prepared according to the procedure described in WO2017133667, in dichloromethane (100 mL) N,N-diisopropylethylamine (6.791 mL, 38.881 mmol, 2 equiv.), PyBOP (20.234 g, 38.881 mmol, 2 equiv.) were added respectively at room temperature. The reaction mixture was stirred for 16 hr at room temperature. After the completion of the reaction, the reaction mixture was diluted with water (50 mL), extracted with dichloromethane (2×100 mL). The combined organic layer was dried over anhydrous sodium sulphate, filtered and concentrated under reduced pressure to get crude residue. The crude product was purified by column chromatography on silica gel and eluting with ethyl acetate/petether (0-50%) to afford Methyl (1S,4R)-4-[[[3-(3,5-difluorophenyl)-5-methyl-2-oxo-5-oxazolidinyl]carbonyl]amino]-2-cyclopentene-1-carboxylate (6 g, yield 81.1%) as a diastereomeric mixture.

¹H NMR δ ppm 1.74-1.76 (s, 3H), 1.93-1.99 (m, 1H), 2.44-2.54 (m, 1H), 3.53-3.56 (m, 1H), 3.75 (m, 3H), 3.80-3.81 (m, 1H), 4.33-4.35 (m, 1H), 5.01-5.05 (m, 1H), 5.85-6.00 (m, 2H), 6.59-6.63 (m, 1H), 7.08-7.16 (m, 3H).

### Synthesis Example 2

### Preparation of (1S,4R)-4-[[[3-(3,5-Difluorophenyl)-5-methyl-2-oxo-5-oxazolidinyl]carbonyl]amino]-2-cyclopentene-1-carboxylic acid (Compound 51)

### Step A: Preparation of (1S,4R)-4-[[[3-(3,5-Difluorophenyl)-5-methyl-2-oxo-5-oxazolidinyl]carbonyl]amino]-2-cyclopentene-1-carboxylic acid

To a stirred solution of methyl (1S,4R)-4-[3-(3,5-difluorophenyl)-5-methyl-2-oxo-1,3-oxazolidine-5-amido]cyclopent-2-ene-1-carboxylate (i.e. the product of Synthesis Example 1) (6 g, 15.775 mmol, 1 equiv.), obtained in Step A, in a solution acetonitrile/water (150 mL, 4:1), triethylamine (10.994 mL, 78.875 mmol, 5 equiv.) and lithium bromide (13.7 g, 157.75 mmol, 10 equiv.) were added respectively at room temperature. The reaction mixture was stirred for 72 hr at room temperature. After completion of the reaction, the reaction mixture was diluted with water (30 mL) and extracted with ethyl acetate (30 mL). The aqueous organic layer was acidified with 1 N HCl solution and extracted with ethyl acetate (3×100 mL). The combined organic layer was dried over anhydrous sodium sulphate and filtered and concentrated under reduced pressure to get crude residue. The crude was purified by column chromatography on silica gel by eluting with methanol/dichloromethane (0-10%) and followed by recrystallization using dichloromethane and hexane to provide (1S,4R)-4-[[[3-(3,5-Difluorophenyl)-5-methyl-2-oxo-5-oxazolidinyl]carbonyl]amino]-2-cyclopentene-1-carboxylic acid (3.01 g, yield 50%, diastereomeric mixture) as a white solid.

¹H NMR (500 MHz, DMSO-d6) δ 1.62 (s, 3H), 1.84-1.91 (m, 1H), 2.37-2.44 (m, 1H), 3.45-3.64 (m, 1H), 3.95-3.98 (d, 1H), 4.21-4.27 (m, 1H), 4.78-4.93 (m, 1H), 5.74-5.79 (m, 1H), 5.89-5.92 (m, 1H), 7.00-7.05 (m, 1H), 7.34-7.36 (m, 2H), 8.30-8.34 (t, 3H), 12.41 (bs, 1H).

### Synthesis Example 3

### Preparation of 2-(Methylthio)ethyl (1S,4R)-4-[[[3-(3,5-difluorophenyl)-5-methyl-2-oxo-5-oxazolidinyl]carbonyl]amino]-2-cyclopentene-1-carboxylate (Compound 48)

### Step A: Preparation of 2-(Methylthio)ethyl (1S,4R)-4-[[[3-(3,5-difluorophenyl)-5-methyl-2-oxo-5-oxazolidinyl]carbonyl]amino]-2-cyclopentene-1-carboxylate

To a stirred solution of (1S,4R)-4-[3-(3,5-difluorophenyl)-5-methyl-2-oxo-1,3-oxazolidine-5-amido]cyclopent-2-ene-1-carboxylic acid (i.e. the product of Synthetic Example 2) (250 mg, 0.682 mmol, 1 equiv.), 2-(methylthio) ethanol (0.094 g, 1.024 mmol, 1.5 equiv.) in dichloromethane (6 mL) was added PyBOP (0.71 g, 1.365 mmol, 2 equiv.) and N,N-diisopropylethylamine (0.362 mL, 2.047 mmol, 3 equiv.) at room temperature. The reaction mixture was stirred at room temperature for 16 h. After completion of the reaction, the reaction mixture was directly concentrated under reduced pressure to get the crude residue. The crude material was purified by column chromatography on silica gel by eluting with ethyl acetate/petether (0%-50%), to get 2-(Methylthio)ethyl (1S,4R)-4-[[[3-(3,5-difluorophenyl)-5-methyl-2-oxo-5-oxazolidinyl]carbonyl]amino]-2-cyclopentene-1-carboxylate (210 mg, yield 69.9%, diastereomeric mixture).

¹H NMR δ 1.74-176 (m, 3H), 1.91-2.02 (m, 1H), 2.15-2.18 (m, 4H), 2.42-2.55 (m, 1H), 2.70-2.81 (m, 3H), 3.54-3.59 (m, 1H), 3.75-3.82 (m, 2H), 4.25-4.38 (m, 4H), 5.03-5.15 (m, 1H), 5.62-6.04 (m, 2H), 6.57-6.65 (m, 1H), 7.05-7.17 (m, 3H).

### Synthesis Example 4

### Preparation of Methyl (4S)-4-[[[3-(3,5-difluorophenyl)-5-methyl-2-oxo-5-oxazolidinyl]carbonyl]amino]-1-cyclopentene-1-carboxylate (Compound 37)

To a stirred solution of 3-(3,5-difluorophenyl)-5-methyl-2-oxo-1,3-oxazolidine-5-carboxylic acid (i.e. the product of Step C in Synthesis Example 1) (350 mg, 1.361 mmol, 1 equiv.) in dichloromethane (9 mL), triflate salt of methyl (4S)-4-amino-1-cyclopentene-1-carboxylate (0.521 g, 2.041 mmol, 1.5 equiv.), prepared according to the procedure described in Org. Lett. 2017, 19, 7, 1602-1605, PyBOP (1.416 g, 2.722 mmol, 2 equiv.), and N,N-diisopropylethylamine (0.528 g, 4.083 mmol, 3 equiv.) were added respectively at room temperature. The reaction mixture was stirred at room temperature for 16 h. After completion of the reaction, the reaction mixture was directly concentrated under reduced pressure to get crude residue. The crude was purified by column chromatography on silica gel by eluting with ethyl acetate/petroleum ether (0%-60%) to get Methyl (4S)-4-[[[3-(3,5-difluorophenyl)-5-methyl-2-oxo-5-oxazolidinyl]carbonyl]amino]-1-cyclopentene-1-carboxylate (350 mg, 0.92 mmol, yield 67.6%, diastereomeric mixture) as a white solid.

¹H NMR δ 1.73-1.74 (s, 3H), 2.37-2.54 (m, 2H), 2.92-3.10 (m, 2H), 3.75-3.77 (s, 3H), 3.79-3.81 (dd, 1H), 4.31-4.34 (dd, 1H), 4.58-4.67 (m, 1H), 6.58-6.66 (m, 1H), 6.71-6.82 (m, 2H), 7.09-7.16 (m, 2H).

### Synthesis Example 5

### Preparation of (4S)-4-[[[3-(3,5-difluorophenyl)-5-methyl-2-oxo-5-oxazolidinyl]carbonyl]amino]-1-cyclopentene-1-carboxylate acid (Compound 38)

Lithium bromide (4.384 g, 50.48 mmol, 12 equiv.) was added portion wise to a stirred solution of Methyl (4S)-4-[[[3-(3,5-difluorophenyl)-5-methyl-2-oxo-5-oxazolidinyl]carbonyl]amino]-1-cyclopentene-1-carboxylate (i.e. the product of Synthesis Example 4) (1.6 g, 4.207 mmol, 1 equiv.) in acetonitrile/water (20 mL, 4:1) mixture at 0° C. followed by triethylamine (0.851 g, 8.413 mmol, 2 equiv.) and stirred for 72 h at room temperature. After completion of the reaction, the reaction mixture was diluted with water and extracted with ethyl acetate (2×100 mL), the aqueous layer was acidified with 1 M HCl solution and extracted with ethyl acetate (2×100 mL), the combined organic layer was dried over sodium sulphate and concentrated under reduced pressure to get (4S)-4-[[[3-(3,5-difluorophenyl)-5-methyl-2-oxo-5-oxazolidinyl]carbonyl]amino]-1-cyclopentene-1-carboxylic acid (550 mg, 1.501 mmol, yield 35.7%, mixture of diastereomer) as an off white solid.

¹H NMR δ 1.75-1.76 (s, 3H), 2.44-2.54 (m, 1H), 2.55-2.66 (m, 1H), 2.96-3.09 (m, 3H), 3.81-3.83 (d, 1H), 4.34-4.36 (d, 1H), 4.58-4.68 (m, 1H), 6.58-6.66 (m, 1H), 6.80-6.87 (m, 1H), 7.08-7.19 (m, 2H).

### Synthesis Example 6

### Preparation of methyl (1R,2S,4R,5S)-4-[[3-(3,5-difluorophenyl)-5-methyl-2-oxo-oxazolidine-5-carbonyl]amino]bicyclo[3.1.0]hexane-2-carboxylate: (Compound No. 176)

To a stirred solution of 3-(3,5-difluorophenyl)-5-methyl-2-oxo-1,3-oxazolidine-5-carboxylic acid (i.e. the product of Step C, Synthesis Example 1; 300 mg, 0.11 mmol, 1 equiv.), methyl (1R,2S,4R,5S)-4-aminobicyclo[3.1.0]hexane-2-carboxylate (271 mg, 0.17 mmol, 1.5 equiv.), prepared according to the procedure described in WO2017133669, in dichloromethane (5 mL) N,N-diisopropylethylamine (0.6 mL, 0.35 mmol, 3 equiv.), PyBOP® ((benzotriazole-1-yloxy)tripyrrolidinophosphonium hexafluorophosphate, (88 mg, 0.17 mmol, 1.5 equiv.) were added respectively at room temperature. The reaction mixture was stirred for 16 h at room temperature. After the completion of the reaction, the reaction mixture was diluted with water (20 mL), extracted with dichloromethane (2×20 mL). The combined organic layer was dried over anhydrous sodium sulphate, filtered, and concentrated under reduced pressure to get crude residue. The crude product was purified by column chromatography on silica gel and eluting with ethyl acetate/petether (0-50%) to afford methyl (1R,2S,4R,5S)-4-[[3-(3,5-difluorophenyl)-5-methyl-2-oxo-oxazolidine-5-carbonyl]amino]bicyclo[3.1.0]hexane-2-carboxylate (0.2 g, yield 43%) as a diastereomeric mixture.

¹H NMR (400 MHz, DMSO-d6): δ 0.30-0.33 (m, 1H), 0.80-0.81 (m, 1H), 1.51-1.59 (m, 6H), 1.80-1.85 (m, 1H), 3.03-3.04 (m, 1H), 3.61 (S, 3H), 3.94-3.97 (m, 1H), 4.18-4.26 (m, 1H), 4.41-4.42 (m, 1H), 7.01 (t, 1H), 7.32-7.38 (m, 2H), 8.20 (t, 1H). MS [M+H]⁺: 395.23

### Synthesis Example 7

### Preparation of methyl 3-[[3-(3,5-difluorophenyl)-5-methyl-2-oxo-oxazolidine-5-carbonyl]amino]cyclobutanecarboxylate (Compound 118)

To a stirred solution of 3-(3,5-difluorophenyl)-5-methyl-2-oxo-1,3-oxazolidine-5-carboxylic acid (i.e. the product of Step C in Synthesis Example 1) (1.2 g, 4.66 mmol, 1 equiv.), methyl (1S,3S)-3-aminocyclobutane-1-carboxylate (0.904 g, 6.999 mmol, 1.5 equiv.) in dichloromethane (24 mL) N,N-diisopropylethylamine (1.206 g, 9.332 mmol, 2 equiv.), PyBOP® ((benzotriazole-1-yloxy)tripyrrolidinophosphonium hexafluorophosphate, (4.856 g, 9.332 mmol, 2 equiv.) were added respectively at room temperature. The reaction mixture was stirred for 16 h at room temperature. After the completion of the reaction, the reaction mixture was diluted with water (50 mL), extracted with dichloromethane (2×50 mL). The combined organic layer was dried over anhydrous sodium sulphate, filtered, and concentrated under reduced pressure to get crude residue. The crude product was purified by column chromatography on silica gel and eluting with ethyl acetate/petether (0-50%) to afford methyl 3-[[3-(3,5-difluorophenyl)-5-methyl-2-oxo-oxazolidine-5-carbonyl]amino]cyclobutanecarboxylate (1.25 g, yield 69.1%) as a diastereomeric mixture.

¹H NMR (500 MHz, CDCl₃): δ 1.73 (S, 3H), 2.20-2.26 (m, 2H), 2.60-2.70 (m, 2H), 2.82-2.89 (m, 1H), 3.70 (s, 3H), 3.78-3.80 (d, 1H), 4.31-4.33 (d, 1H), 4.37-4.40 (m, 1H), 6.59-6.63 (m, 1H), 6.92-6.94 (bd, 1H), 7.10-7.16 (m, 2H). MS [M+H]⁺-369.3 (ES+)

### Synthesis Example 8

### Preparation of methyl (1S,4R)-4-[[1-(3,5-difluorophenyl)-4-methyl-2-oxo-imidazolidine-4-carbonyl]amino]cyclopent-2-ene-1-carboxylate

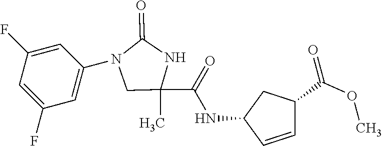 [SMILES: COC(=O)[C@@H]1C=C[C@H](NC(=O)C2(C)CN(c3cc(F)cc(F)c3)C(=O)N2)C1]

### Step A: Preparation of ethyl 3-(3,5-difluoroanilino)-2-methyl-2-nitro-propanoate

To a stirred solution of ethyl-2-nitropropanoate (1 g, 6.8 mmol) in methanol (10 mL) was added compound 3,5-difluoroaniline (0.877 g, 6.802 mmol), and aqueous 37% formalin (0.66 g, 8.163 mmol) at 25° C. The reaction mixture was stirred at 80° C. for 3 h. Thin layer chromatography analysis showed completion of the reaction. The reaction mixture was concentrated under reduced pressure to give the crude product, which was charged on a silica gel column. Elution of the column with 5% ethyl acetate in petroleum ether afforded ethyl 3-(3,5-difluoroanilino)-2-methyl-2-nitro-propanoate as a colorless liquid (0.7 g, 36.8% yield).

¹H NMR (400 MHz, d6-DMSO) δ 6.48 (t, 1H), 6.39 (dd, 2H), 6.28 (td, 1H), 4.20 (q, 2H), 4.04 (dd, 1H), 3.87 (dd, 1H), 1.78 (s, 3H), 1.19 (t, 3H). MS (CI) m/e=289 (M+1).

### Step B: Preparation of ethyl 2-amino-3-(3,5-difluoroanilino)-2-methyl-propanoate

To a solution of ethyl 3-(3,5-difluoroanilino)-2-methyl-2-nitro-propanoate (1.5 g, 5.2 mmol) in ethanol (30 mL) was added Raney-Nickel (0.457 g, 7.812 mmol). The reaction mixture was stirred at 25° C. under a hydrogen atmosphere at 50 psi (about 2.07e+005 Newtons/square meter) for 12 h. Thin layer chromatography analysis showed completion of the reaction. The reaction mixture was filtered through a Celite® (diatomaceous earth filter aid) pad and the filtrate was evaporated under reduced pressure to afford the crude product which was then charged onto a silica gel column. Elution of the column with 20% ethyl acetate in petroleum ether afforded ethyl 2-amino-3-(3,5-difluoroanilino)-2-methyl-propanoate as an off-white solid (0.9 g, 67% yield).

¹H NMR (400 MHz, d6-DMSO) δ 6.28 (dd, 2H), 6.21-6.15 (m, 2H), 4.05-3.95 (m, 2H), 3.30 (m, 1H), 3.08 (dd, 1H), 2.04 (br s, 2H), 1.23 (s, 3H), 1.13 (t, 3H). MS (CI) m/e=259 (M+1).

### Step C: Preparation of ethyl 1-(3,5-difluorophenyl)-4-methyl-2-oxo-imidazolidine-4-carboxylate

To stirred solution of ethyl 2-amino-3-(3,5-difluoroanilino)-2-methyl-propanoate (i.e. the product of Step B, 3.1 g, 12.02 mmol) in dichloromethane (30 mL) was added triethylamine (1.8 g, 18.02 mmol), and triphosgene (3.55 g, 12.02 mmol), at 0° C. The resulting mixture was stirred at 25° C. for 12 h. Thin layer chromatography analysis showed completion of the reaction. The mixture was diluted with cold water (20 mL), extracted with dichloromethane (2×50 mL) and the organic layer was dried over anhydrous sodium sulfate and concentrated under reduced pressure to afford the crude product. Purification on a silica gel column eluting with 15% ethyl acetate in petroleum ether afforded pure ethyl 1-(3,5-difluorophenyl)-4-methyl-2-oxo-imidazolidine-4-carboxylate as an off-white solid (2.3 g, 67% yield).

¹H NMR (400 MHz, d6-DMSO) δ 8.06 (s, 1H), 7.30 (dd, 2H), 6.82 (td, 1H), 4.18 (q, 2H), 4.12 (d, 1H), 3.75 (d, 1H), 1.48 (s, 3H), 1.21 (t, 3H). MS (CI) m/e=285 (M+1).

### Step D: Preparation of 1-(3,5-difluorophenyl)-4-methyl-2-oxo-imidazolidine-4-carboxylic acid

To stirred solution of ethyl 1-(3,5-difluorophenyl)-4-methyl-2-oxo-imidazolidine-4-carboxylate (i.e. the product of Step C; 0.3 g, 1.06 mmol) in methanol (9 mL) and water (3 mL) was added LiOH·H₂O (88 mg, 2.112 mmol) at 25° C. The reaction mixture was stirred at 80° C. for 2 h. Thin layer chromatography analysis showed completion of the reaction. The reaction mixture was concentrated under reduced pressure and then diluted with water (20 mL). Acidification to pH~4 using citric acid resulted in the formation of a solid that was filtered and dried under vacuum to afford 1-(3,5-difluorophenyl)-4-methyl-2-oxo-imidazolidine-4-carboxylic acid as an off-white solid (0.210 g, 77% yield).

¹H NMR (400 MHz, d6-DMSO) δ 13.24 (br s, 1H), 7.97 (s, 1H), 7.30 (dt, 2H), 6.80 (tt, 1H), 4.09 (d, 1H), 3.70 (d, 1H), 1.46 (s, 3H). MS (CI) m/e=257 (M+1).

### Step E: Preparation of methyl (1S,4R)-4-[[1-(3,5-difluorophenyl)-4-methyl-2-oxo-imidazolidine-4-carbonyl]amino]cyclopent-2-ene-1-carboxylate

To a solution of 1-(3,5-difluorophenyl)-4-methyl-2-oxo-imidazolidine-4-carboxylic acid (i.e. the product of Step D, 240 mg, 0.936 mmol), methyl (1S,4R)-4-aminocyclopent-2-ene-1-carboxylate (244 mg, 1.72 mmol), in dichloromethane (10 mL) were added N,N-diisopropylethylamine (0.67 mL, 3.86 mmol) and PyBOP® ((benzotriazole-1-yloxy)tripyrrolidinophosphonium hexafluorophosphate, 0.761 g, 1.46 mmol) at 0° C. The reaction mixture was stirred at 25° C. for 16 h. Thin layer chromatography analysis showed completion of the reaction. The resulting mixture was diluted with cold water (50 mL) and extracted with ethyl acetate (2×50 mL), the combined organic layers were dried over anhydrous sodium sulfate, filtered, and concentrated under reduced pressure to afford the crude product. Purification by chromatography on silica gel, eluting with 15% ethyl acetate in petroleum ether gave methyl (1S,4R)-4-[[1-(3,5-difluorophenyl)-4-methyl-2-oxo-imidazolidine-4-carbonyl]amino]cyclopent-2-ene-1-carboxylate off-white solid (186 mg, 52% yield).

¹H NMR (400 MHz, d6-DMSO) δ 7.92 (t, 1H), 7.69 (s, 1H), 7.30 (dt, 2H), 6.81 (tt, 1H), 5.92-5.88 (m, 1H), 5.82-5.77 (m, 1H), 4.81 (br s, 1H), 4.00 (dd, 1H), 3.70 (d, 1H), 3.64 (d, 3H), 3.60-3.53 (m, 1H), 1.85-1.75 (m, 1H), 1.43 (s, 3H). MS (CI) m/e=380 (M+1).

By the procedures described herein together with methods known in the art, the following compounds of Tables 1 to 200 can be prepared. The following abbreviations are used in the Tables which follow: t means tertiary, s means secondary, n means normal, i means iso, c means cyclo, Me means methyl, Et means ethyl, Pr means propyl, Bu means butyl, i-Pr means isopropyl, c-Pr means cyclopropyl, t-Bu means tertiary butyl, Ph means phenyl, OMe means methoxy, OEt means ethoxy, SMe means methylthio, —CN means cyano, —NO₂ means nitro, TMS means trimethylsilyl, SOMe means methylsulfinyl, C₂F₅ means CF₂CF₃, SO₂Me means methylsulfonyl and the number before the “-” followed by an atom, indicates the position of the atom, for example, when (R)ₙ is 3-F, the substituent F is at the 3-position of the phenyl ring.

TABLE 1

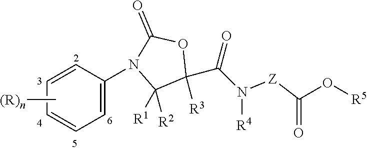

R¹ = R² = R⁴ = H, R³ = CH₃, and Z = Z-1, and the remaining
variables are defined below

| (R)ₙ | R⁵ |
|---|---|
| 3-F | H |
| 3-F | CH₃ |
| 3-F | CH₂CH₃ |
| 3-F | CH₂CH₂CH₃ |
| 3-F | CH(CH₃)₂ |
| 3-F | C(CH₃)₃ |
| 3-F | CH₂CF₃ |
| 3-F | CH₂CH₂CF₃ |
| 3-F | CH₂CN |
| 3-F | CH₂CH₂OCH₃ |
| 3-F | CH₂CH₂SCH₃ |
| 3-F | CH₂CH₂SOCH₃ |
| 3-F | CH₂CH₂SO₂CH₃ |
| 3-F | CH₂CH(CH₃)₂ |
| 3-F | CH₂Ph |
| 3-F | CH₂4-F-Ph |
| 3-F | c-Pr |
| 3-F | c-Bu |
| 3-F | c-Pent |
| 3-F | c-Hex |
| 3-F | c-Hept |
| 3-F | Ph |
| 3-F | CH₂CO₂CH₃ |
| 3-F | CH₂CO₂CH₂CH₃ |
| 3-F | CH₂COCH₃ |
| 3-F, 5-F | H |
| 3-F, 5-F | CH₃ |
| 3-F, 5-F | CH₂CH₃ |
| 3-F, 5-F | CH₂CH₂CH₃ |
| 3-F, 5-F | CH(CH₃)₂ |
| 3-F, 5-F | C(CH₃)₃ |
| 3-F, 5-F | CH₂CF₃ |
| 3-F, 5-F | CH₂CH₂CF₃ |
| 3-F, 5-F | CH₂CN |
| 3-F, 5-F | CH₂CH₂OCH₃ |
| 3-F, 5-F | CH₂CH₂SCH₃ |
| 3-F, 5-F | CH₂CH₂SOCH₃ |
| 3-F, 5-F | CH₂CH₂SO₂CH₃ |
| 3-F, 5-F | CH₂CH(CH₃)₂ |
| 3-F, 5-F | CH₂Ph |
| 3-F, 5-F | CH₂4-F-Ph |
| 3-F, 5-F | c-Pr |
| 3-F, 5-F | c-Bu |
| 3-F, 5-F | c-Pent |
| 3-F, 5-F | c-Hex |
| 3-F, 5-F | c-Hept |
| 3-F, 5-F | Ph |
| 3-F, 5-F | CH₂CO₂CH₃ |
| 3-F, 5-F | CH₂CO₂CH₂CH₃ |
| 3-F, 5-F | CH₂COCH₃ |
| 3-Cl | H |
| 3-Cl | CH₃ |
| 3-Cl | CH₂CH₃ |
| 3-Cl | CH₂CH₂CH₃ |
| 3-Cl | CH(CH₃)₂ |
| 3-Cl | C(CH₃)₃ |
| 3-Cl | CH₂CF₃ |
| 3-Cl | CH₂CH₂CF₃ |
| 3-Cl | CH₂CN |
| 3-Cl | CH₂CH₂OCH₃ |
| 3-Cl | CH₂CH₂SCH₃ |
| 3-Cl | CH₂CH₂SOCH₃ |
| 3-Cl | CH₂CH₂SO₂CH₃ |
| 3-Cl | CH₂CH(CH₃)₂ |
| 3-Cl | CH₂Ph |
| 3-Cl | CH₂4-F-Ph |
| 3-Cl | c-Pr |
| 3-Cl | c-Bu |
| 3-Cl | c-Pent |
| 3-Cl | c-Hex |
| 3-Cl | c-Hept |
| 3-Cl | Ph |
| 3-Cl | CH₂CO₂CH₃ |
| 3-Cl | CH₂CO₂CH₂CH₃ |
| 3-Cl | CH₂COCH₃ |
| 3-Cl, 5-Cl | H |
| 3-Cl, 5-Cl | CH₃ |
| 3-Cl, 5-Cl | CH₂CH₃ |
| 3-Cl, 5-Cl | CH₂CH₂CH₃ |
| 3-Cl, 5-Cl | CH(CH₃)₂ |
| 3-Cl, 5-Cl | C(CH₃)₃ |
| 3-Cl, 5-Cl | CH₂CF₃ |
| 3-Cl, 5-Cl | CH₂CH₂CF₃ |
| 3-Cl, 5-Cl | CH₂CN |
| 3-Cl, 5-Cl | CH₂CH₂OCH₃ |
| 3-Cl, 5-Cl | CH₂CH₂SCH₃ |
| 3-Cl, 5-Cl | CH₂CH₂SOCH₃ |
| 3-Cl, 5-Cl | CH₂CH₂SO₂CH₃ |
| 3-Cl, 5-Cl | CH₂CH(CH₃)₂ |
| 3-Cl, 5-Cl | CH₂Ph |
| 3-Cl, 5-Cl | CH₂4-F-Ph |
| 3-Cl, 5-Cl | c-Pr |
| 3-Cl, 5-Cl | c-Bu |
| 3-Cl, 5-Cl | c-Pent |
| 3-Cl, 5-Cl | c-Hex |
| 3-Cl, 5-Cl | c-Hept |
| 3-Cl, 5-Cl | Ph |
| 3-Cl, 5-Cl | CH₂CO₂CH₃ |
| 3-Cl, 5-Cl | CH₂CO₂CH₂CH₃ |
| 3-Cl, 5-Cl | CH₂COCH₃ |
| 3-F, 4-F, 5-F | H |
| 3-F, 4-F, 5-F | CH₃ |
| 3-F, 4-F, 5-F | CH₂CH₃ |
| 3-F, 4-F, 5-F | CH₂CH₂CH₃ |
| 3-F, 4-F, 5-F | CH(CH₃)₂ |
| 3-F, 4-F, 5-F | C(CH₃)₃ |
| 3-F, 4-F, 5-F | CH₂CF₃ |
| 3-F, 4-F, 5-F | CH₂CH₂CF₃ |
| 3-F, 4-F, 5-F | CH₂CN |
| 3-F, 4-F, 5-F | CH₂CH₂OCH₃ |
| 3-F, 4-F, 5-F | CH₂CH₂SCH₃ |
| 3-F, 4-F, 5-F | CH₂CH₂SOCH₃ |
| 3-F, 4-F, 5-F | CH₂CH₂SO₂CH₃ |
| 3-F, 4-F, 5-F | CH₂CH(CH₃)₂ |
| 3-F, 4-F, 5-F | CH₂Ph |
| 3-F, 4-F, 5-F | CH₂4-F-Ph |
| 3-F, 4-F, 5-F | c-Pr |
| 3-F, 4-F, 5-F | c-Bu |
| 3-F, 4-F, 5-F | c-Pent |
| 3-F, 4-F, 5-F | c-Hex |
| 3-F, 4-F, 5-F | c-Hept |
| 3-F, 4-F, 5-F | Ph |
| 3-F, 4-F, 5-F | CH₂CO₂CH₃ |
| 3-F, 4-F, 5-F | CH₂CO₂CH₂CH₃ |
| 3-F, 4-F, 5-F | CH₂COCH₃ |
| 3-Cl, 4-F, 5-Cl | H |
| 3-Cl, 4-F, 5-Cl | CH₃ |
| 3-Cl, 4-F, 5-Cl | CH₂CH₃ |
| 3-Cl, 4-F, 5-Cl | CH₂CH₂CH₃ |
| 3-Cl, 4-F, 5-Cl | CH(CH₃)₂ |
| 3-Cl, 4-F, 5-Cl | C(CH₃)₃ |
| 3-Cl, 4-F, 5-Cl | CH₂CF₃ |
| 3-Cl, 4-F, 5-Cl | CH₂CH₂CF₃ |
| 3-Cl, 4-F, 5-Cl | CH₂CN |
| 3-Cl, 4-F, 5-Cl | CH₂CH₂OCH₃ |
| 3-Cl, 4-F, 5-Cl | CH₂CH₂SCH₃ |
| 3-Cl, 4-F, 5-Cl | CH₂CH₂SOCH₃ |
| 3-Cl, 4-F, 5-Cl | CH₂CH₂SO₂CH₃ |
| 3-Cl, 4-F, 5-Cl | CH₂CH(CH₃)₂ |
| 3-Cl, 4-F, 5-Cl | CH₂Ph |
| 3-Cl, 4-F, 5-Cl | CH₂4-F-Ph |
| 3-Cl, 4-F, 5-Cl | c-Pr |
| 3-Cl, 4-F, 5-Cl | c-Bu |
| 3-Cl, 4-F, 5-Cl | c-Pent |
| 3-Cl, 4-F, 5-Cl | c-Hex |
| 3-Cl, 4-F, 5-Cl | c-Hept |
| 3-Cl, 4-F, 5-Cl | Ph |
| 3-Cl, 4-F, 5-Cl | CH₂CO₂CH₃ |
| 3-Cl, 4-F, 5-Cl | CH₂CO₂CH₂CH₃ |
| 3-Cl, 4-F, 5-Cl | CH₂COCH₃ |
| 3-CN | H |
| 3-CN | CH₃ |
| 3-CN | CH₂CH₃ |
| 3-CN | CH₂CH₂CH₃ |
| 3-CN | CH(CH₃)₂ |
| 3-CN | C(CH₃)₃ |
| 3-CN | CH₂CF₃ |
| 3-CN | CH₂CH₂CF₃ |
| 3-CN | CH₂CN |
| 3-CN | CH₂CH₂OCH₃ |
| 3-CN | CH₂CH₂SCH₃ |
| 3-CN | CH₂CH₂SOCH₃ |
| 3-CN | CH₂CH₂SO₂CH₃ |
| 3-CN | CH₂CH(CH₃)₂ |
| 3-CN | CH₂Ph |
| 3-CN | CH₂4-F-Ph |
| 3-CN | c-Pr |
| 3-CN | c-Bu |
| 3-CN | c-Pent |
| 3-CN | c-Hex |
| 3-CN | c-Hept |
| 3-CN | Ph |
| 3-CN | CH₂CO₂CH₃ |
| 3-CN | CH₂CO₂CH₂CH₃ |
| 3-CN | CH₂COCH₃ |
| 3-CN, 5-F | H |
| 3-CN, 5-F | CH₃ |
| 3-CN, 5-F | CH₂CH₃ |
| 3-CN, 5-F | CH₂CH₂CH₃ |
| 3-CN, 5-F | CH(CH₃)₂ |
| 3-CN, 5-F | C(CH₃)₃ |
| 3-CN, 5-F | CH₂CF₃ |
| 3-CN, 5-F | CH₂CH₂CF₃ |
| 3-CN, 5-F | CH₂CN |
| 3-CN, 5-F | CH₂CH₂OCH₃ |
| 3-CN, 5-F | CH₂CH₂SCH₃ |
| 3-CN, 5-F | CH₂CH₂SOCH₃ |
| 3-CN, 5-F | CH₂CH₂SO₂CH₃ |
| 3-CN, 5-F | CH₂CH(CH₃)₂ |
| 3-CN, 5-F | CH₂Ph |
| 3-CN, 5-F | CH₂4-F-Ph |
| 3-CN, 5-F | c-Pr |
| 3-CN, 5-F | c-Bu |
| 3-CN, 5-F | c-Pent |
| 3-CN, 5-F | c-Hex |
| 3-CN, 5-F | c-Hept |
| 3-CN, 5-F | Ph |
| 3-CN, 5-F | CH₂CO₂CH₃ |
| 3-CN, 5-F | CH₂CO₂CH₂CH₃ |
| 3-CN, 5-F | CH₂COCH₃ |
| 3-CF₃ | H |
| 3-CF₃ | CH₃ |
| 3-CF₃ | CH₂CH₃ |
| 3-CF₃ | CH₂CH₂CH₃ |
| 3-CF₃ | CH(CH₃)₂ |
| 3-CF₃ | C(CH₃)₃ |
| 3-CF₃ | CH₂CF₃ |
| 3-CF₃ | CH₂CH₂CF₃ |
| 3-CF₃ | CH₂CN |
| 3-CF₃ | CH₂CH₂OCH₃ |
| 3-CF₃ | CH₂CH₂SCH₃ |
| 3-CF₃ | CH₂CH₂SOCH₃ |
| 3-CF₃ | CH₂CH₂SO₂CH₃ |
| 3-CF₃ | CH₂CH(CH₃)₂ |
| 3-CF₃ | CH₂Ph |
| 3-CF₃ | CH₂4-F-Ph |
| 3-CF₃ | c-Pr |
| 3-CF₃ | c-Bu |
| 3-CF₃ | c-Pent |
| 3-CF₃ | c-Hex |
| 3-CF₃ | c-Hept |
| 3-CF₃ | Ph |
| 3-CF₃ | CH₂CO₂CH₃ |
| 3-CF₃ | CH₂CO₂CH₂CH₃ |
| 3-CF₃ | CH₂COCH₃ |
| 3-CF₃, 5-F | H |
| 3-CF₃, 5-F | CH₃ |
| 3-CF₃, 5-F | CH₂CH₃ |
| 3-CF₃, 5-F | CH₂CH₂CH₃ |
| 3-CF₃, 5-F | CH(CH₃)₂ |
| 3-CF₃, 5-F | C(CH₃)₃ |
| 3-CF₃, 5-F | CH₂CF₃ |
| 3-CF₃, 5-F | CH₂CH₂CF₃ |
| 3-CF₃, 5-F | CH₂CN |
| 3-CF₃, 5-F | CH₂CH₂OCH₃ |
| 3-CF₃, 5-F | CH₂CH₂SCH₃ |
| 3-CF₃, 5-F | CH₂CH₂SOCH₃ |
| 3-CF₃, 5-F | CH₂CH₂SO₂CH₃ |
| 3-CF₃, 5-F | CH₂CH(CH₃)₂ |
| 3-CF₃, 5-F | CH₂Ph |
| 3-CF₃, 5-F | CH₂4-F-Ph |
| 3-CF₃, 5-F | c-Pr |
| 3-CF₃, 5-F | c-Bu |
| 3-CF₃, 5-F | c-Pent |
| 3-CF₃, 5-F | c-Hex |
| 3-CF₃, 5-F | c-Hept |
| 3-CF₃, 5-F | Ph |
| 3-CF₃, 5-F | CH₂CO₂CH₃ |
| 3-CF₃, 5-F | CH₂CO₂CH₂CH₃ |
| 3-CF₃, 5-F | CH₂COCH₃ |
| 3-CF₃, 5-Cl | H |
| 3-CF₃, 5-Cl | CH₃ |
| 3-CF₃, 5-Cl | CH₂CH₃ |
| 3-CF₃, 5-Cl | CH₂CH₂CH₃ |
| 3-CF₃, 5-Cl | CH(CH₃)₂ |
| 3-CF₃, 5-Cl | C(CH₃)₃ |
| 3-CF₃, 5-Cl | CH₂CF₃ |
| 3-CF₃, 5-Cl | CH₂CH₂CF₃ |
| 3-CF₃, 5-Cl | CH₂CN |
| 3-CF₃, 5-Cl | CH₂CH₂OCH₃ |
| 3-CF₃, 5-Cl | CH₂CH₂SCH₃ |
| 3-CF₃, 5-Cl | CH₂CH₂SOCH₃ |
| 3-CF₃, 5-Cl | CH₂CH₂SO₂CH₃ |
| 3-CF₃, 5-Cl | CH₂CH(CH₃)₂ |
| 3-CF₃, 5-Cl | CH₂Ph |
| 3-CF₃, 5-Cl | CH₂4-F-Ph |
| 3-CF₃, 5-Cl | c-Pr |
| 3-CF₃, 5-Cl | c-Bu |
| 3-CF₃, 5-Cl | c-Pent |
| 3-CF₃, 5-Cl | c-Hex |
| 3-CF₃, 5-Cl | c-Hept |
| 3-CF₃, 5-Cl | Ph |
| 3-CF₃, 5-Cl | CH₂CO₂CH₃ |
| 3-CF₃, 5-Cl | CH₂CO2CH₂CH₃ |
| 3-CF₃, 5-Cl | CH₂COCH₃ |
| 3-OCF₃ | H |
| 3-OCF₃ | CH₃ |
| 3-OCF₃ | CH₂CH₃ |
| 3-OCF₃ | CH₂CH₂CH₃ |
| 3-OCF₃ | CH(CH₃)₂ |
| 3-OCF₃ | C(CH₃)₃ |
| 3-OCF₃ | CH₂CF₃ |
| 3-OCF₃ | CH₂CH₂CF₃ |
| 3-OCF₃ | CH₂CN |
| 3-OCF₃ | CH₂CH₂OCH₃ |
| 3-OCF₃ | CH₂CH₂SCH₃ |
| 3-OCF₃ | CH₂CH₂SOCH₃ |
| 3-OCF₃ | CH₂CH₂SO₂CH₃ |
| 3-OCF₃ | CH₂CH(CH₃)₂ |
| 3-OCF₃ | CH₂Ph |
| 3-OCF₃ | CH₂4-F-Ph |
| 3-OCF₃ | c-Pr |
| 3-OCF₃ | c-Bu |
| 3-OCF₃ | c-Pent |
| 3-OCF₃ | c-Hex |
| 3-OCF₃ | c-Hept |
| 3-OCF₃ | Ph |
| 3-OCF₃ | CH₂CO₂CH₃ |
| 3-OCF₃ | CH₂CO₂CH₂CH₃ |
| 3-OCF₃ | CH₂COCH₃ |
| 3-OCF₃, 5-F | H |
| 3-OCF₃, 5-F | CH₃ |
| 3-OCF₃, 5-F | CH₂CH₃ |
| 3-OCF₃, 5-F | CH₂CH₂CH₃ |
| 3-OCF₃, 5-F | CH(CH₃)₂ |
| 3-OCF₃, 5-F | C(CH₃)₃ |
| 3-OCF₃, 5-F | CH₂CF₃ |
| 3-OCF₃, 5-F | CH₂CH₂CF₃ |
| 3-OCF₃, 5-F | CH₂CN |
| 3-OCF₃, 5-F | CH₂CH₂OCH₃ |
| 3-OCF₃, 5-F | CH₂CH₂SCH₃ |
| 3-OCF₃, 5-F | CH₂CH₂SOCH₃ |
| 3-OCF₃, 5-F | CH₂CH₂SO₂CH₃ |
| 3-OCF₃, 5-F | CH₂CH(CH₃)₂ |
| 3-OCF₃, 5-F | CH₂Ph |
| 3-OCF₃, 5-F | CH₂4-F-Ph |
| 3-OCF₃, 5-F | c-Pr |
| 3-OCF₃, 5-F | c-Bu |
| 3-OCF₃, 5-F | c-Pent |
| 3-OCF₃, 5-F | c-Hex |
| 3-OCF₃, 5-F | c-Hept |
| 3-OCF₃, 5-F | Ph |
| 3-OCF₃, 5-F | CH₂CO₂CH₃ |
| 3-OCF₃, 5-F | CH₂CO₂CH₂CH₃ |
| 3-OCF₃, 5-F | CH₂COCH₃ |
| 3-OCF₃, 5-Cl | H |
| 3-OCF₃, 5-Cl | CH₃ |
| 3-OCF₃, 5-Cl | CH₂CH₃ |
| 3-OCF₃, 5-Cl | CH₂CH₂CH₃ |
| 3-OCF₃, 5-Cl | CH(CH₃)₂ |
| 3-OCF₃, 5-Cl | C(CH₃)₃ |
| 3-OCF₃, 5-Cl | CH₂CF₃ |
| 3-OCF₃, 5-Cl | CH₂CH₂CF₃ |
| 3-OCF₃, 5-Cl | CH₂CN |
| 3-OCF₃, 5-Cl | CH₂CH₂OCH₃ |
| 3-OCF₃, 5-Cl | CH₂CH₂SCH₃ |
| 3-OCF₃, 5-Cl | CH₂CH₂SOCH₃ |
| 3-OCF₃, 5-Cl | CH₂CH₂SO₂CH₃ |
| 3-OCF₃, 5-Cl | CH₂CH(CH₃)₂ |
| 3-OCF₃, 5-Cl | CH₂Ph |
| 3-OCF₃, 5-Cl | CH₂4-F-Ph |
| 3-OCF₃, 5-Cl | c-Pr |
| 3-OCF₃, 5-Cl | c-Bu |
| 3-OCF₃, 5-Cl | c-Pent |
| 3-OCF3, 5-Cl | c-Hex |
| 3-OCF3, 5-Cl | c-Hept |
| 3-OCF3, 5-Cl | Ph |
| 3-OCF3, 5-Cl | CH₂CO₂CH₃ |
| 3-OCF3, 5-Cl | CH₂CO₂CH₂CH₃ |
| 3-OCF3, 5-Cl | CH₂COCH₃ |

The present disclosure also includes Tables 2-100, each of which is constructed the same as Table 1 above, except that the row heading in Table 1 (i.e. R¹═R²═R⁴═H, R³═CH₃, and Z═Z-1) is replaced with the respective row heading shown below.

| TABLE | Header Row Variable |
|---|---|
| 2 | R¹ = R² = R⁴ = H, R³ = CH₂CH₃, and Z = Z-1 |
| 3 | R¹ = R² = R⁴ = H, R³ = CH═CH₂, and Z = Z-1 |
| 4 | R¹ = R² = R⁴ = H, R³ = CF₃, and Z = Z-1 |
| 5 | R¹ = R² = R⁴ = H, R³ = OCH₃, and Z = Z-1 |
| 6 | R¹ = R² = R⁴ = H, R³ = c-Pr, and Z = Z-1 |
| 7 | R¹ = R² = R⁴ = H, R³ = CH₂Cl, and Z = Z-1 |
| 8 | R¹ = R² = R⁴ = H, R³ = CH₃, and Z = Z-4 |
| 9 | R¹ = R² = R⁴ = H, R³ = CH₂CH₃, and Z = Z-4 |
| 10 | R¹ = R² = R⁴ = H, R³ = CH═CH₂, and Z = Z-4 |
| 11 | R¹ = R² = R⁴ = H, R³ = CF₃, and Z = Z-4 |
| 12 | R¹ = R² = R⁴ = H, R³ = OCH₃, and Z = Z-4 |
| 13 | R¹ = R² = R⁴ = H, R³ = c-Pr, and Z = Z-4 |
| 14 | R¹ = R² = R⁴ = H, R³ = CH₂Cl, and Z = Z-4 |
| 15 | R¹ = R² = R⁴ = H, R³ = CH₃, and Z = Z-6 |
| 16 | R¹ = R² = R⁴ = H, R³ = CH₂CH₃, and Z = Z-6 |
| 17 | R¹ = R² = R⁴ = H, R³ = CH═CH₂, and Z = Z-6 |
| 18 | R¹ = R² = R⁴ = H, R³ = CF₃, and Z = Z-6 |
| 19 | R¹ = R² = R⁴ = H, R³ = OCH₃, and Z = Z-6 |
| 20 | R¹ = R² = R⁴ = H, R³ = c-Pr, and Z = Z-6 |
| 21 | R¹ = R² = R⁴ = H, R³ = CH₂Cl, and Z = Z-6 |
| 22 | R¹ = R² = R⁴ = H, R³ = CH₃, and Z = Z-12 |
| 23 | R¹ = R² = R⁴ = H, R³ = CH₂CH₃, and Z = Z-12 |
| 24 | R¹ = R² = R⁴ = H, R³ = CH═CH₂, and Z = Z-12 |
| 25 | R¹ = R² = R⁴ = H, R³ = CF₃, and Z = Z-12 |
| 26 | R¹ = R² = R⁴ = H, R³ = OCH₃, and Z = Z-12 |
| 27 | R¹ = R² = R⁴ = H, R³ = c-Pr, and Z = Z-12 |
| 28 | R¹ = R² = R⁴ = H, R³ = CH₂Cl, and Z = Z-12 |
| 29 | R¹ = R² = R⁴ = H, R³ = CH₃, and Z = Z-22 |
| 30 | R¹ = R² = R⁴ = H, R³ = CH₂CH₃, and Z = Z-22 |
| 31 | R¹ = R² = R⁴ = H, R³ = CH═CH₂, and Z = Z-22 |
| 32 | R¹ = R² = R⁴ = H, R³ = CF₃, and Z = Z-22 |
| 33 | R¹ = R² = R⁴ = H, R³ = OCH₃, and Z = Z-22 |
| 34 | R¹ = R² = R⁴ = H, R³ = c-Pr, and Z = Z-22 |
| 35 | R¹ = R² = R⁴ = H, R³ = CH₂Cl, and Z = Z-22 |
| 36 | R¹ = R² = R⁴ = H, R³ = CH₃, and Z = Z-24 |
| 37 | R¹ = R² = R⁴ = H, R³ = CH₃, and Z = Z-30 |
| 38 | R¹ = R² = R⁴ = H, R³ = CH₂CH₃, and Z = Z-30 |
| 39 | R¹ = R² = R⁴ = H, R³ = CH═CH₂, and Z = Z-30 |
| 40 | R¹ = R² = R⁴ = H, R³ = CF₃, and Z = Z-30 |
| 41 | R¹ = R² = R⁴ = H, R³ = OCH₃, and Z = Z-30 |
| 42 | R¹ = R² = R⁴ = H, R³ = c-Pr, and Z = Z-30 |
| 43 | R¹ = R² = R⁴ = H, R³ = CH₂Cl, and Z = Z-30 |
| 44 | R¹ = R² = R⁴ = H, R³ = CH₃, and Z = Z-34 |
| 45 | R¹ = R² = R⁴ = H, R³ = CH₃, and Z = Z-36 |
| 46 | R¹ = R² = R⁴ = H, R³ = CH₂CH₃, and Z = Z-36 |
| 47 | R¹ = R² = R⁴ = H, R³ = CH═CH₂, and Z = Z-36 |
| 48 | R¹ = R² = R⁴ = H, R³ = CF₃, and Z = Z-36 |
| 49 | R¹ = R² = R⁴ = H, R³ = OCH₃, and Z = Z-36 |
| 50 | R¹ = R² = R⁴ = H, R³ = c-Pr, and Z = Z-36 |
| 51 | R¹ = R² = R⁴ = H, R³ = CH₂Cl, and Z = Z-36 |
| 52 | R¹ = R² = R⁴ = H, R³ = CH₃, and Z = Z-42 |
| 53 | R¹ = R² = R⁴ = H, R³ = CH₂CH₃, and Z = Z-42 |
| 54 | R¹ = R² = R⁴ = H, R³ = CH═CH₂, and Z = Z-42 |
| 55 | R¹ = R² = R⁴ = H, R³ = CF₃, and Z = Z-42 |
| 56 | R¹ = R² = R⁴ = H, R³ = OCH₃, and Z = Z-42 |
| 57 | R¹ = R² = R⁴ = H, R³ = c-Pr, and Z = Z-42 |
| 58 | R¹ = R² = R⁴ = H, R³ = CH₂Cl, and Z = Z-42 |
| 59 | R¹ = R² = R⁴ = H, R³ = CH₃, and Z = Z-47 |
| 60 | R¹ = R² = R⁴ = H, R³ = CH₂CH₃, and Z = Z-47 |
| 61 | R¹ = R² = R⁴ = H, R³ = CH═CH₂, and Z = Z-47 |
| 62 | R¹ = R² = R⁴ = H, R³ = CF₃, and Z = Z-47 |
| 63 | R¹ = R² = R⁴ = H, R³ = OCH₃, and Z = Z-47 |
| 64 | R¹ = R² = R⁴ = H, R³ = c-Pr, and Z = Z-47 |
| 65 | R¹ = R² = R⁴ = H, R³ = CH₂Cl, and Z = Z-47 |
| 66 | R¹ = R² = R⁴ = H, R³ = CH₃, and Z = Z-48 |
| 67 | R¹ = R² = R⁴ = H, R³ = CH₂CH₃, and Z = Z-48 |
| 68 | R¹ = R² = R⁴ = H, R³ = CH═CH₂, and Z = Z-48 |
| 69 | R¹ = R² = R⁴ = H, R³ = CF₃, and Z = Z-48 |
| 70 | R¹ = R² = R⁴ = H, R³ = OCH₃, and Z = Z-48 |
| 71 | R¹ = R² = R⁴ = H, R³ = c-Pr, and Z = Z-48 |
| 72 | R¹ = R² = R⁴ = H, R³ = CH₂Cl, and Z = Z-48 |
| 73 | R¹ = R² = R⁴ = H, R³ = CH₃, and Z = Z-53 |
| 74 | R¹ = R² = R⁴ = H, R³ = CH₂CH₃, and Z = Z-53 |
| 75 | R¹ = R² = R⁴ = H, R³ = CH═CH₂, and Z = Z-53 |
| 76 | R¹ = R² = R⁴ = H, R³ = CF₃, and Z = Z-53 |
| 77 | R¹ = R² = R⁴ = H, R³ = OCH₃, and Z = Z-53 |
| 78 | R¹ = R² = R⁴ = H, R³ = c-Pr, and Z = Z-53 |
| 79 | R¹ = R² = R⁴ = H, R³ = CH₂Cl, and Z = Z-53 |
| 80 | R¹ = R² = R⁴ = H, R³ = CH₃, and Z = Z-54 |
| 81 | R¹ = R² = R⁴ = H, R³ = CH₂CH₃, and Z = Z-54 |
| 82 | R¹ = R² = R⁴ = H, R³ = CH═CH₂, and Z = Z-54 |
| 83 | R¹ = R² = R⁴ = H, R³ = CF₃, and Z = Z-55 |
| 84 | R¹ = R² = R⁴ = H, R³ = OCH₃, and Z = Z-55 |
| 85 | R¹ = R² = R⁴ = H, R³ = c-Pr, and Z = Z-55 |
| 86 | R¹ = R² = R⁴ = H, R³ = CH₂Cl, and Z = Z-55 |
| 87 | R¹ = R² = R⁴ = H, R³ = CH₃, and Z = Z-57 |
| 88 | R¹ = R² = R⁴ = H, R³ = CH₂CH₃, and Z = Z-57 |
| 89 | R¹ = R² = R⁴ = H, R³ = CH═CH₂, and Z = Z-57 |
| 90 | R¹ = R² = R⁴ = H, R³ = CF₃, and Z = Z-57 |
| 91 | R¹ = R² = R⁴ = H, R³ = OCH₃, and Z = Z-57 |
| 92 | R¹ = R² = R⁴ = H, R³ = c-Pr, and Z = Z-57 |
| 93 | R¹ = R² = R⁴ = H, R³ = CH₂Cl, and Z = Z-57 |
| 94 | R¹ = R² = R⁴ = H, R³ = CH₃, and Z = Z-62 |
| 95 | R¹ = R² = R⁴ = H, R³ = CH₂CH₃, and Z = Z-62 |
| 96 | R¹ = R² = R⁴ = H, R³ = CH═CH₂, and Z = Z-62 |
| 97 | R¹ = R² = R⁴ = H, R³ = CF₃, and Z = Z-62 |
| 98 | R¹ = R² = R⁴ = H, R³ = OCH₃, and Z = Z-62 |
| 99 | R¹ = R² = R⁴ = H, R³ = c-Pr, and Z = Z-62 |
| 100 | R¹ = R² = R⁴ = H, R³ = CH₂Cl, and Z = Z-62 |

TABLE 101

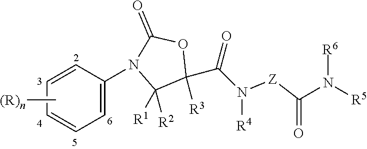

R¹ = R² = R⁴ = H, R³ = CH₃, and Z = Z-1 and the remaining
variables are defined below

| (R)ₙ | R⁵, R⁶ |
|---|---|
| 3-F | CH₃, H |
| 3-F | CH₂CH₃, H |
| 3-F | c-Pr, H |
| 3-F | H, H |
| 3-F | OCH₃, H |
| 3-F | OCH₂CH₃, H |
| 3-F | SO₂CH₃, H |
| 3-F | SO₂CF₃, H |
| 3-F | SO₂CH₂CH₃, H |
| 3-F | SO₂CH₂CH₂CH₃, H |
| 3-F | SO₂c-Pr, H |
| 3-F | SO₂Ph, H |
| 3-F | COCH₃, H |
| 3-F | COCF₃, H |
| 3-F | COPh, H |
| 3-F | SO₂N(CH₃)₂, H |
| 3-F | CON(CH₃)₂, H |
| 3-F | —CH₂CH₂CH₂CH₂— |
| 3-F | —CH₂CH₂CH₂CH₂CH₂— |
| 3-F | —CH₂CH₂OCH₂CH₂— |
| 3-F | —COCH₂CH₂CH₂— |
| 3-F | —COCH₂CH₂CH₂CH₂CH₂— |
| 3-F | CH₂CO₂CH₃ |
| 3-F | CH₂CO₂CH₂CH₃ |
| 3-F | CH₂COCH₃ |
| 3-F, 5-F | CH₃, H |
| 3-F, 5-F | CH₂CH₃, H |
| 3-F, 5-F | c-Pr, H |
| 3-F, 5-F | H, H |
| 3-F, 5-F | OCH₃, H |
| 3-F, 5-F | OCH₂CH₃, H |
| 3-F, 5-F | SO₂CH₃, H |
| 3-F, 5-F | CH₂CO₂CH₂CH₃ |
| 3-F, 5-F | CH₂COCH₃ |
| 3-F, 4-F, 5-F | CH₃, H |
| 3-F, 4-F, 5-F | CH₂CH₃, H |
| 3-F, 4-F, 5-F | c-Pr, H |
| 3-F, 4-F, 5-F | H, H |
| 3-F, 4-F, 5-F | OCH₃, H |
| 3-F, 4-F, 5-F | OCH₂CH₃, H |
| 3-F, 4-F, 5-F | SO₂CH₃, H |
| 3-F, 4-F, 5-F | SO₂CF₃, H |
| 3-F, 4-F, 5-F | SO₂CH₂CH₃, H |
| 3-F, 4-F, 5-F | SO₂CH₂CH₂CH₃, H |
| 3-F, 4-F, 5-F | SO₂c-Pr, H |
| 3-F, 4-F, 5-F | SO₂Ph, H |
| 3-F, 4-F, 5-F | COCH₃, H |
| 3-F, 4-F, 5-F | COCF₃, H |
| 3-F, 4-F, 5-F | COPh, H |
| 3-F, 4-F, 5-F | SO₂N(CH₃)₂, H |
| 3-F, 4-F, 5-F | CON(CH₃)₂, H |
| 3-F, 4-F, 5-F | —CH₂CH₂CH₂CH₂— |
| 3-F, 4-F, 5-F | —CH₂CH₂CH₂CH₂CH₂— |
| 3-F, 4-F, 5-F | —CH₂CH₂OCH₂CH₂— |
| 3-F, 4-F, 5-F | —COCH₂CH₂CH₂— |
| 3-F, 4-F, 5-F | —COCH₂CH₂CH₂CH₂CH₂— |
| 3-F, 4-F, 5-F | CH₂CO₂CH₃ |
| 3-F, 4-F, 5-F | CH₂CO₂CH₂CH₃ |
| 3-F, 4-F, 5-F | CH₂COCH₃ |
| 3-Cl | CH₃, H |
| 3-Cl | CH₂CH₃, H |
| 3-Cl | c-Pr, H |
| 3-Cl | H, H |
| 3-Cl | OCH₃, H |
| 3-Cl | OCH₂CH₃, H |
| 3-Cl | SO₂CH₃, H |
| 3-Cl | SO₂CF₃, H |
| 3-Cl | SO₂CH₂CH₃, H |
| 3-Cl | SO₂CH₂CH₂CH₃, H |
| 3-Cl | SO₂c-Pr, H |
| 3-Cl | SO₂Ph, H |
| 3-Cl | COCH₃, H |
| 3-Cl | COCF₃, H |
| 3-Cl | COPh, H |
| 3-Cl | SO₂N(CH₃)₂, H |
| 3-Cl | CON(CH₃)₂, H |
| 3-Cl | —CH₂CH₂CH₂CH₂— |
| 3-Cl | —CH₂CH₂CH₂CH₂CH₂— |
| 3-Cl | —CH₂CH₂OCH₂CH₂— |
| 3-Cl | —COCH₂CH₂CH₂— |
| 3-Cl | —COCH₂CH₂CH₂CH₂CH₂— |
| 3-Cl | CH₂CO₂CH₃ |
| 3-Cl | CH₂CO₂CH₂CH₃ |
| 3-Cl | CH₂COCH₃ |
| 3-Cl, 5-Cl | CH₃, H |
| 3-Cl, 5-Cl | CH₂CH₃, H |
| 3-Cl, 5-Cl | c-Pr, H |
| 3-Cl, 5-Cl | H, H |
| 3-Cl, 5-Cl | OCH₃, H |
| 3-Cl, 5-Cl | OCH₂CH₃, H |
| 3-Cl, 5-Cl | SO₂CH₃, H |
| 3-Cl, 5-Cl | SO₂CF₃, H |
| 3-Cl, 5-Cl | SO₂CH₂CH₃, H |
| 3-Cl, 5-Cl | SO₂CH₂CH₂CH₃, H |
| 3-Cl, 5-Cl | SO₂c-Pr, H |
| 3-Cl, 5-Cl | SO₂Ph, H |
| 3-Cl, 5-Cl | COCH₃, H |
| 3-Cl, 5-Cl | COCF₃, H |
| 3-Cl, 5-Cl | COPh, H |
| 3-Cl, 5-Cl | SO₂N(CH₃)₂, H |
| 3-Cl, 5-Cl | CON(CH₃)₂, H |
| 3-Cl, 5-Cl | —CH₂CH₂CH₂CH₂— |
| 3-Cl, 5-Cl | —CH₂CH₂CH₂CH₂CH₂— |
| 3-Cl, 5-Cl | —CH₂CH₂OCH₂CH₂— |
| 3-Cl, 5-Cl | —COCH₂CH₂CH₂— |
| 3-Cl, 5-Cl | —COCH₂CH₂CH₂CH₂CH₂— |
| 3-Cl, 5-Cl | CH₂CO₂CH₃ |
| 3-Cl, 5-Cl | CH₂CO₂CH₂CH₃ |
| 3-Cl, 5-Cl | CH₂COCH₃ |
| 3-Cl, 4-F, 5-Cl | CH₃, H |
| 3-Cl, 4-F, 5-Cl | CH₂CH₃, H |
| 3-Cl, 4-F, 5-Cl | c-Pr, H |
| 3-Cl, 4-F, 5-Cl | H, H |
| 3-Cl, 4-F, 5-Cl | OCH₃, H |
| 3-Cl, 4-F, 5-Cl | OCH₂CH₃, H |
| 3-Cl, 4-F, 5-Cl | SO₂CH₃, H |
| 3-Cl, 4-F, 5-Cl | SO₂CF₃, H |
| 3-Cl, 4-F, 5-Cl | SO₂CH₂CH₃, H |
| 3-Cl, 4-F, 5-Cl | SO₂CH₂CH₂CH₃, H |
| 3-Cl, 4-F, 5-Cl | SO₂c-Pr, H |
| 3-Cl, 4-F, 5-Cl | SO₂Ph, H |
| 3-Cl, 4-F, 5-Cl | COCH₃, H |
| 3-Cl, 4-F, 5-Cl | COCF₃, H |
| 3-Cl, 4-F, 5-Cl | COPh, H |
| 3-Cl, 4-F, 5-Cl | SO₂N(CH₃)₂, H |
| 3-Cl, 4-F, 5-Cl | CON(CH₃)₂, H |
| 3-Cl, 4-F, 5-Cl | —CH₂CH₂CH₂CH₂— |
| 3-Cl, 4-F, 5-Cl | —CH₂CH₂CH₂CH₂CH₂— |
| 3-Cl, 4-F, 5-Cl | —CH₂CH₂OCH₂CH₂— |
| 3-Cl, 4-F, 5-Cl | —COCH₂CH₂CH₂— |
| 3-Cl, 4-F, 5-Cl | —COCH₂CH₂CH₂CH₂CH₂— |
| 3-Cl, 4-F, 5-Cl | CH₂CO₂CH₃ |
| 3-Cl, 4-F, 5-Cl | CH₂CO₂CH₂CH₃ |
| 3-Cl, 4-F, 5-Cl | CH₂COCH₃ |
| 3-CF₃ | CH₃, H |
| 3-CF₃ | CH₂CH₃, H |
| 3-CF₃ | c-Pr, H |
| 3-CF₃ | H, H |
| 3-CF₃ | OCH₃, H |
| 3-CF₃ | OCH₂CH₃, H |
| 3-CF₃ | SO₂CH₃, H |
| 3-CF₃ | SO₂CF₃, H |
| 3-CF₃ | SO₂CH₂CH₃, H |
| 3-CF₃ | SO₂CH₂CH₂CH₃, H |
| 3-CF₃ | SO₂c-Pr, H |
| 3-CF₃ | SO₂Ph, H |
| 3-CF₃ | COCH₃, H |
| 3-CF₃ | COCF₃, H |
| 3-CF₃ | COPh, H |
| 3-CF₃ | SO₂N(CH₃)₂, H |
| 3-CF₃ | CON(CH₃)₂, H |
| 3-CF₃ | —CH₂CH₂CH₂CH₂— |
| 3-CF₃ | —CH₂CH₂CH₂CH₂CH₂— |
| 3-CF₃ | —CH₂CH₂OCH₂CH₂— |
| 3-CF₃ | —COCH₂CH₂CH₂— |
| 3-CF₃ | —COCH₂CH₂CH₂CH₂CH₂— |
| 3-CF₃ | CH₂CO₂CH₃ |
| 3-CF₃ | CH₂CO₂CH₂CH₃ |
| 3-CF₃ | CH₂COCH₃ |
| 3-CF₃, 5-F | CH₃, H |
| 3-CF₃, 5-F | CH₂CH₃, H |
| 3-CF₃, 5-F | c-Pr, H |
| 3-CF₃, 5-F | H, H |
| 3-CF₃, 5-F | OCH₃, H |
| 3-CF₃, 5-F | OCH₂CH₃, H |
| 3-CF₃, 5-F | SO₂CH₃, H |
| 3-CF₃, 5-F | SO₂CF₃, H |
| 3-CF₃, 5-F | SO₂CH₂CH₃, H |
| 3-CF₃, 5-F | SO₂CH₂CH₂CH₃, H |
| 3-CF₃, 5-F | SO₂c-Pr, H |
| 3-CF₃, 5-F | SO₂Ph, H |
| 3-CF₃, 5-F | COCH₃, H |
| 3-CF₃, 5-F | COCF₃, H |
| 3-CF₃, 5-F | COPh, H |
| 3-CF₃, 5-F | SO₂N(CH₃)₂, H |
| 3-CF₃, 5-F | CON(CH₃)₂, H |
| 3-CF₃, 5-F | —CH₂CH₂CH₂CH₂— |
| 3-CF₃, 5-F | —CH₂CH₂CH₂CH₂CH₂— |
| 3-CF₃, 5-F | —CH₂CH₂OCH₂CH₂— |
| 3-CF₃, 5-F | —COCH₂CH₂CH₂— |
| 3-CF₃, 5-F | —COCH₂CH₂CH₂CH₂CH₂— |
| 3-CF₃, 5-F | CH₂CO₂CH₃ |
| 3-CF₃, 5-F | CH₂CO₂CH₂CH₃ |
| 3-CF₃, 5-F | CH₂COCH₃ |
| 3-CF₃, 5-Cl | CH₃, H |
| 3-CF₃, 5-Cl | CH₂CH₃, H |
| 3-CF₃, 5-Cl | c-Pr, H |
| 3-CF₃, 5-Cl | H, H |
| 3-CF₃, 5-Cl | OCH₃, H |
| 3-CF₃, 5-Cl | OCH₂CH₃, H |
| 3-CF₃, 5-Cl | SO₂CH₃, H |
| 3-CF₃, 5-Cl | SO₂CF₃, H |
| 3-CF₃, 5-Cl | SO₂CH₂CH₃, H |
| 3-CF₃, 5-Cl | SO₂CH₂CH₂CH₃, H |
| 3-CF₃, 5-Cl | SO₂c-Pr, H |
| 3-CF₃, 5-Cl | SO₂Ph, H |
| 3-CF₃, 5-Cl | COCH₃, H |
| 3-CF₃, 5-Cl | COCF₃, H |
| 3-CF₃, 5-Cl | COPh, H |
| 3-CF₃, 5-Cl | SO₂N(CH₃)₂, H |
| 3-CF₃, 5-Cl | CON(CH₃)₂, H |
| 3-CF₃, 5-Cl | —CH₂CH₂CH₂CH₂— |
| 3-CF₃, 5-Cl | —CH₂CH₂CH₂CH₂CH₂— |
| 3-CF₃, 5-Cl | —CH₂CH₂OCH₂CH₂— |
| 3-CF₃, 5-Cl | —COCH₂CH₂CH₂— |
| 3-CF₃, 5-Cl | —COCH₂CH₂CH₂CH₂CH₂— |
| 3-CF₃, 5-Cl | CH₂CO₂CH₃ |
| 3-CF₃, 5-Cl | CH₂CO₂CH₂CH₃ |
| 3-CF₃, 5-Cl | CH₂COCH₃ |
| 3-OCF₃, 5-F | CH₃, H |
| 3-OCF₃, 5-F | CH₂CH₃, H |
| 3-OCF₃, 5-F | c-Pr, H |
| 3-OCF₃, 5-F | H, H |
| 3-OCF₃, 5-F | OCH₃, H |
| 3-OCF₃, 5-F | OCH₂CH₃, H |
| 3-OCF₃, 5-F | SO₂CH₃, H |
| 3-OCF₃, 5-F | SO₂CF₃, H |
| 3-OCF₃, 5-F | SO₂CH₂CH₃, H |
| 3-OCF₃, 5-F | SO₂CH₂CH₂CH₃, H |
| 3-OCF₃, 5-F | SO₂c-Pr, H |
| 3-OCF₃, 5-F | SO₂Ph, H |
| 3-OCF₃, 5-F | COCH₃, H |
| 3-OCF₃, 5-F | COCF₃, H |
| 3-OCF₃, 5-F | COPh, H |
| 3-OCF₃, 5-F | SO₂N(CH₃)₂, H |
| 3-OCF₃, 5-F | CON(CH₃)₂, H |
| 3-OCF₃, 5-F | —CH₂CH₂CH₂CH₂— |
| 3-OCF₃, 5-F | —CH₂CH₂CH₂CH₂CH₂— |
| 3-OCF₃, 5-F | —CH₂CH₂OCH₂CH₂— |
| 3-OCF₃, 5-F | —COCH₂CH₂CH₂— |
| 3-OCF₃, 5-F | —COCH₂CH₂CH₂CH₂CH₂— |
| 3-OCF₃, 5-F | CH₂CO₂CH₃ |
| 3-OCF₃, 5-F | CH₂CO₂CH₂CH₃ |
| 3-OCF₃, 5-F | CH₂COCH₃ |
| 3-OCF₃, 5-Cl | CH₃, H |
| 3-OCF₃, 5-Cl | CH₂CH₃, H |
| 3-OCF₃, 5-Cl | c-Pr, H |
| 3-OCF₃, 5-Cl | H, H |
| 3-OCF₃, 5-Cl | OCH₃, H |
| 3-OCF₃, 5-Cl | OCH₂CH₃, H |
| 3-OCF₃, 5-Cl | SO₂CH₃, H |
| 3-OCF₃, 5-Cl | SO₂CF₃, H |
| 3-OCF₃, 5-Cl | SO₂CH₂CH₃, H |
| 3-OCF₃, 5-Cl | SO₂CH₂CH₂CH₃, H |
| 3-OCF₃, 5-Cl | SO₂c-Pr, H |
| 3-OCF₃, 5-Cl | SO₂Ph, H |
| 3-OCF₃, 5-Cl | COCH₃, H |
| 3-OCF₃, 5-Cl | COCF₃, H |
| 3-OCF₃, 5-Cl | COPh, H |
| 3-OCF₃, 5-Cl | SO₂N(CH₃)₂, H |
| 3-OCF₃, 5-Cl | CON(CH₃)₂, H |
| 3-OCF₃, 5-Cl | —CH₂CH₂CH₂CH₂— |
| 3-OCF₃, 5-Cl | —CH₂CH₂CH₂CH₂CH₂— |
| 3-OCF₃, 5-Cl | —CH₂CH₂OCH₂CH₂— |
| 3-OCF₃, 5-Cl | —COCH₂CH₂CH₂— |
| 3-OCF₃, 5-Cl | —COCH₂CH₂CH₂CH₂CH₂— |
| 3-OCF₃, 5-Cl | CH₂CO₂CH₃ |
| 3-OCF₃, 5-Cl | CH₂CO₂CH₂CH₃ |
| 3-OCF₃, 5-Cl | CH₂COCH₃ |

The present disclosure also includes Tables 102-200, each of which is constructed the same as Table 101 above, except that the row heading in Table 101 (i.e. R¹═R²═R⁴═H, R³═CH₃, and Z═Z-1) is replaced with the respective row heading shown below.

| TABLE | Header Row Variable |
|---|---|
| 102 | R¹ = R² = R⁴ = H, R³ = CH₂CH₃, and Z = Z-1 |
| 103 | R¹ = R² = R⁴ = H, R³ = CH═CH₂, and Z = Z-1 |
| 104 | R¹ = R² = R⁴ = H, R³ = CF₃, and Z = Z-1 |
| 105 | R¹ = R² = R⁴ = H, R³ = OCH₃, and Z = Z-1 |
| 106 | R¹ = R² = R⁴ = H, R³ = c-Pr, and Z = Z-1 |
| 107 | R¹ = R² = R⁴ = H, R³ = CH₂Cl, and Z = Z-1 |
| 108 | R¹ = R² = R⁴ = H, R³ = CH₃, and Z = Z-4 |
| 109 | R¹ = R² = R⁴ = H, R³ = CH₂CH₃, and Z = Z-4 |
| 110 | R¹ = R² = R⁴ = H, R³ = CH═CH₂, and Z = Z-4 |
| 111 | R¹ = R² = R⁴ = H, R³ = CF₃, and Z = Z-4 |
| 112 | R¹ = R² = R⁴ = H, R³ = OCH₃, and Z = Z-4 |
| 113 | R¹ = R² = R⁴ = H, R³ = c-Pr, and Z = Z-4 |
| 114 | R¹ = R² = R⁴ = H, R³ = CH₂Cl, and Z = Z-4 |
| 115 | R¹ = R² = R⁴ = H, R³ = CH₃, and Z = Z-6 |
| 116 | R¹ = R² = R⁴ = H, R³ = CH₂CH₃, and Z = Z-6 |
| 117 | R¹ = R² = R⁴ = H, R³ = CH═CH₂, and Z = Z-6 |
| 118 | R¹ = R² = R⁴ = H, R³ = CF₃, and Z = Z-6 |
| 119 | R¹ = R² = R⁴ = H, R³ = OCH₃, and Z = Z-6 |
| 120 | R¹ = R² = R⁴ = H, R³ = c-Pr, and Z = Z-6 |
| 121 | R¹ = R² = R⁴ = H, R³ = CH₂Cl, and Z = Z-6 |
| 122 | R¹ = R² = R⁴ = H, R³ = CH₃, and Z = Z-12 |
| 123 | R¹ = R² = R⁴ = H, R³ = CH₂CH₃, and Z = Z-12 |
| 124 | R¹ = R² = R⁴ = H, R³ = CH═CH₂, and Z = Z-12 |
| 125 | R¹ = R² = R⁴ = H, R³ = CF₃, and Z = Z-12 |
| 126 | R¹ = R² = R⁴ = H, R³ = OCH₃, and Z = Z-12 |
| 127 | R¹ = R² = R⁴ = H, R³ = c-Pr, and Z = Z-12 |
| 128 | R¹ = R² = R⁴ = H, R³ = CH₂Cl, and Z = Z-12 |
| 129 | R¹ = R² = R⁴ = H, R³ = CH₃, and Z = Z-22 |
| 130 | R¹ = R² = R⁴ = H, R³ = CH₂CH₃, and Z = Z-22 |
| 131 | R¹ = R² = R⁴ = H, R³ = CH═CH₂, and Z = Z-22 |
| 132 | R¹ = R² = R⁴ = H, R³ = CF₃, and Z = Z-22 |
| 133 | R¹ = R² = R⁴ = H, R³ = OCH₃, and Z = Z-22 |
| 134 | R¹ = R² = R⁴ = H, R³ = c-Pr, and Z = Z-22 |
| 135 | R¹ = R² = R⁴ = H, R³ = CH₂Cl, and Z = Z-22 |
| 136 | R¹ = R² = R⁴ = H, R³ = CH₃, and Z = Z-24 |
| 137 | R¹ = R² = R⁴ = H, R³ = CH₃, and Z = Z-30 |
| 138 | R¹ = R² = R⁴ = H, R³ = CH₂CH₃, and Z = Z-30 |
| 139 | R¹ = R² = R⁴ = H, R³ = CH═CH₂, and Z = Z-30 |
| 140 | R¹ = R² = R⁴ = H, R³ = CF₃, and Z = Z-30 |
| 141 | R¹ = R² = R⁴ = H, R³ = OCH₃, and Z = Z-30 |
| 142 | R¹ = R² = R⁴ = H, R³ = c-Pr, and Z = Z-30 |
| 143 | R¹ = R² = R⁴ = H, R³ = CH₂Cl, and Z = Z-30 |
| 144 | R¹ = R² = R⁴ = H, R³ = CH₃, and Z = Z-34 |
| 145 | R¹ = R² = R⁴ = H, R³ = CH₃, and Z = Z-36 |
| 146 | R¹ = R² = R⁴ = H, R³ = CH₂CH₃, and Z = Z-36 |
| 147 | R¹ = R² = R⁴ = H, R³ = CH═CH₂, and Z = Z-36 |
| 148 | R¹ = R² = R⁴ = H, R³ = CF₃, and Z = Z-36 |
| 149 | R¹ = R² = R⁴ = H, R³ = OCH₃, and Z = Z-36 |
| 150 | R¹ = R² = R⁴ = H, R³ = c-Pr, and Z = Z-36 |
| 151 | R¹ = R² = R⁴ = H, R³ = CH₂Cl, and Z = Z-36 |
| 152 | R¹ = R² = R⁴ = H, R³ = CH₃, and Z = Z-42 |
| 153 | R¹ = R² = R⁴ = H, R³ = CH₂CH₃, and Z = Z-42 |
| 154 | R¹ = R² = R⁴ = H, R³ = CH═CH₂, and Z = Z-42 |
| 155 | R¹ = R² = R⁴ = H, R³ = CF₃, and Z = Z-42 |
| 156 | R¹ = R² = R⁴ = H, R³ = OCH₃, and Z = Z-42 |
| 157 | R¹ = R² = R⁴ = H, R³ = c-Pr, and Z = Z-42 |
| 158 | R¹ = R² = R⁴ = H, R³ = CH₂Cl, and Z = Z-42 |
| 159 | R¹ = R² = R⁴ = H, R³ = CH₃, and Z = Z-47 |
| 160 | R¹ = R² = R⁴ = H, R³ = CH₂CH₃, and Z = Z-47 |
| 161 | R¹ = R² = R⁴ = H, R³ = CH═CH₂, and Z = Z-47 |
| 162 | R¹ = R² = R⁴ = H, R³ = CF₃, and Z = Z-47 |
| 163 | R¹ = R² = R⁴ = H, R³ = OCH₃, and Z = Z-47 |
| 164 | R¹ = R² = R⁴ = H, R³ = c-Pr, and Z = Z-47 |
| 165 | R¹ = R² = R⁴ = H, R³ = CH₂Cl, and Z = Z-47 |
| 166 | R¹ = R² = R⁴ = H, R³ = CH₃, and Z = Z-48 |
| 167 | R¹ = R² = R⁴ = H, R³ = CH₂CH₃, and Z = Z-48 |
| 168 | R¹ = R² = R⁴ = H, R³ = CH═CH₂, and Z = Z-48 |
| 169 | R¹ = R² = R⁴ = H, R³ = CF₃, and Z = Z-48 |
| 170 | R¹ = R² = R⁴ = H, R³ = OCH₃, and Z = Z-48 |
| 171 | R¹ = R² = R⁴ = H, R³ = c-Pr, and Z = Z-48 |
| 172 | R¹ = R² = R⁴ = H, R³ = CH₂Cl, and Z = Z-48 |
| 173 | R¹ = R² = R⁴ = H, R³ = CH₃, and Z = Z-53 |
| 174 | R¹ = R² = R⁴ = H, R³ = CH₂CH₃, and Z = Z-53 |
| 175 | R¹ = R² = R⁴ = H, R³ = CH═CH₂, and Z = Z-53 |
| 176 | R¹ = R² = R⁴ = H, R³ = CF₃, and Z = Z-53 |
| 177 | R¹ = R² = R⁴ = H, R³ = OCH₃, and Z = Z-53 |
| 178 | R¹ = R² = R⁴ = H, R³ = c-Pr, and Z = Z-53 |
| 179 | R¹ = R² = R⁴ = H, R³ = CH₂Cl, and Z = Z-53 |
| 180 | R¹ = R² = R⁴ = H, R³ = CH₃, and Z = Z-54 |
| 181 | R¹ = R² = R⁴ = H, R³ = CH₂CH₃, and Z = Z-54 |
| 182 | R¹ = R² = R⁴ = H, R³ = CH═CH₂, and Z = Z-54 |
| 183 | R¹ = R² = R⁴ = H, R³ = CF₃, and Z = Z-55 |
| 184 | R¹ = R² = R⁴ = H, R³ = OCH₃, and Z = Z-55 |
| 185 | R¹ = R² = R⁴ = H, R³ = c-Pr, and Z = Z-55 |
| 186 | R¹ = R² = R⁴ = H, R³ = CH₂Cl, and Z = Z-55 |
| 187 | R¹ = R² = R⁴ = H, R³ = CH₃, and Z = Z-57 |
| 188 | R¹ = R² = R⁴ = H, R³ = CH₂CH₃, and Z = Z-57 |
| 189 | R¹ = R² = R⁴ = H, R³ = CH═CH₂, and Z = Z-57 |
| 190 | R¹ = R² = R⁴ = H, R³ = CF₃, and Z = Z-57 |
| 191 | R¹ = R² = R⁴ = H, R³ = OCH₃, and Z = Z-57 |
| 192 | R¹ = R² = R⁴ = H, R³ = c-Pr, and Z = Z-57 |
| 193 | R¹ = R² = R⁴ = H, R³ = CH₂Cl, and Z = Z-57 |
| 194 | R¹ = R² = R⁴ = H, R³ = CH₃, and Z = Z-62 |
| 195 | R¹ = R² = R⁴ = H, R³ = CH₂CH₃, and Z = Z-62 |
| 196 | R¹ = R² = R⁴ = H, R³ = CH═CH₂, and Z = Z-62 |
| 197 | R¹ = R² = R⁴ = H, R³ = CF₃, and Z = Z-62 |
| 198 | R¹ = R² = R⁴ = H, R³ = OCH₃, and Z = Z-62 |
| 199 | R¹ = R² = R⁴ = H, R³ = c-Pr, and Z = Z-62 |
| 200 | R¹ = R² = R⁴ = H, R³ = CH₂Cl, and Z = Z-62 |

### Table 201

Table 201 is constructed the same as TABLE 1 except the structure is replaced with the following structure:

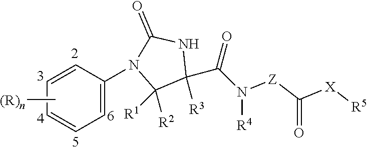

where R¹, R², R³, R⁴ and Z are as defined in TABLE 1, and the remaining variables are as defined in TABLE 1.

The present disclosure also includes TABLES 202-300, each of which is constructed the same as TABLE 201 above, except that the row heading in Table 1 (i.e. R¹═R²═R⁴═H, R³═CH₃, and Z═Z-1) is replaced with the respective row heading shown in TABLES 2-100 above.

### Table 301

Table 301 is constructed the same as TABLE 101 except the structure is replaced with the following structure

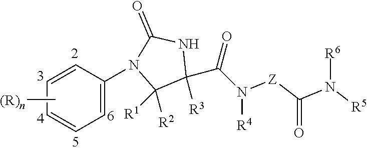

where R¹, R², R³, R⁴ and Z are as defined in TABLE 1, and the remaining variables are as defined in TABLE 101.

The present disclosure also includes TABLES 302-400, each of which is constructed the same as Table 301 above, except that the row heading in Table 301 (i.e. R¹═R²═R⁴═H, R³═CH₃, and Z═Z-1) is replaced with the respective row heading shown in TABLES 101-200.

### Formulation/Utility

A compound of this invention will generally be used as a herbicidal active ingredient in a composition, i.e. formulation, with at least one additional component selected from the group consisting of surfactants, solid diluents and liquid diluents, which serves as a carrier. The formulation or composition ingredients are selected to be consistent with the physical properties of the active ingredient, mode of application and environmental factors such as soil type, moisture and temperature.

Useful formulations include both liquid and solid compositions. Liquid compositions include solutions (including emulsifiable concentrates), suspensions, emulsions (including microemulsions, oil-in-water emulsions, flowable concentrates and/or suspoemulsions) and the like, which optionally can be thickened into gels. The general types of aqueous liquid compositions are soluble concentrate, suspension concentrate, capsule suspension, concentrated emulsion, microemulsion, oil-in-water emulsion, flowable concentrate and suspo-emulsion. The general types of nonaqueous liquid compositions are emulsifiable concentrate, microemulsifiable concentrate, dispersible concentrate and oil dispersion.

The general types of solid compositions are dusts, powders, granules, pellets, prills, pastilles, tablets, filled films (including seed coatings) and the like, which can be water-dispersible (“wettable”) or water-soluble. Films and coatings formed from film-forming solutions or flowable suspensions are particularly useful for seed treatment. Active ingredient can be (micro) encapsulated and further formed into a suspension or solid formulation; alternatively the entire formulation of active ingredient can be encapsulated (or “overcoated”). Encapsulation can control or delay release of the active ingredient. An emulsifiable granule combines the advantages of both an emulsifiable concentrate formulation and a dry granular formulation. High-strength compositions are primarily used as intermediates for further formulation.

Sprayable formulations are typically extended in a suitable medium before spraying. Such liquid and solid formulations are formulated to be readily diluted in the spray medium, usually water, but occasionally another suitable medium like an aromatic or paraffinic hydrocarbon or vegetable oil. Spray volumes can range from about from about one to several thousand liters per hectare, but more typically are in the range from about ten to several hundred liters per hectare. Sprayable formulations can be tank mixed with water or another suitable medium for foliar treatment by aerial or ground application, or for application to the growing medium of the plant. Liquid and dry formulations can be metered directly into drip irrigation systems or metered into the furrow during planting.

The formulations will typically contain effective amounts of active ingredient, diluent and surfactant within the following approximate ranges which add up to 100 percent by weight.

| | Weight Percent | | |
|---|---|---|---|
| | Active | | |
| | Ingredient | Diluent | Surfactant |
| Water-Dispersible and Water- | 0.001-90 | 0-99.999 | 0-15 |
| soluble Granules, Tablets and | | | |
| Powders | | | |
| Oil Dispersions, Suspensions, | 1-50 | 40-99 | 0-50 |
| Emulsions, Solutions (including | | | |
| Emulsifiable Concentrates) | | | |
| Dusts | 1-25 | 70-99 | 0-5 |
| Granules and Pellets | 0.001-99 | 5-99.999 | 0-15 |
| High Strength Compositions | 90-99 | 0-10 | 0-2 |

Solid diluents include, for example, clays such as bentonite, montmorillonite, attapulgite and kaolin, gypsum, cellulose, titanium dioxide, zinc oxide, starch, dextrin, sugars (e.g., lactose, sucrose), silica, talc, mica, diatomaceous earth, urea, calcium carbonate, sodium carbonate and bicarbonate, and sodium sulfate. Typical solid diluents are described in Watkins et al., Handbook of Insecticide Dust Diluents and Carriers, 2nd Ed., Dorland Books, Caldwell, New Jersey.

Liquid diluents include, for example, water, N,N-dimethylalkanamides (e.g., N,N-dimethylformamide), limonene, dimethyl sulfoxide, N-alkylpyrrolidones (e.g., N-methylpyrrolidinone), alkyl phosphates (e.g., triethyl phosphate), ethylene glycol, triethylene glycol, propylene glycol, dipropylene glycol, polypropylene glycol, propylene carbonate, butylene carbonate, paraffins (e.g., white mineral oils, normal paraffins, isoparaffins), alkylbenzenes, alkylnaphthalenes, glycerine, glycerol triacetate, sorbitol, aromatic hydrocarbons, dearomatized aliphatics, alkylbenzenes, alkylnaphthalenes, ketones such as cyclohexanone, 2-heptanone, isophorone and 4-hydroxy-4-methyl-2-pentanone, acetates such as isoamyl acetate, hexyl acetate, heptyl acetate, octyl acetate, nonyl acetate, tridecyl acetate and isobornyl acetate, other esters such as alkylated lactate esters, dibasic esters, alkyl and aryl benzoates and γ-butyrolactone, and alcohols, which can be linear, branched, saturated or unsaturated, such as methanol, ethanol, n-propanol, isopropyl alcohol, n-butanol, isobutyl alcohol, n-hexanol, 2-ethylhexanol, n-octanol, decanol, isodecyl alcohol, isooctadecanol, cetyl alcohol, lauryl alcohol, tridecyl alcohol, oleyl alcohol, cyclohexanol, tetrahydrofurfuryl alcohol, diacetone alcohol, cresol and benzyl alcohol. Liquid diluents also include glycerol esters saturated and unsaturated fatty acids (typically C₆-C₂₂), such as plant seed and fruit oils (e.g., oils of olive, castor, linseed, sesame, corn (maize), peanut, sunflower, grapeseed, safflower, cottonseed, soybean, rapeseed, coconut and palm kernel), animal-sourced fats (e.g., beef tallow, pork tallow, lard, cod liver oil, fish oil), and mixtures thereof. Liquid diluents also include alkylated fatty acids (e.g., methylated, ethylated, butylated) wherein the fatty acids may be obtained by hydrolysis of glycerol esters from plant and animal sources, and can be purified by distillation. Typical liquid diluents are described in Marsden, Solvents Guide, 2nd Ed., Interscience, New York, 1950.

The solid and liquid compositions of the present invention often include one or more surfactants. When added to a liquid, surfactants (also known as “surface-active agents”) generally modify, most often reduce, the surface tension of the liquid. Depending on the nature of the hydrophilic and lipophilic groups in a surfactant molecule, surfactants can be useful as wetting agents, dispersants, emulsifiers or defoaming agents.

Surfactants can be classified as nonionic, anionic or cationic. Nonionic surfactants useful for the present compositions include, but are not limited to: alcohol alkoxylates such as alcohol alkoxylates based on natural and synthetic alcohols (which may be branched or linear) and prepared from the alcohols and ethylene oxide, propylene oxide, butylene oxide or mixtures thereof; amine ethoxylates, alkanolamides and ethoxylated alkanolamides; alkoxylated triglycerides such as ethoxylated soybean, castor and rapeseed oils; alkylphenol alkoxylates such as octylphenol ethoxylates, nonylphenol ethoxylates, dinonyl phenol ethoxylates and dodecyl phenol ethoxylates (prepared from the phenols and ethylene oxide, propylene oxide, butylene oxide or mixtures thereof); block polymers prepared from ethylene oxide or propylene oxide and reverse block polymers where the terminal blocks are prepared from propylene oxide; ethoxylated fatty acids; ethoxylated fatty esters and oils; ethoxylated methyl esters; ethoxylated tristyrylphenol (including those prepared from ethylene oxide, propylene oxide, butylene oxide or mixtures thereof); fatty acid esters, glycerol esters, lanolin-based derivatives, polyethoxylate esters such as polyethoxylated sorbitan fatty acid esters, polyethoxylated sorbitol fatty acid esters and polyethoxylated glycerol fatty acid esters; other sorbitan derivatives such as sorbitan esters; polymeric surfactants such as random copolymers, block copolymers, alkyd peg (polyethylene glycol) resins, graft or comb polymers and star polymers; polyethylene glycols (pegs); polyethylene glycol fatty acid esters; silicone-based surfactants; and sugar-derivatives such as sucrose esters, alkyl polyglycosides and alkyl polysaccharides.

Useful anionic surfactants include, but are not limited to: alkylaryl sulfonic acids and their salts; carboxylated alcohol or alkylphenol ethoxylates; diphenyl sulfonate derivatives; lignin and lignin derivatives such as lignosulfonates; maleic or succinic acids or their anhydrides; olefin sulfonates; phosphate esters such as phosphate esters of alcohol alkoxylates, phosphate esters of alkylphenol alkoxylates and phosphate esters of styryl phenol ethoxylates; protein-based surfactants; sarcosine derivatives; styryl phenol ether sulfate; sulfates and sulfonates of oils and fatty acids; sulfates and sulfonates of ethoxylated alkylphenols; sulfates of alcohols; sulfates of ethoxylated alcohols; sulfonates of amines and amides such as N,N-alkyltaurates; sulfonates of benzene, cumene, toluene, xylene, and dodecyl and tridecylbenzenes; sulfonates of condensed naphthalenes; sulfonates of naphthalene and alkyl naphthalene; sulfonates of fractionated petroleum; sulfosuccinamates; and sulfosuccinates and their derivatives such as dialkyl sulfosuccinate salts.

Useful cationic surfactants include, but are not limited to: amides and ethoxylated amides; amines such as N-alkyl propanediamines, tripropylenetriamines and dipropylenetetramines, and ethoxylated amines, ethoxylated diamines and propoxylated amines (prepared from the amines and ethylene oxide, propylene oxide, butylene oxide or mixtures thereof); amine salts such as amine acetates and diamine salts; quaternary ammonium salts such as quaternary salts, ethoxylated quaternary salts and diquaternary salts; and amine oxides such as alkyldimethylamine oxides and bis-(2-hydroxyethyl)-alkylamine oxides.

Also useful for the present compositions are mixtures of nonionic and anionic surfactants or mixtures of nonionic and cationic surfactants. Nonionic, anionic and cationic surfactants and their recommended uses are disclosed in a variety of published references including Mccutcheon's Emulsifiers and Detergents, annual American and International Editions published by Mccutcheon's Division, The Manufacturing Confectioner Publishing Co.; Sisely and Wood, Encyclopedia of Surface Active Agents, Chemical Publ. Co., Inc., New York, 1964; and A. S. Davidson and B. Milwidsky, Synthetic Detergents, Seventh Edition, John Wiley and Sons, New York, 1987.

Compositions of this invention may also contain formulation auxiliaries and additives, known to those skilled in the art as formulation aids (some of which may be considered to also function as solid diluents, liquid diluents or surfactants). Such formulation auxiliaries and additives may control: pH (buffers), foaming during processing (antifoams such polyorganosiloxanes), sedimentation of active ingredients (suspending agents), viscosity (thixotropic thickeners), in-container microbial growth (antimicrobials), product freezing (antifreezes), color (dyes/pigment dispersions), wash-off (film formers or stickers), evaporation (evaporation retardants), and other formulation attributes. Film formers include, for example, polyvinyl acetates, polyvinyl acetate copolymers, polyvinylpyrrolidone-vinyl acetate copolymer, polyvinyl alcohols, polyvinyl alcohol copolymers and waxes. Examples of formulation auxiliaries and additives include those listed in McCutcheon's Volume 2: Functional Materials, annual International and North American editions published by Mccutcheon's Division, The Manufacturing Confectioner Publishing Co.; and PCT Publication WO 03/024222.

The compound of Formula 1 and any other active ingredients are typically incorporated into the present compositions by dissolving the active ingredient in a solvent or by grinding in a liquid or dry diluent. Solutions, including emulsifiable concentrates, can be prepared by simply mixing the ingredients. If the solvent of a liquid composition intended for use as an emulsifiable concentrate is water-immiscible, an emulsifier is typically added to emulsify the active-containing solvent upon dilution with water. Active ingredient slurries, with particle diameters of up to 2,000 μm can be wet milled using media mills to obtain particles with average diameters below 3 μm. Aqueous slurries can be made into finished suspension concentrates (see, for example, U.S. Pat. No. 3,060,084) or further processed by spray drying to form water-dispersible granules. Dry formulations usually require dry milling processes, which produce average particle diameters in the 2 to 10 μm range. Dusts and powders can be prepared by blending and usually grinding (such as with a hammer mill or fluid-energy mill). Granules and pellets can be prepared by spraying the active material upon preformed granular carriers or by agglomeration techniques. See Browning, “Agglomeration”, Chemical Engineering, Dec. 4, 1967, pp 147-48, Perry's Chemical Engineer's Handbook, 4th Ed., McGraw-Hill, New York, 1963, pages 8-57 and following, and WO 91/13546. Pellets can be prepared as described in U.S. Pat. No. 4,172,714. Water-dispersible and water-soluble granules can be prepared as taught in U.S. Pat. Nos. 4,144,050, 3,920,442 and DE 3,246,493. Tablets can be prepared as taught in U.S. Pat. Nos. 5,180,587, 5,232,701 and 5,208,030. Films can be prepared as taught in GB 2,095,558 and U.S. Pat. No. 3,299,566.

For further information regarding the art of formulation, see T. S. Woods, “The Formulator's Toolbox-Product Forms for Modern Agriculture” in Pesticide Chemistry and Bioscience, The Food-Environment Challenge, T. Brooks and T. R. Roberts, Eds., Proceedings of the 9th International Congress on Pesticide Chemistry, The Royal Society of Chemistry, Cambridge, 1999, pp. 120-133. See also U.S. Pat. No. 3,235,361, Col. 6, line 16 through Col. 7, line 19 and Examples 10-41; U.S. Pat. No. 3,309,192, Col. 5, line 43 through Col. 7, line 62 and Examples 8, 12, 15, 39, 41, 52, 53, 58, 132, 138-140, 162-164, 166, 167 and 169-182; U.S. Pat. No. 2,891,855, Col. 3, line 66 through Col. 5, line 17 and Examples 1-4; Klingman, Weed Control as a Science, John Wiley and Sons, Inc., New York, 1961, pp 81-96; Hance et al., Weed Control Handbook, 8th Ed., Blackwell Scientific Publications, Oxford, 1989; and Developments in formulation technology, PJB Publications, Richmond, U K, 2000.

In the following Examples, all percentages are by weight and all formulations are prepared in conventional ways. Compound numbers refer to compounds in Index Table A. Without further elaboration, it is believed that one skilled in the art using the preceding description can utilize the present invention to its fullest extent. The following Examples are, therefore, to be construed as merely illustrative, and not limiting of the disclosure in any way whatsoever. Percentages are by weight except where otherwise indicated.

### Example A

High Strength Concentrate

| | |
|---|---|
| Compound 1 | 98.5% |
| silica aerogel | 0.5% |
| synthetic amorphous fine silica | 1.0% |

### Example B

Wettable Powder

| | |
|---|---|
| Compound 1 | 65.0% |
| dodecylphenol polyethylene glycol ether | 2.0% |
| sodium ligninsulfonate | 4.0% |
| sodium silicoaluminate | 6.0% |
| montmorillonite (calcined) | 23.0% |

### Example C

Granule

| | |
|---|---|
| Compound 1 | 10.0% |
| attapulgite granules (low volatile matter, 0.71/0.30 mm; | 90.0% |
| U.S.S. No. 25-50 sieves) | |

### Example D

Extruded Pellet

| | |
|---|---|
| Compound 1 | 25.0% |
| anhydrous sodium sulfate | 10.0% |
| crude calcium ligninsulfonate | 5.0% |
| sodium alkylnaphthalenesulfonate | 1.0% |
| calcium/magnesium bentonite | 59.0% |

### Example E

Emulsifiable Concentrate

| | |
|---|---|
| Compound 1 | 10.0% |
| polyoxyethylene sorbitol hexoleate | 20.0% |
| C₆—C₁₀ fatty acid methyl ester | 70.0% |

### Example F

Microemulsion

| | |
|---|---|
| Compound 1 | 5.0% |
| polyvinylpyrrolidone-vinyl acetate copolymer | 30.0% |
| alkylpolyglycoside | 30.0% |
| glyceryl monooleate | 15.0% |
| water | 20.0% |

### Example G

Suspension Concentrate

| | |
|---|---|
| Compound 1 | 35% |
| butyl polyoxyethylene/polypropylene block copolymer | 4.0% |
| stearic acid/polyethylene glycol copolymer | 1.0% |
| styrene acrylic polymer | 1.0% |
| xanthan gum | 0.1% |
| propylene glycol | 5.0% |
| silicone based defoamer | 0.1% |
| 1,2-benzisothiazolin-3-one | 0.1% |
| water | 53.7% |

### Example H

Emulsion in Water

| | |
|---|---|
| Compound 1 | 10.0% |
| butyl polyoxyethylene/polypropylene block copolymer | 4.0% |
| stearic acid/polyethylene glycol copolymer | 1.0% |
| styrene acrylic polymer | 1.0% |
| xanthan gum | 0.1% |
| propylene glycol | 5.0% |
| silicone based defoamer | 0.1% |
| 1,2-benzisothiazolin-3-one | 0.1% |
| aromatic petroleum based hydrocarbon | 20.0 |
| water | 58.7% |

### Example I

Oil Dispersion

| | |
|---|---|
| Compound 1 | 25% |
| polyoxyethylene sorbitol hexaoleate | 15% |
| organically modified bentonite clay | 2.5% |
| fatty acid methyl ester | 57.5% |

The present disclosure also includes Examples A through I above except that “Compound 1” is replaced with any one of “Compound 2” through “Compound 204”.

Test results indicate that the compounds of the present invention are highly active preemergent and/or postemergent herbicides and/or plant growth regulants. The compounds of the inention generally show highest activity for postemergence weed control (i.e. applied after weed seedlings emerge from the soil) and preemergence weed control (i.e. applied before weed seedlings emerge from the soil). Many of them have utility for broad-spectrum pre- and/or postemergence weed control in areas where complete control of all vegetation is desired such as around fuel storage tanks, industrial storage areas, parking lots, drive-in theaters, air fields, river banks, irrigation and other waterways, around billboards and highway and railroad structures. Many of the compounds of this invention, by virtue of selective metabolism in crops versus weeds, or by selective activity at the locus of physiological inhibition in crops and weeds, or by selective placement on or within the environment of a mixture of crops and weeds, are useful for the selective control of grass and broadleaf weeds within a crop/weed mixture. One skilled in the art will recognize that the preferred combination of these selectivity factors within a compound or group of compounds can readily be determined by performing routine biological and/or biochemical assays. Compounds of this invention may show tolerance to important agronomic crops including, but is not limited to, alfalfa, barley, cotton, wheat, rape, sugar beets, corn (maize), sorghum, soybeans, rice, oats, peanuts, vegetables, tomato, potato, perennial plantation crops including coffee, cocoa, oil palm, rubber, sugarcane, citrus, grapes, fruit trees, nut trees, banana, plantain, pineapple, hops, tea and forests such as eucalyptus and conifers (e.g., loblolly pine), and turf species (e.g., Kentucky bluegrass, St. Augustine grass, Kentucky fescue and Bermuda grass). Compounds of this invention can be used in crops genetically transformed or bred to incorporate resistance to herbicides, express proteins toxic to invertebrate pests (such as Bacillus thuringiensis toxin), and/or express other useful traits. Those skilled in the art will appreciate that not all compounds are equally effective against all weeds. Alternatively, the subject compounds are useful to modify plant growth.

As the compounds of the invention have both preemergent and postemergent herbicidal activity, to control undesired vegetation by killing or injuring the vegetation or reducing its growth, the compounds can be usefully applied by a variety of methods involving contacting a herbicidally effective amount of a compound of the invention, or a composition comprising said compound and at least one of a surfactant, a solid diluent or a liquid diluent, to the foliage or other part of the undesired vegetation or to the environment of the undesired vegetation such as the soil or water in which the undesired vegetation is growing or which surrounds the seed or other propagule of the undesired vegetation. Undesired vegetation includes at least one selected from the group consisting of grass weeds and broadleaf weeds. Undesired vegetation is selected from the group consisting of annual bluegrass, Benghal dayflower, blackgrass, black nightshade, broadleaf signalgrass, Canada thistle, cheat, common cocklebur (Xanthium pensylvanicum), common ragweed, corn poppies, field violet, giant foxtail, goosegrass, green foxtail, guinea grass, hairy beggarticks, herbicide-resistant black grass, horseweed, Italian rye grass, jimsonweed, Johnson grass (Sorghum halepense), large crabgrass, little seed canary grass, morning glory, Pennsylvania smartweed, pitted morning glory, prickly sida, quackgrass, redroot pigweed, shattercane, shepherd's purse, silky windgrass, sunflower (as weed in potato), wild buckwheat (Polygonum convolvulus), wild mustard (Brassica kaber), wild oat (Avena fatua), wild pointsettia, yellow foxtail, and yellow nutsedge (Cyperus esculentus).

A herbicidally effective amount of the compounds of this invention is determined by a number of factors. These factors include: formulation selected, method of application, amount and type of vegetation present, growing conditions, etc. In general, a herbicidally effective amount of compounds of this invention is about 0.001 to 20 kg/ha with a preferred range of about 0.004 to 1 kg/ha. One skilled in the art can easily determine the herbicidally effective amount necessary for the desired level of weed control.

In one common embodiment, a compound of the invention is applied, typically in a formulated composition, to a locus comprising desired vegetation (e.g., crops) and undesired vegetation (i.e. weeds), both of which may be seeds, seedlings and/or larger plants, in contact with a growth medium (e.g., soil). In this locus, a composition comprising a compound of the invention can be directly applied to a plant or a part thereof, particularly of the undesired vegetation, and/or to the growth medium in contact with the plant.

Although most typically, compounds of the invention are used to control undesired vegetation, contact of desired vegetation in the treated locus with compounds of the invention may result in super-additive or enhanced effects with genetic traits in the desired vegetation, including traits incorporated through genetic modification. For example, resistance to phytophagous insect pests or plant diseases, tolerance to biotic/abiotic stresses or storage stability may be greater than expected from the genetic traits in the desired vegetation.

Compounds of this invention can also be mixed with one or more other biologically active compounds or agents including herbicides, herbicide safeners, fungicides, insecticides, nematocides, bactericides, acaricides, growth regulators such as insect molting inhibitors and rooting stimulants, chemosterilants, semiochemicals, repellents, attractants, pheromones, feeding stimulants, plant nutrients, other biologically active compounds or entomopathogenic bacteria, virus or fungi to form a multi-component pesticide giving an even broader spectrum of agricultural protection. Mixtures of the compounds of the invention with other herbicides can broaden the spectrum of activity against additional weed species, and suppress the proliferation of any resistant biotypes. Thus the present invention also pertains to a composition comprising a compound of Formula 1 (in a herbicidally effective amount) and at least one additional biologically active compound or agent (in a biologically effective amount) and can further comprise at least one of a surfactant, a solid diluent or a liquid diluent. The other biologically active compounds or agents can be formulated in compositions comprising at least one of a surfactant, solid or liquid diluent. For mixtures of the present invention, one or more other biologically active compounds or agents can be formulated together with a compound of Formula 1, to form a premix, or one or more other biologically active compounds or agents can be formulated separately from the compound of Formula 1, and the formulations combined together before application (e.g., in a spray tank) or, alternatively, applied in succession.

A mixture of one or more of the following herbicides with a compound of this invention may be particularly useful for weed control: acetochlor, acifluorfen and its sodium salt, aclonifen, acrolein (2-propenal), alachlor, alloxydim, ametryn, amicarbazone, amidosulfuron, aminocyclopyrachlor and its esters (e.g., methyl, ethyl) and salts (e.g., sodium, potassium), 4-amino-3-chloro-5-fluoro-6-(7-fluoro-1H-indol-6-yl)-2-Pyridinecarboxylic 2-propyn-1-yl ester (CAS No. 2251111-17-6), 4-amino-3-chloro-5-fluoro-6-(7-fluoro-1H-indol-6-yl)-2-Pyridinecarboxylic cyanomethyl ester (CAS No. 2251111-18-7), aminopyralid, amitrole, ammonium sulfamate, 2,5-anhydro-3,4-dideoxy-4-[[[(5S)-3-(3,5-difluorophenyl)-5-ethenyl-4,5-dihydro-5-isoxazolyl]carbonyl]amino]-threo-Pentonic methyl ester (CAS No. 27499989-21-6), anilofos, anisiflupurin, asulam, atrazine, azimsulfuron, bixlozone, beflubutamid, beflubutamid-M, benazolin, benazolin-ethyl, bencarbazone, benfluralin, benfuresate, benquitrione, bensulfuron-methyl, bensulide, bentazone, benzobicyclon, benzofenap, bicyclopyrone, bifenox, bilanafos, bispyribac and its sodium salt, bromacil, bromobutide, bromofenoxim, bromoxynil, bromoxynil octanoate, butachlor, butafenacil, butamifos, butralin, butroxydim, butylate, bipyrazone, cafenstrole, carbetamide, 1-(2-carboxyethyl)-4-(2-pyrimidinyl)pyridazinium (CAS No. 2285384-11-2) and salts thereof, carfentrazone-ethyl, catechin, chlomethoxyfen, chloramben, chlorbromuron, chlorflurenol-methyl, chloridazon, chlorimuron-ethyl, 3-[2-chloro-5-[3,6-dihydro-3-methyl-2,6-dioxo-4-(trifluoromethyl)-1(2H)-pyrimidinyl]-4-fluorophenyl]-4,5-dihydro-5-methyl-5-Isoxazolecarboxylic ethyl ester (CAS No. 1949837-17-5), chlorotoluron, chlorpropham, chlorsulfuron, chlorthal-dimethyl, chlorthiamid, cinidon-ethyl, cinmethylin, cinosulfuron, clacyfos, clefoxydim, clethodim, clodinafop-propargyl, clomazone, clomeprop, clopyralid, clopyralid-olamine, cloransulam-methyl, cumyluron, cyanazine, cycloate, cyclopyrimorate, cyclosulfamuron, cycloxydim, cyhalofop-butyl, 2,4-D and its butotyl, butyl, isoctyl and isopropyl esters and its dimethylammonium, diolamine and trolamine salts, cyprafluone, daimuron, dalapon, dalapon-sodium, dazomet, 2,4-DB and its dimethylammonium, potassium and sodium salts, desmedipham, desmetryn, dicamba and its diglycolammonium, dimethylammonium, potassium and sodium salts, dichlobenil, dichlorprop, diclofop-methyl, diclosulam, difenzoquat metilsulfate, diflufenican, diflufenzopyr, dimefuron, dimepiperate, dimesulfazet, dimethachlor, dimethametryn, dimethenamid, dimethenamid-P, dimethipin, dimethylarsinic acid and its sodium salt, dinitramine, dinoterb, dioxopyritrione, diphenamid, diquat dibromide, dithiopyr, diuron, DNOC, endothal, EPTC, epyrifenacil, esprocarb, ethalfluralin, ethametsulfuron-methyl, ethiozin, ethofumesate, ethoxyfen, ethoxysulfuron, etobenzanid, fenoxaprop-ethyl, fenoxaprop-P-ethyl, fenoxasulfone, fenpyrazone, fenquinotrione, fentrazamide, fenuron, fenuron-TCA, flamprop-methyl, flamprop-M-isopropyl, flamprop-M-methyl, flazasulfuron, florasulam, fluazifop-butyl, fluazifop-P-butyl, fluazolate, flucarbazone, flucetosulfuron, fluchloralin, fluchloraminopyr, flufenacet, flufenoximacil, flufenpyr, flufenpyr-ethyl, flumetsulam, flumiclorac-pentyl, flumioxazin, fluometuron, fluoroglycofen-ethyl, flupoxam, flupyrsulfuron-methyl and its sodium salt, flurenol, flurenol-butyl, fluridone, flurochloridone, fluroxypyr, flurtamone, flusulfinam, fluthiacet-methyl, fomesafen, foramsulfuron, fosamine-ammonium, glufosinate, glufosinate-ammonium, glufosinate-P, glyphosate and its salts such as ammonium, isopropylammonium, potassium, sodium (including sesquisodium) and trimesium (alternatively named sulfosate), halauxifen, halauxifen-methyl, halosulfuron-methyl, haloxyfop-etotyl, haloxyfop-methyl, hexazinone, hydantocidin, imazamethabenz-methyl, imazamox, imazapic, imazapyr, imazaquin, imazaquin-ammonium, imazethapyr, imazethapyr-ammonium, imazosulfuron, indanofan, indaziflam, iofensulfuron, iodosulfuron-methyl, ioxynil, ioxynil octanoate, ioxynil-sodium, ipfencarbazone, isoproturon, isouron, isoxaben, isoxaflutole, isoxachlortole, lactofen, lenacil, linuron, maleic hydrazide, MCPA and its salts (e.g., MCPA-dimethylammonium, MCPA-potassium and MCPA-sodium, esters (e.g., MCPA-2-ethylhexyl, MCPA-butotyl) and thioesters (e.g., MCPA-thioethyl), MCPB and its salts (e.g., MCPB-sodium) and esters (e.g., MCPB-ethyl), mecoprop, mecoprop-P, mefenacet, mefluidide, mesosulfuron-methyl, mesotrione, metam-sodium, metamifop, metamitron, metazachlor, metazosulfuron, methabenzthiazuron, methylarsonic acid and its calcium, monoammonium, monosodium and disodium salts, methyldymron, metobenzuron, metobromuron, metolachlor, S-metolachlor, metosulam, metoxuron, metribuzin, metsulfuron-methyl, molinate, monolinuron, naproanilide, napropamide, napropamide-M, naptalam, neburon, nicosulfuron, norflurazon, orbencarb, orthosulfamuron, oryzalin, oxadiargyl, oxadiazon, oxasulfuron, oxaziclomefone, oxyfluorfen, paraquat dichloride, pebulate, pelargonic acid, pendimethalin, penoxsulam, pentanochlor, pentoxazone, perfluidone, pethoxamid, pethoxyamid, phenmedipham, picloram, picloram-potassium, picolinafen, pinoxaden, piperophos, pretilachlor, primisulfuron-methyl, prodiamine, profoxydim, prometon, prometryn, propachlor, propanil, propaquizafop, propazine, propham, propisochlor, propoxycarbazone, propyrisulfuron, propyzamide, prosulfocarb, prosulfuron, pyraclonil, pyraflufen-ethyl, pyrasulfotole, pyrazogyl, pyrazolynate, pyrazoxyfen, pyrazosulfuron-ethyl, pyribenzoxim, pyributicarb, pyridate, pyriflubenzoxim, pyriftalid, pyriminobac-methyl, pyrimisulfan, pyrithiobac, pyrithiobac-sodium, pyroxasulfone, pyroxsulam, quinclorac, quinmerac, quinoclamine, quizalofop-ethyl, quizalofop-P-ethyl, quizalofop-P-tefuryl, rimisoxafen, rimsulfuron, saflufenacil, sethoxydim, siduron, simazine, simetryn, sulcotrione, sulfentrazone, sulfometuron-methyl, sulfosulfuron, 2,3,6-TBA, TCA, TCA-sodium, tebutam, tebuthiuron, tefuryltrione, tembotrione, tepraloxydim, terbacil, terbumeton, terbuthylazine, terbutryn, tetflupyrolimet, thenylchlor, thiazopyr, thiencarbazone, thifensulfuron-methyl, thiobencarb, tiafenacil, tiocarbazil, tolpyralate, topramezone, tralkoxydim, tri-allate, triafamone, triasulfuron, triaziflam, tribenuron-methyl, triclopyr, triclopyr-butotyl, triclopyr-triethylammonium, tridiphane, trietazine, trifloxysulfuron, trifludimoxazin, trifluralin, triflusulfuron-methyl, tripyrasulfone, tritosulfuron, vernolate, 3-(2-chloro-3,6-difluorophenyl)-4-hydroxy-1-methyl-1,5-naphthyridin-2(1H)-one, 5-chloro-3-[(2-hydroxy-6-oxo-1-cyclohexen-1-yl)carbonyl]-1-(4-methoxyphenyl)-2(1H)-quinoxalinone, 2-chloro-N-(1-methyl-1H-tetrazol-5-yl)-6-(trifluoromethyl)-3-pyridinecarboxamide, 7-(3,5-dichloro-4-pyridinyl)-5-(2,2-difluoroethyl)-8-hydroxypyrido[2,3-b]pyrazin-6(5)-one), 4-(2,6-diethyl-4-methylphenyl)-5-hydroxy-2,6-dimethyl-3(2H)-pyridazinone), 5-[[(2,6-difluorophenyl) methoxy]methyl]-4,5-dihydro-5-methyl-3-(3-methyl-2-thienyl) isoxazole (previously methioxolin), 4-(4-fluorophenyl)-6-[(2-hydroxy-6-oxo-1-cyclohexen-1-yl)carbonyl]-2-methyl-1,2,4-triazine-3,5(2H,4H)-dione, methyl 4-amino-3-chloro-6-(4-chloro-2-fluoro-3-methoxyphenyl)-5-fluoro-2-pyridinecarboxylate, 2-methyl-3-(methylsulfonyl)-N-(1-methyl-1H-tetrazol-5-yl)-4-(trifluoromethyl)benzamide and 2-methyl-N-(4-methyl-1,2,5-oxadiazol-3-yl)-3-(methylsulfinyl)-4-(trifluoromethyl)benzamide. Other herbicides also include bioherbicides such as Alternaria destruens Simmons, Colletotrichum gloeosporiodes (Penz.) Penz. & Sacc., Drechsiera monoceras (MTB-951), Myrothecium verrucaria (Albertini & Schweinitz) Ditmar: Fries, Phytophthora palmivora (Butl.) Butl. and Puccinia thlaspeos Schub.

Preferred for better control of undesired vegetation (e.g., lower use rate such as from enhanced effects, broader spectrum of weeds controlled, or enhanced crop safety) or for preventing the development of resistant weeds are mixtures of a compound of this invention with a herbicide selected from the group consisting of atrazine, azimsulfuron, S-beflubutamid, benzisothiazolinone, carfentrazone-ethyl, chlorimuron-ethyl, chlorsulfuron-methyl, clomazone, clopyralid potassium, cloransulam-methyl, 2-[(2,4-dichlorophenyl)methyl]-4,4-dimethyl-3-isoxazolidinone, 2-[(2,5-dichlorophenyl)methyl]-4,4-dimethyl-3-isoxazolidinone, ethametsulfuron-methyl, flumetsulam, 4-(4-fluorophenyl)-6-[(2-hydroxy-6-oxo-1-cyclohexen-1-yl)carbonyl]-2-methyl-1,2,4-triazine-3,5-(2H,4H)-dione, flupyrsulfuron-methyl, fluthiacet-methyl, fomesafen, imazethapyr, lenacil, mesotrione, metribuzin, metsulfuron-methyl, pethoxamid, picloram, pyroxasulfone, quinclorac, rimsulfuron, S-metolachlor, sulfentrazone, thifensulfuron-methyl, triflusulfuron-methyl and tribenuron-methyl.

Compounds of this invention can also be used in combination with plant growth regulators such as aviglycine, N-(phenylmethyl)-1H-purin-6-amine, epocholeone, gibberellic acid, gibberellin A₄ and A₇, harpin protein, mepiquat chloride, prohexadione calcium, prohydrojasmon, sodium nitrophenolate and trinexapac-methyl, and plant growth modifying organisms such as Bacillus cereus strain BP01.

General references for agricultural protectants (i.e. herbicides, herbicide safeners, insecticides, fungicides, nematocides, acaricides and biological agents) include The Pesticide Manual, 13th Edition, C. D. S. Tomlin, Ed., British Crop Protection Council, Farnham, Surrey, U.K., 2003 and The BioPesticide Manual, 2nd Edition, L. G. Copping, Ed., British Crop Protection Council, Farnham, Surrey, U.K., 2001.

For embodiments where one or more of these various mixing partners are used, the mixing partners are typically used in the amounts similar to amounts customary when the mixture partners are used alone. More particularly in mixtures, active ingredients are often applied at an application rate between one-half and the full application rate specified on product labels for use of active ingredient alone. These amounts are listed in references such as The Pesticide Manual and The BioPesticide Manual. The weight ratio of these various mixing partners (in total) to the compound of Formula 1 is typically between about 1:3000 and about 3000:1. Of note are weight ratios between about 1:300 and about 300:1 (for example ratios between about 1:30 and about 30:1). One skilled in the art can easily determine through simple experimentation the biologically effective amounts of active ingredients necessary for the desired spectrum of biological activity. It will be evident that including these additional components may expand the spectrum of weeds controlled beyond the spectrum controlled by the compound of Formula 1 alone.

In certain instances, combinations of a compound of this invention with other biologically active (particularly herbicidal) compounds or agents (i.e. active ingredients) can result in a greater-than-additive (i.e. enhanced) effect on weeds and/or a less-than-additive effect (i.e. safening) on crops or other desirable plants. Reducing the quantity of active ingredients released in the environment while ensuring effective pest control is always desirable. Ability to use greater amounts of active ingredients to provide more effective weed control without excessive crop injury is also desirable. When the enhanced effects of herbicidal mixtures of active ingredients occurs on weeds at application rates giving agronomically satisfactory levels of weed control, such combinations can be advantageous for reducing crop production cost and decreasing environmental load. When safening of herbicidal active ingredients occurs on crops, such combinations can be advantageous for increasing crop protection by reducing weed competition.

Of note is a combination of a compound of the invention with at least one other herbicidal active ingredient. Of particular note is such a combination where the other herbicidal active ingredient has different site of action from the compound of the invention. In certain instances, a combination with at least one other herbicidal active ingredient having a similar spectrum of control but a different site of action will be particularly advantageous for resistance management. Thus, a composition of the present invention can further comprise (in a herbicidally effective amount) at least one additional herbicidal active ingredient having a similar spectrum of control but a different site of action.

Compounds of this invention can also be used in combination with herbicide safeners such as allidochlor, benoxacor, cloquintocet-mexyl, cumyluron, cyometrinil, cyprosulfonamide, daimuron, dichlormid, dicyclonon, dietholate, dimepiperate, fenchlorazole-ethyl, fenclorim, flurazole, fluxofenim, furilazole, isoxadifen-ethyl, mefenpyr-diethyl, mephenate, methoxyphenone naphthalic anhydride (1,8-naphthalic anhydride), oxabetrinil, N-(aminocarbonyl)-2-methylbenzenesulfonamide, N-(aminocarbonyl)-2-fluorobenzenesulfonamide, 1-bromo-4-[(chloromethyl) sulfonyl]benzene (BCS), 4-(dichloroacetyl)-1-oxa-4-azospiro[4.5]decane (MON 4660), 2-(dichloromethyl)-2-methyl-1,3-dioxolane (MG 191), ethyl 1,6-dihydro-1-(2-methoxyphenyl)-6-oxo-2-phenyl-5-pyrimidinecarboxylate, 2-hydroxy-N,N-dimethyl-6-(trifluoromethyl)pyridine-3-carboxamide, and 3-oxo-1-cyclohexen-1-yl 1-(3,4-dimethylphenyl)-1,6-dihydro-6-oxo-2-phenyl-5-pyrimidinecarboxylate, 2,2-dichloro-1-(2,2,5-trimethyl-3-oxazolidinyl)-ethanone and 2-methoxy-N-[[4-[[(methylamino)carbonyl]amino]phenyl]sulfonyl]-benzamide to increase safety to certain crops. Antidotally effective amounts of the herbicide safeners can be applied at the same time as the compounds of this invention, or applied as seed treatments. Therefore an aspect of the present invention relates to a herbicidal mixture comprising a compound of this invention and an antidotally effective amount of a herbicide safener. Seed treatment is particularly useful for selective weed control, because it physically restricts antidoting to the crop plants. Therefore a particularly useful embodiment of the present invention is a method for selectively controlling the growth of undesired vegetation in a crop comprising contacting the locus of the crop with a herbicidally effective amount of a compound of this invention wherein seed from which the crop is grown is treated with an antidotally effective amount of safener. Antidotally effective amounts of safeners can be easily determined by one skilled in the art through simple experimentation.

Compounds of the invention can also be mixed with: (1) polynucleotides including but not limited to DNA, RNA, and/or chemically modified nucleotides influencing the amount of a particular target through down regulation, interference, suppression or silencing of the genetically derived transcript that render a herbicidal effect; or (2) polynucleotides including but not limited to DNA, RNA, and/or chemically modified nucleotides influencing the amount of a particular target through down regulation, interference, suppression or silencing of the genetically derived transcript that render a safening effect.

Of note is a composition comprising a compound of the invention (in a herbicidally effective amount), at least one additional active ingredient selected from the group consisting of other herbicides and herbicide safeners (in an effective amount), and at least one component selected from the group consisting of surfactants, solid diluents and liquid diluents.

Table A1 lists specific combinations of a Component (a) with Component (b) illustrative of the mixtures, compositions and methods of the present invention. Compound No. (Compound Number) (i.e. Compound 1) in the Component (a) column is identified in Index Table A. The second column of Table A1 lists the specific Component (b) compound (e.g., “2,4-D” in the first line). The third, fourth and fifth columns of Table A1 lists ranges of weight ratios for rates at which the Component (a) compound is typically applied to a field-grown crop relative to Component (b) (i.e. (a):(b)). Thus, for example, the first line of Table A1 specifically discloses the combination of Component (a) (i.e. Compound 1 in Index Table A) with 2,4-D is typically applied in a weight ratio between 1:384-6:1. The remaining lines of Table A1 are to be construed similarly.

TABLE A1

| Component (a) | | Typical | More Typical | Most Typical |
|---|---|---|---|---|
| (Compound No.) | Component (b) | Weight Ratio | Weight Ratio | Weight Ratio |
| 1 | 2,4-D | 1:384-6:1 | 1:128-2:1 | 1:12-1:2 |
| 1 | Acetochlor | 1:1536-2:1 | 1:512-1:2 | 1:48-1:6 |
| 1 | Acifluorfen | 1:192-12:1 | 1:64-4:1 | 1:6-2:1 |
| 1 | Aclonifen | 1:1714-2:1 | 1:571-1:3 | 1:53-1:6 |
| 1 | Alachlor | 1:1536-2:1 | 1:512-1:2 | 1:48-1:6 |
| 1 | Ametryn | 1:768-3:1 | 1:256-1:1 | 1:24-1:3 |
| 1 | Amicarbazone | 1:384-6:1 | 1:128-2:1 | 1:12-1:2 |
| 1 | Amidosulfuron | 1:13-168:1 | 1:4-56:1 | 2:1-21:1 |
| 1 | Aminocyclopyrachlor | 1:96-24:1 | 1:32-8:1 | 1:3-3:1 |
| 1 | Aminopyralid | 1:41-56:1 | 1:13-19:1 | 1:1-7:1 |
| 1 | Amitrole | 1:1536-2:1 | 1:512-1:2 | 1:48-1:6 |
| 1 | Anilofos | 1:192-12:1 | 1:64-4:1 | 1:6-2:1 |
| 1 | Asulam | 1:1920-2:1 | 1:640-1:3 | 1:60-1:7 |
| 1 | Atrazine | 1:384-6:1 | 1:128-2:1 | 1:12-1:2 |
| 1 | Azimsulfuron | 1:13-168:1 | 1:4-56:1 | 2:1-21:1 |
| 1 | Beflubutamid | 1:685-4:1 | 1:228-2:1 | 1:21-1:3 |
| 1 | Benfuresate | 1:1234-2:1 | 1:411-1:2 | 1:38-1:5 |
| 1 | Bensulfuron-methyl | 1:51-45:1 | 1:17-15:1 | 1:1-6:1 |
| 1 | Bentazone | 1:384-6:1 | 1:128-2:1 | 1:12-1:2 |
| 1 | Benzobicyclon | 1:171-14:1 | 1:57-5:1 | 1:5-2:1 |
| 1 | Benzofenap | 1:514-5:1 | 1:171-2:1 | 1:16-1:2 |
| 1 | Bicyclopyrone | 1:85-27:1 | 1:28-9:1 | 1:2-4:1 |
| 1 | Bifenox | 1:514-5:1 | 1:171-2:1 | 1:16-1:2 |
| 1 | Bispyribac-sodium | 1:20-112:1 | 1:6-38:1 | 1:1-14:1 |
| 1 | Bromacil | 1:768-3:1 | 1:256-1:1 | 1:24-1:3 |
| 1 | Bromobutide | 1:768-3:1 | 1:256-1:1 | 1:24-1:3 |
| 1 | Bromoxynil | 1:192-12:1 | 1:64-4:1 | 1:6-2:1 |
| 1 | Butachlor | 1:1536-2:1 | 1:512-1:2 | 1:48-1:6 |
| 1 | Butafenacil | 1:85-27:1 | 1:28-9:1 | 1:2-4:1 |
| 1 | Butylate | 1:3085-1:2 | 1:1028-1:5 | 1:96-1:11 |
| 1 | Carfenstrole | 1:384-6:1 | 1:128-2:1 | 1:12-1:2 |
| 1 | Carfentrazone-ethyl | 1:257-9:1 | 1:85-3:1 | 1:8-2:1 |
| 1 | Chlorimuron-ethyl | 1:17-135:1 | 1:5-45:1 | 1:1-17:1 |
| 1 | Chlorotoluron | 1:1536-2:1 | 1:512-1:2 | 1:48-1:6 |
| 1 | Chlorsulfuron | 1:13-168:1 | 1:4-56:1 | 2:1-21:1 |
| 1 | Cincosulfuron | 1:34-68:1 | 1:11-23:1 | 1:1-9:1 |
| 1 | Cinidon-ethyl | 1:768-3:1 | 1:256-1:1 | 1:24-1:3 |
| 1 | Cinmethylin | 1:68-34:1 | 1:22-12:1 | 1:2-5:1 |
| 1 | Clacyfos | 1:384-6:1 | 1:128-2:1 | 1:12-1:2 |
| 1 | Clethodim | 1:96-24:1 | 1:32-8:1 | 1:3-3:1 |
| 1 | Clodinafop-propargyl | 1:41-56:1 | 1:13-19:1 | 1:1-7:1 |
| 1 | Clomazone | 1:768-3:1 | 1:256-1:1 | 1:24-1:3 |
| 1 | Clomeprop | 1:342-7:1 | 1:114-3:1 | 1:10-1:2 |
| 1 | Clopyralid | 1:384-6:1 | 1:128-2:1 | 1:12-1:2 |
| 1 | Cloransulam-methyl | 1:24-96:1 | 1:8-32:1 | 1:1-12:1 |
| 1 | Cumyluron | 1:768-3:1 | 1:256-1:1 | 1:24-1:3 |
| 1 | Cyanazine | 1:768-3:1 | 1:256-1:1 | 1:24-1:3 |
| 1 | Cyclopyrimorate | 1:34-68:1 | 1:11-23:1 | 1:1-9:1 |
| 1 | Cyclosulfamuron | 1:34-68:1 | 1:11-23:1 | 1:1-9:1 |
| 1 | Cycloxydim | 1:192-12:1 | 1:64-4:1 | 1:6-2:1 |
| 1 | Cyhalofop | 1:51-45:1 | 1:17-15:1 | 1:1-6:1 |
| 1 | Daimuron | 1:384-6:1 | 1:128-2:1 | 1:12-1:2 |
| 1 | Desmedipham | 1:644-4:1 | 1:214-2:1 | 1:20-1:3 |
| 1 | Dicamba | 1:384-6:1 | 1:128-2:1 | 1:12-1:2 |
| 1 | Dichlobenil | 1:2742-1:2 | 1:914-1:4 | 1:85-1:10 |
| 1 | Dichlorprop | 1:1851-2:1 | 1:617-1:3 | 1:57-1:7 |
| 1 | Diclofop-methyl | 1:768-3:1 | 1:256-1:1 | 1:24-1:3 |
| 1 | Diclosulam | 1:20-112:1 | 1:6-38:1 | 1:1-14:1 |
| 1 | Difenzoquat | 1:576-4:1 | 1:192-2:1 | 1:18-1:2 |
| 1 | Diflufenican | 1:1714-2:1 | 1:571-1:3 | 1:53-1:6 |
| 1 | Diflufenzopyr | 1:24-96:1 | 1:8-32:1 | 1:1-12:1 |
| 1 | Dimethachlor | 1:1536-2:1 | 1:512-1:2 | 1:48-1:6 |
| 1 | Dimethametryn | 1:384-6:1 | 1:128-2:1 | 1:12-1:2 |
| 1 | Dimethenamid-P | 1:768-3:1 | 1:256-1:1 | 1:24-1:3 |
| 1 | Dithiopyr | 1:384-6:1 | 1:128-2:1 | 1:12-1:2 |
| 1 | Diuron | 1:768-3:1 | 1:256-1:1 | 1:24-1:3 |
| 1 | EPTC | 1:1536-2:1 | 1:512-1:2 | 1:48-1:6 |
| 1 | Esprocarb | 1:2742-1:2 | 1:914-1:4 | 1:85-1:10 |
| 1 | Ethalfluralin | 1:768-3:1 | 1:256-1:1 | 1:24-1:3 |
| 1 | Ethametsulfuron-methyl | 1:34-68:1 | 1:11-23:1 | 1:1-9:1 |
| 1 | Ethoxyfen | 1:17-135:1 | 1:5-45:1 | 1:1-17:1 |
| 1 | Ethoxysulfuron | 1:41-56:1 | 1:13-19:1 | 1:1-7:1 |
| 1 | Etobenzanid | 1:514-5:1 | 1:171-2:1 | 1:16-1:2 |
| 1 | Fenoxaprop-ethyl | 1:240-10:1 | 1:80-4:1 | 1:7-2:1 |
| 1 | Fenoxasulfone | 1:171-14:1 | 1:57-5:1 | 1:5-2:1 |
| 1 | Fenquinotrione | 1:85-27:1 | 1:28-9:1 | 1:2-4:1 |
| 1 | Fentrazamide | 1:34-68:1 | 1:11-23:1 | 1:1-9:1 |
| 1 | Flazasulfuron | 1:34-68:1 | 1:11-23:1 | 1:1-9:1 |
| 1 | Florasulam | 1:5-420:1 | 1:1-140:1 | 5:1-53:1 |
| 1 | Fluazifop-butyl | 1:384-6:1 | 1:128-2:1 | 1:12-1:2 |
| 1 | Flucarbazone | 1:17-135:1 | 1:5-45:1 | 1:1-17:1 |
| 1 | Flucetosulfuron | 1:17-135:1 | 1:5-45:1 | 1:1-17:1 |
| 1 | Flufenacet | 1:514-5:1 | 1:171-2:1 | 1:16-1:2 |
| 1 | Flumetsulam | 1:48-48:1 | 1:16-16:1 | 1:1-6:1 |
| 1 | Flumiclorac-pentyl | 1:20-112:1 | 1:6-38:1 | 1:1-14:1 |
| 1 | Flumioxazin | 1:51-45:1 | 1:17-15:1 | 1:1-6:1 |
| 1 | Fluometuron | 1:768-3:1 | 1:256-1:1 | 1:24-1:3 |
| 1 | Flupyrsulfuron-methyl | 1:6-336:1 | 1:2-112:1 | 4:1-42:1 |
| 1 | Fluridone | 1:768-3:1 | 1:256-1:1 | 1:24-1:3 |
| 1 | Fluroxypyr | 1:192-12:1 | 1:64-4:1 | 1:6-2:1 |
| 1 | Flurtamone | 1:1714-2:1 | 1:571-1:3 | 1:53-1:6 |
| 1 | Fluthiacet-methyl | 1:96-42:1 | 1:32-14:1 | 1:1-6:1 |
| 1 | Fomesafen | 1:192-12:1 | 1:64-4:1 | 1:6-2:1 |
| 1 | Foramsulfuron | 1:27-84:1 | 1:9-28:1 | 1:1-11:1 |
| 1 | Glufosinate | 1:576-4:1 | 1:192-2:1 | 1:18-1:2 |
| 1 | Glyphosate | 1:576-4:1 | 1:192-2:1 | 1:18-1:2 |
| 1 | Halosulfuron-methyl | 1:34-68:1 | 1:11-23:1 | 1:1-9:1 |
| 1 | Halauxifen | 1:41-56:1 | 1:13-19:1 | 1:1-7:1 |
| 1 | Halauxifen methyl | 1:41-56:1 | 1:13-19:1 | 1:1-7:1 |
| 1 | Haloxyfop-methyl | 1:68-34:1 | 1:22-12:1 | 1:2-5:1 |
| 1 | Hexazinone | 1:384-6:1 | 1:128-2:1 | 1:12-1:2 |
| 1 | Imazamox | 1:27-84:1 | 1:9-28:1 | 1:1-11:1 |
| 1 | Imazapic | 1:41-56:1 | 1:13-19:1 | 1:1-7:1 |
| 1 | Imazapyr | 1:171-14:1 | 1:57-5:1 | 1:5-2:1 |
| 1 | Imazaquin | 1:68-34:1 | 1:22-12:1 | 1:2-5:1 |
| 1 | Imazethabenz-methyl | 1:342-7:1 | 1:114-3:1 | 1:10-1:2 |
| 1 | Imazethapyr | 1:48-48:1 | 1:16-16:1 | 1:1-6:1 |
| 1 | Imazosulfuron | 1:54-42:1 | 1:18-14:1 | 1:1-6:1 |
| 1 | Indanofan | 1:685-4:1 | 1:228-2:1 | 1:21-1:3 |
| 1 | Indaziflam | 1:51-45:1 | 1:17-15:1 | 1:1-6:1 |
| 1 | Iodosulfuron-methyl | 1:6-336:1 | 1:2-112:1 | 4:1-42:1 |
| 1 | Ioxynil | 1:384-6:1 | 1:128-2:1 | 1:12-1:2 |
| 1 | Ipfencarbazone | 1:171-14:1 | 1:57-5:1 | 1:5-2:1 |
| 1 | Isoproturon | 1:768-3:1 | 1:256-1:1 | 1:24-1:3 |
| 1 | Isoxaben | 1:576-4:1 | 1:192-2:1 | 1:18-1:2 |
| 1 | Isoxaflutole | 1:120-20:1 | 1:40-7:1 | 1:3-3:1 |
| 1 | Lactofen | 1:85-27:1 | 1:28-9:1 | 1:2-4:1 |
| 1 | Lenacil | 1:768-3:1 | 1:256-1:1 | 1:24-1:3 |
| 1 | Linuron | 1:768-3:1 | 1:256-1:1 | 1:24-1:3 |
| 1 | MCPA | 1:384-6:1 | 1:128-2:1 | 1:12-1:2 |
| 1 | MCPB | 1:576-4:1 | 1:192-2:1 | 1:18-1:2 |
| 1 | Mecoprop | 1:1536-2:1 | 1:512-1:2 | 1:48-1:6 |
| 1 | Mefenacet | 1:768-3:1 | 1:256-1:1 | 1:24-1:3 |
| 1 | Mefluidide | 1:384-6:1 | 1:128-2:1 | 1:12-1:2 |
| 1 | Mesosulfuron-methyl | 1:10-224:1 | 1:3-75:1 | 3:1-28:1 |
| 1 | Mesotrione | 1:85-27:1 | 1:28-9:1 | 1:2-4:1 |
| 1 | Metamifop | 1:85-27:1 | 1:28-9:1 | 1:2-4:1 |
| 1 | Metazachlor | 1:768-3:1 | 1:256-1:1 | 1:24-1:3 |
| 1 | Metazosulfuron | 1:51-45:1 | 1:17-15:1 | 1:1-6:1 |
| 1 | Methabenzthiazuron | 1:1536-2:1 | 1:512-1:2 | 1:48-1:6 |
| 1 | Metolachlor | 1:1536-2:1 | 1:512-1:2 | 1:48-1:6 |
| 1 | Metosulam | 1:17-135:1 | 1:5-45:1 | 1:1-17:1 |
| 1 | Metribuzin | 1:384-6:1 | 1:128-2:1 | 1:12-1:2 |
| 1 | Metsulfuron-methyl | 1:4-560:1 | 1:1-187:1 | 7:1-70:1 |
| 1 | Molinate | 1:2057-2:1 | 1:685-1:3 | 1:64-1:8 |
| 1 | Napropamide | 1:768-3:1 | 1:256-1:1 | 1:24-1:3 |
| 1 | Napropamide-M | 1:384-6:1 | 1:128-2:1 | 1:12-1:2 |
| 1 | Naptalam | 1:384-6:1 | 1:128-2:1 | 1:12-1:2 |
| 1 | Nicosulfuron | 1:24-96:1 | 1:8-32:1 | 1:1-12:1 |
| 1 | Norflurazon | 1:2304-1:1 | 1:768-1:3 | 1:72-1:8 |
| 1 | Orbencarb | 1:2742-1:2 | 1:914-1:4 | 1:85-1:10 |
| 1 | Orthosulfamuron | 1:41-56:1 | 1:13-19:1 | 1:1-7:1 |
| 1 | Oryzalin | 1:1028-3:1 | 1:342-1:2 | 1:32-1:4 |
| 1 | Oxadiargy1 | 1:768-3:1 | 1:256-1:1 | 1:24-1:3 |
| 1 | Oxadiazon | 1:1097-3:1 | 1:365-1:2 | 1:34-1:4 |
| 1 | Oxasulfuron | 1:54-42:1 | 1:18-14:1 | 1:1-6:1 |
| 1 | Oxaziclomefone | 1:85-27:1 | 1:28-9:1 | 1:2-4:1 |
| 1 | Oxyfluorfen | 1:768-3:1 | 1:256-1:1 | 1:24-1:3 |
| 1 | Paraquat | 1:384-6:1 | 1:128-2:1 | 1:12-1:2 |
| 1 | Pendimethalin | 1:768-3:1 | 1:256-1:1 | 1:24-1:3 |
| 1 | Penoxsulam | 1:20-112:1 | 1:6-38:1 | 1:1-14:1 |
| 1 | Penthoxamid | 1:768-3:1 | 1:256-1:1 | 1:24-1:3 |
| 1 | Pentoxazone | 1:205-12:1 | 1:68-4:1 | 1:6-2:1 |
| 1 | Phenmedipham | 1:205-12:1 | 1:68-4:1 | 1:6-2:1 |
| 1 | Picloram | 1:192-12:1 | 1:64-4:1 | 1:6-2:1 |
| 1 | Picolinafen | 1:68-34:1 | 1:22-12:1 | 1:2-5:1 |
| 1 | Pinoxaden | 1:51-45:1 | 1:17-15:1 | 1:1-6:1 |
| 1 | Pretilachlor | 1:384-6:1 | 1:128-2:1 | 1:12-1:2 |
| 1 | Primisulfuron-methyl | 1:17-135:1 | 1:5-45:1 | 1:1-17:1 |
| 1 | Prodiamine | 1:768-3:1 | 1:256-1:1 | 1:24-1:3 |
| 1 | Profoxydim | 1:85-27:1 | 1:28-9:1 | 1:2-4:1 |
| 1 | Prometryn | 1:768-3:1 | 1:256-1:1 | 1:24-1:3 |
| 1 | Propachlor | 1:2304-1:1 | 1:768-1:3 | 1:72-1:8 |
| 1 | Propanil | 1:768-3:1 | 1:256-1:1 | 1:24-1:3 |
| 1 | Propaquizafop | 1:96-24:1 | 1:32-8:1 | 1:3-3:1 |
| 1 | Propoxycarbazone | 1:34-68:1 | 1:11-23:1 | 1:1-9:1 |
| 1 | Propyrisulfuron | 1:34-68:1 | 1:11-23:1 | 1:1-9:1 |
| 1 | Propyzamide | 1:768-3:1 | 1:256-1:1 | 1:24-1:3 |
| 1 | Prosulfocarb | 1:2400-1:2 | 1:800-1:4 | 1:75-1:9 |
| 1 | Prosulfuron | 1:13-168:1 | 1:4-56:1 | 2:1-21:1 |
| 1 | Pyraclonil | 1:85-27:1 | 1:28-9:1 | 1:2-4:1 |
| 1 | Pyraflufen-ethyl | 1:10-224:1 | 1:3-75:1 | 3:1-28:1 |
| 1 | Pyrasulfotole | 1:27-84:1 | 1:9-28:1 | 1:1-11:1 |
| 1 | Pyrazolynate | 1:1714-2:1 | 1:571-1:3 | 1:53-1:6 |
| 1 | Pyrazosulfuron-ethyl | 1:20-112:1 | 1:6-38:1 | 1:1-14:1 |
| 1 | Pyrazoxyfen | 1:10-224:1 | 1:3-75:1 | 3:1-28:1 |
| 1 | Pyribenzoxim | 1:20-112:1 | 1:6-38:1 | 1:1-14:1 |
| 1 | Pyributicarb | 1:768-3:1 | 1:256-1:1 | 1:24-1:3 |
| 1 | Pyridate | 1:576-4:1 | 1:192-2:1 | 1:18-1:2 |
| 1 | Pyriftalid | 1:20-112:1 | 1:6-38:1 | 1:1-14:1 |
| 1 | Pyriminobac-methyl | 1:41-56:1 | 1:13-19:1 | 1:1-7:1 |
| 1 | Pyrimisulfan | 1:34-68:1 | 1:11-23:1 | 1:1-9:1 |
| 1 | Pyrithiobac | 1:48-48:1 | 1:16-16:1 | 1:1-6:1 |
| 1 | Pyroxasulfone | 1:171-14:1 | 1:57-5:1 | 1:5-2:1 |
| 1 | Pyroxsulam | 1:10-224:1 | 1:3-75:1 | 3:1-28:1 |
| 1 | Quinclorac | 1:384-6:1 | 1:128-2:1 | 1:12-1:2 |
| 1 | Quizalofop-ethyl | 1:85-27:1 | 1:28-9:1 | 1:2-4:1 |
| 1 | Rimsulfuron | 1:27-84:1 | 1:9-28:1 | 1:1-11:1 |
| 1 | Saflufenacil | 1:51-45:1 | 1:17-15:1 | 1:1-6:1 |
| 1 | Sethoxydim | 1:192-12:1 | 1:64-4:1 | 1:6-2:1 |
| 1 | Simazine | 1:768-3:1 | 1:256-1:1 | 1:24-1:3 |
| 1 | Sulcotrione | 1:240-10:1 | 1:80-4:1 | 1:7-2:1 |
| 1 | Sulfentrazone | 1:294-8:1 | 1:98-3:1 | 1:9-1:2 |
| 1 | Sulfometuron-methyl | 1:68-34:1 | 1:22-12:1 | 1:2-5:1 |
| 1 | Sulfosulfuron | 1:17-135:1 | 1:5-45:1 | 1:1-17:1 |
| 1 | Tebuthiuron | 1:768-3:1 | 1:256-1:1 | 1:24-1:3 |
| 1 | Tefuryltrione | 1:85-27:1 | 1:28-9:1 | 1:2-4:1 |
| 1 | Tembotrione | 1:63-37:1 | 1:21-13:1 | 1:1-5:1 |
| 1 | Tepraloxydim | 1:51-45:1 | 1:17-15:1 | 1:1-6:1 |
| 1 | Terbacil | 1:576-4:1 | 1:192-2:1 | 1:18-1:2 |
| 1 | Terbuthylazine | 1:1714-2:1 | 1:571-1:3 | 1:53-1:6 |
| 1 | Terbutryn | 1:384-6:1 | 1:128-2:1 | 1:12-1:2 |
| 1 | Thenylchlor | 1:171-14:1 | 1:57-5:1 | 1:5-2:1 |
| 1 | Thiazopyr | 1:768-3:1 | 1:256-1:1 | 1:24-1:3 |
| 1 | Thiencarbazone | 1:6-336:1 | 1:2-112:1 | 4:1-42:1 |
| 1 | Thifensulfuron-methyl | 1:10-224:1 | 1:3-75:1 | 3:1-28:1 |
| 1 | Tiafenacil | 1:85-27:1 | 1:28-9:1 | 1:2-4:1 |
| 1 | Thiobencarb | 1:1536-2:1 | 1:512-1:2 | 1:48-1:6 |
| 1 | Topramezone | 1:13-168:1 | 1:4-56:1 | 2:1-21:1 |
| 1 | Tralkoxydim | 1:137-17:1 | 1:45-6:1 | 1:4-3:1 |
| 1 | Triallate | 1:1536-2:1 | 1:512-1:2 | 1:48-1:6 |
| 1 | Triasulfuron | 1:10-224:1 | 1:3-75:1 | 3:1-28:1 |
| 1 | Triaziflam | 1:342-7:1 | 1:114-3:1 | 1:10-1:2 |
| 1 | Tribenuron-methyl | 1:6-336:1 | 1:2-112:1 | 4:1-42:1 |
| 1 | Triclopyr | 1:384-6:1 | 1:128-2:1 | 1:12-1:2 |
| 1 | Trifloxysulfuron | 1:5-420:1 | 1:1-140:1 | 5:1-53:1 |
| 1 | Trifluralin | 1:576-4:1 | 1:192-2:1 | 1:18-1:2 |
| 1 | Triflusulfuron-methyl | 1:34-68:1 | 1:11-23:1 | 1:1-9:1 |
| 1 | Tritosulfuron | 1:27-84:1 | 1:9-28:1 | 1:1-11:1 |

Table A2 is constructed the same as Table A1 above except that those entries below the “Component (a)” column heading are replaced with the respective Component (a) Column Entry shown below. Compound No. in the Component (a) column is identified in Index Table A. Thus, for example, in Table A2 the entries below the “Component (a)” column heading all recite “Compound 2” (i.e. Compound 2 identified in Index Table A), and the first line below the column headings in Table A2 specifically discloses a mixture of Compound 2 with 2,4-D. Tables A3 through A204 are constructed similarly.

| Table | Component (a) |
|---|---|
| Number | Column Entries |
| A2 | Compound 2 |
| A3 | Compound 3 |
| A4 | Compound 4 |
| A5 | Compound 5 |
| A6 | Compound 6 |
| A7 | Compound 7 |
| A8 | Compound 8 |
| A9 | Compound 9 |
| A10 | Compound 10 |
| A11 | Compound 11 |
| A12 | Compound 12 |
| A13 | Compound 13 |
| A14 | Compound 14 |
| A15 | Compound 15 |
| A16 | Compound 16 |
| A17 | Compound 17 |
| A18 | Compound 18 |
| A19 | Compound 19 |
| A20 | Compound 20 |
| A21 | Compound 21 |
| A22 | Compound 22 |
| A23 | Compound 23 |
| A24 | Compound 24 |
| A25 | Compound 25 |
| A26 | Compound 26 |
| A27 | Compound 27 |
| A28 | Compound 28 |
| A29 | Compound 29 |
| A30 | Compound 30 |
| A31 | Compound 31 |
| A32 | Compound 32 |
| A33 | Compound 33 |
| A34 | Compound 34 |
| A35 | Compound 35 |
| A36 | Compound 36 |
| A37 | Compound 37 |
| A38 | Compound 38 |
| A39 | Compound 39 |
| A40 | Compound 40 |
| A41 | Compound 31 |
| A42 | Compound 32 |
| A43 | Compound 33 |
| A44 | Compound 34 |
| A45 | Compound 35 |
| A46 | Compound 36 |
| A47 | Compound 37 |
| A48 | Compound 38 |
| A49 | Compound 39 |
| A50 | Compound 50 |
| A51 | Compound 51 |
| A52 | Compound 52 |
| A53 | Compound 53 |
| A54 | Compound 54 |
| A55 | Compound 55 |
| A56 | Compound 56 |
| A57 | Compound 57 |
| A58 | Compound 58 |
| A59 | Compound 59 |
| A60 | Compound 60 |
| A61 | Compound 61 |
| A62 | Compound 62 |
| A63 | Compound 63 |
| A64 | Compound 64 |
| A65 | Compound 65 |
| A66 | Compound 66 |
| A67 | Compound 67 |
| A68 | Compound 68 |
| A69 | Compound 69 |
| A70 | Compound 70 |
| A71 | Compound 71 |
| A72 | Compound 72 |
| A73 | Compound 73 |
| A74 | Compound 74 |
| A75 | Compound 75 |
| A76 | Compound 76 |
| A77 | Compound 77 |
| A78 | Compound 78 |
| A79 | Compound 79 |
| A80 | Compound 80 |
| A81 | Compound 81 |
| A82 | Compound 82 |
| A83 | Compound 83 |
| A84 | Compound 84 |
| A85 | Compound 85 |
| A86 | Compound 86 |
| A87 | Compound 87 |
| A88 | Compound 88 |
| A89 | Compound 89 |
| A90 | Compound 90 |
| A91 | Compound 91 |
| A92 | Compound 92 |
| A93 | Compound 93 |
| A94 | Compound 94 |
| A95 | Compound 95 |
| A96 | Compound 96 |
| A97 | Compound 97 |
| A98 | Compound 98 |
| A99 | Compound 99 |
| A100 | Compound 100 |
| A101 | Compound 101 |
| A102 | Compound 102 |
| A103 | Compound 103 |
| A104 | Compound 104 |
| A105 | Compound 105 |
| A106 | Compound 106 |
| A107 | Compound 107 |
| A108 | Compound 108 |
| A109 | Compound 109 |
| A110 | Compound 110 |
| A111 | Compound 111 |
| A112 | Compound 112 |
| A113 | Compound 113 |
| A114 | Compound 114 |
| A115 | Compound 115 |
| A116 | Compound 116 |
| A117 | Compound 117 |
| A118 | Compound 118 |
| A119 | Compound 119 |
| A120 | Compound 120 |
| A121 | Compound 121 |
| A122 | Compound 122 |
| A123 | Compound 123 |
| A124 | Compound 124 |
| A125 | Compound 125 |
| A126 | Compound 126 |
| A127 | Compound 127 |
| A128 | Compound 128 |
| A129 | Compound 129 |
| A130 | Compound 130 |
| A131 | Compound 131 |
| A132 | Compound 132 |
| A133 | Compound 133 |
| A134 | Compound 134 |
| A135 | Compound 135 |
| A136 | Compound 136 |
| A137 | Compound 137 |
| A138 | Compound 138 |
| A139 | Compound 139 |
| A140 | Compound 140 |
| A141 | Compound 141 |
| A142 | Compound 142 |
| A143 | Compound 143 |
| A144 | Compound 144 |
| A145 | Compound 145 |
| A146 | Compound 146 |
| A147 | Compound 147 |
| A148 | Compound 148 |
| A149 | Compound 149 |
| A150 | Compound 150 |
| A151 | Compound 151 |
| A152 | Compound 152 |
| A153 | Compound 153 |
| A154 | Compound 154 |
| A155 | Compound 155 |
| A156 | Compound 156 |
| A157 | Compound 157 |
| A158 | Compound 158 |
| A159 | Compound 159 |
| A160 | Compound 160 |
| A161 | Compound 161 |
| A162 | Compound 162 |
| A163 | Compound 163 |
| A164 | Compound 164 |
| A165 | Compound 165 |
| A166 | Compound 166 |
| A167 | Compound 167 |
| A168 | Compound 168 |
| A169 | Compound 169 |
| A170 | Compound 170 |
| A171 | Compound 171 |
| A172 | Compound 172 |
| A173 | Compound 173 |
| A174 | Compound 174 |
| A175 | Compound 175 |
| A176 | Compound 176 |
| A177 | Compound 177 |
| A178 | Compound 178 |
| A179 | Compound 179 |
| A180 | Compound 180 |
| A181 | Compound 181 |
| A182 | Compound 182 |
| A183 | Compound 183 |
| A184 | Compound 184 |
| A185 | Compound 185 |
| A186 | Compound 186 |
| A187 | Compound 187 |
| A188 | Compound 188 |
| A189 | Compound 189 |
| A190 | Compound 190 |
| A191 | Compound 191 |
| A192 | Compound 192 |
| A193 | Compound 193 |
| A194 | Compound 194 |
| A195 | Compound 195 |
| A196 | Compound 196 |
| A197 | Compound 197 |
| A198 | Compound 198 |
| A199 | Compound 199 |
| A200 | Compound 200 |
| A201 | Compound 201 |
| A202 | Compound 202 |
| A203 | Compound 203 |
| A204 | Compound 204 |

Preferred for better control of undesired vegetation (e.g., lower use rate such as from enhanced effects, broader spectrum of weeds controlled, or enhanced crop safety) or for preventing the development of resistant weeds are mixtures of a compound of this invention with a herbicide selected from the group consisting of chlorimuron-ethyl, nicosulfuron, mesotrione, thifensulfuron-methyl, flupyrsulfuron-methyl, tribenuron, pyroxasulfone, pinoxaden, tembotrione, pyroxsulam, metolachlor and S-metolachlor.

The following TESTS demonstrate the control efficacy of compounds of this invention on specific pathogens. The pathogen control protection afforded by the compounds is not limited, however, to these species. See Index Tables A through H below for compound descriptions. The abbreviation “Cmpd.” stands for “Compound”, and the abbreviation “Ex.” stands for “Example” and is followed by a number indicating in which example the compound is prepared. The numerical value reported in the column “MS” is the molecular weight of the highest isotopic abundance positively charged parent ion (M+1) formed by addition of H+ (molecular weight of 1) to the molecule having the highest isotopic abundance, or the highest isotopic abundance positively charged parent ion (M+23) formed by addition of Na+ (molecular weight of 23) or the highest isotopic abundance negatively charged ion (M−1) formed by loss of H+ (molecular weight of 1). “Pyr” means “Pyridine”. “Przl” means “Pyrazol”. “Thzl” means “Thiazole”. “Trzl” means “Triazole”. “Imdzl” mean “Imidazol”. “c-pent” mean “cyclopentyl”. The presence of molecular ions containing one or more higher atomic weight isotopes of lower abundance (e.g., ³⁷Cl, ⁸¹Br) is not reported. The reported MS peaks were observed by mass spectrometry using electrospray ionization (ESI) or atmospheric pressure chemical ionization (APCI). “Stereo (*)” refers to the stereochemistry at the position marked with an asterisk (“*”).

INDEX TABLE A

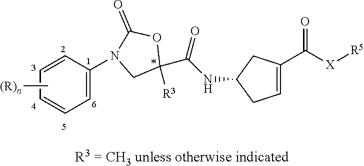

| | | | | | | MS (M − 1) |
|---|---|---|---|---|---|---|
| Cmpd. No. | (R)ₙ | X | R⁵ | Stereochemistry(*) | MS (M + 1) | or M.P. |
| 1 | 3,5-diF | O | CH₂CF₃ | (1′) or (1″) R | | 447.3 |
| 5 | 3,5-diF | O | i-Pr | Racemic | 409.5 | |
| 15 | 3,5-diF | O | Et | Racemic | 395.3 | |
| 16 | 3,5-diF | O | i-Bu | Racemic | | 421.4 |
| 20 | 3,5-diF | O | Me | (1′) or (1″) S | | 379.3 |
| | | | | Enantiomer | | |
| 21 | 3,5-diF | O | Me | (1′) or (1″) R | | 379.4 |
| | | | | Enantiomer | | |
| 22 | 3,5-diF | O | (CH₂)₂SMe | (1′) or (1″) S | | 439.3 |
| | | | | Enantiomer | | |
| 23 | 3,5-diF | O | (CH₂)₂SMe | (1′) or (1″) R | | 439.4 |
| | | | | Enantiomer | | |
| 24 | 3,5-diF | O | CH₂CF₃ | (1′) or (1″) S | | 447.3 |
| | | | | Enantiomer | | |
| 37 | 3,5-diF | O | Me | Racemic | | 379.3 |
| 38 | 3,5-diF | O | H | Racemic | | 365.3 |
| 58 | 3,5-diCl | O | Me | Racemic | 413.09 | |
| 60 | 3,5-diCl | O | Et | Racemic | 427.16 | |
| 70 | 3,5-diCl | O | i-pr | Racemic | 441.19 | |
| 71 | 3,5-diCl | O | CF₃CH₂ | Racemic | 481.16 | |
| 72 | 3,5-diCl | O | H | Racemic | | 399.2 |
| 74 | 3,5-diCl | NH | SO₂CF₃ | Racemic | | 530.2 |
| 75 | 3,5-diCl | NH | SO₂-c-pr | Racemic | | 502.3 |
| 76 | 3,5-diCl | NH | SO₂-n-pr | Racemic | | 504.3 |
| 77 | 3,5-diCl | NH | SO₂Et | Racemic | | 488.3 |
| 78 | 3,5-diCl | NH | SO₂Me | Racemic | | 474.2 |
| 90 | 3,5-diF | NH | SO₂-c-pr | racemic | | 142-145 |
| 91 | 3,5-diF | NH | SO₂-t-bu | racemic | | 147-150 |
| 94 | 3,5-diF | O | H | R isomer | 503.3 (ES+, | 365.3 (ES−) |
| | | | | | M + 23) | |
| 95 | 3,5-diCl | O | CH₂CF₃ | S isomer | 502.3 (ES+, | |
| | | | | | M + 23) | |
| 96 | 3,5-diCl | O | CH₂CF₃ | R isomer | | |
| 97 | 3,5-diF | O | i-Bu | S isomer | | 421.2 (ES−) |
| 98 | 3,5-diF | O | i-Bu | R isomer | | 421.3 (ES−) |
| 99 | 3,5-diF | O | i-pr | S isomer | | 407.3 (ES−) |
| 100 | 3,5-diF | O | i-pr | R isomer | | 407.2 (ES−) |
| 103 | 3,5-diF | NH | SO₂CH₃ | racemic | | 115-118 |
| 104 | 3,5-diF | NH | SO₂Ph | racemic | | 153-156 |
| 105 | 3,5-diCl | O | i-pr | S isomer | | 439.2 (ES−) |
| 106 | 3,5-diCl | O | i-pr | R isomer | | 439.2 (ES−) |
| 107 | 3,5-diF | O | CH₂C(═O)OMe | S isomer | 439.2 (ES+) | |
| 108 | 3,5-diF | O | CH₂C(═O)OMe | R isomer | 439.3 (ES+) | |
| 109 | 3,5-diCl | O | CH₂C(═O)OMe | S isomer | 471.2 (ES+) | |
| 110 | 3,5-diCl | O | CH₂C(═O)OMe | R isomer | 471.2 (ES+) | |
| 111 | 3,5-diF | O | Et | S isomer | 395.3 (ES+) | |
| 112 | 3,5-diF | O | Et | R isomer | 395.3 (ES+) | |
| 113 | 3,5-diCl | O | Et | S isomer | 429.3 (ES+) | |
| 114 | 3,5-diCl | O | Et | R isomer | 429.3 (ES+) | |
| 129 | 3,5-diCl,4-F | O | Me | racemic | 431.43 (ES+) | |
| 133 | 3-Cl,5-F | O | Et | R isomer | | 132-135 |
| 134 | 3-Cl,5-F | O | Et | S isomer | | 106-109 |
| 135 | 3-Cl,5-F | O | CH₂CH₂SMe | R isomer | | 105-108 |
| 136 | 3-Cl,5-F | O | 3-Cl,5-F | S isomer | | 103-106 |
| 140 | 3-Cl,5-F | O | Me | R isomer | | 171-174 |
| 141 | 3-Cl,5-F | O | CH₂CF₃ | R isomer | | 463.12 (ES−) |
| 142 | 3-Cl,5-F | O | CH₂CF₃ | S isomer | | 177-180 |
| 143 | 3-Cl,5-F | O | i-Bu | S isomer | | 87-90 |
| 144 | 3-Cl,5-F | O | CH₂c-pr | R isomer | | 141-144 |
| 146 | 3,5-diCl | O | CH₂CH₂SMe | S isomer | 495.3 (AP+, | 471.2 (ES−) |
| | | | | | M + 23) | |
| 147 | 3,5-diCl | O | CH₂CH₂SMe | R isomer | 435.3 (AP+, | |
| | | | | | M + 23) | |
| 148 | 3,5-diCl | O | Me | S isomer | 435.3 (ES+, | |
| | | | | | M + 23) | |
| 149 | 3,5-diCl | O | Me | R isomer | | |
| 151 | 3,5-diF | O | CH₂CHF₂ | S isomer | | 429.2 (ES−) |
| 152 | 3,5-diF | O | CH₂CHF₂ | R isomer | | 429.2 (ES−) |
| 153 | 3,5-diCl | O | CH₂CHF₂ | S isomer | 485.3 (ES, | |
| | | | | | M + 23) | |
| 154 | 3,5-diCl | O | CH₂CHF₂ | R isomer | 485.3 (ES, | |
| | | | | | M + 23) | |
| 155 | 3,5-diCl | O | i-Bu | S isomer | | 453.2 (ES−) |
| 156 | 3,5-diCl | O | i-Bu | R isomer | | 453.2 (ES−) |
| 157 | 3,5-diCl | O | H | S isomer | | 397.1 (ES−) |
| 158 | 3,5-diCl | O | H | R isomer | | 397.0 (ES−) |
| 159 | 3-Cl,5-F | O | H | R isomer | | 167-170 |
| 160 | 3-Cl,5-F | O | H | S isomer | | 381.12 (ES−) |
| 163 | 3,4,5-diF | O | Me | Racemic | | 128-131 |
| 169 | 3,5-diF | O | Me | Racemic | 393.1 | |
| R³ = CHCH₂ | | | | | | |
| 191 | 3,5-diCl | O | Me | Racemic | 489.2 | |
| | | | | | (M + 23) | |
| 192 | 3,5-diF | O | CH₂CN | R-isomer | 428.2 | |
| | | | | | (M + 23) | |
| 193 | 3,5-diCl | O | CH₂CN | 460.2 | 460.2 | |
| | | | | | (M + 23) | |
| 197 | 3,5-diCl | O | CH₂CH₂CF₃ | R-isomer | 495.2 | |
| 198 | 3,5-diCl | O | CH₂c-pr | R-isomer | 453.3 | |
| 203 | 3,5-diF | O | CH₂CH₂CF₃ | R-isomer | 485.3 M + 23) | |
| 204 | 3,5-diF | O | CH₂c-pr | R-isomer | 421.3 | |

Stereochemistry* of (1′) and (1″) are defined as below.

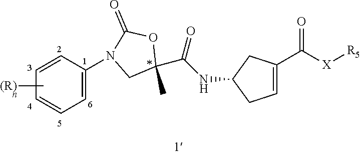

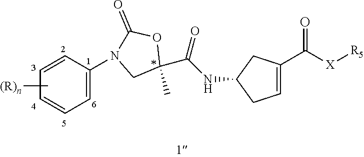

INDEX TABLE B

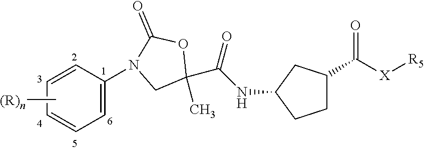

| Cmpd. | | | | | MS | MS | MS |
|---|---|---|---|---|---|---|---|
| No. | (R)ₙ | X | R⁵ | Stereochemistry(*) | (M + 1) | (M − 1) | (M + 23) |
| 2 | 3-Cl-5-F | O | i-pr | Racemic | | 427.1 | |
| 7 | 3-Cl-5-F | O | H | Racemic | 385.1 | | |
| 9 | 3,5-diF | O | H | Racemic | 369.1 | | |
| 10 | 3,5-diF | O | Me | Racemic | 383.1 | | |
| 11 | 3,5-diF | O | Et | Racemic | 397.1 | | |
| 12 | 3,5-diF | O | i-pr | Racemic | 411.29 | | |
| 13 | 3,5-diF | NH | SO₂Me | Racemic | 446.24 | | |
| 14 | 3,5-diF | NH | Me | Racemic | 382.19 | | |
| 18 | 3,5-diF | O | Me | Racemic | 399.37 | | |
| 19 | 3-Cl-5-F | O | Et | Racemic | 413.5 | | |
| 73 | 3,5-diCl | O | Me | Racemic | | | 439.3 |
| 200 | 3,5-diCl | O | Me | R-isomer | 415.1 | | |
| 201 | 3,5-diF | O | Me | R-isomer | 383.4 | | |

INDEX TABLE C

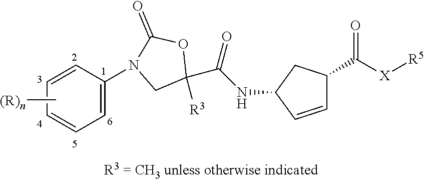

| | | | | | MS | MS | MS | M.P. |
|---|---|---|---|---|---|---|---|---|
| Cmpd. No. | (R)ₙ | X | R⁵ | Stereochem(*) | (M + 1) | (M − 1) | (M + 23) | (° C.) |
| 3 | 3-Cl-5-F | O | Et | Racemic | 411.10 | | | |
| 4 | 3-Cl-5-F | O | i-pr | Racemic | 425.20 | | | |
| 6 | 3-Cl-5-F | O | H | Racemic | 383.13 | | | |
| 17 | 3-Cl-5-F | O | Me | Racemic | 397.1 | | | |
| 25 | 3,5-diF | NH | OMe | Racemic | 396.5 | | | |
| 26 | 3,5-diF | NH | Me | Racemic | 380.3 | | | |
| 27* | 3,5-diF | O | Me | Racemic | 381.5 | | | |
| 28* | 3,5-diF | O | Et | Racemic | | 393.3 | | |
| 30 | 3,5-diF | NH | SO₂Me | Racemic | | | 466.5 | |
| 34 | 3,5-diF | O | n-pr | Racemic | 409.5 | | | |
| 35 | 3,5-diF | O | CH₂CN | Racemic | 406.3 | | | |
| 36 | 3,5-diF | O | (CH₂)₂OMe | Racemic | 425.5 | | | |
| 42 | 3,5-diCl | O | Me | Racemic | 413.4 | | | |
| 43 | 3,5-diCl | O | i-pr | Racemic | 441.3 | | | |
| 44 | 3,5-diCl | O | Et | Racemic | 427.5 | | | |
| 45 | 3,5-diCl | O | (CH₂)₂SMe | Racemic | | | 495.4 | |
| 46 | 3,5-diCl | O | (CH₂)₂SO₂Me | Racemic | 505.4 | | | |
| 47 | 3,5-diCl | NH | Me | Racemic | 412.3 | | | |
| 48 | 3,5-diF | O | (CH₂)₂SMe | Racemic | 441.4 | | | |
| 49 | 3,5-diF | O | (CH₂)₂SO2Me | Racemic | | | 495.5 | |
| 50 | 3,5-diF | NH | SO₂(n-pr) | Racemic | | | 494.5 | |
| 51 | 3,5-diF | O | H | Racemic | 367.31 | | | |
| 52 | 3,5-diF | O | i-pr | Racemic | 409.5 | | | |
| 53 | 3,5-diF | O | CH₂CO₂Et | Racemic | 453.5 | | | |
| 54 | 3,5-diF | O | CH₂(4-F—Ph) | Racemic | | | 497.3 | |
| 55 | 3,5-diF | O | CH₂CF₃ | Racemic | 449.5 | | | |
| 56 | 3,5-diF | O | i-Bu | Racemic | 423.5 | | | |
| 57 | 3,5-diF | O | c-pent | Racemic | | | 457.3 | |
| 59 | 3,5-diCl | O | c-pent | Racemic | 467.30 | | | |
| 61 | 3,5-diCl | O | Et | Racemic | | 425.33 | | |
| 62 | 3,5-diCl | O | H | Racemic | 399.11 | | | |
| 63 | 3,5-diCl | O | CH₂Ph | Racemic | 413.05 | | | |
| 64 | 3,5-diCl | O | CF₃CH₂ | Racemic | 481.42 | | | |
| 65 | 3,5-diCl | O | CH₂CO₂Me | Racemic | 471.14 | | | |
| 66 | 3,5-diCl | O | i-Bu | Racemic | | 453.2 | | |
| 67 | 3,5-diCl | O | CH₂COMe | Racemic | 455.15 | | | |
| 68 | 3,5-diF | O | NCHPh | Racemic | | | 492.5 | |
| 69 | 3,5-diCl | O | (CH₂)₂SMe | Racemic | | 471.19 | | |
| 79 | 3,5-diCl | O | SO₂-n-pr | Racemic | | | | 101-104 |
| 80 | 3,5-diF | NH | SO₂-t-Bu | Racemic | | | | 217-220 |
| 81 | 3,5-diF | NH | SO₂Et | Racemic | | | | 126-129 |
| 82 | 3,5-diCl | NH | SO₂Me | Racemic | | | | 167-170 |
| 83 | 3,5-diCl | NH | SO₂Et | Racemic | | | | 187-191 |
| 84 | 3,5-diCl | NH | SO₂CF₃ | Racemic | | | | 193-196 |
| 85 | 3,5-diCl | NH | SO₂-c-pr | Racemic | | | | 220-222 |
| 86 | 3,5-diCl | NH | SO₂Ph | Racemic | | | | 176-179 |
| 87 | 3,5-diF | NH | SO₂-c-pr | Racemic | | | | 170-173 |
| 88 | 3,5-diF | NH | SO₂Ph | Racemic | | | | 105-108 |
| 89 | 3,5-diF | O | Me | R isomer | | | | 114-117 |
| 92 | 3,5-diF | O | Me | S isomer | 381.23 | | | |
| | | | | | (ES+) | | | |
| 93 | | NH | SO₂CF₃ | racemic | | | | 229-231 |
| 101 | 3-OCF₃ | O | Me | racemic | 427.0 | | | |
| | | | | | (ES−) | | | |
| 102 | 3-OCF₃ | O | Et | racemic | 443.1 | | | |
| | | | | | (ES+) | | | |
| 115 | 3,5-diMe | O | Me | racemic | 373.2 | | | |
| | | | | | (ES+) | | | |
| 116 | 3,5-diMe | O | Et | racemic | 387.2 | | | |
| | | | | | (ES+) | | | |
| 123 | 3,4,5-F | O | H | racemic | 385.13 | | | |
| | | | | | (ES+) | | | |
| 124 | 3,5-diCl,4-F | O | H | racemic | | | | 169-172 |
| 126 | 3,5-diMe | O | H | racemic | 359.0 | | | |
| | | | | | (ES+) | | | |
| 131 | 3,5-diF | O | H | racemic | | | | 167-170 |
| 137 | 3,5-diF | N | OH | racemic | 382.3 | | | |
| | | | | | (ES+) | | | |
| 138 | 3,5-diF | O | H | R isomer | 365.3 | | | |
| | | | | | (ES+) | | | |
| 150 | 3,5-diCl,4-F | O | Me | racemic | 431.13 | | | |
| | | | | | (ES+) | | | |
| 161 | 3,5-diF | O | Me | Racemic | | | | 142-145 |
| R³ = CF₃ | | | | | | | | |
| 162 | 3,4,5-diF | O | Me | Racemic | | | | 113-116 |
| 165 | 3,5-diF | O | Me | Racemic | 393.4 | | | |
| R³ = CHCH₂ | | | | | | | | |
| 166 | 3,5-diF | O | H | Racemic | 379.2 | | | |
| R³ = CHCH₂ | | | | | | | | |
| 167 | 3,5-diF | O | Me | 1′ or 1″ | | | | 415.2 |
| R³ = CHCH₂ | | | | | | | | |
| 168 | 3,5-diF | O | Me | 1′ or 1″ | | | | 415.2 |
| R³ = CHCH₂ | | | | | | | | |
| 194 | 3,5-diF | NH | SO₂Et | R isomer | 458.2 | | | |
| | | | | | (ES+) | | | |
| 195 | 3,5-diCl | NH | SO₂Et | R-isomer | 490.3 | | | |
| 202 | 3,5-diCl | O(CF₃) | Me | Racemice | 467.2 | | | |
| | | | | | (ES+) | | | |

INDEX TABLE D

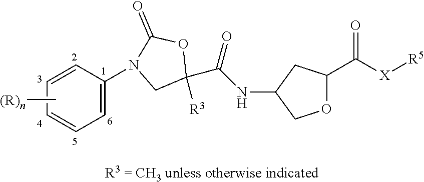

| | | | | | MS |
|---|---|---|---|---|---|
| Cmpd. No. | (R)ₙ | X | R⁵ | Stereochemistry(*) | (M + 1) |
| 8 | 3,5-diF | NH | Me | Racemic | 384.16 |
| 29 | 3,5-diF | O | Me | Racemic | 385.18 |
| 31 | 3,5-diF | O | Et | Racemic | 399.12 |
| 32 | 3,5-diF | O | i-pr | Racemic | 413.21 |
| 33 | 3,5-diF | O | H | Racemic | 446.24 |
| 39 | 3,5-diF | NH | OMe | Racemic | 400.20 |
| 40 | 3,5-diF | NH | OMe | Racemic | 400.16 |
| 41 | 3,5-diF | NH | OMe | Racemic | 400.23 |
| 170 | 3,5-diF | O | Me | | |
| (R³ = CHCH₂) | | | | | |
| 171 | 3,5-diF | O | Me | | |
| (R³ = CHCH₂) | | | | | |
| 187 | 3,5-diCl | O | Me | | |
| 188 | 3,5-diCl | O | Me | | |

INDEX TABLE E

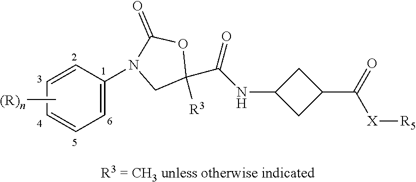

| | | | | | | | MS (M −1) or |
|---|---|---|---|---|---|---|---|
| Cmpd. No. | (R)ₙ | R³ | X | R⁵ | Stereochemistry(*) | MS (M + 1) | M.P. |
| 117 | 3,5-diF | Me | O | Me | Racemic (c) | 369.3 (ES−) | |
| 118 | 3,5-diF | Me | O | Me | Racemic (c) | 369.3 (ES−) | |
| 119 | 3,5-diF | Me | O | H | Racemic (c) | 355.2 (ES−) | |
| 120 | 3,5-diF | Me | O | CH₂CH₂SMe | Racemic (c) | 427.4 (ES−) | |
| 121 | 3,5-diF | Me | O | CH₂CF₃ | Racemic (c) | 437.4 (ES−) | |
| 122 | 3,5-diF | Me | O | SO₂Ph | Racemic (c) | 494.2 (ES−) | |
| 125 | 3,5-DiF | Me | O | Et | Racemic (c) | 383.2 (ES−) | |
| 130 | 3,5-diF | Me | O | Me | R isomer (c) | 367.4 (ES−) | |
| 139 | 3-Cl,5-F | Me | O | H | Racemic (c) | | 239-241 |
| 145 | 3-Cl,5-F | Me | O | CH₃ | Racemic | | 127-130 |
| 172 | 3,5-diF | CHCH₂ | O | Me | Racemic (c) | 381.3 | |
| R³ = CHCH₂ | | | | | | | |
| 173 | 3,5-diF | CHCH₂ | O | Me | 1′ or 1″ (c) | 381.2 | |
| R³ = CHCH₂ | | | | | | | |
| 174 | 3,5-diF | CHCH₂ | O | Me | 1′ or 1″ (c) | 381.2 | |
| R³ = CHCH₂ | | | | | | | |
| 175 | 3,5-diF | CHCH₂ | O | Me | Racemic (t) | 381.2 | |
| R³ = CHCH₂ | | | | | | | |
| 183 | 3,5-diF | Me | O | CH₂CF₃ | R isomer (c) | 437.1 | |
| 184 | 3,5-diCl | Me | O | CH₂CF₃ | R isomer (c) | 469.2 | |
| 185 | 3,5-diF | Me | O | CH₂CHF₂ | R isomer (c) | 419.2 | |
| 186 | 3,5-diCl | Me | O | CH₂CHF₂ | R isomer (c) | 451.2 | |
| 189 | 3,5-diF | Me | O | CH₂CH₂SMe | R isomer (c) | 481.3 | |
| | | | | | | (M + 23) | |
| 190 | 3,5-diCl | Me | O | CH₂CH₂SMe | R isomer (c) | 483.2 | |
| | | | | | | (M + 23) | |
| 196 | 3,5-diF | CF₃ | O | Me | Racemic (c) | 423.2 | |
| R³ = CF₃ | | | | | | | |
| 199 | 3,5-diCl | CF₃ | O | Me | Racemic (c) | 455.0 | |
| R³ = CF₃ | | | | | | | |

(c) means the cyclobutyl ring is in the 1,4-cis configuration
(t) means the cyclobutyl ring is in the 1,4-trans configuration

INDEX TABLE F

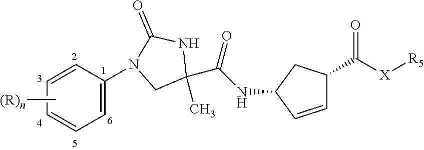

| Cmpd. | | | | | MS | |
|---|---|---|---|---|---|---|
| No. | (R)ₙ | X | R⁵ | Stereo(*) | (M + 1) | M.P |
| 179 | 3,5-diF | O | H | | | 179-182 |
| 180 | 3,5-diF | O | Me | | | 227-230 |

INDEX TABLE G

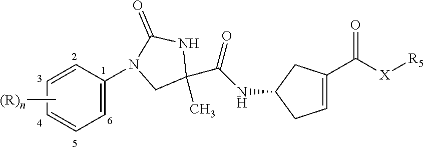

| Cmpd. No. | (R)ₙ | X | R⁵ | MS (M + 1) | M.P. |
|---|---|---|---|---|---|
| 181 | 3,5-diF | O | Me | 380.6 (Cl) | |
| 182 | 3,5-diF | O | H | | 137-140 |

INDEX TABLE H

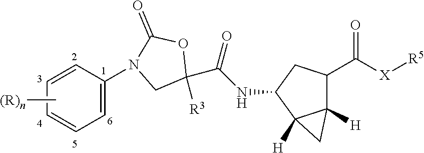

| Cmpd. No. | (R)ₙ | R³ | X | R³ | R⁵ | Stereo(*) | MS (M + 1) |
|---|---|---|---|---|---|---|---|
| 176 | 3,5-diF | Me | O | Me | Me | Racemic | 395.2 |
| 177 | 3,5-diF | CF₃ | O | CF₃ | Me | Racemic | 449.2 |
| 178 | 3,5-diF | CF₃ | O | CF₃ | H | Racemic | 435.2 |

### BIOLOGICAL EXAMPLES OF THE INVENTION

### Test A

Seeds of plant species selected from barnyardgrass (Echinochloa crus-galli), blackgrass (Alopecurus myosuroides), corn (Zea mays), foxtail, giant (giant foxtail, Setaria faberi), green foxtail (Setaria viridis), goosegrass (Eleusine indica), kochia (Bassia scoparia), oat, wild (wild oat, Avena fatua), palmer (palmer amaranth, palmer pigweed, Amaranthus palmeri), ragweed (common ragweed, Ambrosia artemisiifolia), ryegrass, Italian (italian ryegrass, Lolium multiflorum), soybean (Glycine max) and wheat (Triticum aestivum) were planted into a blend of loam soil and sand and treated preemergence with a directed soil spray using test chemicals formulated in a non-phytotoxic solvent mixture which included a surfactant.

At the same time, plants selected from these crops and weed species and also galium (catchweed bedstraw, Galium aparine) and horseweed (Erigeron canadensis) were planted in pots containing the same blend of loam soil and sand and treated with postemergence applications of test chemicals formulated in the same manner. Plants ranged in height from 2 to 10 cm and were in the one- to two-leaf stage for the postemergence treatment. Treated plants and untreated controls were maintained in a greenhouse for 10 days, after which time all treated plants were compared to untreated controls and visually evaluated for injury. Plant response ratings, summarized in Table A, are based on a 0 to 100 scale where 0 is no effect and 100 is complete control. A dash (-) response means no test result.

TABLE A

| | |
|---|---|
| 1000 g ai/ha | Compounds |

| Preemergence | 26 | 27 | 28 | 48 | 50 | 52 |
|---|---|---|---|---|---|---|
| Barnyardgrass | — | — | — | 100 | 90 | 100 |
| Blackgrass | 100 | — | 90 | 90 | 90 | 90 |
| Ragweed | 90 | 50 | 80 | 90 | 50 | 90 |
| Foxtail, Giant | 100 | 100 | 100 | 100 | 90 | 100 |
| Goosegrass | 100 | 100 | 100 | — | — | — |
| Ryegrass, Italian | 100 | 100 | 100 | 100 | 100 | 100 |
| Kochia | 90 | 100 | 100 | 100 | 100 | 100 |
| Amaranth, Palmer | 100 | 100 | 100 | 100 | 100 | 100 |

| | |
|---|---|
| 1000 g ai/ha | Compounds |

| Preemergence | 179 | 180 | 181 | 182 |
|---|---|---|---|---|
| Barnyardgrass | 100 | 100 | 80 | 80 |
| Blackgrass | — | — | 80 | 90 |
| Foxtail, Green | 100 | 100 | 90 | 100 |
| Kochia | 100 | 100 | 90 | 90 |
| Pigweed, Palmer | 60 | 100 | 90 | 90 |
| Ragweed | — | — | 0 | 30 |
| Ryegrass, Italian | 100 | 100 | 0 | 90 |

| 500 g ai/ha | Compounds | | | | | | | | | | | | |
|---|---|---|---|---|---|---|---|---|---|---|---|---|---|
| Preemergence | 25 | 30 | 34 | 35 | 36 | 42 | 43 | 44 | 45 | 46 | 47 | 49 | 51 |
| Barnyardgrass | — | 100 | 100 | 100 | 100 | 100 | 100 | 100 | 100 | 100 | 100 | 100 | 100 |
| Blackgrass | 90 | 90 | 100 | 90 | 100 | 100 | 100 | 100 | 90 | 90 | 90 | 80 | 90 |
| Ragweed | 30 | 60 | — | — | — | 80 | 80 | 60 | 70 | 70 | 60 | 90 | 80 |
| Foxtail, Giant | 90 | 100 | — | — | — | — | — | — | — | — | — | 100 | 100 |
| Goosegrass | 100 | — | — | — | — | — | — | — | — | — | — | — | — |
| Foxtail, Green | — | — | 100 | 100 | 100 | 100 | 100 | 100 | 100 | 0 | 100 | — | — |
| Ryegrass, Italian | 90 | 90 | 100 | 100 | 100 | 100 | 90 | 100 | 90 | 90 | 90 | 100 | 100 |
| Kochia | 90 | 90 | 90 | 90 | 100 | 90 | 90 | 100 | 100 | 90 | 100 | 100 | 100 |
| Corn | 90 | 80 | 100 | 100 | 90 | 90 | 90 | 90 | 60 | 90 | 70 | 90 | 90 |
| Soybean | 70 | 80 | 80 | 90 | 80 | 70 | 60 | 70 | 60 | 60 | 0 | 90 | 100 |
| Amaranth, Palmer | 80 | 100 | 100 | 100 | 100 | 100 | 100 | 100 | 100 | 100 | 100 | 100 | 100 |
| Oat, Wild | 90 | 90 | 90 | 90 | 90 | 90 | 90 | 90 | 90 | 90 | 90 | 90 | 90 |
| Wheat | 100 | 100 | 100 | 100 | 100 | 90 | 90 | 90 | 90 | 90 | 80 | 100 | 100 |

| 500 g ai/ha | Compound |
|---|---|
| Preemergence | 176 |
| Barnyardgrass | 100 |
| Blackgrass | 100 |
| Corn | 100 |
| Foxtail, Green | 100 |
| Kochia | 100 |
| Oat, Wild | 90 |
| Ragweed | 0 |
| Ryegrass, Italian | 100 |
| Soybean | 80 |
| Wheat | 100 |

| 125 g ai/ha | Compounds | | | | | | | | | | | | | |
|---|---|---|---|---|---|---|---|---|---|---|---|---|---|---|
| Preemergence | 1 | 2 | 3 | 4 | 5 | 6 | 7 | 8 | 9 | 10 | 11 | 12 | 13 | 14 |
| Barnyardgrass | 100 | 90 | 100 | 100 | 100 | 100 | 90 | 10 | 50 | 90 | 100 | 90 | 20 | 0 |
| Blackgrass | 70 | 50 | 70 | 60 | 40 | 60 | 70 | 50 | 80 | 90 | 90 | 80 | 50 | 0 |
| Ragweed | 90 | 0 | 30 | 0 | 0 | 40 | 0 | 0 | 0 | 0 | 70 | 0 | 20 | 30 |
| Foxtail, Giant | — | — | — | — | — | — | — | — | — | — | — | — | — | — |
| Goosegrass | — | — | — | — | — | — | — | — | — | — | — | — | — | — |
| Foxtail, Green | 100 | 90 | 100 | 100 | 100 | 100 | 100 | 40 | 90 | 90 | 100 | 50 | 50 | 0 |
| Ryegrass, Italian | 90 | 80 | 90 | 80 | — | — | — | 60 | 90 | 90 | 90 | 90 | 90 | 0 |
| Kochia | 100 | 70 | 100 | 90 | 90 | 100 | 80 | 70 | 30 | 90 | 50 | 70 | 0 | 0 |
| Corn | 90 | 70 | 50 | 70 | 50 | 90 | 80 | 0 | 80 | 70 | 50 | 50 | 0 | 0 |
| Soybean | 90 | 20 | 80 | 70 | 60 | 80 | 60 | 0 | 80 | 50 | 20 | 0 | 0 | 0 |
| Amaranth, Palmer | 100 | 80 | 100 | 100 | 100 | 90 | 90 | 0 | 10 | 40 | 30 | 0 | 0 | 0 |
| Oat, Wild | 80 | 70 | 90 | 80 | 90 | 90 | 70 | 20 | 90 | 80 | 80 | 90 | 20 | 0 |
| Wheat | 100 | 90 | 90 | 90 | 80 | 90 | 90 | 80 | 90 | 90 | 100 | 90 | 80 | 0 |

| 125 g ai/ha | Compounds | | | | | | | | | | | | | |
|---|---|---|---|---|---|---|---|---|---|---|---|---|---|---|
| Preemergence | 15 | 16 | 17 | 18 | 19 | 20 | 21 | 22 | 23 | 24 | 25 | 29 | 30 | 31 |
| Barnyardgrass | 100 | 90 | 100 | 90 | 80 | 0 | 100 | 0 | 100 | 10 | — | 20 | 60 | 0 |
| Blackgrass | 40 | 40 | 70 | 60 | 50 | 0 | 90 | 20 | 90 | 0 | 40 | 50 | 80 | 30 |
| Ragweed | 0 | 20 | 40 | 0 | 0 | 0 | 30 | 0 | 70 | 10 | 0 | 0 | 40 | 0 |
| Foxtail, Giant | — | — | — | — | — | — | — | — | — | — | 20 | — | 30 | — |
| Goosegrass | — | — | — | — | — | — | — | — | — | — | 30 | — | — | — |
| Foxtail, Green | 100 | 100 | 100 | 90 | 90 | 0 | 100 | 30 | 100 | 50 | — | 0 | — | 0 |
| Ryegrass, Italian | 90 | 90 | 90 | 90 | 80 | 20 | 90 | 50 | 90 | 0 | 60 | 30 | 70 | 60 |
| Kochia | 90 | 40 | 100 | 70 | 90 | 0 | 90 | 0 | 90 | 0 | 30 | 0 | 90 | 0 |
| Corn | 60 | 70 | 80 | 70 | 50 | 0 | 90 | 20 | 80 | 0 | 50 | 0 | 70 | 0 |
| Soybean | 30 | 0 | 80 | 0 | 20 | 60 | 80 | 10 | 70 | 10 | 0 | 0 | 0 | 0 |
| Amaranth, Palmer | 100 | 80 | 100 | 90 | 90 | 0 | 100 | 80 | 90 | 70 | 10 | 0 | 80 | 10 |
| Oat, Wild | 50 | 50 | 80 | 80 | 60 | 0 | 80 | 20 | 60 | 20 | 80 | 60 | 60 | 30 |
| Wheat | 90 | 80 | 90 | 90 | 90 | 20 | 90 | 60 | 90 | 70 | 60 | 80 | 90 | 90 |

| 125 g ai/ha | Compounds | | | | | | | | | | | | | |
|---|---|---|---|---|---|---|---|---|---|---|---|---|---|---|
| Preemergence | 32 | 33 | 34 | 35 | 36 | 37 | 38 | 39 | 40 | 41 | 42 | 43 | 44 | 45 |
| Barnyardgrass | 0 | 0 | 100 | 100 | 100 | 90 | 40 | 20 | 30 | 0 | 100 | 100 | 100 | 100 |
| Blackgrass | 0 | 70 | 90 | 70 | 80 | 20 | 50 | 60 | 80 | 0 | 90 | 90 | 90 | 90 |
| Ragweed | 0 | 0 | — | — | — | 0 | 0 | 0 | 0 | 0 | 20 | 30 | 10 | 20 |
| Foxtail, Giant | — | — | — | — | — | — | — | — | — | — | — | — | — | — |
| Goosegrass | — | — | — | — | — | — | — | — | — | — | — | — | — | — |
| Foxtail, Green | 0 | 0 | 100 | 90 | 100 | 90 | 20 | 0 | 20 | 0 | 100 | 90 | 90 | 90 |
| Ryegrass, Italian | 80 | 70 | 100 | 100 | 100 | 90 | 90 | 40 | 90 | 0 | 90 | 90 | 90 | 90 |
| Kochia | 20 | 0 | 90 | 90 | 90 | 20 | 20 | 20 | 0 | 0 | 90 | 80 | 100 | 20 |
| Corn | 10 | 10 | 90 | 90 | 90 | 80 | 90 | 0 | 0 | 0 | 90 | 80 | 70 | 20 |
| Soybean | 0 | 0 | 70 | 80 | 60 | 70 | 80 | 10 | 10 | 0 | 40 | 40 | 20 | 30 |
| Amaranth, Palmer | 0 | 0 | 90 | 100 | 80 | 80 | 70 | 0 | 0 | 0 | 100 | 100 | 100 | 100 |
| Oat, Wild | 40 | 90 | 90 | 90 | 90 | 60 | 60 | 90 | 90 | 0 | 90 | 90 | 80 | 80 |
| Wheat | 70 | 90 | 90 | 90 | 90 | 80 | 90 | 90 | 90 | 0 | 90 | 90 | 90 | 90 |

| 125 g ai/ha | Compounds | | | | | | | | | | | | | |
|---|---|---|---|---|---|---|---|---|---|---|---|---|---|---|
| Preemergence | 46 | 47 | 49 | 51 | 53 | 54 | 55 | 56 | 57 | 58 | 59 | 60 | 62 | 63 |
| Barnyardgrass | 80 | 40 | 90 | 100 | 100 | 100 | 100 | 100 | 100 | 90 | 100 | 100 | 100 | 90 |
| Blackgrass | 80 | 10 | 70 | 80 | 90 | 60 | 90 | 90 | 90 | 30 | 60 | 50 | 70 | 50 |
| Ragweed | 30 | 40 | 40 | 70 | 20 | 0 | 0 | 0 | 0 | 30 | 60 | 60 | 20 | 0 |
| Foxtail, Giant | — | — | 90 | 90 | — | — | — | — | — | — | — | — | — | — |
| Goosegrass | — | — | — | — | — | — | — | — | — | — | — | — | — | — |
| Foxtail, Green | 0 | 30 | — | — | 20 | 30 | 90 | 90 | 50 | — | — | — | 90 | 90 |
| Ryegrass, Italian | 80 | 70 | 80 | 90 | 100 | 100 | 90 | 90 | 100 | 80 | 80 | 90 | 80 | 70 |
| Kochia | 60 | 90 | 90 | 90 | 90 | 90 | 90 | 90 | 90 | 70 | 80 | 90 | 90 | 90 |
| Corn | 0 | 30 | 80 | 90 | 60 | 30 | 40 | 90 | 90 | 60 | 50 | 60 | 80 | 30 |
| Soybean | 10 | 10 | 80 | 70 | 70 | 70 | 80 | 80 | 80 | 50 | 60 | 40 | 60 | 20 |
| Amaranth, Palmer | 80 | 80 | 90 | 90 | 60 | 100 | 70 | 70 | 70 | 100 | 100 | 100 | 100 | 100 |
| Oat, Wild | 70 | 90 | 80 | 90 | 90 | 90 | 90 | 90 | 90 | 30 | 80 | 20 | 70 | 60 |
| Wheat | 70 | 20 | 90 | 100 | 90 | 90 | 100 | 100 | 100 | 90 | 70 | 80 | 90 | 50 |

| 125 g ai/ha | Compounds | | | | | | | | | | | | | |
|---|---|---|---|---|---|---|---|---|---|---|---|---|---|---|
| Preemergence | 64 | 65 | 66 | 67 | 68 | 69 | 70 | 71 | 72 | 73 | 75 | 79 | 80 | 81 |
| Barnyardgrass | 90 | 90 | 90 | 90 | 100 | 90 | 90 | 90 | 90 | 90 | 80 | 90 | 40 | 90 |
| Blackgrass | 70 | 70 | 80 | 70 | 70 | 60 | 30 | 50 | 30 | 50 | 40 | 30 | 30 | 70 |
| Ragweed | 80 | 70 | 70 | 60 | 20 | 20 | 10 | 30 | 40 | 10 | 0 | 20 | 0 | 10 |
| Foxtail, Giant | — | — | — | — | — | — | — | — | — | — | — | — | — | — |
| Goosegrass | — | — | — | — | — | — | — | — | — | — | — | — | — | — |
| Foxtail, Green | 90 | 90 | 100 | 90 | — | — | — | — | — | — | — | — | — | — |
| Ryegrass, Italian | 90 | 90 | 90 | 80 | 80 | 80 | 70 | 70 | 80 | 80 | 70 | 80 | 60 | 80 |
| Kochia | 90 | 10 | 30 | 80 | 90 | 90 | 40 | 90 | 90 | 90 | 90 | 70 | 70 | 90 |
| Corn | 80 | 70 | 60 | 70 | 80 | 60 | 20 | 50 | 60 | 40 | 0 | 30 | 0 | 80 |
| Soybean | 40 | 70 | 0 | 30 | 70 | 50 | 30 | 70 | 80 | 20 | 10 | 70 | 10 | 70 |
| Amaranth, Palmer | 100 | 100 | 100 | 100 | 100 | 90 | 90 | 100 | 100 | 90 | 90 | 90 | 50 | 90 |
| Oat, Wild | 50 | 80 | 90 | 70 | 90 | 60 | 20 | 30 | 40 | 0 | 0 | 30 | 30 | 70 |
| Wheat | 90 | 90 | 70 | 90 | 90 | 70 | 70 | 90 | 80 | 40 | 40 | 80 | 50 | 90 |

| | |
|---|---|
| 125 g ai/ha | Compounds |

| Preemergence | 83 | 85 | 98 |
|---|---|---|---|
| Barnyardgrass | 70 | 90 | 70 |
| Blackgrass | 40 | 20 | 0 |
| Ragweed | 20 | 20 | 50 |
| Foxtail, Giant | — | — | — |
| Goosegrass | — | — | — |
| Foxtail, Green | — | — | — |
| Ryegrass, Italian | 80 | 80 | 80 |
| Kochia | 90 | 90 | 90 |
| Corn | 60 | 30 | 20 |
| Soybean | 10 | 10 | 10 |
| Amaranth, Palmer | 100 | 90 | 100 |
| Oat, Wild | 60 | 50 | 20 |
| Wheat | 60 | 90 | 70 |

| 125 g ai/ha | Compound |
|---|---|
| Preemergence | 176 |
| Barnyardgrass | 100 |
| Blackgrass | 90 |
| Corn | 70 |
| Foxtail, Green | 90 |
| Kochia | 90 |
| Oat, Wild | 90 |
| Ragweed | 0 |
| Ryegrass, Italian | 100 |
| Soybean | 70 |
| Wheat | 100 |

| 62 g ai/ha | Compounds | | | | | | | | | | | | | |
|---|---|---|---|---|---|---|---|---|---|---|---|---|---|---|
| Preemergence | 89 | 90 | 91 | 92 | 93 | 94 | 95 | 96 | 97 | 98 | 99 | 100 | 101 | 102 |
| Barnyardgrass | 100 | 0 | 10 | 0 | 0 | 100 | 0 | 60 | 0 | 90 | 0 | 100 | 60 | 0 |
| Blackgrass | 70 | 0 | 0 | 0 | 20 | 60 | 0 | 0 | 0 | 50 | 0 | 60 | 50 | 30 |
| Corn | 100 | 0 | 0 | 0 | 0 | 80 | 0 | 40 | 0 | 90 | 0 | 30 | 50 | 10 |
| Foxtail, Green | 100 | 10 | 0 | 0 | 0 | 90 | 10 | 90 | 60 | 100 | 40 | 100 | 0 | 10 |
| Kochia | 100 | 0 | 0 | 0 | 40 | 90 | 0 | 0 | 0 | 90 | 0 | 100 | 0 | — |
| Oat, Wild | 90 | 0 | 0 | 0 | 0 | 50 | 0 | 20 | 0 | 40 | 50 | 0 | 80 | 30 |
| Pigweed, Palmer | 100 | 20 | 0 | 70 | 20 | 60 | 0 | 90 | 0 | 50 | 0 | 100 | 0 | 0 |
| Ragweed | 30 | 0 | 0 | 0 | 0 | 40 | 0 | 0 | 0 | 0 | 0 | 0 | 10 | 0 |
| Ryegrass, Italian | 90 | 0 | 0 | 0 | 0 | 70 | 0 | 60 | 0 | 80 | 0 | 80 | 80 | 70 |
| Soybean | 80 | 10 | 0 | 0 | 0 | 30 | 0 | 30 | 0 | 50 | 0 | 40 | 0 | 0 |
| Wheat | 90 | 0 | 0 | 0 | 0 | 90 | 80 | 0 | 10 | 90 | 0 | 90 | 40 | 20 |

| 62 g ai/ha | Compounds | | | | | | | | | | | | | |
|---|---|---|---|---|---|---|---|---|---|---|---|---|---|---|
| Preemergence | 103 | 104 | 105 | 106 | 107 | 108 | 109 | 110 | 111 | 112 | 113 | 114 | 115 | 116 |
| Barnyardgrass | 10 | 20 | 0 | 0 | 10 | 100 | 0 | 100 | 0 | 100 | 0 | 90 | 100 | 100 |
| Blackgrass | 0 | 0 | 0 | 0 | 0 | 30 | 0 | 30 | 0 | 30 | 0 | 20 | 60 | 70 |
| Corn | 10 | 0 | 0 | 0 | 0 | 60 | 0 | 30 | 0 | 60 | 0 | 30 | 20 | 10 |
| Foxtail, Green | 40 | 30 | 0 | 0 | 0 | 100 | 0 | 90 | 0 | 100 | 0 | 90 | 30 | 80 |
| Kochia | 50 | 50 | 0 | 0 | 0 | 50 | 0 | 0 | 0 | 70 | 0 | 0 | 100 | 100 |
| Oat, Wild | 0 | 0 | 0 | 0 | 0 | 70 | 0 | 0 | 0 | 0 | 0 | 20 | 40 | 50 |
| Pigweed, Palmer | 80 | 30 | 10 | 0 | 0 | 90 | 0 | 100 | — | 100 | 20 | 90 | 90 | 100 |
| Ragweed | 0 | 20 | 0 | 0 | 0 | 0 | 0 | 0 | 0 | 0 | 0 | 0 | 0 | 0 |
| Ryegrass, Italian | 40 | 20 | 0 | 0 | 40 | 90 | 0 | 60 | 0 | 80 | 0 | 80 | 30 | 70 |
| Soybean | 20 | 0 | 0 | 0 | 0 | 10 | 0 | 0 | 0 | 0 | 0 | 10 | 0 | 0 |
| Wheat | 60 | 20 | 0 | 0 | 10 | 90 | 0 | 80 | 0 | 70 | 0 | 40 | 30 | 30 |
| 62 g ai/ha | Compounds | | | | | | | | | | | | | |
| Preemergence | 117 | 118 | 119 | 120 | 121 | 122 | 124 | 125 | 126 | 127 | 128 | 129 | 130 | 131 |
| Barnyardgrass | 10 | 80 | 80 | 60 | 80 | 0 | 40 | 50 | 90 | 0 | 100 | 30 | 70 | 80 |
| Blackgrass | 60 | 90 | 80 | 80 | 90 | 60 | 30 | 90 | 40 | 0 | 50 | 0 | 90 | 90 |
| Corn | 0 | 90 | 70 | 80 | 90 | 0 | 50 | 90 | 40 | 0 | 80 | 30 | 90 | 80 |
| Foxtail, Green | 80 | 90 | 90 | 20 | 90 | 0 | 80 | 90 | 10 | 0 | 100 | 0 | 90 | 100 |
| Kochia | 0 | 60 | 80 | 30 | 70 | 50 | 10 | 90 | — | 0 | 0 | 0 | 90 | 90 |
| Oat, Wild | 90 | 90 | 90 | 90 | 90 | 90 | 30 | 90 | 30 | 0 | 80 | 0 | 90 | 60 |
| Pigweed, Palmer | 10 | 50 | 10 | 20 | 70 | 20 | 30 | 50 | 50 | 0 | 100 | 20 | 30 | 80 |
| Ragweed | 0 | 0 | 0 | 0 | 0 | 0 | 0 | 20 | 0 | 0 | 0 | 0 | 0 | 10 |
| Ryegrass, Italian | 70 | 80 | 80 | 80 | 90 | 60 | 70 | 90 | 40 | 0 | 90 | 40 | 90 | 90 |
| Soybean | 0 | 30 | 20 | 0 | 0 | 10 | 0 | 0 | 0 | 0 | 0 | 0 | 40 | 80 |
| Wheat | 20 | 80 | 60 | 70 | 80 | 50 | 90 | 60 | 90 | 0 | 100 | 20 | 90 | 90 |
| 62 g ai/ha | Compounds | | | | | | | | | | | | | |
| Preemergence | 132 | 133 | 134 | 135 | 136 | 137 | 138 | 139 | 140 | 141 | 142 | 143 | 144 | 145 |
| Barnyardgrass | 0 | 80 | 0 | 50 | 0 | 60 | 100 | 70 | 100 | 70 | 0 | 0 | 100 | 80 |
| Blackgrass | 0 | 20 | 30 | 20 | 0 | 50 | 70 | 80 | 50 | 80 | 0 | 0 | 0 | 80 |
| Corn | 20 | — | 0 | 70 | 0 | 10 | 90 | 90 | 60 | 70 | 0 | 0 | 50 | 80 |
| Foxtail, Green | 30 | 90 | 0 | 80 | 0 | 30 | 90 | 90 | 90 | 90 | 0 | 0 | 90 | 90 |
| Kochia | 60 | 20 | 0 | 20 | 0 | 90 | 90 | 90 | 100 | 80 | 0 | 0 | 40 | 0 |
| Oat, Wild | 0 | 0 | 0 | 30 | 0 | 30 | 90 | 80 | 20 | 40 | 0 | 0 | 20 | 80 |
| Pigweed, Palmer | 20 | 90 | — | — | — | 70 | 90 | — | 90 | — | — | 0 | 80 | 0 |
| Ragweed | 0 | 0 | 0 | 0 | 0 | 0 | 40 | 0 | 0 | 0 | 0 | 0 | 0 | 0 |
| Ryegrass, Italian | 20 | 80 | 0 | 90 | 0 | 60 | 90 | 90 | 90 | 90 | 0 | 0 | 60 | 100 |
| Soybean | 0 | 0 | 0 | 20 | 0 | 50 | 80 | 0 | 80 | 70 | 0 | 0 | 10 | 50 |
| Wheat | 90 | 90 | 0 | 90 | 0 | 90 | 90 | 90 | 80 | 90 | 0 | 0 | 70 | 90 |
| 62 g ai/ha | Compounds | | | | | | | | | | | | | |
| Preemergence | 146 | 147 | 148 | 149 | 150 | 151 | 152 | 153 | 154 | 155 | 156 | 157 | 158 | 159 |
| Barnyardgrass | 0 | 90 | 0 | 90 | 90 | 0 | 100 | 20 | 90 | 10 | 50 | 30 | 90 | 80 |
| Blackgrass | 0 | 0 | 0 | 0 | 50 | 0 | 0 | 0 | 0 | 0 | 30 | 0 | 50 | 0 |
| Corn | 0 | 30 | 0 | 30 | 70 | 0 | 60 | 0 | 30 | 0 | 50 | 0 | 60 | 50 |
| Foxtail, Green | 0 | 90 | 0 | 90 | 90 | 0 | 100 | 0 | 90 | 30 | 90 | 0 | 90 | 100 |
| Kochia | 0 | 90 | — | 50 | 90 | 0 | 90 | 0 | 90 | 20 | 70 | 0 | 80 | 90 |
| Oat, Wild | 0 | 20 | 0 | 20 | 10 | 0 | 70 | 0 | 20 | 0 | 0 | 0 | 30 | 20 |
| Pigweed, Palmer | 0 | 100 | 0 | 100 | 90 | 0 | 90 | 0 | 100 | 0 | 80 | 0 | 90 | 100 |
| Ragweed | 0 | 30 | 0 | 30 | 0 | 0 | 30 | 0 | 50 | 0 | 0 | 0 | 70 | 0 |
| Ryegrass, Italian | 20 | 60 | 0 | 80 | 50 | 0 | 90 | 0 | 80 | 0 | 30 | 0 | 70 | 90 |
| Soybean | 0 | 20 | 20 | 0 | 0 | 0 | 60 | 10 | 50 | 0 | 0 | 0 | 20 | 50 |
| Wheat | 0 | 70 | 0 | 70 | 80 | 0 | 90 | 0 | 60 | 10 | 50 | 0 | 80 | 90 |
| 62 g ai/ha | Compounds | | | | | | | | | | | | | |
| Preemergence | 160 | 161 | 162 | 163 | 164 | 165 | 166 | 167 | 168 | 169 | 170 | 171 | 172 | 173 |
| Barnyardgrass | 0 | 100 | 80 | 20 | 90 | 100 | 100 | 100 | 0 | 90 | 0 | 90 | 90 | 100 |
| Blackgrass | 0 | 90 | 60 | 0 | 80 | 50 | 70 | 70 | 0 | 30 | 0 | 90 | 90 | 90 |
| Corn | 0 | 80 | 70 | 70 | 80 | 60 | 60 | 90 | 0 | 60 | 0 | 40 | 90 | 90 |
| Foxtail, Green | 0 | 100 | 30 | 20 | 100 | 100 | 50 | 100 | 0 | 90 | 0 | 90 | 100 | 100 |
| Kochia | 0 | 50 | 40 | 0 | 70 | 90 | 60 | 100 | 0 | 40 | 0 | 40 | 100 | 80 |
| Oat, Wild | 0 | 90 | 90 | 20 | 80 | 30 | 60 | 90 | 0 | 50 | 0 | 90 | 90 | 90 |
| Pigweed, Palmer | 0 | 100 | 30 | 0 | 90 | 100 | 100 | 100 | 0 | 50 | 0 | 70 | 60 | 90 |
| Ragweed | 0 | 50 | 10 | 10 | 0 | 70 | 30 | 90 | 0 | 10 | 0 | 0 | 0 | 0 |
| Ryegrass, Italian | 0 | 90 | 80 | 80 | 90 | 80 | 90 | 90 | 0 | 90 | 20 | 90 | 90 | 90 |
| Soybean | 0 | 50 | 50 | 0 | 20 | 90 | 90 | 80 | 0 | 20 | 0 | 20 | 70 | 70 |
| Wheat | 0 | 90 | 90 | 80 | 90 | 90 | 90 | 90 | 0 | 90 | 0 | 90 | 100 | 100 |

| | |
|---|---|
| 62 g ai/ha | Compounds |

| Preemergence | 174 | 175 | 177 |
|---|---|---|---|
| Barnyardgrass | 0 | 50 | 90 |
| Blackgrass | 0 | 60 | 90 |
| Corn | 0 | 0 | 10 |
| Foxtail, Green | 0 | 90 | 100 |
| Kochia | 0 | 0 | 80 |
| Oat, Wild | 0 | 0 | 90 |
| Pigweed, Palmer | 0 | 0 | 100 |
| Ragweed | 0 | 0 | 20 |
| Ryegrass, Italian | 0 | 90 | 90 |
| Soybean | 0 | 0 | 0 |
| Wheat | 0 | 90 | 100 |

| 31 g ai/ha | Compounds | | | | | | | | | | | | | |
|---|---|---|---|---|---|---|---|---|---|---|---|---|---|---|
| Preemergence | 1 | 2 | 3 | 4 | 5 | 6 | 7 | 8 | 9 | 10 | 11 | 12 | 13 | 14 |
| Barnyardgrass | 90 | 10 | 90 | 50 | 0 | 20 | 0 | 0 | 0 | 10 | 0 | 0 | 0 | 0 |
| Blackgrass | 60 | 0 | 0 | 0 | 30 | 30 | 20 | 0 | 0 | 30 | 20 | 20 | 20 | 0 |
| Ragweed | 20 | 30 | 10 | 0 | 0 | 0 | 0 | 60 | 0 | 0 | 0 | 0 | 40 | 0 |
| Foxtail, Green | 100 | 0 | 30 | 0 | 20 | 50 | 20 | 0 | 0 | 0 | 0 | 0 | 0 | 0 |
| Ryegrass, Italian | 80 | 20 | 50 | 50 | — | — | — | 0 | 0 | 10 | 40 | 0 | 20 | 0 |
| Kochia | 50 | 0 | 70 | 40 | 0 | 100 | 0 | 0 | 0 | 20 | 0 | 20 | 0 | 0 |
| Corn | 60 | 20 | 50 | 40 | 0 | 30 | 0 | 0 | 10 | 0 | 0 | 0 | 0 | 0 |
| Soybean | 70 | 0 | 20 | 0 | 0 | 50 | 20 | 0 | 0 | 10 | 0 | 10 | 0 | 0 |
| Amaranth, Palmer | 90 | 20 | 80 | 30 | 20 | 60 | 0 | 0 | 0 | 0 | 0 | 0 | 0 | 0 |
| Oat, Wild | 60 | 0 | 10 | 50 | 20 | 30 | 20 | 0 | 0 | 50 | 20 | 0 | 0 | 0 |
| Wheat | 90 | 50 | 50 | 40 | 0 | 50 | 90 | 0 | 80 | 90 | 90 | 50 | 0 | 0 |

| | |
|---|---|
| 31 g ai/ha | Compounds |

| Preemergence | 15 | 16 | 17 | 18 | 19 | 20 | 21 | 22 | 23 | 24 | 29 | 31 | 32 | 33 |
|---|---|---|---|---|---|---|---|---|---|---|---|---|---|---|
| Barnyardgrass | 0 | 0 | 80 | 30 | 30 | 0 | 50 | 0 | 60 | 10 | 0 | 0 | 0 | 0 |
| Blackgrass | 0 | 0 | 20 | 0 | 0 | 0 | 0 | 0 | 80 | 0 | 0 | 20 | 0 | 20 |
| Ragweed | 0 | 0 | 20 | 20 | 0 | 30 | 30 | 0 | 0 | 0 | 0 | 0 | 0 | 0 |
| Foxtail, Green | 30 | 0 | 50 | 40 | 50 | 0 | 90 | 0 | 70 | 50 | 0 | 0 | 0 | 0 |
| Ryegrass, Italian | 80 | 40 | 90 | 40 | 80 | 0 | 50 | 0 | 80 | 0 | 0 | 0 | 0 | 0 |
| Kochia | 0 | 0 | 50 | 20 | 0 | 0 | 0 | 0 | 0 | 0 | 0 | 0 | 0 | 0 |
| Corn | 20 | 20 | 20 | 0 | 10 | 0 | 60 | 20 | 30 | 0 | 0 | 0 | 0 | 0 |
| Soybean | 0 | 0 | 0 | 0 | 0 | 20 | 50 | 0 | 10 | 0 | 0 | 0 | 20 | 0 |
| Amaranth, Palmer | 50 | 0 | 70 | 10 | 30 | 0 | 40 | 0 | 30 | 30 | 0 | 0 | 0 | 0 |
| Oat, Wild | 0 | 0 | 10 | 0 | 0 | 0 | 60 | 0 | 20 | 0 | 0 | 0 | 0 | 0 |
| Wheat | 50 | 50 | 80 | 70 | 70 | 0 | 80 | 20 | 80 | 0 | 0 | 0 | 0 | 30 |

| 31 g ai/ha | Compounds | | | | | | | | | | | | | |
|---|---|---|---|---|---|---|---|---|---|---|---|---|---|---|
| Preemergence | 37 | 38 | 39 | 40 | 41 | 53 | 54 | 55 | 56 | 57 | 58 | 59 | 60 | 62 |
| Barnyardgrass | 30 | 0 | 0 | 0 | 0 | 30 | 30 | 20 | 30 | 50 | 80 | 60 | 60 | 80 |
| Blackgrass | 0 | 0 | 0 | 50 | 0 | 50 | 20 | 20 | 30 | 70 | 10 | 0 | 0 | 30 |
| Ragweed | 60 | 0 | 0 | 0 | 0 | 0 | 0 | 0 | 0 | 0 | 0 | 30 | 30 | 30 |
| Foxtail, Green | 0 | 0 | 0 | 0 | 0 | 0 | 0 | 0 | 40 | 0 | — | — | — | 80 |
| Ryegrass, Italian | 70 | 0 | 0 | 20 | 0 | 70 | 50 | 80 | 80 | 50 | 60 | 60 | 60 | 60 |
| Kochia | 0 | 0 | 0 | 0 | 0 | 0 | 0 | 0 | 0 | 0 | 10 | 40 | 40 | 30 |
| Corn | 10 | 0 | 0 | 0 | 0 | 0 | 0 | 20 | 10 | 10 | 10 | 0 | 0 | 30 |
| Soybean | 10 | 10 | 0 | 0 | 0 | 30 | 10 | 40 | 30 | 50 | 20 | 30 | 10 | 0 |
| Amaranth, Palmer | 10 | 0 | 0 | 0 | 0 | 30 | 10 | 20 | 0 | 10 | 80 | 100 | 100 | 90 |
| Oat, Wild | 20 | 0 | 0 | 50 | 0 | 50 | 0 | 60 | 90 | 90 | 10 | 10 | 0 | 40 |
| Wheat | 40 | 40 | 0 | 50 | 0 | 70 | 80 | 90 | 90 | 90 | 20 | 30 | 20 | 80 |

| 31 g ai/ha | Compounds | | | | | | | | | | | | | |
|---|---|---|---|---|---|---|---|---|---|---|---|---|---|---|
| Preemergence | 63 | 64 | 65 | 66 | 67 | 68 | 69 | 70 | 71 | 72 | 73 | 75 | 79 | 80 |
| Barnyardgrass | 30 | 80 | 90 | 50 | 50 | 90 | 60 | 30 | 50 | 60 | 20 | 0 | 30 | 0 |
| Blackgrass | 30 | 20 | 50 | 30 | 20 | 50 | 40 | 10 | 0 | 0 | 10 | 20 | 10 | 0 |
| Ragweed | 20 | 0 | 10 | 0 | 0 | 0 | 0 | 10 | 0 | 0 | 0 | 10 | 0 | 10 |
| Foxtail, Green | 70 | 70 | 90 | 20 | 20 | — | — | — | — | — | — | — | — | — |
| Ryegrass, Italian | 40 | 50 | 70 | 50 | 70 | 70 | 30 | 0 | 40 | 40 | 20 | 10 | 20 | 0 |
| Kochia | 0 | 50 | 10 | 0 | 70 | 80 | 40 | 0 | 70 | 80 | 30 | 50 | 30 | 0 |
| Corn | 10 | 0 | 0 | 0 | 0 | 0 | 0 | 0 | 30 | 0 | 0 | 0 | 0 | 0 |
| Soybean | 0 | 0 | 10 | 0 | 0 | 0 | 20 | 0 | 10 | 0 | 0 | 30 | 0 | 0 |
| Amaranth, Palmer | 70 | 100 | 100 | 90 | 90 | 100 | 90 | 30 | 90 | 90 | 20 | 50 | 80 | 0 |
| Oat, Wild | 0 | 20 | 20 | 0 | 40 | 60 | 0 | 0 | 20 | 10 | 0 | 0 | 20 | 0 |
| Wheat | 10 | 60 | 50 | 10 | 30 | 80 | 30 | 0 | 60 | 40 | 0 | 0 | 10 | 0 |

| | |
|---|---|
| 31 g ai/ha | Compounds |

| Preemergence | 81 | 83 | 85 | 86 |
|---|---|---|---|---|
| Barnyardgrass | 90 | 30 | 20 | 30 |
| Blackgrass | 30 | 0 | 0 | 0 |
| Ragweed | 20 | 0 | 30 | 10 |
| Foxtail, Green | — | — | — | — |
| Ryegrass, Italian | 60 | 60 | 60 | 0 |
| Kochia | 70 | 0 | 20 | 40 |
| Corn | 0 | 0 | 0 | 0 |
| Soybean | 10 | 40 | 0 | 0 |
| Amaranth, Palmer | 50 | 70 | 80 | 70 |
| Oat, Wild | 40 | 0 | 20 | 0 |
| Wheat | 40 | 0 | 0 | 0 |

| | |
|---|---|
| 31 g ai/ha | Compounds |

| Preemergence | 87 | 88 |
|---|---|---|
| Barnyardgrass | 90 | 40 |
| Blackgrass | 20 | 30 |
| Corn | 0 | 0 |
| Foxtail, Green | 20 | 30 |
| Kochia | 10 | 60 |
| Oat, Wild | 0 | 0 |
| Pigweed, Palmer | 90 | 80 |
| Ragweed | 10 | 40 |
| Ryegrass, Italian | 30 | 50 |
| Soybean | 0 | 0 |
| Wheat | 20 | 10 |

| 16 g ai/ha | Compounds | | | | | | | | | | | | | |
|---|---|---|---|---|---|---|---|---|---|---|---|---|---|---|
| Preemergence | 89 | 90 | 91 | 92 | 93 | 94 | 95 | 96 | 97 | 98 | 99 | 100 | 101 | 102 |
| Barnyardgrass | 70 | 0 | 0 | 0 | 0 | 40 | 0 | 10 | 0 | 10 | 0 | 30 | 0 | 0 |
| Blackgrass | 30 | 0 | 0 | 0 | 0 | 30 | 0 | 0 | 0 | 10 | 0 | 0 | 0 | 0 |
| Corn | 30 | 0 | 0 | 0 | 0 | 40 | 0 | 0 | 0 | 0 | 0 | 30 | 0 | 0 |
| Foxtail, Green | 10 | 0 | 0 | 0 | 0 | 10 | 0 | 10 | 20 | 70 | 0 | 80 | 0 | 0 |
| Kochia | 90 | 0 | 0 | 0 | 20 | 0 | 0 | 0 | 0 | 80 | 0 | 0 | 0 | 0 |
| Oat, Wild | 80 | 0 | 0 | 0 | 0 | 10 | 0 | 0 | 0 | 0 | 20 | 0 | 0 | 0 |
| Pigweed, Palmer | 80 | 10 | 0 | 0 | — | 20 | 0 | 100 | 0 | 10 | 0 | 90 | 0 | 0 |
| Ragweed | 0 | 0 | 0 | 0 | 0 | 20 | 0 | 0 | 0 | 0 | 0 | 0 | 0 | 0 |
| Ryegrass, Italian | 80 | 0 | 0 | 0 | 0 | 60 | 0 | 20 | 0 | 0 | 0 | 60 | 0 | 0 |
| Soybean | 40 | 0 | 0 | 0 | 0 | 10 | 0 | 10 | 0 | 0 | 0 | 0 | 0 | 0 |
| Wheat | 90 | 0 | 0 | 0 | 0 | 60 | 40 | 0 | 0 | 30 | 0 | 50 | 0 | 0 |

| 16 g ai/ha | Compounds | | | | | | | | | | | | | |
|---|---|---|---|---|---|---|---|---|---|---|---|---|---|---|
| Preemergence | 103 | 104 | 105 | 106 | 107 | 108 | 109 | 110 | 111 | 112 | 113 | 114 | 115 | 116 |
| Barnyardgrass | 0 | 0 | 0 | 0 | 0 | 20 | 0 | 50 | 0 | 20 | 0 | 0 | 30 | 30 |
| Blackgrass | 0 | 0 | 0 | 0 | 0 | 0 | 0 | 0 | 0 | 0 | 0 | 0 | 20 | 0 |
| Corn | 0 | 0 | 0 | 0 | 0 | 30 | 0 | 0 | 0 | 20 | 0 | 0 | 0 | 0 |
| Foxtail, Green | 20 | 0 | 0 | 0 | 0 | 30 | 0 | 0 | 0 | 20 | 0 | 0 | 0 | 0 |
| Kochia | 0 | 0 | 0 | 0 | 0 | 0 | 0 | 0 | 0 | 0 | 0 | 0 | — | — |
| Oat, Wild | 0 | 0 | 0 | 0 | 0 | 0 | 0 | 0 | 0 | 0 | 0 | 0 | 0 | 0 |
| Pigweed, Palmer | 0 | 0 | — | 0 | 0 | 30 | 0 | 80 | 0 | 0 | 0 | 40 | 20 | 10 |
| Ragweed | 0 | 20 | 0 | 0 | 0 | 0 | 0 | 0 | 0 | 0 | 0 | 0 | 0 | 0 |
| Ryegrass, Italian | 0 | 0 | 0 | 0 | 10 | 70 | 0 | 0 | 0 | 60 | 0 | 0 | 10 | 0 |
| Soybean | 20 | 0 | 0 | 0 | 0 | 0 | 0 | 10 | 100 | 10 | 0 | 0 | 0 | 0 |
| Wheat | 0 | 0 | 0 | 0 | 0 | 90 | 0 | 40 | 0 | 40 | 0 | 0 | 0 | 0 |
| 16 g ai/ha | Compounds | | | | | | | | | | | | | |
| Preemergence | 117 | 118 | 119 | 120 | 121 | 122 | 123 | 124 | 125 | 126 | 127 | 128 | 129 | 130 |
| Barnyardgrass | 0 | 10 | 0 | 0 | 0 | 0 | 0 | 0 | 0 | 10 | 0 | 0 | 0 | 0 |
| Blackgrass | 30 | 30 | 40 | 40 | 50 | 40 | 0 | 20 | 50 | 0 | 0 | 0 | 0 | 70 |
| Corn | 0 | 30 | 20 | 0 | 20 | 0 | 0 | 0 | 0 | 0 | 0 | 10 | 0 | 10 |
| Foxtail, Green | 0 | 60 | 20 | 20 | 0 | 0 | 0 | 0 | 10 | 0 | 0 | 100 | 0 | 50 |
| Kochia | 0 | 0 | 0 | 0 | 0 | 0 | 0 | 0 | 0 | 0 | 0 | 0 | — | 0 |
| Oat, Wild | 40 | 90 | 90 | 90 | 90 | 60 | 10 | 0 | 90 | 0 | 0 | 30 | 0 | 50 |
| Pigweed, Palmer | 0 | 20 | 10 | 0 | 0 | 0 | 0 | 0 | 20 | 20 | 0 | 10 | 0 | 10 |
| Ragweed | 0 | 0 | 0 | 10 | 0 | 0 | 0 | 0 | 0 | 0 | 0 | 0 | 0 | 0 |
| Ryegrass, Italian | 0 | 20 | 30 | 10 | 50 | 0 | 0 | 0 | 0 | 0 | 0 | 60 | 0 | 40 |
| Soybean | 0 | 0 | 0 | 0 | 0 | 0 | 0 | 0 | 10 | 0 | 0 | 0 | 0 | 0 |
| Wheat | 0 | 20 | 10 | 20 | 40 | 30 | 80 | 20 | 30 | 20 | 0 | 90 | 0 | 90 |
| 16 g ai/ha | Compounds | | | | | | | | | | | | | |
| Preemergence | 131 | 132 | 133 | 134 | 135 | 136 | 137 | 138 | 139 | 140 | 141 | 142 | 143 | 144 |
| Barnyardgrass | 0 | 0 | 0 | 0 | 20 | 0 | 0 | 60 | 10 | 50 | 0 | 0 | 0 | 0 |
| Blackgrass | 30 | 0 | 0 | 0 | 0 | 0 | 0 | 40 | 20 | 0 | 0 | 0 | 0 | 0 |
| Corn | 10 | 0 | 0 | 0 | 0 | 0 | 0 | 60 | 0 | 10 | 10 | 0 | 0 | 0 |
| Foxtail, Green | 0 | 0 | 0 | 0 | 0 | 0 | 0 | 20 | 30 | 80 | 20 | 0 | 0 | 0 |
| Kochia | 0 | 0 | 0 | 0 | 0 | 0 | 0 | 90 | 0 | 50 | 0 | 0 | 0 | 0 |
| Oat, Wild | 0 | 0 | 0 | 0 | 20 | 0 | 0 | 50 | 20 | 20 | 0 | 0 | 0 | 0 |
| Pigweed, Palmer | 0 | — | — | — | — | — | 20 | 30 | — | — | — | — | 0 | 50 |
| Ragweed | 0 | 0 | 0 | 0 | 0 | 0 | 0 | 0 | 0 | 0 | 0 | 0 | 0 | 0 |
| Ryegrass, Italian | 0 | 0 | 10 | 0 | 0 | 0 | 30 | 70 | 30 | 50 | 50 | 0 | 0 | 10 |
| Soybean | 0 | 0 | 0 | 0 | 20 | 0 | 0 | 30 | 0 | 0 | 0 | — | 0 | 0 |
| Wheat | 30 | 80 | 0 | 0 | 10 | 0 | 20 | 50 | 90 | 40 | 80 | 0 | 0 | 0 |
| 16 g ai/ha | Compounds | | | | | | | | | | | | | |
| Preemergence | 145 | 146 | 147 | 148 | 149 | 150 | 151 | 152 | 153 | 154 | 155 | 156 | 157 | 158 |
| Barnyardgrass | 20 | 0 | 30 | 0 | 60 | 0 | 0 | 30 | 0 | 30 | 0 | 0 | 0 | 40 |
| Blackgrass | 0 | 0 | 0 | 0 | 0 | 0 | 0 | 0 | 0 | 0 | 0 | 0 | 0 | 0 |
| Corn | 10 | 0 | 30 | 0 | 0 | 0 | 0 | 10 | 0 | 20 | 0 | 0 | 0 | 0 |
| Foxtail, Green | 10 | 0 | 0 | 0 | 30 | 0 | 0 | 30 | 0 | 40 | 30 | 40 | 0 | 0 |
| Kochia | 0 | 0 | 20 | — | 0 | 20 | 0 | 90 | 0 | 20 | — | 20 | 0 | 30 |
| Oat, Wild | 20 | 0 | 0 | 0 | 0 | 0 | 0 | 0 | 0 | 0 | 0 | 0 | 0 | 10 |
| Pigweed, Palmer | 0 | 0 | 50 | 0 | 90 | 10 | 0 | 0 | 0 | 60 | 0 | 20 | 0 | 50 |
| Ragweed | 0 | 0 | 0 | 0 | 0 | 0 | 0 | 0 | 0 | 0 | 0 | 0 | 0 | 0 |
| Ryegrass, Italian | 30 | 0 | 20 | 0 | 20 | 10 | 0 | 70 | 0 | 30 | 0 | 0 | 0 | 0 |
| Soybean | 0 | 0 | 10 | 0 | 0 | 0 | 0 | 0 | 0 | 0 | 0 | 0 | 0 | 0 |
| Wheat | 80 | 0 | 20 | 0 | 20 | 0 | 0 | 70 | 0 | 10 | 0 | 10 | 0 | 40 |
| 16 g ai/ha | Compounds | | | | | | | | | | | | | |
| Preemergence | 159 | 160 | 161 | 162 | 163 | 164 | 165 | 166 | 167 | 168 | 169 | 170 | 171 | 172 |
| Barnyardgrass | 0 | 0 | 80 | 0 | 0 | 0 | 30 | 40 | 60 | 0 | 10 | 0 | 0 | 0 |
| Blackgrass | 0 | 0 | 70 | 0 | 0 | 0 | 20 | 10 | 30 | 0 | 0 | 0 | 20 | 50 |
| Corn | 20 | 0 | 30 | 0 | 0 | 50 | 30 | 20 | 40 | 0 | 0 | 0 | 0 | 80 |
| Foxtail, Green | 20 | 0 | 30 | 0 | 0 | 0 | 20 | 0 | 90 | 0 | 0 | 0 | 0 | 40 |
| Kochia | 0 | 0 | 0 | 0 | 0 | 20 | 80 | 0 | 70 | 0 | 0 | 0 | 30 | 0 |
| Oat, Wild | 0 | 0 | 30 | 20 | 0 | 60 | 0 | 0 | 50 | 0 | 20 | 0 | 20 | 30 |
| Pigweed, Palmer | 30 | 0 | 90 | 0 | 0 | 10 | 90 | 20 | 100 | 0 | 0 | 0 | 0 | 10 |
| Ragweed | 0 | 0 | 10 | 0 | 0 | 0 | 20 | 0 | 60 | 0 | 0 | 0 | 0 | 0 |
| Ryegrass, Italian | 30 | 0 | 90 | 0 | 0 | 0 | 20 | 0 | 80 | 0 | 90 | 10 | 20 | 40 |
| Soybean | 0 | 0 | 0 | 10 | 0 | 10 | 70 | 20 | 70 | 0 | 10 | 0 | 0 | 0 |
| Wheat | 50 | 0 | 80 | 20 | 20 | 80 | 70 | 60 | 70 | 0 | 80 | 0 | 60 | 90 |

| | |
|---|---|
| 16 g ai/ha | Compounds |

| Preemergence | 173 | 174 | 175 | 177 |
|---|---|---|---|---|
| Barnyardgrass | 40 | 0 | 0 | 60 |
| Blackgrass | 90 | 0 | 40 | 60 |
| Corn | 50 | 0 | 0 | 10 |
| Foxtail, Green | 90 | 0 | 0 | 90 |
| Kochia | 10 | 0 | 0 | 0 |
| Oat, Wild | 70 | 0 | 0 | 0 |
| Pigweed, Palmer | 20 | 0 | 0 | 90 |
| Ragweed | 0 | 0 | 0 | 0 |
| Ryegrass, Italian | 80 | 0 | 10 | 80 |
| Soybean | 30 | 0 | 0 | 0 |
| Wheat | 90 | 0 | 40 | 40 |

| | |
|---|---|
| 8 g ai/ha | Compounds |

| Preemergence | 87 | 88 |
|---|---|---|
| Barnyardgrass | 0 | 10 |
| Blackgrass | 10 | 0 |
| Corn | 0 | 0 |
| Foxtail, Green | 30 | 20 |
| Kochia | 0 | 0 |
| Oat, Wild | 0 | 0 |
| Pigweed, Palmer | 10 | 50 |
| Ragweed | 10 | 0 |
| Ryegrass, Italian | 0 | 10 |
| Soybean | 0 | 0 |
| Wheat | 0 | 0 |

| | |
|---|---|
| 1000 g ai/ha | Compounds |

| Postemergence | 26 | 27 | 28 | 48 | 50 | 52 |
|---|---|---|---|---|---|---|
| Barnyardgrass | — | — | — | 90 | 90 | 100 |
| Blackgrass | 90 | 90 | 100 | 100 | 90 | 90 |
| Horseweed | 70 | 80 | 80 | 70 | 70 | 100 |
| Ragweed | 50 | 80 | 70 | 70 | 70 | 70 |
| Galium | 70 | — | 70 | 80 | 80 | 80 |
| Foxtail, Giant | 90 | 90 | 90 | 90 | 90 | 90 |
| Goosegrass | 90 | 90 | 90 | — | — | — |
| Ryegrass, Italian | 70 | 100 | 80 | 90 | 100 | 60 |
| Kochia | 80 | 80 | 80 | 70 | 70 | 70 |
| Amaranth, Palmer | 90 | 90 | 90 | 90 | 90 | 90 |

| | |
|---|---|
| 1000 g ai/ha | Compounds |

| Postemergence | 179 | 180 | 181 | 182 |
|---|---|---|---|---|
| Barnyardgrass | 100 | 100 | 90 | 90 |
| Blackgrass | 100 | 90 | 90 | 90 |
| Foxtail, Green | 90 | 90 | 90 | 90 |
| Galium | 90 | 90 | 90 | 90 |
| Horseweed | 80 | 90 | 10 | 80 |
| Kochia | 90 | 80 | 80 | 80 |
| Pigweed, Palmer | 90 | 90 | 90 | 90 |
| Ragweed | 80 | 70 | 60 | 80 |
| Ryegrass, Italian | 100 | 100 | 100 | 100 |

| 500 g ai/ha | Compounds | | | | | | | | | | | | |
|---|---|---|---|---|---|---|---|---|---|---|---|---|---|
| Postemergence | 25 | 30 | 34 | 35 | 36 | 42 | 43 | 44 | 45 | 46 | 47 | 49 | 51 |
| Barnyardgrass | — | 100 | 100 | 100 | 100 | 100 | 100 | 100 | 100 | 100 | 80 | 100 | 100 |
| Blackgrass | 90 | 100 | 100 | 100 | 100 | 100 | 100 | 100 | 100 | 100 | 70 | 100 | 100 |
| Horseweed | 80 | 80 | 90 | 90 | 90 | 90 | 80 | 90 | 80 | 80 | 40 | — | 100 |
| Ragweed | 80 | 80 | 80 | 80 | 80 | 80 | 80 | 80 | 80 | 80 | 50 | 70 | 70 |
| Galium | 80 | 80 | 80 | 80 | 80 | 80 | 70 | 70 | 70 | 70 | 80 | 80 | 70 |
| Foxtail, Giant | 90 | 90 | — | — | — | — | — | — | — | — | — | 90 | 90 |
| Goosegrass | 90 | — | — | — | — | — | — | — | — | — | — | — | — |
| Foxtail, Green | — | — | 90 | 90 | 90 | 90 | 90 | 90 | 90 | 90 | 90 | — | — |
| Ryegrass, Italian | 60 | 100 | 90 | 90 | 80 | 90 | 90 | 80 | 80 | 70 | 70 | 90 | 70 |
| Kochia | 90 | 80 | 70 | 70 | 70 | 80 | 80 | 80 | 80 | 80 | 80 | 80 | 80 |
| Corn | 100 | 90 | 100 | 100 | 100 | 100 | 100 | 100 | 100 | 100 | 90 | 100 | 90 |
| Soybean | 80 | 70 | 80 | 90 | 80 | 80 | 80 | 80 | 90 | 90 | 50 | 90 | 100 |
| Amaranth, Palmer | 90 | 90 | 100 | 100 | 100 | 100 | 100 | 100 | 100 | 90 | 100 | 100 | 90 |
| Oat, Wild | 100 | 100 | 100 | 100 | 100 | 90 | 90 | 90 | 90 | 90 | 20 | 90 | 90 |
| Wheat | 80 | 80 | 80 | 80 | 80 | 90 | 90 | 90 | 90 | 90 | 10 | 90 | 90 |

| 500 g ai/ha | Compound |
|---|---|
| Postemergence | 176 |
| Barnyardgrass | 100 |
| Blackgrass | 90 |
| Corn | 100 |
| Foxtail, Green | 90 |
| Galium | 100 |
| Horseweed | 30 |
| Kochia | 90 |
| Oat, Wild | 90 |
| Pigweed, Palmer | 90 |
| Ragweed | 80 |
| Ryegrass, Italian | 100 |
| Soybean | 70 |
| Wheat | 90 |
| 125 g ai/ha | Compound |
| Postemergence | 176 |
| Barnyardgrass | 100 |
| Blackgrass | 90 |
| Corn | 100 |
| Foxtail, Green | 90 |
| Galium | 90 |
| Horseweed | 0 |
| Kochia | 90 |
| Oat, Wild | 90 |
| Pigweed, Palmer | 80 |
| Ragweed | 70 |
| Ryegrass, Italian | 100 |
| Soybean | 70 |
| Wheat | 90 |

| 125 g ai/ha | Compounds | | | | | | | | | | | | | |
|---|---|---|---|---|---|---|---|---|---|---|---|---|---|---|
| Postemergence | 1 | 2 | 3 | 4 | 5 | 6 | 7 | 8 | 9 | 10 | 11 | 12 | 13 | 14 |
| Barnyardgrass | 100 | 100 | 100 | 100 | 100 | 100 | 100 | 0 | 100 | 100 | 100 | 100 | 70 | 0 |
| Blackgrass | 100 | 70 | 90 | 90 | 90 | 90 | 70 | 90 | 70 | 90 | 90 | 80 | 60 | 0 |
| Horseweed | 80 | 0 | 80 | 70 | 50 | 90 | 40 | 10 | 10 | 40 | 20 | 10 | 20 | 0 |
| Ragweed | 70 | 50 | 80 | 80 | 70 | 80 | 20 | 0 | 0 | 0 | 0 | 0 | 20 | 0 |
| Galium | 90 | 90 | 90 | 90 | 80 | 80 | 80 | — | — | — | — | — | 70 | 0 |
| Foxtail, Giant | — | — | — | — | — | — | — | — | — | — | — | — | — | — |
| Goosegrass | — | — | — | — | — | — | — | — | — | — | — | — | — | — |
| Foxtail, Green | 90 | 90 | 90 | 90 | 90 | 90 | 90 | 0 | 90 | 90 | 90 | 60 | 50 | 0 |
| Ryegrass, Italian | 80 | 90 | 90 | 90 | 90 | 90 | 90 | 0 | 80 | 80 | 70 | 80 | 70 | 0 |
| Kochia | 90 | 80 | 70 | 80 | 80 | 80 | 80 | 20 | 80 | 70 | 80 | 80 | 50 | 0 |
| Corn | 100 | 100 | 100 | 100 | 100 | 100 | 100 | 0 | 100 | 100 | 80 | 50 | 30 | 20 |
| Soybean | 90 | 70 | 90 | 80 | 70 | 80 | 60 | 0 | 70 | 70 | 60 | 50 | 50 | 0 |
| Amaranth, Palmer | 90 | 50 | 70 | 90 | 50 | 90 | 80 | 0 | 90 | 80 | 60 | 20 | 90 | 0 |
| Oat, Wild | 100 | 90 | 90 | 90 | 90 | 100 | 100 | 0 | 90 | 90 | 90 | 90 | 50 | 0 |
| Wheat | 90 | 90 | 90 | 90 | 90 | 90 | 90 | 20 | 90 | 90 | 90 | 60 | 70 | 0 |

| 125 g ai/ha | Compounds | | | | | | | | | | | | | |
|---|---|---|---|---|---|---|---|---|---|---|---|---|---|---|
| Postemergence | 15 | 16 | 17 | 18 | 19 | 20 | 21 | 22 | 23 | 24 | 25 | 29 | 30 | 31 |
| Barnyardgrass | 100 | 100 | 100 | 100 | 100 | 90 | 100 | 90 | 100 | 80 | — | 80 | 100 | 90 |
| Blackgrass | 100 | 100 | 90 | 80 | 90 | 60 | 100 | 80 | 100 | 60 | 80 | 90 | 70 | 100 |
| Horseweed | 0 | 20 | 70 | 20 | 0 | 0 | 70 | 30 | 90 | 10 | 60 | 20 | 30 | 0 |
| Ragweed | 50 | 50 | 80 | 0 | 40 | 30 | 70 | 50 | 70 | 30 | 60 | 30 | 20 | 50 |
| Galium | 90 | 90 | 80 | 90 | 80 | 80 | 80 | 80 | 90 | 80 | 80 | 80 | 80 | 70 |
| Foxtail, Giant | — | — | — | — | — | — | — | — | — | — | 90 | — | 90 | — |
| Goosegrass | — | — | — | — | — | — | — | — | — | — | 90 | — | — | — |
| Foxtail, Green | 90 | 90 | 90 | 90 | 90 | 80 | 90 | 80 | 90 | 60 | — | 80 | — | 80 |
| Ryegrass, Italian | 90 | 90 | 90 | 100 | 90 | 80 | 40 | 70 | 70 | 60 | 60 | 90 | 80 | 90 |
| Kochia | 70 | 70 | 70 | 70 | 70 | 50 | 80 | 90 | 90 | 90 | 80 | 40 | 70 | 70 |
| Corn | 100 | 100 | 100 | 100 | 100 | 100 | 100 | 100 | 100 | 90 | 100 | 70 | 60 | 50 |
| Soybean | 70 | 60 | 90 | 60 | 60 | 90 | 90 | 90 | 90 | 60 | 70 | 50 | 60 | 60 |
| Amaranth, Palmer | 90 | 70 | 90 | 90 | 90 | 30 | 90 | 80 | 90 | 40 | 90 | 60 | 90 | 20 |
| Oat, Wild | 90 | 90 | 90 | 90 | 90 | 60 | 100 | 70 | 100 | 60 | 90 | 90 | 100 | 90 |
| Wheat | 90 | 90 | 90 | 90 | 90 | 80 | 90 | 90 | 90 | 90 | 80 | 90 | 80 | 90 |

| 125 g ai/ha | Compounds | | | | | | | | | | | | | |
|---|---|---|---|---|---|---|---|---|---|---|---|---|---|---|
| Postemergence | 32 | 33 | 34 | 35 | 36 | 37 | 38 | 39 | 40 | 41 | 42 | 43 | 44 | 45 |
| Barnyardgrass | 90 | 90 | 100 | 100 | 100 | 100 | 100 | 90 | 100 | 30 | 100 | 100 | 100 | 100 |
| Blackgrass | 100 | 100 | 90 | 90 | 100 | 90 | 90 | 100 | 100 | 90 | 100 | 100 | 100 | 100 |
| Horseweed | 0 | 30 | 90 | 80 | 70 | 70 | 70 | 0 | 0 | 0 | 80 | 60 | 90 | 80 |
| Ragweed | 50 | 50 | 70 | 70 | 60 | 50 | 50 | 20 | 20 | 0 | 70 | 60 | 70 | 70 |
| Galium | 80 | 70 | 80 | 80 | 70 | — | — | — | — | — | 70 | 70 | 70 | 70 |
| Foxtail, Giant | — | — | — | — | — | — | — | — | — | — | — | — | — | — |
| Goosegrass | — | — | — | — | — | — | — | — | — | — | — | — | — | — |
| Foxtail, Green | 40 | 40 | 90 | 90 | 80 | 90 | 90 | 90 | 90 | 30 | 90 | 90 | 90 | 80 |
| Ryegrass, Italian | 80 | 80 | 80 | 90 | 90 | 90 | 90 | 80 | 60 | 50 | 90 | 90 | 90 | 80 |
| Kochia | 50 | 60 | 70 | 70 | 60 | 80 | 80 | 50 | 70 | 0 | 80 | 80 | 80 | 80 |
| Corn | 50 | 90 | 100 | 100 | 100 | 100 | 100 | 100 | 100 | 0 | 100 | 100 | 100 | 100 |
| Soybean | 60 | 60 | 70 | 70 | 70 | 80 | 80 | 60 | 60 | 0 | 90 | 80 | 90 | 90 |
| Amaranth, Palmer | 30 | 40 | 80 | 80 | 60 | 90 | 100 | 20 | 30 | 0 | 100 | 100 | 100 | 100 |
| Oat, Wild | 90 | 80 | 100 | 100 | 90 | 90 | 90 | 90 | 90 | 40 | 90 | 90 | 90 | 90 |
| Wheat | 90 | 90 | 70 | 70 | 70 | 90 | 90 | 90 | 80 | 80 | 90 | 90 | 90 | 90 |

| 125 g ai/ha | Compounds | | | | | | | | | | | | | |
|---|---|---|---|---|---|---|---|---|---|---|---|---|---|---|
| Postemergence | 46 | 47 | 49 | 51 | 53 | 54 | 55 | 56 | 57 | 58 | 59 | 60 | 62 | 63 |
| Barnyardgrass | 100 | 0 | 100 | 100 | 100 | 100 | 100 | 100 | 100 | 90 | 90 | 90 | 100 | 100 |
| Blackgrass | 100 | 20 | 90 | 90 | 80 | 90 | 100 | 90 | 90 | 100 | 100 | 100 | 100 | 100 |
| Horseweed | 80 | 50 | — | 80 | 70 | 80 | 80 | 80 | 60 | 90 | 70 | 90 | 70 | 80 |
| Ragweed | 60 | 40 | 70 | 60 | 80 | 60 | 60 | 70 | 60 | 70 | 80 | 20 | 90 | 80 |
| Galium | 80 | 80 | 70 | 70 | 80 | 80 | 80 | 80 | 80 | 90 | 90 | 90 | 80 | 80 |
| Foxtail, Giant | — | — | 90 | 90 | — | — | — | — | — | — | — | — | — | — |
| Goosegrass | — | — | — | — | — | — | — | — | — | — | — | — | — | — |
| Foxtail, Green | 80 | 20 | — | — | 70 | 60 | 80 | 90 | 90 | — | — | — | 90 | 90 |
| Ryegrass, Italian | 70 | 30 | 80 | 70 | 90 | 90 | 90 | 90 | 90 | 90 | 90 | 100 | 100 | 90 |
| Kochia | 80 | 70 | 70 | 80 | 80 | 90 | 80 | 80 | 80 | 90 | 90 | 80 | 80 | 80 |
| Corn | 100 | 80 | 100 | 90 | 100 | 100 | 100 | 100 | 100 | 100 | 100 | 100 | 100 | 100 |
| Soybean | 80 | 30 | 90 | 70 | 70 | 60 | 70 | 70 | 70 | 90 | 100 | 100 | 90 | 90 |
| Amaranth, Palmer | 90 | 80 | 90 | 90 | 90 | 100 | 90 | 80 | 70 | 100 | 100 | 100 | 90 | 90 |
| Oat, Wild | 80 | 20 | 90 | 90 | 90 | 100 | 90 | 90 | 90 | 70 | 90 | 90 | 90 | 90 |
| Wheat | 90 | 10 | 90 | 90 | 90 | 90 | 90 | 90 | 90 | 90 | 90 | 90 | 90 | 90 |

| 125 g ai/ha | Compounds | | | | | | | | | | | | | |
|---|---|---|---|---|---|---|---|---|---|---|---|---|---|---|
| Postemergence | 64 | 65 | 66 | 67 | 68 | 69 | 70 | 71 | 72 | 73 | 75 | 79 | 80 | 81 |
| Barnyardgrass | 100 | 100 | 100 | 100 | 90 | 90 | 90 | 90 | 90 | 90 | 90 | 90 | 90 | 90 |
| Blackgrass | 90 | 100 | 90 | 100 | 90 | 90 | 80 | 90 | 90 | 90 | 80 | 90 | 70 | 50 |
| Horseweed | 80 | 70 | 70 | 70 | 90 | 80 | 0 | 70 | 80 | 20 | 70 | 70 | 70 | 70 |
| Ragweed | 80 | 80 | 80 | 80 | 80 | 70 | 50 | 60 | 70 | 50 | 60 | 60 | 40 | 50 |
| Galium | 80 | 90 | 80 | 90 | 90 | 80 | 80 | 80 | 80 | 80 | 80 | 80 | 80 | 80 |
| Foxtail, Giant | — | — | — | — | — | — | — | — | — | — | — | — | — | — |
| Goosegrass | — | — | — | — | — | — | — | — | — | — | — | — | — | — |
| Foxtail, Green | 90 | 90 | 90 | 90 | — | — | — | — | — | — | — | — | — | — |
| Ryegrass, Italian | 90 | 100 | 100 | 90 | 100 | 70 | 70 | 70 | 70 | 70 | 70 | 60 | 80 | 70 |
| Kochia | 80 | 90 | 80 | 70 | 90 | 80 | 80 | 90 | 80 | 80 | 80 | 80 | 80 | 90 |
| Corn | 100 | 100 | 100 | 100 | 100 | 100 | 100 | 100 | 100 | 100 | 90 | 100 | 100 | 100 |
| Soybean | 90 | 80 | 80 | 80 | 100 | 90 | 90 | 90 | 90 | 70 | 50 | 90 | 80 | 50 |
| Amaranth, Palmer | 90 | 90 | 90 | 90 | 90 | 90 | 90 | 90 | 90 | 90 | 90 | 90 | 70 | 70 |
| Oat, Wild | 90 | 90 | 90 | 90 | 90 | 90 | 80 | 90 | 60 | 90 | 90 | 90 | 90 | 90 |
| Wheat | 90 | 90 | 90 | 90 | 90 | 90 | 90 | 90 | 90 | 90 | 80 | 90 | 90 | 90 |

| 125 g ai/ha | Compounds | | |
|---|---|---|---|
| Postemergence | 83 | 85 | 86 |
| Barnyardgrass | 90 | 90 | 90 |
| Blackgrass | 90 | 80 | 70 |
| Horseweed | 70 | 60 | 50 |
| Ragweed | 80 | 60 | 80 |
| Galium | 80 | 80 | 80 |
| Foxtail, Giant | — | — | — |
| Goosegrass | — | — | — |
| Foxtail, Green | — | — | — |
| Ryegrass, Italian | 70 | 70 | 70 |
| Kochia | 70 | 70 | 90 |
| Corn | 90 | 90 | 100 |
| Soybean | 60 | 60 | 60 |
| Amaranth, Palmer | 90 | 90 | 90 |
| Oat, Wild | 90 | 90 | 90 |
| Wheat | 90 | 90 | 80 |

| 62 g ai/ha | Compounds | | | | | | | | | | | | | |
|---|---|---|---|---|---|---|---|---|---|---|---|---|---|---|
| Postemergence | 89 | 90 | 91 | 92 | 93 | 94 | 95 | 96 | 97 | 98 | 99 | 100 | 101 | 102 |
| Barnyardgrass | 100 | 90 | 40 | 0 | 30 | 100 | 20 | 100 | 20 | 100 | 0 | 100 | 90 | 80 |
| Blackgrass | 90 | 80 | 10 | 0 | 40 | 90 | 20 | 90 | 0 | 90 | 0 | 90 | 90 | 90 |
| Corn | 100 | 100 | 30 | 0 | 0 | 100 | 0 | 100 | 0 | 100 | 0 | 100 | 90 | 60 |
| Foxtail, Green | 90 | 80 | 20 | 0 | 0 | 90 | 10 | 90 | 0 | 90 | 0 | 90 | 50 | 20 |
| Galium | 100 | — | 10 | 0 | 60 | 90 | 20 | 90 | 60 | 90 | 20 | 90 | 60 | 70 |
| Horseweed | 80 | 60 | 0 | 0 | 0 | 60 | 0 | 70 | 0 | 60 | 0 | 40 | 10 | 10 |
| Kochia | 90 | 80 | 40 | 0 | 50 | 90 | 20 | 80 | 70 | 90 | 0 | 90 | 60 | 70 |
| Oat, Wild | 90 | 50 | 20 | 0 | 70 | 90 | 0 | 80 | 0 | 100 | 0 | 100 | 90 | 60 |
| Pigweed, Palmer | 90 | 70 | 50 | 0 | 60 | 90 | 10 | 90 | 10 | 90 | 0 | 80 | 10 | 10 |
| Ragweed | 70 | 30 | 20 | 20 | 20 | 50 | 0 | 60 | 20 | 60 | 0 | 40 | 30 | 20 |
| Ryegrass, Italian | 90 | 70 | 40 | 0 | 40 | 90 | 0 | 90 | 0 | 90 | 0 | 90 | 70 | 70 |
| Soybean | 50 | 40 | 30 | 0 | 20 | 70 | 0 | 80 | 40 | 60 | 20 | 70 | 60 | 50 |
| Wheat | 90 | 70 | 50 | 0 | 0 | 90 | 10 | 90 | 10 | 80 | 10 | 80 | 80 | 20 |

| 62 g ai/ha | Compounds | | | | | | | | | | | | | |
|---|---|---|---|---|---|---|---|---|---|---|---|---|---|---|
| Postemergence | 103 | 104 | 105 | 106 | 107 | 108 | 109 | 110 | 111 | 112 | 113 | 114 | 115 | 116 |
| Barnyardgrass | 100 | 100 | 30 | 0 | 80 | 100 | 20 | 100 | 20 | 100 | 10 | 100 | 100 | 100 |
| Blackgrass | 40 | 70 | 10 | 0 | 60 | 90 | 30 | 100 | 0 | 80 | 20 | 80 | 90 | 90 |
| Corn | 80 | 90 | 10 | 0 | 100 | 100 | 0 | 100 | 0 | 100 | 20 | 100 | 100 | 100 |
| Foxtail, Green | 90 | 90 | 0 | 0 | 20 | 90 | 0 | 90 | 0 | 90 | 20 | 90 | 30 | 30 |
| Galium | 60 | 70 | — | — | — | — | — | — | — | — | — | — | 70 | 70 |
| Horseweed | 0 | 30 | 20 | 0 | 30 | 70 | 20 | 80 | — | 0 | — | 70 | 0 | 0 |
| Kochia | 80 | 90 | 0 | 0 | 10 | 90 | 0 | 90 | 0 | 90 | 0 | 90 | 70 | 60 |
| Oat, Wild | 20 | 30 | 0 | 0 | 20 | 80 | 0 | 30 | 0 | 90 | 0 | 40 | 90 | 90 |
| Pigweed, Palmer | 70 | 90 | 10 | 0 | 30 | 90 | 40 | 100 | 10 | 80 | 10 | 90 | 10 | 40 |
| Ragweed | 30 | 60 | 0 | 0 | 20 | 40 | 0 | 80 | 0 | 50 | 30 | 50 | 30 | 30 |
| Ryegrass, Italian | 30 | 70 | 0 | 0 | 60 | 80 | 0 | 70 | 0 | 90 | 0 | 80 | 70 | 70 |
| Soybean | 40 | 60 | 0 | 0 | 40 | 70 | 0 | 70 | 0 | 80 | 0 | 70 | 60 | 60 |
| Wheat | 70 | 90 | 0 | 0 | 50 | 90 | 0 | 90 | 20 | 90 | 0 | 90 | 80 | 90 |
| 62 g ai/ha | Compounds | | | | | | | | | | | | | |
| Postemergence | 117 | 118 | 119 | 120 | 121 | 122 | 124 | 125 | 126 | 127 | 128 | 129 | 130 | 131 |
| Barnyardgrass | 70 | 100 | 100 | 100 | 100 | 80 | 90 | 100 | 90 | 30 | 90 | 100 | 80 | 100 |
| Blackgrass | 90 | 100 | 100 | 90 | 100 | 90 | 90 | 90 | 90 | 70 | 90 | 40 | 90 | 90 |
| Corn | 100 | 100 | 100 | 100 | 100 | 100 | 100 | 100 | 100 | 0 | 100 | 100 | 100 | 100 |
| Foxtail, Green | 90 | 90 | 90 | 90 | 90 | 90 | 80 | 90 | 30 | 0 | 90 | 90 | 90 | 90 |
| Galium | 60 | 60 | 60 | 70 | 60 | 60 | 90 | 60 | 80 | 60 | 90 | 80 | 90 | 90 |
| Horseweed | 0 | 0 | 0 | 10 | 10 | 0 | 60 | 20 | 20 | 0 | 70 | 60 | 30 | 80 |
| Kochia | 0 | 70 | 80 | 80 | 70 | 80 | 90 | 80 | 30 | 0 | 80 | 80 | 90 | 50 |
| Oat, Wild | 80 | 90 | 90 | 80 | 90 | 90 | 90 | 90 | 90 | 20 | 90 | 20 | 90 | 90 |
| Pigweed, Palmer | 10 | 70 | 50 | 70 | 70 | 70 | 70 | 70 | 30 | 0 | 80 | 70 | 90 | 80 |
| Ragweed | 40 | 50 | 20 | 40 | 0 | 40 | 20 | 50 | 30 | 10 | 70 | 30 | 30 | 70 |
| Ryegrass, Italian | 80 | 80 | 60 | 70 | 70 | 70 | 90 | 70 | 60 | 0 | 90 | 60 | 90 | 90 |
| Soybean | 60 | 60 | 80 | 80 | 70 | 70 | 70 | 70 | 60 | 0 | 90 | 70 | 70 | 80 |
| Wheat | 90 | 90 | 90 | 90 | 90 | 90 | 90 | 90 | 90 | 0 | 90 | 80 | 90 | 90 |
| 62 g ai/ha | Compounds | | | | | | | | | | | | | |
| Postemergence | 132 | 133 | 134 | 135 | 136 | 137 | 138 | 139 | 140 | 141 | 142 | 143 | 144 | 145 |
| Barnyardgrass | 100 | 100 | 0 | 100 | 0 | 70 | 80 | 100 | 100 | 100 | 0 | 0 | 100 | 100 |
| Blackgrass | 80 | 90 | 0 | 100 | 0 | 60 | 90 | 100 | 100 | 90 | 0 | 0 | 100 | 100 |
| Corn | 90 | 100 | 0 | 100 | 0 | 50 | 100 | 100 | 100 | 100 | 0 | 0 | 100 | 100 |
| Foxtail, Green | 90 | 90 | 0 | 90 | 0 | 10 | 90 | 90 | 90 | 90 | 0 | 0 | 90 | 90 |
| Galium | 90 | 70 | 0 | 80 | 0 | — | 90 | 80 | 90 | 80 | 0 | 0 | — | — |
| Horseweed | 70 | 80 | 0 | 90 | 0 | 50 | 80 | 50 | 90 | 80 | 0 | 0 | 80 | 30 |
| Kochia | 80 | 90 | 0 | 90 | 0 | 50 | 80 | 90 | 90 | 90 | 0 | 0 | 80 | 90 |
| Oat, Wild | 90 | 70 | 0 | 90 | 0 | 30 | 90 | 90 | 90 | 80 | 0 | 0 | 40 | 90 |
| Pigweed, Palmer | 90 | 90 | 0 | 90 | 0 | 30 | 90 | 70 | 80 | 90 | 0 | 0 | 90 | 70 |
| Ragweed | 20 | 70 | 0 | 70 | 0 | 0 | 80 | 40 | 70 | 60 | 0 | 0 | 50 | 20 |
| Ryegrass, Italian | 90 | 90 | 0 | 90 | 0 | 30 | 90 | 70 | 80 | 80 | 0 | 0 | 90 | 90 |
| Soybean | 90 | 70 | 0 | 80 | 0 | 60 | 60 | 60 | 70 | 70 | 0 | 0 | 80 | 80 |
| Wheat | 90 | 90 | 0 | 90 | 0 | 70 | 90 | 90 | 90 | 90 | 0 | 0 | 90 | 90 |
| 62 g ai/ha | Compounds | | | | | | | | | | | | | |
| Postemergence | 146 | 147 | 148 | 149 | 150 | 151 | 152 | 153 | 154 | 155 | 156 | 157 | 158 | 159 |
| Barnyardgrass | 20 | 100 | 90 | 100 | 100 | 20 | 100 | 0 | 100 | 10 | 100 | 40 | 100 | 100 |
| Blackgrass | 0 | 90 | 70 | 90 | 90 | 0 | 100 | 90 | 0 | 0 | 90 | 30 | 100 | 90 |
| Corn | 0 | 100 | 70 | 100 | 100 | 0 | 100 | 0 | 100 | 0 | 100 | 0 | 100 | 100 |
| Foxtail, Green | 0 | 90 | 30 | 90 | 90 | 0 | 90 | 0 | 90 | 20 | 90 | 10 | 90 | 90 |
| Galium | — | — | — | — | 80 | — | — | — | — | 70 | 80 | 70 | 90 | — |
| Horseweed | 10 | 60 | 0 | 70 | 60 | 0 | 70 | 0 | 70 | 0 | 80 | 0 | 70 | 80 |
| Kochia | 0 | 90 | 80 | 90 | 70 | 0 | 90 | 0 | 90 | 40 | 80 | 60 | 90 | 80 |
| Oat, Wild | 0 | 70 | 10 | 90 | 90 | 0 | 90 | 0 | 90 | 0 | 70 | 0 | 90 | 30 |
| Pigweed, Palmer | 0 | 90 | 80 | 90 | 60 | 0 | 90 | 0 | 90 | 0 | 90 | 50 | 90 | 90 |
| Ragweed | 0 | 70 | 0 | 50 | 50 | 0 | 50 | 0 | 90 | 30 | 50 | 30 | 60 | 70 |
| Ryegrass, Italian | 0 | 80 | 50 | 80 | 80 | 0 | 80 | 0 | 80 | 0 | 70 | 60 | 80 | 100 |
| Soybean | 0 | 80 | 50 | 90 | 70 | 0 | 80 | 0 | 60 | 20 | 60 | 60 | 90 | 80 |
| Wheat | 0 | 90 | 30 | 90 | 90 | 0 | 90 | 0 | 90 | 0 | 70 | 30 | 80 | 90 |
| 62 g ai/ha | Compounds | | | | | | | | | | | | | |
| Postemergence | 160 | 161 | 162 | 163 | 164 | 165 | 166 | 167 | 168 | 169 | 170 | 171 | 172 | 173 |
| Barnyardgrass | 0 | 100 | 100 | 100 | 100 | 100 | 100 | 90 | 20 | 100 | 30 | 80 | 90 | 90 |
| Blackgrass | 0 | 90 | 100 | 70 | 90 | 80 | 90 | 90 | 0 | 100 | 0 | 70 | 90 | 90 |
| Corn | 0 | 100 | 100 | 100 | 100 | 100 | 100 | 100 | 10 | 100 | 0 | 90 | 100 | 100 |
| Foxtail, Green | 0 | 90 | 80 | 90 | 90 | 90 | 90 | 90 | 0 | 90 | 0 | 90 | 90 | 90 |
| Galium | 0 | 90 | 60 | 70 | 70 | 70 | 90 | 80 | 0 | 80 | 40 | 80 | 90 | 80 |
| Horseweed | 0 | 70 | — | — | — | — | 80 | 80 | 0 | — | — | — | 10 | 50 |
| Kochia | 0 | 30 | 70 | 90 | 90 | 90 | 80 | 80 | 0 | 80 | 0 | 80 | 90 | 90 |
| Oat, Wild | 0 | 90 | 90 | 90 | 90 | 90 | 90 | 90 | 0 | 90 | 0 | 90 | 90 | 90 |
| Pigweed, Palmer | 0 | 70 | 50 | 80 | 60 | 70 | 90 | 90 | 0 | 70 | 0 | 80 | 60 | 90 |
| Ragweed | 0 | 70 | 40 | 0 | 60 | 70 | 70 | 80 | 0 | 70 | 0 | 30 | 20 | 80 |
| Ryegrass, Italian | 0 | 80 | 70 | 80 | 80 | 80 | 90 | 90 | 0 | 90 | 0 | 70 | 90 | 90 |
| Soybean | 0 | 70 | 70 | 70 | 70 | 80 | 100 | 80 | 0 | 70 | 30 | 80 | 80 | 50 |
| Wheat | 0 | 90 | 90 | 70 | 90 | 90 | 90 | 90 | 0 | 90 | 0 | 90 | 90 | 90 |

| 62 g ai/ha | Compounds | | |
|---|---|---|---|
| Postemergence | 174 | 175 | 177 |
| Barnyardgrass | 20 | 70 | 80 |
| Blackgrass | 0 | 90 | 90 |
| Corn | 0 | 20 | 90 |
| Foxtail, Green | 0 | 90 | 90 |
| Galium | 0 | 90 | 80 |
| Horseweed | 0 | 0 | 10 |
| Kochia | 0 | 0 | 70 |
| Oat, Wild | 0 | 80 | 90 |
| Pigweed, Palmer | 0 | 10 | — |
| Ragweed | 0 | 0 | 20 |
| Ryegrass, Italian | 0 | 100 | 80 |
| Soybean | 0 | 60 | 50 |
| Wheat | 0 | 70 | 90 |

| 31 g ai/ha | Compounds | | | | | | | | | | | | | |
|---|---|---|---|---|---|---|---|---|---|---|---|---|---|---|
| Postemergence | 1 | 2 | 3 | 4 | 5 | 6 | 7 | 8 | 9 | 10 | 11 | 12 | 13 | 14 |
| Barnyardgrass | 100 | 50 | 100 | 100 | 100 | 100 | 100 | 0 | 70 | 60 | 60 | 10 | 10 | 0 |
| Blackgrass | 90 | 60 | 80 | 80 | 50 | 80 | 60 | 0 | 60 | 50 | 30 | 50 | 10 | 0 |
| Horseweed | 70 | — | 50 | 0 | 10 | 40 | 0 | 30 | 0 | 30 | 30 | 0 | 0 | 0 |
| Ragweed | 60 | 20 | 50 | 60 | 50 | 60 | 0 | 0 | 0 | 0 | 0 | 0 | 0 | 0 |
| Galium | 90 | 60 | 80 | 80 | 80 | 80 | 80 | — | — | — | — | — | 0 | 0 |
| Foxtail, Green | 90 | 0 | 90 | 90 | 30 | 90 | 50 | 0 | 30 | 20 | 30 | 20 | 0 | 0 |
| Ryegrass, Italian | 80 | 50 | 80 | 60 | 60 | 80 | 80 | 0 | 50 | 60 | 50 | 60 | 20 | 0 |
| Kochia | 70 | 0 | 70 | 70 | 30 | 80 | 70 | 0 | 50 | 20 | 20 | 20 | 0 | 0 |
| Corn | 100 | 40 | 100 | 90 | 60 | 100 | 90 | 0 | 20 | 40 | 10 | 20 | 0 | 0 |
| Soybean | 80 | 20 | 90 | 80 | 60 | 80 | 50 | 0 | 60 | 50 | 50 | 30 | 0 | 0 |
| Amaranth, Palmer | 90 | 0 | 50 | 50 | 40 | 90 | 50 | 0 | 10 | 10 | 30 | 0 | 10 | 0 |
| Oat, Wild | 90 | 70 | 90 | 90 | 90 | 90 | 90 | 0 | 90 | 90 | 80 | 80 | 10 | 0 |
| Wheat | 90 | 80 | 90 | 90 | 90 | 90 | 90 | 0 | 90 | 80 | 80 | 50 | 50 | 0 |

| 31 g ai/ha | Compounds | | | | | | | | | | | | | |
|---|---|---|---|---|---|---|---|---|---|---|---|---|---|---|
| Postemergence | 15 | 16 | 17 | 18 | 19 | 20 | 21 | 22 | 23 | 24 | 29 | 31 | 32 | 33 |
| Barnyardgrass | 100 | 100 | 100 | 70 | 60 | 0 | 100 | 70 | 100 | 20 | 10 | 10 | 0 | 10 |
| Blackgrass | 70 | 90 | 60 | 70 | 70 | 0 | 90 | 60 | 90 | 0 | 90 | 90 | 80 | 70 |
| Horseweed | 0 | 0 | 30 | 20 | 0 | 0 | 20 | 0 | 80 | 20 | 0 | 0 | 0 | 0 |
| Ragweed | 40 | 40 | 30 | 0 | 0 | 0 | 40 | 0 | 60 | 20 | 30 | 30 | 30 | 20 |
| Galium | 80 | 90 | 80 | 80 | 80 | 20 | 80 | 60 | 80 | 40 | 50 | 60 | 50 | 20 |
| Foxtail, Green | 90 | 90 | 80 | 40 | 40 | 20 | 90 | 20 | 90 | 0 | 0 | 0 | 0 | 10 |
| Ryegrass, Italian | 80 | 70 | 90 | 80 | 90 | 0 | 90 | 60 | 80 | 40 | 50 | 60 | 20 | 20 |
| Kochia | 70 | 70 | 60 | 10 | 10 | 0 | 80 | 60 | 80 | 20 | 20 | 0 | 40 | 20 |
| Corn | 100 | 90 | 70 | 50 | 50 | 40 | 100 | 50 | 100 | 10 | 0 | 0 | 0 | 10 |
| Soybean | 60 | 60 | 50 | 50 | 50 | 50 | 80 | 70 | 80 | 0 | 0 | 10 | 20 | 30 |
| Amaranth, Palmer | 50 | 20 | 70 | 10 | 30 | 0 | 60 | 20 | 80 | 0 | 0 | 0 | 0 | 0 |
| Oat, Wild | 90 | 60 | 90 | 70 | 90 | 20 | 90 | 40 | 90 | 10 | 80 | 80 | 80 | 60 |
| Wheat | 90 | 80 | 90 | 90 | 80 | 0 | 90 | 90 | 90 | 30 | 80 | 90 | 80 | 90 |

| 31 g ai/ha | Compounds | | | | | | | | | | | | | |
|---|---|---|---|---|---|---|---|---|---|---|---|---|---|---|
| Postemergence | 37 | 38 | 39 | 40 | 41 | 53 | 54 | 55 | 56 | 57 | 58 | 59 | 60 | 62 |
| Barnyardgrass | 100 | 90 | 10 | 10 | 0 | 100 | 70 | 90 | 90 | 100 | 90 | 90 | 90 | 100 |
| Blackgrass | 80 | 40 | 90 | 100 | 0 | 70 | 80 | 50 | 60 | 90 | 90 | 100 | 90 | 90 |
| Horseweed | 10 | 30 | 10 | 30 | 0 | 60 | 20 | 70 | 20 | 0 | 80 | 20 | 70 | 50 |
| Ragweed | 20 | 20 | 0 | 0 | 0 | 50 | 50 | 50 | 50 | 40 | 30 | 30 | 0 | 40 |
| Galium | — | — | — | — | — | 80 | 80 | 80 | 80 | 80 | 90 | 90 | 90 | 80 |
| Foxtail, Green | 90 | 90 | 20 | 0 | 20 | 50 | 40 | 20 | 30 | 30 | — | — | — | 90 |
| Ryegrass, Italian | 80 | 70 | 30 | 20 | 0 | 90 | 70 | 90 | 90 | 90 | 90 | 90 | 90 | 90 |
| Kochia | 80 | 80 | 20 | 0 | 0 | 80 | 70 | 80 | 70 | 80 | 80 | 80 | 80 | 70 |
| Corn | 100 | 90 | 0 | 20 | 0 | 100 | 90 | 100 | 90 | 90 | 100 | 90 | 90 | 100 |
| Soybean | 70 | 60 | 20 | 0 | 0 | 70 | 60 | 70 | 70 | 60 | 90 | 90 | 90 | 80 |
| Amaranth, Palmer | 70 | 40 | 0 | 0 | 0 | 70 | 90 | 80 | 90 | 20 | 90 | 90 | 90 | 90 |
| Oat, Wild | 90 | 90 | 90 | 90 | 10 | 90 | 90 | 90 | 90 | 90 | 30 | 60 | 10 | 90 |
| Wheat | 90 | 90 | 90 | 70 | 20 | 90 | 90 | 80 | 90 | 90 | 60 | 90 | 40 | 90 |

| 31 g ai/ha | Compounds | | | | | | | | | | | | | |
|---|---|---|---|---|---|---|---|---|---|---|---|---|---|---|
| Postemergence | 63 | 64 | 65 | 66 | 67 | 68 | 69 | 70 | 71 | 72 | 73 | 75 | 79 | 80 |
| Barnyardgrass | 100 | 100 | 100 | 100 | 100 | 90 | 90 | 90 | 90 | 90 | 90 | 80 | 90 | 80 |
| Blackgrass | 90 | 90 | 90 | 90 | 90 | 70 | 90 | 50 | 80 | 90 | 70 | 0 | 60 | 30 |
| Horseweed | 50 | 50 | 70 | 40 | 50 | 50 | 70 | 0 | 10 | 70 | 0 | 40 | 40 | 10 |
| Ragweed | 60 | 50 | 80 | 70 | 50 | 0 | 50 | 0 | 60 | 50 | 10 | 0 | 50 | 0 |
| Galium | 80 | 80 | 80 | 70 | 80 | 90 | 80 | 80 | 80 | 80 | 70 | 40 | 80 | 80 |
| Foxtail, Green | 90 | 90 | 90 | 70 | 90 | — | — | — | — | — | — | — | — | — |
| Ryegrass, Italian | 90 | 80 | 100 | 70 | 70 | 90 | 70 | 70 | 60 | 70 | 70 | 50 | 50 | 70 |
| Kochia | 70 | 80 | 80 | 70 | 70 | 80 | 80 | 80 | 80 | 80 | 50 | 80 | 70 | 70 |
| Corn | 100 | 90 | 100 | 90 | 100 | 80 | 100 | 90 | 100 | 100 | 90 | 70 | 90 | 30 |
| Soybean | 80 | 80 | 80 | 80 | 80 | 90 | 60 | 80 | 70 | 80 | 40 | 20 | 40 | 70 |
| Amaranth, Palmer | 90 | 90 | 90 | 90 | 80 | 90 | 90 | 90 | 90 | 90 | 50 | 70 | 70 | 50 |
| Oat, Wild | 90 | 90 | 90 | 90 | 90 | 30 | 90 | 30 | 40 | 40 | 80 | 10 | 90 | 90 |
| Wheat | 90 | 90 | 90 | 90 | 90 | 80 | 90 | 80 | 80 | 80 | 40 | 70 | 80 | 60 |

| | |
|---|---|
| 31 g ai/ha | Compounds |

| Postemergence | 81 | 83 | 85 | 86 |
|---|---|---|---|---|
| Barnyardgrass | 90 | 90 | 90 | 90 |
| Blackgrass | 0 | 60 | 60 | 30 |
| Horseweed | 30 | 20 | 60 | 40 |
| Ragweed | 20 | 60 | 30 | 60 |
| Galium | 80 | 80 | 50 | 70 |
| Foxtail, Green | — | — | — | — |
| Ryegrass, Italian | 50 | 60 | 60 | 60 |
| Kochia | 80 | 70 | 50 | 80 |
| Corn | 60 | 20 | 20 | 90 |
| Soybean | 30 | 20 | 40 | 20 |
| Amaranth, Palmer | 70 | 80 | 40 | 90 |
| Oat, Wild | 80 | 80 | 80 | 60 |
| Wheat | 90 | 90 | 80 | 60 |

| | |
|---|---|
| 31 g ai/ha | Compounds |

| Postemergence | 87 | 88 |
|---|---|---|
| Barnyardgrass | 100 | 100 |
| Blackgrass | 30 | 50 |
| Corn | 80 | 90 |
| Foxtail, Green | 40 | 50 |
| Galium | 70 | 70 |
| Horseweed | 30 | 50 |
| Kochia | 80 | 80 |
| Oat, Wild | 90 | 90 |
| Pigweed, Palmer | 70 | 90 |
| Ragweed | 50 | 60 |
| Ryegrass, Italian | 50 | 50 |
| Soybean | 50 | 50 |
| Wheat | 100 | 40 |

| 16 g ai/ha | Compounds | | | | | | | | | | | | | |
|---|---|---|---|---|---|---|---|---|---|---|---|---|---|---|
| Postemergence | 89 | 90 | 91 | 92 | 93 | 94 | 95 | 96 | 97 | 98 | 99 | 100 | 101 | 102 |
| Barnyardgrass | 90 | 30 | 0 | 0 | 0 | 100 | 0 | 100 | 0 | 100 | 0 | 50 | 60 | 30 |
| Blackgrass | 90 | 10 | 0 | 0 | 0 | 80 | 0 | 80 | 0 | 80 | 0 | 60 | 80 | 50 |
| Corn | 90 | 30 | 0 | 0 | 0 | 100 | 0 | 100 | 0 | 90 | 0 | 100 | 50 | 0 |
| Foxtail, Green | 60 | 0 | 0 | 0 | 0 | 60 | 0 | 90 | 0 | 80 | 0 | 80 | 10 | 10 |
| Galium | 100 | 40 | 0 | 0 | 10 | 70 | 0 | 70 | 20 | 80 | 20 | 70 | 50 | 50 |
| Horseweed | 20 | 0 | 10 | 0 | 0 | 20 | 0 | 40 | 0 | 20 | 0 | 0 | 10 | 20 |
| Kochia | 80 | 70 | 0 | 0 | 0 | 80 | 0 | 70 | 30 | 80 | 0 | 80 | 10 | 0 |
| Oat, Wild | 90 | 20 | 0 | 0 | 20 | 40 | 0 | 50 | 0 | 60 | 0 | 70 | 70 | 20 |
| Pigweed, Palmer | 70 | 0 | 0 | 0 | 0 | 50 | 0 | 70 | 0 | 70 | 0 | 20 | 0 | 0 |
| Ragweed | 50 | 0 | 0 | 0 | 0 | 50 | 0 | 40 | 0 | 50 | 0 | 0 | 0 | 0 |
| Ryegrass, Italian | 90 | 20 | 0 | 0 | 0 | 70 | 0 | 80 | 0 | 80 | 0 | 70 | 50 | 20 |
| Soybean | 50 | 30 | 0 | 0 | 0 | 60 | 0 | 80 | 0 | 50 | 0 | 60 | 40 | 30 |
| Wheat | 90 | 40 | 0 | 0 | 0 | 60 | 0 | 30 | 0 | 70 | 0 | 70 | 30 | 20 |

| 16 g ai/ha | Compounds | | | | | | | | | | | | | |
|---|---|---|---|---|---|---|---|---|---|---|---|---|---|---|
| Postemergence | 103 | 104 | 105 | 106 | 107 | 108 | 109 | 110 | 111 | 112 | 113 | 114 | 115 | 116 |
| Barnyardgrass | 50 | 50 | 0 | 0 | 0 | 90 | 0 | 90 | 0 | 100 | 0 | 100 | 70 | 70 |
| Blackgrass | 0 | 0 | 0 | 0 | 0 | 70 | 0 | 80 | 0 | 70 | 0 | 50 | 70 | 80 |
| Corn | 10 | 0 | 0 | 0 | 0 | 100 | 0 | 100 | 0 | 100 | 0 | 100 | 60 | 40 |
| Foxtail, Green | 0 | 40 | 0 | 0 | 0 | 60 | 0 | 90 | 0 | 90 | 0 | 80 | 10 | 10 |
| Galium | 10 | 30 | — | — | — | — | — | — | — | — | — | — | 60 | 60 |
| Horseweed | 0 | 0 | — | 0 | 0 | 30 | 0 | 70 | — | 0 | 0 | 20 | 10 | 0 |
| Kochia | 20 | 30 | 0 | 0 | 0 | 80 | 0 | 90 | 0 | 60 | 0 | 80 | 0 | 20 |
| Oat, Wild | 0 | 0 | 0 | 0 | 0 | 30 | 0 | 20 | 0 | 50 | 0 | 30 | 80 | 60 |
| Pigweed, Palmer | 60 | 80 | 0 | 0 | 0 | 70 | 0 | 90 | 0 | 30 | 0 | 90 | 10 | 0 |
| Ragweed | 10 | 40 | 0 | 0 | 0 | 20 | 0 | 50 | 0 | 50 | 0 | 10 | 0 | 0 |
| Ryegrass, Italian | 0 | 50 | 0 | 0 | 0 | 60 | 0 | 50 | 0 | 60 | 0 | 50 | 40 | 20 |
| Soybean | 10 | 20 | 0 | 0 | 0 | 60 | 0 | 30 | 0 | 60 | 0 | 50 | 40 | 50 |
| Wheat | 20 | 30 | 0 | 0 | 0 | 50 | 0 | 50 | 0 | 90 | 0 | 80 | 30 | 40 |
| 16 g ai/ha | Compounds | | | | | | | | | | | | | |
| Postemergence | 117 | 118 | 119 | 120 | 121 | 122 | 123 | 124 | 125 | 126 | 127 | 128 | 129 | 130 |
| Barnyardgrass | 0 | 50 | 70 | 50 | 60 | 40 | 60 | 70 | 60 | 50 | 0 | 90 | 80 | 70 |
| Blackgrass | 70 | 90 | 80 | 70 | 90 | 70 | 90 | 80 | 90 | 60 | 0 | 80 | 20 | 90 |
| Corn | 0 | 90 | 100 | 90 | 100 | 70 | 70 | 60 | 90 | 20 | 0 | 100 | 70 | 100 |
| Foxtail, Green | 30 | 90 | 70 | 60 | 70 | 60 | 30 | 10 | 80 | 0 | 0 | 90 | 20 | 90 |
| Galium | 50 | 50 | 20 | 20 | 30 | 30 | 30 | 70 | 30 | 70 | 20 | 90 | 70 | 70 |
| Horseweed | 0 | 0 | 0 | 10 | 0 | 0 | 0 | 0 | 0 | 0 | 0 | 30 | 0 | 10 |
| Kochia | 0 | 30 | 10 | 0 | 10 | 10 | 0 | 30 | 30 | 0 | 0 | 60 | 50 | 80 |
| Oat, Wild | 10 | 30 | 40 | 30 | 40 | 20 | 60 | 30 | 50 | 30 | 0 | 90 | 10 | 90 |
| Pigweed, Palmer | 0 | 20 | 20 | 10 | 0 | 20 | 20 | 50 | 10 | 10 | 0 | 60 | 50 | 80 |
| Ragweed | 0 | 30 | 0 | 20 | 0 | 0 | 0 | 0 | 20 | 0 | 10 | 10 | 0 | 20 |
| Ryegrass, Italian | 60 | 20 | 0 | 10 | 20 | 0 | 60 | 30 | 20 | 30 | 0 | 70 | 30 | 70 |
| Soybean | 40 | 60 | 50 | 50 | 50 | 20 | 60 | 40 | 50 | 0 | 0 | 70 | 20 | 60 |
| Wheat | 50 | 80 | 80 | 90 | 90 | 70 | 80 | 80 | 80 | 30 | 0 | 90 | 10 | 90 |
| 16 g ai/ha | Compounds | | | | | | | | | | | | | |
| Postemergence | 131 | 132 | 133 | 134 | 135 | 136 | 137 | 138 | 139 | 140 | 141 | 142 | 143 | 144 |
| Barnyardgrass | 90 | 90 | 100 | 0 | 100 | 0 | 0 | 70 | 90 | 100 | 100 | 0 | 0 | 90 |
| Blackgrass | 90 | 30 | 70 | 0 | 90 | 0 | 0 | 70 | 90 | 90 | 90 | 0 | 0 | 70 |
| Corn | 50 | 60 | 100 | 0 | 100 | 0 | 10 | 100 | 100 | 100 | 100 | 0 | 0 | 100 |
| Foxtail, Green | 30 | 60 | 90 | 0 | 90 | 0 | 0 | 80 | 90 | 90 | 90 | 0 | 0 | 90 |
| Galium | 70 | 40 | 60 | 0 | 70 | 0 | — | 90 | 50 | 70 | 80 | 0 | 0 | — |
| Horseweed | 40 | 10 | 0 | 0 | 70 | 0 | 0 | 50 | 0 | 80 | 60 | 0 | 0 | 50 |
| Kochia | 20 | 50 | 80 | 0 | 90 | 0 | 0 | 80 | 70 | 90 | 70 | 0 | 0 | 80 |
| Oat, Wild | 90 | 90 | 40 | 0 | 30 | 0 | 0 | 90 | 70 | 60 | 20 | 0 | 0 | 20 |
| Pigweed, Palmer | 20 | 60 | 90 | 0 | 70 | 0 | 0 | 90 | 20 | 60 | 70 | 0 | 0 | 70 |
| Ragweed | 0 | 0 | 30 | 0 | 40 | 0 | 0 | 60 | 40 | 50 | 30 | 0 | 0 | 20 |
| Ryegrass, Italian | 10 | 70 | 60 | 0 | 40 | 0 | 0 | 60 | 30 | 60 | 20 | 0 | 0 | 60 |
| Soybean | 80 | 40 | 60 | 0 | 60 | 0 | 0 | 60 | 60 | 70 | 70 | 0 | 0 | 80 |
| Wheat | 90 | 90 | 80 | 0 | 80 | 0 | 0 | 90 | 90 | 80 | 90 | 0 | 0 | 60 |
| 16 g ai/ha | Compounds | | | | | | | | | | | | | |
| Postemergence | 145 | 146 | 147 | 148 | 149 | 150 | 151 | 152 | 153 | 154 | 155 | 156 | 157 | 158 |
| Barnyardgrass | 100 | 0 | 100 | 0 | 100 | 90 | 0 | 100 | 0 | 100 | 0 | 100 | 0 | 90 |
| Blackgrass | 90 | 0 | 90 | 0 | 90 | 80 | 0 | 70 | 80 | 0 | 0 | 70 | 0 | 80 |
| Corn | 100 | 0 | 100 | 0 | 100 | 100 | 0 | 100 | 0 | 100 | 0 | 90 | 0 | 100 |
| Foxtail, Green | 90 | 0 | 90 | 0 | 90 | 20 | 0 | 90 | 0 | 80 | 0 | 80 | 0 | 90 |
| Galium | — | 0 | 70 | 40 | 60 | 70 | 0 | 70 | 0 | 50 | 0 | 80 | 30 | 90 |
| Horseweed | 30 | 0 | 60 | 0 | 50 | 20 | 0 | 30 | 20 | 70 | 0 | 30 | 0 | 30 |
| Kochia | 60 | 0 | 90 | 0 | 80 | 20 | 0 | 90 | 0 | 90 | 0 | 80 | 50 | 80 |
| Oat, Wild | 90 | 0 | 20 | 0 | 30 | 60 | 0 | 80 | 0 | 20 | 0 | 20 | 0 | 20 |
| Pigweed, Palmer | 10 | 0 | 90 | 10 | 90 | 40 | 0 | 60 | 0 | 90 | 0 | 90 | 0 | 90 |
| Ragweed | 0 | 0 | 0 | 0 | 20 | 0 | 0 | 20 | 0 | 40 | 0 | 30 | 0 | 50 |
| Ryegrass, Italian | 70 | 0 | 50 | 0 | 80 | 50 | 0 | 70 | 0 | 60 | 0 | 60 | 0 | 80 |
| Soybean | 50 | 0 | 50 | 0 | 60 | 60 | 0 | 60 | 0 | 60 | 0 | 50 | 40 | 60 |
| Wheat | 90 | 0 | 40 | 10 | 80 | 70 | 0 | 80 | 0 | 70 | 0 | 50 | 0 | 70 |
| 16 g ai/ha | Compounds | | | | | | | | | | | | | |
| Postemergence | 159 | 160 | 161 | 162 | 163 | 164 | 165 | 166 | 167 | 168 | 169 | 170 | 171 | 172 |
| Barnyardgrass | 100 | 0 | 80 | 50 | 70 | 100 | 100 | 90 | 90 | 10 | 100 | 0 | 70 | 80 |
| Blackgrass | 80 | 0 | 90 | 90 | 20 | 90 | 80 | 70 | 80 | 0 | 100 | 0 | 70 | 90 |
| Corn | 100 | 0 | 100 | 100 | 90 | 90 | 100 | 90 | 100 | 0 | 100 | 0 | 80 | 100 |
| Foxtail, Green | 70 | 0 | 60 | 30 | 50 | 90 | 60 | 30 | 90 | 0 | 90 | 0 | 80 | 80 |
| Galium | — | — | 80 | 30 | 40 | 70 | 60 | 70 | 80 | 0 | 70 | 0 | 80 | 90 |
| Horseweed | 50 | 0 | 60 | — | — | — | — | 50 | 80 | 0 | — | — | — | 0 |
| Kochia | 70 | 0 | 0 | 0 | 20 | 40 | 70 | 80 | 80 | 0 | 80 | 0 | 60 | 90 |
| Oat, Wild | 30 | 0 | 90 | 80 | 30 | 80 | 90 | 90 | 90 | 0 | 90 | 0 | 90 | 90 |
| Pigweed, Palmer | 80 | 0 | 40 | 10 | 10 | 40 | 20 | 90 | 90 | 0 | 20 | 0 | 60 | 10 |
| Ragweed | 40 | 0 | 50 | 20 | 0 | 50 | 60 | 40 | 70 | 0 | 0 | 0 | 30 | 10 |
| Ryegrass, Italian | 60 | 0 | 70 | 30 | 60 | 70 | 70 | 60 | 80 | 0 | 80 | 0 | 20 | 80 |
| Soybean | 80 | 0 | 60 | 60 | 50 | 60 | 70 | 80 | 80 | 0 | 60 | 0 | 70 | 80 |
| Wheat | 30 | 0 | 90 | 90 | 30 | 80 | 90 | 90 | 90 | 0 | 90 | 0 | 80 | 90 |

| | |
|---|---|
| 16 g ai/ha | Compounds |

| Postemergence | 173 | 174 | 175 | 177 |
|---|---|---|---|---|
| Barnyardgrass | 80 | 10 | 0 | 70 |
| Blackgrass | 90 | 0 | 60 | 80 |
| Corn | 100 | 0 | 0 | 30 |
| Foxtail, Green | 90 | 0 | 30 | 30 |
| Galium | 80 | 0 | 90 | 90 |
| Horseweed | 0 | 0 | 0 | 0 |
| Kochia | 90 | 0 | 0 | 30 |
| Oat, Wild | 90 | 0 | 60 | 70 |
| Pigweed, Palmer | 90 | 0 | 0 | — |
| Ragweed | 0 | 0 | 0 | 0 |
| Ryegrass, Italian | 80 | 0 | 70 | 70 |
| Soybean | 50 | 0 | 0 | 20 |
| Wheat | 90 | 0 | 30 | 80 |

| | |
|---|---|
| 8 g ai/ha | Compounds |

| Postemergence | 87 | 88 |
|---|---|---|
| Barnyardgrass | 50 | 10 |
| Blackgrass | 0 | 0 |
| Corn | 0 | 40 |
| Foxtail, Green | 0 | 0 |
| Galium | 50 | 30 |
| Horseweed | 20 | 0 |
| Kochia | 30 | 30 |
| Oat, Wild | 20 | 20 |
| Pigweed, Palmer | 30 | 40 |
| Ragweed | 10 | 50 |
| Ryegrass, Italian | 20 | 0 |
| Soybean | 20 | 0 |
| Wheat | 0 | 20 |

### Test B

Plant species in the flooded paddy test selected from barnyardgrass (Echinochloa crusgalli), ducksalad (Heteranthera limosa), rice (Oryza sativa), and sedge, umbrella (small-flower umbrella sedge, Cyperus difformis) were grown to the 2-leaf stage for testing. At time of treatment, test pots were flooded to 3 cm above the soil surface, treated by application of test compounds directly to the paddy water, and then maintained at that water depth for the duration of the test. Treated plants and controls were maintained in a greenhouse for 13 days, after which time all species were compared to controls and visually evaluated. Plant response ratings, summarized in Table B, are based on a scale of 0 to 100 where 0 is no effect and 100 is complete control. A dash (-) response means no test result.

TABLE B

| 250 g ai/ha | Compounds | | | | | | | | | | | | | |
|---|---|---|---|---|---|---|---|---|---|---|---|---|---|---|
| Flood | 1 | 2 | 3 | 4 | 5 | 6 | 7 | 8 | 9 | 10 | 11 | 12 | 13 | 14 |
| Barnyardgrass | 95 | 95 | 95 | 90 | 65 | 70 | 60 | 0 | 0 | 50 | 50 | 0 | 0 | 0 |
| Ducksalad | 60 | 65 | 90 | 70 | 80 | 95 | 60 | 0 | 0 | 100 | 65 | 0 | 0 | 0 |
| Rice | 80 | 20 | 70 | 20 | 20 | 35 | 25 | 0 | 0 | 10 | 25 | 0 | 0 | 0 |
| Sedge, Umbrella | 95 | 75 | 100 | 95 | 95 | 95 | 90 | 0 | 0 | 100 | 60 | 0 | 0 | 0 |

| 250 g ai/ha | Compounds | | | | | | | | | | | | | |
|---|---|---|---|---|---|---|---|---|---|---|---|---|---|---|
| Flood | 15 | 16 | 17 | 18 | 19 | 20 | 21 | 22 | 23 | 24 | 26 | 27 | 28 | 29 |
| Barnyardgrass | 60 | 65 | 70 | 65 | 65 | 0 | 90 | 0 | 95 | 0 | 45 | 90 | 85 | 0 |
| Ducksalad | 80 | 70 | 70 | 50 | 70 | 0 | 90 | 0 | 70 | 0 | 95 | 85 | 75 | 0 |
| Rice | 65 | 65 | 10 | 65 | 0 | 0 | 90 | 0 | 85 | 0 | 35 | 35 | 70 | 0 |
| Sedge, Umbrella | 70 | 65 | 60 | 65 | 60 | 0 | 95 | 0 | 86 | 0 | 95 | 50 | 65 | 0 |

| 250 g ai/ha | Compounds | | | | | | | | | | | | | |
|---|---|---|---|---|---|---|---|---|---|---|---|---|---|---|
| Flood | 30 | 31 | 32 | 33 | 34 | 35 | 36 | 37 | 38 | 39 | 42 | 43 | 44 | 45 |
| Barnyardgrass | 15 | 0 | 0 | 20 | 90 | 95 | 60 | 75 | 55 | 0 | 95 | 85 | 80 | 75 |
| Ducksalad | 65 | 0 | 0 | 0 | 75 | 90 | 90 | 95 | 55 | 0 | 90 | 90 | 90 | 100 |
| Rice | 0 | 0 | 0 | 0 | 10 | 50 | 55 | 65 | 70 | 0 | 15 | 20 | 15 | 15 |
| Sedge, Umbrella | 95 | 0 | 0 | 40 | 75 | 90 | 95 | 100 | 95 | 0 | 95 | 95 | 95 | 100 |

| 250 g ai/ha | Compounds | | | | | | | | | | | | | |
|---|---|---|---|---|---|---|---|---|---|---|---|---|---|---|
| Flood | 46 | 47 | 48 | 49 | 50 | 51 | 52 | 53 | 54 | 55 | 56 | 57 | 58 | 59 |
| Barnyardgrass | 70 | 0 | 70 | 80 | 60 | 75 | 75 | 90 | 90 | 90 | 95 | 90 | 60 | 90 |
| Ducksalad | 90 | 30 | 70 | 90 | 70 | 60 | 60 | 90 | 95 | 85 | 90 | 100 | 95 | 85 |
| Rice | 10 | 0 | 65 | 55 | 75 | 60 | 65 | 20 | 15 | 15 | 15 | 20 | 70 | 50 |
| Sedge, Umbrella | 95 | 0 | 80 | 95 | 60 | 60 | 90 | 60 | 95 | 45 | 50 | 55 | 98 | 98 |

| 250 g ai/ha | Compounds | | | | | | | | | | | | |
|---|---|---|---|---|---|---|---|---|---|---|---|---|---|
| Flood | 60 | 62 | 63 | 64 | 65 | 66 | 67 | 68 | 69 | 70 | 71 | 72 | 73 |
| Barnyardgrass | 70 | 50 | 80 | 50 | 75 | 75 | 65 | 85 | 85 | 50 | 80 | 55 | 30 |
| Ducksalad | 95 | 90 | 95 | 90 | 95 | 95 | 98 | 95 | 60 | 65 | 70 | 75 | 40 |
| Rice | 65 | 15 | 60 | 15 | 60 | 15 | 30 | 40 | 50 | 60 | 85 | 85 | 15 |
| Sedge, Umbrella | 100 | 98 | 98 | 75 | 100 | 100 | 100 | 98 | 95 | 98 | 100 | 98 | 75 |

| 250 g ai/ha | Compounds | | | | | | | | | | | | | | | |
|---|---|---|---|---|---|---|---|---|---|---|---|---|---|---|---|---|
| Flood | 87 | 88 | 89 | 90 | 91 | 92 | 93 | 94 | 95 | 96 | 97 | 98 | 99 | 100 | 101 | 102 |
| Barnyardgrass | 0 | 15 | 95 | 0 | 0 | 0 | 0 | 95 | 0 | 95 | 0 | 65 | 0 | 70 | 0 | 0 |
| Ducksalad | 0 | 70 | 70 | 0 | 0 | 0 | 0 | 95 | 0 | 70 | 0 | 75 | 0 | 75 | 0 | 0 |
| Rice | 0 | 25 | 25 | 0 | 0 | 0 | 0 | 75 | 0 | 25 | 0 | 70 | 0 | 70 | 0 | 0 |
| Sedge, Umbrella | 0 | 80 | 95 | 0 | 0 | 0 | 0 | 95 | 0 | 95 | 0 | 98 | 0 | 95 | 0 | 0 |

| 250 g ai/ha | Compounds | | | | | | | | | | | | | |
|---|---|---|---|---|---|---|---|---|---|---|---|---|---|---|
| Flood | 103 | 104 | 105 | 106 | 107 | 108 | 109 | 110 | 111 | 112 | 113 | 114 | 115 | 116 |
| Barnyardgrass | 0 | 0 | 0 | 65 | 0 | 50 | 0 | 60 | 0 | 80 | 0 | 90 | 65 | 75 |
| Ducksalad | 0 | 0 | 0 | 85 | 0 | 70 | 0 | 95 | 0 | 95 | 0 | 80 | 80 | 45 |
| Rice | 0 | 0 | 0 | 10 | 0 | 85 | 0 | 40 | 0 | 85 | 0 | 55 | 25 | 50 |
| Sedge, Umbrella | 0 | 0 | 0 | 98 | 0 | 70 | 0 | 98 | 0 | 95 | 0 | 98 | 0 | 55 |
| 250 g ai/ha | Compounds | | | | | | | | | | | | | |
| Flood | 117 | 118 | 119 | 120 | 121 | 122 | 123 | 124 | 125 | 126 | 127 | 128 | 129 | 130 |
| Barnyardgrass | 0 | 0 | 0 | 0 | 0 | 0 | 0 | 10 | 0 | 25 | 0 | 65 | 50 | 80 |
| Ducksalad | 0 | 0 | 0 | 0 | 0 | 0 | 55 | 85 | 0 | 55 | 0 | 80 | 70 | 100 |
| Rice | 0 | 0 | 0 | 0 | 0 | 0 | 10 | 15 | 0 | 5 | 0 | 85 | 40 | 60 |
| Sedge, Umbrella | 0 | 0 | 0 | 0 | 0 | 0 | 0 | 30 | 0 | 35 | 0 | 100 | 90 | 90 |
| 250 g ai/ha | Compounds | | | | | | | | | | | | | |
| Flood | 131 | 132 | 133 | 134 | 135 | 136 | 137 | 138 | 139 | 140 | 141 | 142 | 143 | 144 |
| Barnyardgrass | 95 | 90 | 95 | 0 | 90 | 0 | 0 | 95 | 0 | 85 | 90 | 0 | 40 | 95 |
| Ducksalad | 80 | 80 | 85 | 0 | 65 | 0 | 0 | 100 | 0 | 65 | 65 | 0 | 60 | 80 |
| Rice | 70 | 90 | 85 | 0 | 65 | 0 | 0 | 95 | 0 | 65 | 50 | 0 | 10 | 50 |
| Sedge, Umbrella | 95 | 95 | 98 | 0 | 95 | 0 | 0 | 95 | 0 | 100 | 85 | 0 | 45 | 100 |
| 250 g ai/ha | Compounds | | | | | | | | | | | | | |
| Flood | 145 | 146 | 147 | 148 | 149 | 150 | 151 | 152 | 153 | 154 | 155 | 156 | 157 | 158 |
| Barnyardgrass | 60 | 0 | 95 | 0 | 95 | 65 | 0 | 90 | 0 | 98 | 0 | 55 | 0 | 90 |
| Ducksalad | 60 | 0 | 70 | 0 | 95 | 85 | 0 | 80 | 0 | 70 | 0 | 75 | 0 | 70 |
| Rice | 40 | 0 | 70 | 0 | 90 | 30 | 0 | 85 | 0 | 95 | 0 | 45 | 0 | 50 |
| Sedge, Umbrella | 50 | 0 | 95 | 0 | 95 | 75 | 0 | 90 | 0 | 95 | 0 | 90 | 0 | 98 |
| 250 g ai/ha | Compounds | | | | | | | | | | | | | |
| Flood | 159 | 160 | 161 | 162 | 163 | 164 | 165 | 166 | 167 | 168 | 169 | 170 | 171 | 172 |
| Barnyardgrass | 95 | 0 | 60 | 20 | 55 | 35 | 95 | 95 | 90 | 0 | 90 | 0 | 60 | 95 |
| Ducksalad | 100 | 55 | 80 | 65 | 85 | 30 | 100 | 85 | 98 | 0 | 100 | 0 | 50 | 80 |
| Rice | 55 | 0 | 85 | 0 | 60 | 5 | 15 | 80 | 98 | 0 | 95 | 0 | 45 | 70 |
| Sedge, Umbrella | 100 | 25 | 95 | 0 | 85 | 80 | 100 | 95 | 98 | 0 | 100 | 0 | 45 | 100 |

| | |
|---|---|
| 250 g ai/ha | Compounds |

| Flood | 173 | 174 | 175 | 176 | 177 | 181 | 182 | 196 | 198 |
|---|---|---|---|---|---|---|---|---|---|
| Barnyardgrass | 90 | 0 | 0 | 55 | 90 | 10 | 20 | 95 | 90 |
| Ducksalad | 95 | 0 | 0 | 65 | 75 | 30 | 60 | 50 | 60 |
| Rice | 75 | 0 | 0 | 35 | 90 | 15 | 15 | 85 | 85 |
| Sedge, Umbrella | 100 | 0 | 0 | 45 | 80 | 0 | 70 | 95 | 75 |

What is claimed is:

1. A compound of Formula 1, all stereoisomers, N-oxides, and salts thereof 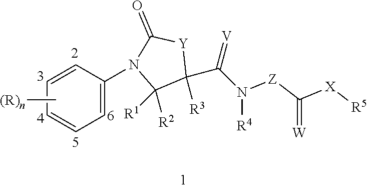
wherein
Y is O or NH;
Z is selected from a group of a fully saturated, or a fully or partly unsaturated, five membered rings as shown below, optionally substituted by group R^{v} 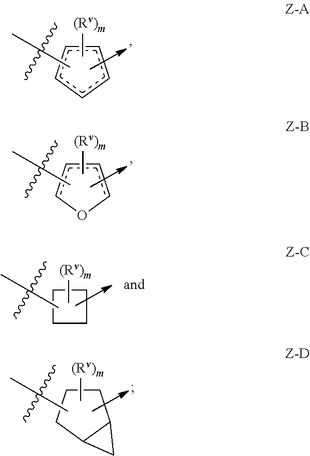
m is 0, 1 or 2;
R^{v} is halogen, cyano, CO₂R⁸, or (C₁-C₂)-alkyl or (C₁-C₂)-alkoxy, each of which is substituted by n radicals independently selected from the group consisting of halogens;
each R is independently H, halogen, cyano, nitro, hydroxy, C₁-C₆ alkyl, C₁-C₆ haloalkyl, C₂-C₆ alkenyl, C₂-C₆ haloalkenyl, C₂-C₆ alkynyl, C₂-C₆ haloalkynyl, C₃-C₇ cycloalkyl, C₃-C₇ halocycloalkyl, C₁-C₆ alkoxy, C₁-C₆ haloalkoxy, S(O)ₚR⁷ or CO₂R⁸;
n is 0, 1, 2, 3, 4 or 5;
p is 0, 1 or 2;
V and W are each independently O or S;
X is a direct bond, O, S or NR⁶;
R¹ and R² are each independently hydrogen, halogen, cyano, C₁-C₆ alkyl, C₁-C₆ haloalkyl, C₁-C₆ cyanoalkyl, C₁-C₆ alkoxy, C₁-C₆ haloalkoxy or C₁-C₆ cyanoalkoxy;
R³ is H, halogen, cyano, nitro, hydroxy, C₁-C₆ alkyl, C₂-C₅ alkenyl, C₂-C₅ alkynyl, C₂-C₅ alkenyloxy, C₂-C₅ alkynyloxy, C₃-C₇ cycloalkoxy, C₃-C₇ cycloalkoxyalkyl, C₃-C₆ cycloalkyl, C₄-C₇ cycloalkylalkyl, C₁-C₆ haloalkyl, C₂-C₅ haloalkenyl, C₂-C₅ haloalkynyl, C₂-C₅ alkoxyalkyl, C₂-C₅ haloalkoxyalkyl, C₁-C₅ alkoxy, C₁-C₆ haloalkoxy, C₁-C₅ alkylthio, C₁-C₄ alkylsulfinyl, C₁-C₄ alkylsulfonyl, C₁-C₄ alkylsulfonate, C₁-C₄ haloalkylthio, C₁-C₄ haloalkylsulfinyl, C₁-C₄ haloalkylsulfonyl or C₂-C₅ alkoxycarbonyl, optionally each of which is further substituted by at least one radical from the group consisting of halogen, cyano, C₁-C₄ alkoxy, C₁-C₅ alkylthio, C₁-C₄ alkylsulfinyl, C₁-C₄ alkylsulfonyl, and hydroxy;
R⁴ is independently H, C₁-C₆ alkyl, C₃-C₇ cycloalkyl, C₁-C₆ haloalkyl, C₁-C₃ alkoxy, C₁-C₃ haloalkoxy; or W¹G¹;
W¹ is a direct bond, C₁-C₄ alkanediyl or C₁-C₄ alkenediyl;
G¹ is S(O)ₚR⁷, SO₂NR¹⁰R¹¹, CO₂R⁸, CONR¹⁰R¹¹ or COR¹²;
R⁵ is H, C₁-C₁₂ alkyl, NH₂, N═CR⁸R¹², C₃-C₇ cycloalkyl, C₃-C₁₂ cycloalkylalkyl, C₂-C₈ alkenyl, C₅-C₆ cycloalkenyl, C₂-C₈ alkynyl, (CH₂)_{q}S(O)ₚR⁷, (CH₂)_{q}OR⁹, (CH₂)_{q}COR¹², each of which is optionally substituted by one or more radicals from the group consisting of halogen, cyano, C₁-C₆ alkyl, C₁-C₆ alkoxy, hydroxy and aryl;
q is 0, 1, 2, 3, 4 or 5;
R⁶ is hydrogen, cyano, OR⁹, S(O)ₚR⁷, SO₂NR¹⁰R¹¹, CO₂R⁸, CONR¹⁰R¹¹, COR¹², NR¹⁰R¹¹, NR¹⁰COR¹², NR¹⁰CONR¹⁰R¹¹, NR¹⁰CO₂R⁸, NR¹⁰SO₂R⁷, NR¹⁰SO₂NR¹⁰R¹¹, C(R⁷)═NOR⁹, optionally substituted aryl, optionally substituted heteroaryl or optionally substituted heterocyclyl; or
R⁶ is C₁-C₁₂ alkyl, C₃-C₈ cycloalkyl, C₄-C₁₂ cycloalkylalkyl, C₂-C₁₂ alkenyl, C₅-C₇ cycloalkenyl or C₂-C₁₂ alkynyl, each of which is optionally substituted by one or more radicals from the group consisting of halogen, cyano, nitro, OR⁹, S(O)ₚR⁷, SO₂NR¹⁰R¹¹, CO₂R⁸, CONR¹⁰R¹¹, COR¹², NR¹⁰R¹¹, NR¹⁰COR¹², NR¹⁰CONR¹⁰R¹¹, NR¹⁰CO₂R⁸, NR¹⁰SO₂R⁷, NR¹⁰SO₂NR¹⁰R¹¹, C(R⁷)═NOR⁹, optionally substituted aryl, optionally substituted heteroaryl or optionally substituted heterocyclyl; or
R⁵ and R⁶ can be taken together with the nitrogen atom to which they are attached to form a 3- to 7-membered ring, containing carbon atoms and optionally 1 to 3 oxygen, sulfur or nitrogen atoms as ring members, wherein up to 2 carbon atom ring members are independently selected from C(═O) and C(═S), and the sulfur atom ring member is selected from S, S(O) or S(O)₂, said ring being optionally substituted with at least one substituent independently selected from the group consisting of halogen, cyano, nitro, C₁-C₆ alkyl, C₁-C₆ haloalkyl, OR⁹, S(O)ₚR⁷, SO₂NR¹⁰R¹¹, CO₂R⁸, CONR¹⁰R¹¹, COR¹², NR¹⁰R¹¹, NR¹⁰COR¹², NR¹⁰CONR¹⁰R¹¹, NR¹⁰CO₂R⁸, NR¹⁰SO₂R⁷, NR¹⁰SO₂NR¹⁰R¹¹, C(R⁷)═NOR⁹;
R⁷, R⁸, R⁹, R¹⁰, R¹¹ and R¹² are each independently H, C₁-C₆ alkyl, C₁-C₆ alkoxy, C₁-C₆ haloalkyl, C₃-C₇ cycloalkyl or aryl, each of which is optionally substituted by one or more radicals from the group consisting of F, Cl, C₁-C₂ alkoxy or aryl.

2. The compound of claim 1 wherein
Y is O;
Z-A is selected from Z-1 through Z-29, Z-B is selected from Z-30 through Z-62, Z-C is selected from Z-63 through 64, and Z-D is selected from Z-65 through Z-74; 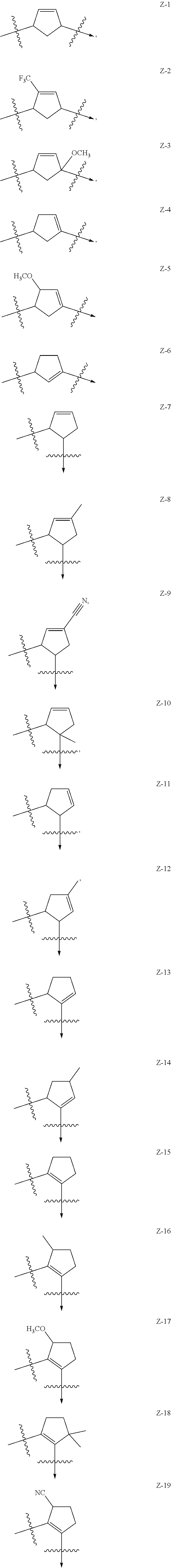 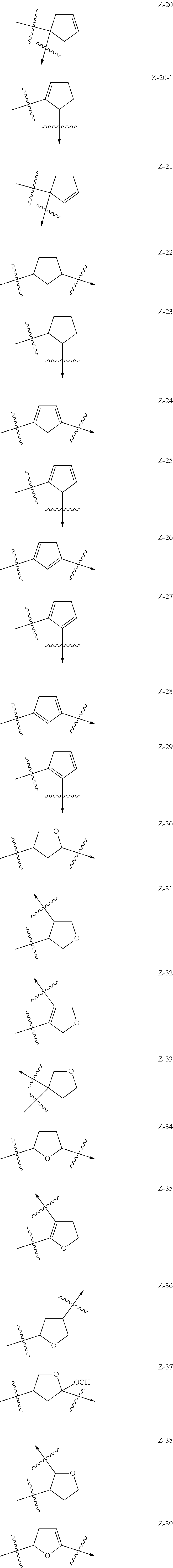 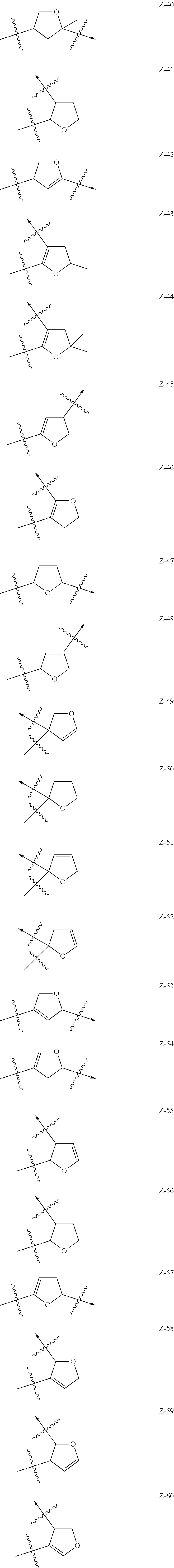 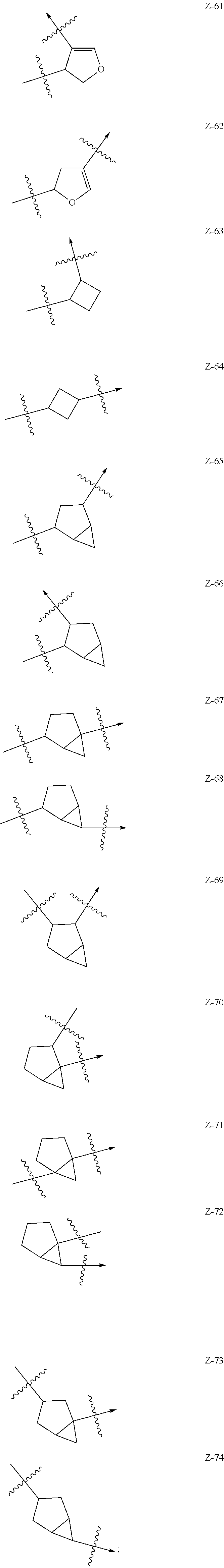
m is 0 or 1;
R is independently H, halogen, cyano, C₁-C₆ alkyl, C₁-C₆ haloalkyl, C₁-C₆ alkoxy or C₁-C₆ haloalkoxy;
n is 1, 2 or 3;
R¹ and R² are each independently hydrogen, halogen, cyano, C₁-C₃ alkyl, C₁-C₃ haloalkyl, C₁-C₃ cyanoalkyl, C₁-C₃ alkoxy, C₁-C₃ haloalkoxy or C₁-C₃ cyanoalkoxy; and
R³ is H, halogen, cyano, hydroxy, C₁-C₃ alkyl, C₂-C₃ alkenyl, C₂-C₃ alkynyl, C₃-C₅ cycloalkoxy, C₃-C₅ cycloalkoxyalkyl, C₃-C₅ cycloalkyl, C₄-C₇ cycloalkylalkyl, C₁-C₃ haloalkyl, C₂-C₃ alkoxyalkyl, C₂-C₃ haloalkoxyalkyl, C₁-C₃ alkoxy, C₁-C₃ haloalkoxy, C₁-C₃ alkylthio, C₁-C₃ alkylsulfinyl or C₁-C₃ alkylsulfonyl.

3. The compound of claim 2 wherein
Z is selected from the group Z-A;
R is independently H, F, Cl, Br, cyano, C₁-C₂ alkyl, C₁-C₂ haloalkyl, C₁-C₂ alkoxy or C₁-C₂ haloalkoxy;
V and W are both O;
X is O or S;
R³ is H, C₁-C₃ alkyl, C₂-C₃ alkenyl, C₂-C₃ alkynyl, C₃-C₅ cycloalkyl, C₄-C₇ cycloalkylalkyl, C₁-C₃ haloalkyl, C₂-C₃ alkoxyalkyl, C₂-C₃ haloalkoxyalkyl, C₁-C₃ alkoxy, C₁-C₃ haloalkoxy;
R⁴ is H, C₁-C₆ alkyl, C₃-C₇ cycloalkyl, C₁-C₆ haloalkyl, C₁-C₃ alkoxy, C₁-C₃ haloalkoxy; and
R⁵ is H, C₁-C₁₂ alkyl, NH₂, N═CR⁸R¹², C₃-C₇ cycloalkyl, C₃-C₁₂ cycloalkylalkyl, C₂-C₈ alkenyl, C₅-C₆ cycloalkenyl, C₂-C₈ alkynyl, (CH₂)_{q}S(O)ₚR⁷, (CH₂)_{q}OR⁹, (CH₂)_{q}COR¹², each of which is optionally substituted by one or more radicals from the group consisting of halogen, cyano, C₁-C₆ alkyl, C₁-C₆ alkoxy, hydroxy and aryl.

4. The compound of claim 3 wherein
Z is Z-1, Z-4, Z-6, Z-12, Z-22 or Z-24;
R is independently H, F, Cl, Me, CF₃, OMe or OCF₃;
n is 2;
R is at 3- and 5-position;
R¹ and R² are each independently hydrogen, halogen, cyano, Me, CF₃, CH₂CN, OMe, OCF₃ or OCH₂CN;
R³ is H, Me, Et, CH═CH₂, C≡CH, cyclopropyl, cyclopropylmethy, CF₃, OMe or OCF₃.
R⁵ is H, Me, Et, c-Pr, c-Bu, CH₂-c-Pr, (CH₂)_{q}S(O)ₚMe, (CH₂)_{q}OMe, (CH₂)_{q}COMe, each of which is optionally substituted by one or more radicals from the group consisting of F, Cl, cyano, C₁-C₃ alkyl, C₁-C₃ alkoxy, hydroxy and Ph; and
R⁷, R⁸, R⁹, R¹⁰, R¹¹ and R¹² are each independently H, C₁-C₃ alkyl, C₁-C₃ alkoxy, C₁-C₃ haloalkyl, C₃-C₅ cycloalkyl or phenyl, each of which is optionally substituted by one or more radicals from the group consisting of F, Cl, C₁-C₂ alkoxy or aryl.

5. The compound of claim 2 wherein
Z is selected from the group Z-A;
R is independently H, F, Cl, Br, cyano, C₁-C₂ alkyl, C₁-C₂ haloalkyl, C₁-C₂ alkoxy or C₁-C₂ haloalkoxy;
V and W are both O;
X is NR⁶;
R³ is H, C₁-C₃ alkyl, C₂-C₃ alkenyl, C₂-C₃ alkynyl, C₃-C₅ cycloalkyl, C₄-C₇ cycloalkylalkyl, C₁-C₃ haloalkyl, C₂-C₃ alkoxyalkyl, C₂-C₃ haloalkoxyalkyl, C₁-C₃ alkoxy, C₁-C₃ haloalkoxy;
R⁴ is H, C₁-C₆ alkyl, C₃-C₇ cycloalkyl, C₁-C₆ haloalkyl, C₁-C₃ alkoxy, C₁-C₃ haloalkoxy.

6. The compound of claim 5 wherein
Z is Z-1, Z-4, Z-6, Z-12, Z-22 or Z-24;
R is independently H, F, Cl, Me, CF₃, OMe or OCF₃;
n is 2;
R is at 3- and 5 position;
R¹ and R² are each independently hydrogen, halogen, cyano, Me, CF₃, CH₂CN, OMe, OCF₃ or OCH₂CN;
R³ is H, Me, Et, CH═CH₂, C≡CH, cyclopropyl, cyclopropylmethy, CF₃, OMe or OCF₃; and
R⁷, R⁸, R⁹, R¹⁰, R¹¹ and R¹² are each independently H, C₁-C₃ alkyl, C₁-C₃ haloalkyl, C₃-C₅ cycloalkyl or phenyl, each of which is optionally substituted by one or more radicals from the group consisting of F, Cl, C₁-C₂ alkoxy or aryl.

7. The compound of claim 5 wherein
R⁵ and R⁶ can be taken together with the nitrogen atom to which they are attached to form a 3- to 7-membered ring, wherein the 3- to 7-membered ring contains carbon atoms and optionally 1 to 2 oxygen, sulfur or nitrogen atoms as ring members, wherein up to 2 carbon atom ring members are independently selected from C(═O) and C(═S), and the sulfur atom ring member is selected from S, S(O) or S(O)₂, said ring being optionally substituted with at least one substituent independently selected from the group consisting of halogen, cyano, C₁-C₃ alkyl, C₁-C₃ haloalkyl.

8. The compound of claim 7 wherein
Z is Z-1, Z-4, Z-6, Z-12, Z-22 or Z-24;
R is independently H, F, Cl, Me, CF₃, OMe or OCF₃;
n is 2;
R is at 3- and 5-position;
R¹ and R² are each independently hydrogen, halogen, cyano, Me, CF₃, CH₂CN, OMe, OCF₃ or OCH₂CN;
R³ is H, Me, Et, CH═CH₂, C≡CH, cyclopropyl, cyclopropylmethy, CF₃, OMe or OCF₃; and
R⁷, R⁸, R⁹, R¹⁰, R¹¹ and R¹² are each independently H, C₁-C₃ alkyl, C₁-C₃ alkoxy, C₁-C₃ haloalkyl, C₃-C₅ cycloalkyl or phenyl, each of which is optionally substituted by one or more radicals from the group consisting of F, Cl, C₁-C₂ alkoxy or aryl.

9. The compound of claim 2 wherein
Z is selected from the group Z-B;
R is independently H, F, Cl, Br, cyano, C₁-C₂ alkyl, C₁-C₂ haloalkyl, C₁-C₂ alkoxy or C₁-C₂ haloalkoxy;
V and W are both O;
X is O or S;
R³ is H, C₁-C₃ alkyl, C₂-C₃ alkenyl, C₂-C₃ alkynyl, C₃-C₅ cycloalkyl, C₄-C₇ cycloalkylalkyl, C₁-C₃ haloalkyl, C₂-C₃ alkoxyalkyl, C₂-C₃ haloalkoxyalkyl, C₁-C₃ alkoxy, C₁-C₃ haloalkoxy;
R⁴ is H, C₁-C₆ alkyl, C₃-C₇ cycloalkyl, C₁-C₆ haloalkyl, C₁-C₃ alkoxy, C₁-C₃ haloalkoxy; and
R⁵ is H, C₁-C₁₂ alkyl, NH₂, N═CR⁸R¹², C₃-C₇ cycloalkyl, C₃-C₁₂ cycloalkylalkyl, C₂-C₈ alkenyl, C₅-C₆ cycloalkenyl, C₂-C₈ alkynyl, (CH₂)_{q}S(O)ₚR⁷, (CH₂)_{q}OR⁹, (CH₂)_{q}COR¹², each of which is optionally substituted by one or more radicals from the group consisting of halogen, cyano, C₁-C₆ alkyl, C₁-C₆ alkoxy, hydroxy and aryl.

10. The compound of claim 9 wherein
Z is Z-30, Z-34, Z-36, Z-42, Z-47, Z-48, Z-53, Z-54, Z-55, Z-57 or Z-62;
R is independently H, F, Cl, Me, CF₃, OMe or OCF₃;
n is 2;
R is at 3- and 5-position;
R¹ and R² are each independently hydrogen, halogen, cyano, Me, CF₃, CH₂CN, OMe, OCF₃ or OCH₂CN;
R³ is H, Me, Et, CH═CH₂, C≡CH, cyclopropyl, cyclopropylmethyl, CF₃, OMe or OCF₃;
R⁵ is H, Me, Et, c-Pr, c-Bu, CH₂-c-Pr, (CH₂)_{q}S(O)ₚMe, (CH₂)_{q}OMe, (CH₂)_{q}COMe, each of which is optionally substituted by one or more radicals from the group consisting of F, Cl, cyano, C₁-C₃ alkyl, C₁-C₃ alkoxy, hydroxy and Ph; and
R⁷, R⁸, R⁹, R¹⁰, R¹¹ and R¹² are each independently H, C₁-C₃ alkyl, C₁-C₃ haloalkyl, C₃-C₅ cycloalkyl or phenyl, each of which is optionally substituted by one or more radicals from the group consisting of F, Cl, C₁-C₂ alkoxy or aryl.

11. The compound of claim 1 selected from the group consisting of
2,2,2-Trifluoroethyl (4S)-4-[[[(5S)-3-(3,5-difluorophenyl)-5-methyl-2-oxo-5-oxazolidinyl]carbonyl]amino]-1-cyclopentene-1-carboxylate;
2,2,2-Trifluoroethyl (4S)-4-[[[(5R)-3-(3,5-difluorophenyl)-5-methyl-2-oxo-5-oxazolidinyl]carbonyl]amino]-1-cyclopentene-1-carboxylate;
2-(Methylthio)ethyl (4S)-4-[[[(5S)-3-(3,5-difluorophenyl)-5-methyl-2-oxo-5-oxazolidinyl]carbonyl]amino]-1-cyclopentene-1-carboxylate;
2-(Methylthio)ethyl (4S)-4-[[[(5R)-3-(3,5-difluorophenyl)-5-methyl-2-oxo-5-oxazolidinyl]carbonyl]amino]-1-cyclopentene-1-carboxylate;
Methyl (4S)-4-[[[(5S)-3-(3,5-difluorophenyl)-5-methyl-2-oxo-5-oxazolidinyl]carbonyl]amino]-1-cyclopentene-1-carboxylate;
Methyl (4S)-4-[[[(5R)-3-(3,5-difluorophenyl)-5-methyl-2-oxo-5-oxazolidinyl]carbonyl]amino]-1-cyclopentene-1-carboxylate;
Methyl (4S)-4-[[[3-(3,5-difluorophenyl)-5-methyl-2-oxo-5-oxazolidinyl]carbonyl]amino]-1-cyclopentene-1-carboxylate;
1-Methylethyl (1S,4R)-4-[[[3-(3-chloro-5-fluorophenyl)-5-methyl-2-oxo-5-oxazolidinyl]carbonyl]amino]-2-cyclopentene-1-carboxylate;
Methyl (1S,4R)-4-[[[3-(3-chloro-5-fluorophenyl)-5-methyl-2-oxo-5-oxazolidinyl]carbonyl]amino]-2-cyclopentene-1-carboxylate;
2-Methylpropyl (4S)-4-[[[3-(3,5-difluorophenyl)-5-methyl-2-oxo-5-oxazolidinyl]carbonyl]amino]-1-cyclopentene-1-carboxylate;
(1S,4R)-4-[[[3-(3,5-Difluorophenyl)-5-methyl-2-oxo-5-oxazolidinyl]carbonyl]amino]-2-cyclopentene-1-carboxylic acid;
(4S)-4-[[[3-(3,5-Difluorophenyl)-5-methyl-2-oxo-5-oxazolidinyl]carbonyl]amino]-1-cyclopentene-1-carboxylic acid;
(1S,4R)-4-[[[3-(3-Chloro-5-fluorophenyl)-5-methyl-2-oxo-5-oxazolidinyl]carbonyl]amino]-2-cyclopentene-1-carboxylic acid;
and
Ethyl (1S,4R)-4-[[[3-(3,5-difluorophenyl)-5-methyl-2-oxo-5-oxazolidinyl]carbonyl]amino]-2-cyclopentene-1-carboxylate.

12. The compound of claim 1 wherein
Y is NH;
Z-A is selected from Z-1 through Z-29; 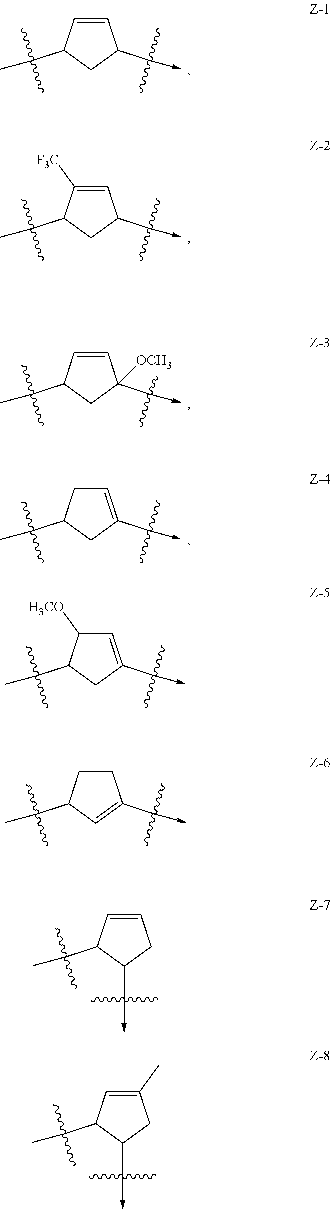 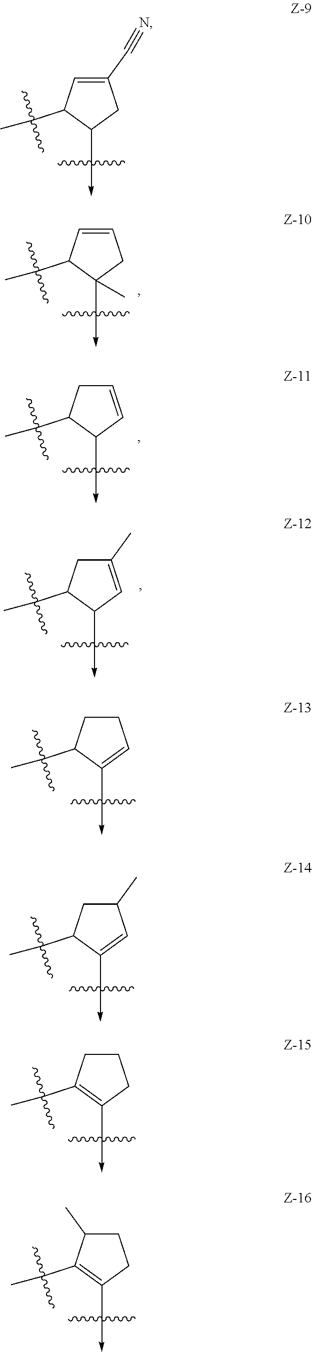 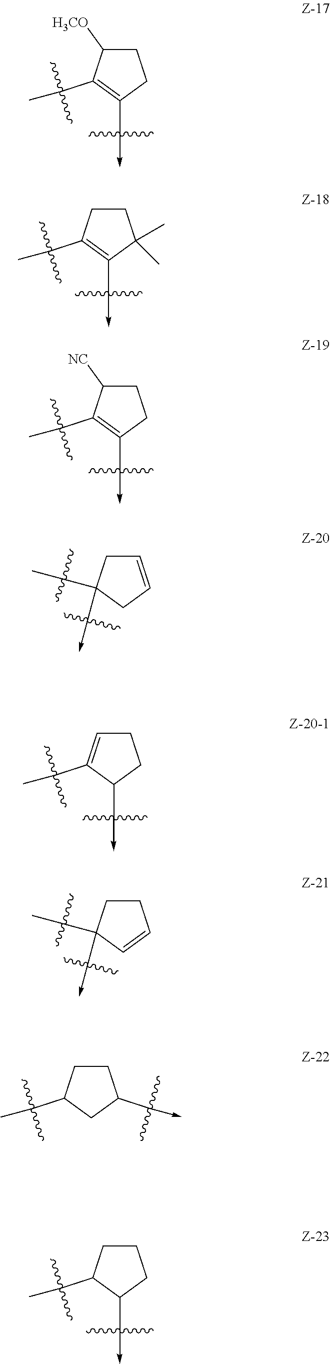 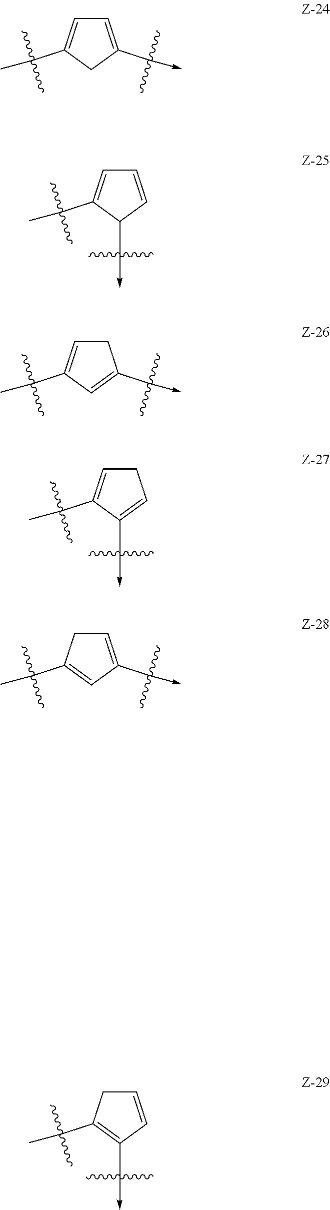 [SMILES: *C1=CC=C(*)C1 ; *C1=CC=CC1* ; *C1=CCC(*)=C1 ; *C1=CCC=C1* ; *C1=CCC(*)=C1 ; *C1=C(*)CC=C1]
m is 0 or 1;
R is independently H, halogen, cyano, C₁-C₆ alkyl, C₁-C₆ haloalkyl, C₁-C₆ alkoxy or C₁-C₆ haloalkoxy;
n is 1, 2 or 3;
R¹ and R² are each independently hydrogen, halogen, cyano, C₁-C₃ alkyl, C₁-C₃ haloalkyl, C₁-C₃ cyanoalkyl, C₁-C₃ alkoxy, C₁-C₃ haloalkoxy or C₁-C₃ cyanoalkoxy; and
R³ is H, halogen, cyano, hydroxy, C₁-C₃ alkyl, C₂-C₃ alkenyl, C₂-C₃ alkynyl, C₃-C₅ cycloalkoxy, C₃-C₅ cycloalkoxyalkyl, C₃-C₅ cycloalkyl, C₄-C₇ cycloalkylalkyl, C₁-C₃ haloalkyl, C₂-C₃ alkoxyalkyl, C₂-C₃ haloalkoxyalkyl, C₁-C₃ alkoxy, C₁-C₃ haloalkoxy, C₁-C₃ alkylthio, C₁-C₃ alkylsulfinyl or C₁-C₃ alkylsulfonyl.

13. The compound of claim 12 wherein
Z is Z-1, Z-4, Z-6, Z-12, Z-22 or Z-24;
R is independently H, F, Cl, Br, cyano, C₁-C₂ alkyl, C₁-C₂ haloalkyl, C₁-C₂ alkoxy or C₁-C₂ haloalkoxy;
V and W are both O;
X is O or S;
R³ is H, C₁-C₃ alkyl, C₂-C₃ alkenyl, C₂-C₃ alkynyl, C₃-C₅ cycloalkyl, C₄-C₇ cycloalkylalkyl, C₁-C₃ haloalkyl, C₂-C₃ alkoxyalkyl, C₂-C₃ haloalkoxyalkyl, C₁-C₃ alkoxy, C₁-C₃ haloalkoxy;
R⁴ is H, C₁-C₆ alkyl, C₃-C₇ cycloalkyl, C₁-C₆ haloalkyl, C₁-C₃ alkoxy, C₁-C₃ haloalkoxy; and
R⁵ is H, C₁-C₁₂ alkyl, NH₂, N═CR⁸R¹², C₃-C₇ cycloalkyl, C₃-C₁₂ cycloalkylalkyl, C₂-C₈ alkenyl, C₅-C₆ cycloalkenyl, C₂-C₈ alkynyl, (CH₂)_{q}S(O)ₚR⁷, (CH₂)_{q}OR⁹, (CH₂)_{q}COR¹², each of which is optionally substituted by one or more radicals from the group consisting of halogen, cyano, C₁-C₆ alkyl, C₁-C₆ alkoxy, hydroxy and aryl.

14. The compound of claim 13 wherein
R is independently H, F, Cl, Me, CF₃, OMe or OCF₃;
n is 2;
R is at 3- and 5-position;
R¹ and R² are each independently hydrogen, halogen, cyano, Me, CF₃, CH₂CN, OMe, OCF₃ or OCH₂CN;
R³ is H, Me, Et, CH═CH₂, C≡CH, cyclopropyl, cyclopropylmethy, CF₃, OMe or OCF₃.
R⁵ is H, Me, Et, c-Pr, c-Bu, CH₂-c-Pr, (CH₂)_{q}S(O)ₚMe, (CH₂)_{q}OMe, (CH₂)_{q}COMe, each of which is optionally substituted by one or more radicals from the group consisting of F, Cl, cyano, C₁-C₃ alkyl, C₁-C₃ alkoxy, hydroxy and Ph; and R⁷, R⁸, R⁹, R¹⁰, R¹¹ and R¹² are each independently H, C₁-C₃ alkyl, C₁-C₃ haloalkyl, C₃-C₅ cycloalkyl or phenyl, each of which is optionally substituted by one or more radicals from the group consisting of F, Cl, C₁-C₂ alkoxy or aryl.

15. A herbicidal composition comprising a compound of any of claims 1 through 14 and at least one component selected from the group consisting of surfactants, solid diluents and liquid diluents.

16. The herbicidal composition of claim 15 further comprising at least one additional active ingredient selected from the group consisting of other herbicides and herbicide safeners, and at least one component selected from the group consisting of surfactants, solid diluents and liquid diluents.

17. A herbicidal mixture comprising (a) a compound of any one of claims 1 through 14, and (b) at least one additional active ingredient selected from (b1) photosystem II inhibitors, (b2) acetohydroxy acid synthase (AHAS) inhibitors, (b3) acetyl-CoA carboxylase (ACCase) inhibitors, (b4) auxin mimics, (b5) 5-enol-pyruvylshikimate-3-phosphate (EPSP) synthase inhibitors, (b6) photosystem I electron diverters, (b7) protoporphyrinogen oxidase (PPO) inhibitors, (b8) glutamine synthetase (GS) inhibitors, (b9) very long chain fatty acid (VLCFA) elongase inhibitors, (b10) auxin transport inhibitors, (b11) phytoene desaturase (PDS) inhibitors, (b12) 4-hydroxyphenyl-pyruvate dioxygenase (HPPD) inhibitors, (b13) homogentisate solanesyltransferase (HST) inhibitors, (b14) cellulose biosynthesis inhibitors, (b15) dehydrooritate dehydrogenase (DHODH) inhibitors, (b16) other herbicides including mitotic disruptors, organic arsenicals, asulam, bromobutide, cinmethylin, cumyluron, dazomet, difenzoquat, dymron, etobenzanid, flurenol, fosamine, fosamine-ammonium, hydantocidin, metam, methyldymron, oleic acid, oxaziclomefone, pelargonic acid and pyributicarb, (b17) herbicide safeners, and salts of compounds of (b1) through (b17).

18. A method for controlling the growth of undesired vegetation comprising contacting the vegetation or its environment with a herbicidally effective amount of a compound of any one of claims 1 through 14.
